(19) United States
(12) Patent Application Publication
(10) Pub. No.: US 2024/0408009 A1
(43) Pub. Date: Dec. 12, 2024

(54) CLINICAL FORMULATIONS OF ANTI-TIGIT ANTIBODIES

(71) Applicant: Genentech, Inc., South San Francisco, CA (US)

(72) Inventors: Ian Chung-ti Shieh, South San Francisco, CA (US); Ann Marie Woys, South San Francisco, CA (US); Katherine Laura Tschudi, South San Francisco, CA (US); Robyn Mariah Suhling, South San Francisco, CA (US); Rucha Sudhakar Sane, South San Francisco, CA (US); Hui Min Phyllis Chan, South San Francisco, CA (US); Xiaohui Wen, South San Francisco, CA (US); Isabelle Anne Rooney, South San Francisco, CA (US)

(21) Appl. No.: 18/749,129

(22) Filed: Jun. 20, 2024

Related U.S. Application Data
(63) Continuation of application No. PCT/US2022/082139, filed on Dec. 21, 2022.
(60) Provisional application No. 63/387,229, filed on Dec. 13, 2022.
(60) Provisional application No. 63/383,451, filed on Nov. 11, 2022.
(60) Provisional application No. 63/292,863, filed on Dec. 22, 2021.

(51) Int. Cl. A61K 9/08; A61K 38/47; A61K 47/18; A61K 47/26; C07K 16/28

(52) U.S. Cl. CPC A61K 9/08; A61K 38/47; A61K 47/183; A61K 47/26; C07K 16/2803; C07K 16/2827; C12Y 302/01035

(57) ABSTRACT

The present disclosure relates to pharmaceutical formulations of anti-TIGIT monoclonal antibodies that are suitable for co administration or co-formulation with anti-PD-L1 monoclonal antibodies. The present disclosure also relates to articles of manufacture comprising such pharmaceutical formulations and methods of treating cancer using the pharmaceutical formulations and to use of the pharmaceutical formulations for the treatment of cancer or the manufacture of a medicament for treating cancer. The present disclosure also relates to articles of manufacture comprising single doses of anti-TIGIT monoclonal antibodies or both anti-TIGIT and anti-PD-L1 monoclonal antibodies and methods of treating cancer using such articles of manufacture and the formulations contained therein.

### CROSS-REFERENCE TO RELATED APPLICATIONS

This application is a continuation of International Application No. PCT/US2022/082139, filed Dec. 21, 2022, which claims priority and benefit of U.S. Provisional Application No. 63/292,863, filed Dec. 22, 2021, U.S. Provisional Application No. 63/383,451, filed Nov. 11, 2022, and U.S. Provisional Application No. 63/387,229, filed Dec. 13, 2022, the contents of each of the foregoing documents are hereby incorporated by reference in their entireties.

### TECHNICAL FIELD

The present disclosure pertains to the field of pharmaceutical formulations comprising anti-TIGIT monoclonal antibodies, which are suitable for co-administration or co-formulation with an anti-PD-L1 monoclonal antibody.

### SEQUENCE LISTING

The instant disclosure contains a Sequence Listing which has been submitted electronically in XML format and is hereby incorporated by reference in its entirety. Said XML copy, created on Jun. 20, 2024, is named 000218-0047-101_SL.xml and is 26,125 bytes in size.

### BACKGROUND

Cancer is a leading cause of death worldwide with approximately 9,958,130 deaths globally in 2020. In North America, the number of new cases were estimated to be 2,556,860 (1,372,000 cases in men and 1,184,860 new cases in women) and 699,274 cancer deaths. Similar data for Central and Eastern Europe estimated in 2020 showing 1,314,193 new cases (657,259 in men; 656,934 in women) and 695,828 cancer deaths (the Global Cancer Observatory, December 2020). For most malignancies, the impact of current therapy on improving the quality of live, slowing progression of disease, prolonging survival, or curing patients is inadequate.

Immunotherapy has become an established strategy for treating cancer, improving the prognosis of many patients suffering from a broad variety of cancers. Further, combinations of immunotherapies have proven more effective at treating cancer. Indeed, the FDA has granted the combination of tiragolumab and atezolizumab Breakthrough Therapy Designation for the first line-treatment of non-small cell lung cancer. Immunotherapies are typically infused into a patient over the course of hours. Patients receiving combination therapies receive two separate infusions, requiring patients to be available for longer periods of time (if the therapies are administered on the same day) or more frequently (if the therapies are administered on separate days). There is a need in the art, therefore, for combination therapies that can be either co-administered or co-formulated. Such co-administration or co-formulation would reduce the burden on patients and improve patient compliance. There is also a need in the art for therapies that can be administered more rapidly than by intravenous infusion, e.g. by subcutaneous injection.

### SUMMARY OF THE DISCLOSURE

In one aspect, the present disclosure provides liquid pharmaceutical formulations comprising an anti-TIGIT monoclonal antibody and an anti-PD-L1 monoclonal antibody. In some embodiments, the liquid pharmaceutical formulation comprises: (a) 18 mg/mL to 176 mg/mL of an anti-TIGIT monoclonal antibody; (b) 54 mg/mL to 137.5 mg/mL of an anti-PD-L1 monoclonal antibody; (c) 5 mM to 30 mM of a histidine buffer; (d) 120 mM to 320 mM of sucrose; and (e) 0.02% (w/v) to 0.08% (w/v) polysorbate 20, wherein the liquid pharmaceutical formulation is characterized by a pH of about 5.4-6.2; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments, the liquid pharmaceutical formulation comprises: (a) 18 mg/mL to 176 mg/mL of an anti-TIGIT monoclonal antibody; (b) 54 mg/mL to 137.5 mg/mL of an anti-PD-L1 monoclonal antibody; (c) 5 mM to 30 mM of a histidine buffer; (d) 120 mM to 320 mM of sucrose; and (e) 0.02% (w/v) to 0.08% (w/v) polysorbate 20, wherein the liquid pharmaceutical formulation is characterized by a pH of about 5.2-6.1. In some embodiments, the formulation comprises: (a) 30 mg/mL to 50 mg/mL of the anti-TIGIT monoclonal antibody; (b) 70 mg/mL to 90 mg/mL of the anti-PD-L1 monoclonal antibody; (c) 15 mM to 25 mM of the histidine buffer; (d) 200 mM to 280 mM of sucrose; and (e) 0.04% (w/v) to 0.08% (w/v) polysorbate 20, wherein the liquid pharmaceutical formulation is characterized by a pH of about 5.5-6.1. In some embodiments, the formulation comprises: (a) 30 mg/mL to 50 mg/mL of the anti-TIGIT monoclonal antibody; (b) 70 mg/mL to 90 mg/mL of the anti-PD-L1 monoclonal antibody; (c) 15 mM to 25 mM of the histidine buffer; (d) 200 mM to 280 mM of sucrose; and (e) 0.04% (w/v) to 0.08% (w/v) polysorbate 20, wherein the liquid pharmaceutical formulation is characterized by a pH of about 5.6-6.0. In some embodiments, the formulation comprises: (a) 36 mg/mL to 44 mg/mL of the anti-TIGIT monoclonal antibody; (b) 72 mg/mL to 88 mg/mL of the anti-PD-L1 monoclonal antibody; (c) 18 mM to 22 mM of the histidine buffer; (d) 220 mM to 260 mM of sucrose; and (e) 0.05% (w/v) to 0.07% (w/v) polysorbate 20, wherein the liquid pharmaceutical formulation is characterized by a pH of about 5.7-5.9. In some embodiments, the formulation comprises: (a) 36 mg/mL to 44 mg/mL of the anti-TIGIT monoclonal antibody; (b) 72 mg/mL to 88 mg/mL of the anti-PD-L1 monoclonal antibody; (c) 15 mM to 25 mM of the histidine buffer; (d) 200 mM to 280 mM of sucrose; and (e) 0.04% (w/v) to 0.08% (w/v) polysorbate 20, wherein the liquid pharmaceutical formulation is characterized by a pH of about 5.5-6.1. In some embodiments, the formulation comprises: (a) 40 mg/mL of the anti-TIGIT monoclonal antibody; (b) 80 mg/mL of the anti-PD-L1 monoclonal antibody; (c) 20 mM of the histidine buffer; (d) 240 mM of sucrose; and (e) 0.06% (w/v) of polysorbate 20, wherein the liquid pharmaceutical formulation is characterized by a pH of about 5.5. In some embodiments, the formulation comprises: (a) 40 mg/mL of the anti-TIGIT monoclonal antibody; (b) 80 mg/ml of the anti-PD-L1 monoclonal antibody; (c) 20 mM of the histidine buffer; (d) 240 mM of sucrose; and (e) 0.06% (w/v) of polysorbate 20, wherein the liquid pharmaceutical formulation is characterized by a pH of about 5.6. In some embodiments, the formulation comprises: (a) 40 mg/mL of the anti-TIGIT monoclonal antibody; (b) 80 mg/mL of the anti-PD-L1 monoclonal antibody; (c) 20 mM of the histidine buffer; (d) 240 mM of sucrose; and (e) 0.06% (w/v) of polysorbate 20, wherein the liquid pharmaceutical formulation is characterized by a pH of about 5.7. In some embodiments, the formulation comprises: (a) 40 mg/mL of the anti-TIGIT monoclonal antibody; (b) 80 mg/mL of the anti-PD-L1 monoclonal antibody; (c) 20 mM of the histidine buffer; (d) 240 mM of sucrose; and (e) 0.06% (w/v) of polysorbate 20, wherein the liquid pharmaceutical formulation is characterized by a pH of about 5.8.

In another aspect, the present disclosure provides liquid pharmaceutical formulations suitable for subcutaneous injection and comprising an anti-TIGIT monoclonal antibody, an anti-PD-L1 monoclonal antibody, and hyaluronidase. In some embodiments, the liquid pharmaceutical formulation comprises: (a) 18 mg/mL to 75 mg/mL of an anti-TIGIT monoclonal antibody; (b) 54 mg/mL to 137.5 mg/mL of an anti-PD-L1 monoclonal antibody; (c) 12 mM to 28 mM of a histidine buffer; (d) 100 mM to 300 mM of sucrose; and (d) 0.02% (w/v) to 0.08% (w/v) polysorbate 20, wherein the liquid pharmaceutical formulation is characterized by a pH of about 5.4-6.2; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In another aspect, the present disclosure provides liquid pharmaceutical formulations suitable for subcutaneous injection and comprising an anti-TIGIT monoclonal antibody, an anti-PD-L1 monoclonal antibody, and hyaluronidase. In some embodiments, the liquid pharmaceutical formulation comprises: (a) 30 mg/mL to 60 mg/mL of an anti-TIGIT monoclonal antibody; (b) 60 mg/mL to 120 mg/mL of an anti-PD-L1 monoclonal antibody; (c) 500 U/mL to 2600 U/mL hyaluronidase; (d) 5 mM to 30 mM of a histidine buffer; (e) 180 mM to 320 mM of sucrose; and (f) 0.03% (w/v) to 0.08% (w/v) polysorbate 20, wherein the liquid pharmaceutical formulation is characterized by a pH of about 5.2-6.1; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments, the formulation comprises: (a) 35 mg/mL to 55 mg/mL of the anti-TIGIT monoclonal antibody; (b) 70 mg/mL to 110 mg/mL of the anti-PD-L1 monoclonal antibody; (c) 1400 U/mL to 2600 U/mL hyaluronidase; (d) 5 mM to 25 mM of the histidine buffer; (e) 180 mM to 320 mM of sucrose; and (f) 0.04% (w/v) to 0.08% (w/v) polysorbate 20, wherein the liquid pharmaceutical formulation is characterized by a pH of about 5.5-5.8. In some embodiments, the formulation comprises: (a) 30 mg/mL to 60 mg/mL of the anti-TIGIT monoclonal antibody; (b) 60 mg/mL to 120 mg/mL of the anti-PD-L1 monoclonal antibody; (c) 1000 U/mL to 3000 U/mL hyaluronidase; (d) 15 mM to 25 mM of the histidine buffer; (e) 200 mM to 280 mM of sucrose; and (f) 0.04% (w/v) to 0.08% (w/v) polysorbate 20, wherein the liquid pharmaceutical formulation is characterized by a pH of about 5.2-6.1. In some embodiments, the formulation comprises: (a) 35 mg/mL to 45 mg/mL of the anti-TIGIT monoclonal antibody; (b) 70 mg/mL to 90 mg/mL of the anti-PD-L1 monoclonal antibody; (c) 1500 U/mL to 2500 U/mL hyaluronidase; (d) 18 mM to 22 mM of the histidine buffer; (3) 220 mM to 260 mM of sucrose; and (f) 0.05% (w/v) to 0.07% (w/v) polysorbate 20, wherein the liquid pharmaceutical formulation is characterized by a pH of about 5.5-5.8. In some embodiments, the formulation comprises: (a) 40 mg/mL to 50 mg/mL of the anti-TIGIT monoclonal antibody; (b) 80 mg/mL to 100 mg/mL of the anti-PD-L1 monoclonal antibody; (c) 1500 U/mL to 2500 U/mL hyaluronidase; (d) 18 mM to 22 mM of the histidine buffer; (e) 220 mM to 260 mM of sucrose; and (f) 0.05% (w/v) to 0.07% (w/v) polysorbate 20, wherein the liquid pharmaceutical formulation is characterized by a pH of about 5.5-5.8.

In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the heavy chain variable region of the anti-PD-L1 monoclonal antibody comprises the amino acid sequence of SEQ ID NO: 16. In some embodiments, the light chain variable region of the anti-PD-L1 monoclonal antibody comprises the amino acid sequence of SEQ ID NO: 17. In some embodiments, the heavy chain variable region of the anti-PD-L1 monoclonal antibody comprises the amino acid sequence of SEQ ID NO: 16 and the light chain variable region of the anti-PD-L1 monoclonal antibody comprises the amino acid sequence of SEQ ID NO: 17. In some embodiments, the anti-PD-L1 monoclonal antibody is an IgG antibody. In some embodiments, the anti-PD-L1 monoclonal antibody is an IgG1 or an IgG4 antibody. In some embodiments, the anti-PD-L1 monoclonal antibody is a full-length antibody. In some embodiments, the anti-PD-L1 monoclonal antibody is a full-length IgG antibody. In some embodiments, the anti-PD-L1 monoclonal antibody is a full-length IgG1 antibody. In some embodiments, the anti-PD-L1 monoclonal antibody is a full-length human IgG1 antibody. In some embodiments, the anti-PD-L1 monoclonal antibody is a full-length humanized IgG1 antibody. The anti-PD-L1 monoclonal antibody may be an antibody fragment. In some embodiments, the anti-PD-L1 monoclonal antibody is a Fab, a Fab′, a F(ab′)₂, a Fv fragment, or a scFv fragment. In some embodiments, the anti-PD-L1 monoclonal antibody is a human antibody. In some embodiments, the anti-PD-L1 monoclonal antibody is a humanized antibody.

In another aspect, the present disclosure provides liquid pharmaceutical formulations comprising an anti-TIGIT monoclonal antibody and suitable for co-administration with an anti-PD-L1 monoclonal antibody. In some embodiments, the anti-PD-L1 monoclonal antibody, is an anti-PD-L1 monoclonal antibody disclosed supra. In some embodiments, the liquid pharmaceutical formulation comprises: (a) 18 mg/mL to 176 mg/mL of an anti-TIGIT monoclonal antibody; (b) 5 mM to 30 mM of histidine acetate; (c) 100 mM to 320 mM of sucrose; and (d) 0.01% (w/v) to 0.08% (w/v) polysorbate 20, wherein the liquid pharmaceutical formulation is characterized by a pH of about 5.0-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In another aspect, the present disclosure provides liquid pharmaceutical formulations comprising an anti-TIGIT monoclonal antibody and suitable for co-administration with an anti-PD-L1 monoclonal antibody. In some embodiments, the anti-PD-L1 monoclonal antibody, is an anti-PD-L1 monoclonal antibody disclosed supra. In some embodiments, the liquid pharmaceutical formulation comprises: (a) 18 mg/mL to 75 mg/mL of an anti-TIGIT monoclonal antibody; (b) 5 mM to 30 mM of histidine acetate; (c) 100 mM to 320 mM of sucrose; and (d) 0.01% (w/v) to 0.08% (w/v) polysorbate 20, wherein the liquid pharmaceutical formulation is characterized by a pH of about 5.0-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments the formulation comprises: (a) 144 mg/mL to 176 mg/mL of the anti-TIGIT monoclonal antibody; (b) 5 mM to 25 mM of the histidine buffer; (c) 180 mM to 320 mM of sucrose; and (d) 0.05% (w/v) to 0.08% (w/v) polysorbate 20, wherein the liquid pharmaceutical formulation is characterized by a pH of about 5.2-5.8. In some embodiments, the formulation comprises: (a) 160 mg/mL of the anti-TIGIT monoclonal antibody; (b) 20 mM of the histidine buffer; (c) 240 mM of sucrose; and (d) 0.06% (w/v) of polysorbate 20, wherein the liquid pharmaceutical formulation is characterized by a pH of about 5.5.

In some embodiments the formulation comprises: (a) 50 mg/mL to 70 mg/mL of the anti-TIGIT monoclonal antibody; (b) 15 mM to 25 mM of the histidine buffer; (c) 200 mM to 280 mM of sucrose; and (d) 0.02% (w/v) to 0.06% (w/v) polysorbate 20, wherein the liquid pharmaceutical formulation is characterized by a pH of about 5.2-5.8. In some embodiments, the formulation comprises: (a) 54 mg/mL to 66 mg/mL of the anti-TIGIT monoclonal antibody; (b) 18 mM to 22 mM of the histidine buffer; (c) 220 mM to 260 mM of sucrose; and (d) 0.03% (w/v) to 0.05% (w/v) of polysorbate 20, wherein the liquid pharmaceutical formulation is characterized by a pH of about 5.4-5.6. In some embodiments, the formulation comprises: (a) 60 mg/mL of the anti-TIGIT monoclonal antibody; (b) 20 mM of the histidine buffer; (c) 240 mM of sucrose; and (d) 0.04% (w/v) of polysorbate 20, wherein the liquid pharmaceutical formulation is characterized by a pH of about 5.5.

In a further aspect, the present disclosure provides liquid pharmaceutical formulations suitable for subcutaneous injection and comprising an anti-TIGIT monoclonal antibody. In some embodiments the liquid pharmaceutical formulation comprises: (a) 18 mg/mL to 176 mg/mL of an anti-TIGIT monoclonal antibody; (b) 500 U/mL to 2600 U/mL of a hyaluronidase; (c) 5 mM to 30 mM of a histidine buffer; (d) 180 mM to 320 mM of sucrose; and (e) 0.03% (w/v) to 0.08% (w/v) polysorbate 20, wherein the liquid pharmaceutical formulation is characterized by a pH of about 5.2-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments, the formulation comprises: (a) 144 mg/mL to 176 mg/ml of the anti-TIGIT monoclonal antibody; (b) 1400 U/mL to 2600 U/mL of the hyaluronidase; (c) 5 mM to 25 mM of the histidine buffer; (d) 180 mM to 320 mM of sucrose; and (e) 0.04% (w/v) to 0.08% (w/v) polysorbate 20, wherein the liquid pharmaceutical formulation is characterized by a pH of about 5.2-5.8. In some embodiments, the formulation comprises: (a) 160 mg/ml of the anti-TIGIT monoclonal antibody; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.5.

In some embodiments, the hyaluronidase is a recombinant human hyaluronidase. In some embodiments, the hyaluronidase is a human soluble PH20 hyaluronidase glycoprotein, such as rHuPH20.

In some embodiments of any of the above formulations the histidine buffer is histidine acetate.

In some embodiments of any of the above formulations, the formulation further comprises a stabilizer. In some embodiments, the stabilizer is selected from the group consisting of methionine, glycine, alanine, proline, taurine, betaine, octopine, glutamate, sarcosine, γ-aminobutyric acid, and trimethylamine N-oxide. In some embodiments, the stabilizer is methionine. In some embodiments, the concentration of the stabilizer is about 0 mM to about 15 mM. In some embodiments, the concentration of the stabilizer is about 5 mM to about 15 mM. In some embodiments, the concentration of the stabilizer is about 10 mM.

In an additional aspect, the present disclosure provides an article of manufacture comprising any of the liquid pharmaceutical formulations disclosed herein. In some embodiments, the article of manufacture is a vial. In some embodiments, the vial is a single dosage vial. In some embodiments, the vial is stoppered with a chlorobutyl elastomer stopper. In some embodiments, the article of manufacture is a pre-filled syringe. In some embodiments, the article of manufacture is a syringe pump. In some embodiments, the article of manufacture is a subcutaneous administration device. In some embodiments, the subcutaneous administration device is selected from the group consisting of a syringe, a syringe pump, an injection device, an infusion pump, an injector pen, a needleless device, an autoinjector, and a subcutaneous patch delivery system. In some embodiments, the subcutaneous administration device is a syringe. In some embodiments, the subcutaneous administration device is a syringe pump. In some embodiments, the subcutaneous administration device is an injection device. In some embodiments, the subcutaneous administration device is an infusion pump. In some embodiments, the subcutaneous administration device is an injector pen. In some embodiments, the subcutaneous administration device is a needleless device. In some embodiments, the subcutaneous administration device is an autoinjector. In some embodiments, the subcutaneous administration device is a subcutaneous patch delivery system.

In some embodiments, the article comprises about 3 mL to about 30 mL of the liquid pharmaceutical formulation. In some embodiments, the article comprises about 3 mL to about 60 mL of the liquid pharmaceutical formulation. In some embodiments, the article comprises about 10 mL of the liquid pharmaceutical formulation. In some embodiments, the article comprises about 7 mL of the liquid pharmaceutical formulation. In some embodiments, the article comprises about 6.5 mL of the liquid pharmaceutical formulation. In some embodiments, the article comprises about 21 mL of the liquid pharmaceutical formulation. In some embodiments, the article comprises about 50 mL of the liquid pharmaceutical formulation.

In an additional aspect, the present disclosure provides an article of manufacture comprising a formulation that comprises 18 mg/mL to 176 mg/mL of an anti-TIGIT monoclonal antibody. In some embodiments, the formulation comprises 144 mg/mL to 176 mg/ml of the anti-TIGIT monoclonal antibody. In some embodiments, the formulation comprises 160 mg/mL of the anti-TIGIT monoclonal antibody. In some embodiments, the article of manufacture further comprises a formulation comprising an anti-PD-L1 monoclonal antibody. In some embodiments, the anti-PD-L1 monoclonal antibody is atezolizumab.

In a further aspect, the present disclosure provides an article of manufacture comprising a formulation that comprises: (a) 30 mg/mL to 60 mg/mL of an anti-TIGIT monoclonal antibody; and (b) 60 mg/mL to 120 mg/mL of an anti-PD-L1 monoclonal antibody. In some embodiments, the formulation comprises (a) 35 mg/mL to 55 mg/mL of the anti-TIGIT monoclonal antibody; and (b) 70 mg/mL to 110 mg/mL of the anti-PD-L1 monoclonal antibody. In some embodiments, the formulation comprises: (a) 30 mg/mL of the anti-TIGIT monoclonal antibody; and (b) 60 mg/mL of the anti-PD-L1 monoclonal antibody. In some embodiments, the formulation comprises: (a) 35 mg/mL of the anti-TIGIT monoclonal antibody; and (b) 70 mg/mL of the anti-PD-L1 monoclonal antibody. In some embodiments, the formulation comprises: (a) 40 mg/mL of the anti-TIGIT monoclonal antibody; and (b) 80 mg/mL of the anti-PD-L1 monoclonal antibody. In some embodiments, the formulation comprises: (a) 45 mg/mL of the anti-TIGIT monoclonal antibody; and (b) 90 mg/mL of the anti-PD-L1 monoclonal antibody. In some embodiments, the formulation comprises: (a) 50 mg/mL of the anti-TIGIT monoclonal antibody; and (b) 100 mg/mL of the anti-PD-L1 monoclonal antibody. In some embodiments, the formulation comprises: (a) 55 mg/mL of the anti-TIGIT monoclonal antibody; and (b) 110 mg/mL of the anti-PD-L1 monoclonal antibody.

In another aspect, the present disclosure provides an article of manufacture comprising a formulation that comprises 880 mg of an anti-TIGIT monoclonal antibody. In some embodiments, the formulation further comprises 1875 mg or 2000 mg of an anti-PD-L1 monoclonal antibody. In some embodiments, the formulation further comprises 1875 mg of an anti-PD-L1 monoclonal antibody. In some embodiments, the formulation further comprises 2000 mg of an anti-PD-L1 monoclonal antibody. In some embodiments, the formulation further comprises an anti-PD-L1 monoclonal antibody. In some embodiments, the formulation further comprises hyaluronidase.

In an additional aspect, the present disclosure provides an article of manufacture comprising 880 mg of an anti-TIGIT monoclonal antibody and 1875 mg or 2000 mg of an anti-PD-L1 monoclonal antibody. In some embodiments, the formulation further comprises hyaluronidase.

In some embodiments, the formulation comprised in an article of manufacture of this disclosure further comprises hyaluronidase. In some embodiments, the formulation comprised in an article of manufacture of this disclosure further comprises hyaluronidase, wherein the hyaluronidase is 500 U/mL to 2600 U/mL. In some embodiments, the hyaluronidase is 1400 U/mL to 2600 U/mL. In some embodiments, the hyaluronidase is 2000 U/mL. In some embodiments, the hyaluronidase is a recombinant human hyaluronidase. In some embodiments, the recombinant hyaluronidase is a human soluble PH20 hyaluronidase glycoprotein, such as rHuPH20.

In some embodiments, an article of manufacture of this disclosure comprises about 3 mL to about 60 mL of the anti-TIGIT monoclonal antibody, and one or more of hyaluronidase, a histidine buffer, sucrose, and polysorbate 20. In some embodiments, an article of manufacture of this disclosure comprises about 10 mL of the anti-TIGIT monoclonal antibody, and one or more of hyaluronidase, a histidine buffer, sucrose, and polysorbate 20. In some embodiments, an article of manufacture of this disclosure comprises about 7 mL of the anti-TIGIT monoclonal antibody, and one or more of hyaluronidase, a histidine buffer, sucrose, and polysorbate 20. In some embodiments, an article of manufacture of this disclosure comprises about 6.5 mL of the anti-TIGIT monoclonal antibody, and one or more of hyaluronidase, a histidine buffer, sucrose, and polysorbate 20. In some embodiments, an article of manufacture of this disclosure comprises about 21 mL of the anti-TIGIT monoclonal antibody, and one or more of hyaluronidase, a histidine buffer, sucrose, and polysorbate 20.

In an additional aspect, the present disclosure provides an article of manufacture comprising a subcutaneous administration device, which contains and delivers to a patient a 880 mg fixed dose of an anti-TIGIT monoclonal antibody. In some embodiments, the subcutaneous administration device further delivers to the patient a 1875 mg or 2000 mg fixed dose of an anti-PD-L1 monoclonal antibody. In some embodiments, the subcutaneous administration device is a syringe pump.

In some embodiments of any of the above aspects, the heavy chain variable region (VH) of the anti-TIGIT monoclonal antibody comprises the amino acid sequence of SEQ ID NO: 7. In some embodiments of any of the above aspects, the light chain variable region (VL) of the anti-TIGIT monoclonal antibody comprises the amino acid sequence of SEQ ID NO: 9. In some embodiments of any of the above aspects, the heavy chain variable region of the anti-TIGIT monoclonal antibody comprises the amino acid sequence of SEQ ID NO: 7 and the light chain variable region of the anti-TIGIT monoclonal antibody comprises the amino acid sequence of SEQ ID NO: 9. In some embodiments of any of the above aspects, the heavy chain of the anti-TIGIT monoclonal antibody comprises the amino acid sequence of SEQ ID NO: 18. In some embodiments of any of the above aspects, the heavy chain of the anti-TIGIT monoclonal antibody comprises the amino acid sequence of SEQ ID NO: 19. In some embodiments of any of the above aspects, the heavy chain of the anti-TIGIT monoclonal antibody comprises the amino acid sequence of SEQ ID NO: 24. In some embodiments of any of the above aspects, the heavy chain of the anti-TIGIT monoclonal antibody comprises the amino acid sequence of SEQ ID NO: 25. In some embodiments of any of the above aspects, the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 18 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 19 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 24 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 25 and the light chain of the anti-TIGIT monoclonal antibody comprises the amino acid sequence of SEQ ID NO: 20. In some embodiments of any of the above aspects, the anti-TIGIT monoclonal antibody is tiragolumab.

In some embodiments of any of the above aspects, the anti-TIGIT monoclonal antibody is an IgG antibody. In some embodiments of any of the above aspects, the anti-TIGIT monoclonal antibody is an IgG1 or an IgG4 antibody. In some embodiments of any of the above aspects, the anti-TIGIT monoclonal antibody is a full-length antibody. In some embodiments of any of the above aspects, the anti-TIGIT monoclonal antibody is a full-length IgG antibody. In some embodiments of any of the above aspects, the anti-TIGIT monoclonal antibody is a full-length IgG1 antibody. In some embodiments of any of the above aspects, the anti-TIGIT monoclonal antibody is a full-length human IgG1 antibody. In some embodiments of any of the above aspects, the anti-TIGIT monoclonal antibody is a full-length humanized IgG1 antibody. In some embodiments of any of the above aspects, the anti-TIGIT monoclonal antibody is an antibody fragment. In some embodiments of any of the above aspects, the anti-TIGIT monoclonal antibody is a Fab, a Fab′, a F(ab′)₂, a Fv, or a scFv fragment. In some embodiments of any of the above aspects, the anti-TIGIT monoclonal antibody is a human antibody. In some embodiments of any of the above aspects, the anti-TIGIT monoclonal antibody is a humanized antibody. In some embodiments of any of the above aspects, the anti-TIGIT monoclonal antibody inhibits or blocks the interaction of CD226 with TIGIT.

In some embodiments of any of the above aspects, the anti-PD-L1 monoclonal antibody comprises a heavy chain variable region comprising a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15. In some embodiments of any of the above aspects, the heavy chain variable region of the anti-PD-L1 monoclonal antibody comprises the amino acid sequence of SEQ ID NO: 16. In some embodiments of any of the above aspects, the light chain variable region of the anti-PD-L1 monoclonal antibody comprises the amino acid sequence of SEQ ID NO: 17. In some embodiments of any of the above aspects, the heavy chain variable region of the anti-PD-L1 monoclonal antibody comprises the amino acid sequence of SEQ ID NO: 16, and the light chain variable region of the anti-PD-L1 monoclonal antibody comprises the amino acid sequence of SEQ ID NO: 17. In some embodiments of any of the above aspects, the heavy chain of the anti-PD-L1 monoclonal antibody comprises the amino acid sequence of SEQ ID NO: 21. In some embodiments of any of the above aspects, the heavy chain of the anti-PD-L1 monoclonal antibody comprises the amino acid sequence of SEQ ID NO: 22. In some embodiments of any of the above aspects, the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23. In some embodiments of any of the above aspects, the anti-PD-L1 antibody is atezolizumab. In some embodiments of any of the above aspects, the anti-PD-L1 monoclonal antibody is an IgG antibody. In some embodiments of any of the above aspects, the anti-PD-L1 monoclonal antibody is an IgG1 or an IgG4 antibody. In some embodiments of any of the above aspects, the anti-PD-L1 monoclonal antibody is a full-length antibody. In some embodiments of any of the above aspects, the anti-PD-L1 antibody is an antibody fragment. In some embodiments of any of the above aspects, the anti-PD-L1 monoclonal antibody is a Fab, a Fab′, a F(ab′)₂, a Fv, or a scFv fragment. In some embodiments of any of the above aspects, the anti-PD-L1 monoclonal antibody is a human antibody. In some embodiments of any of the above aspects, the anti-PD-L1 monoclonal antibody is a humanized antibody.

In an additional aspect, the present disclosure provides article of manufacture comprising a subcutaneous administration device, which contains and delivers to a patient a 880 mg fixed dose of tiragolumab. In some embodiments, the subcutaneous administration device further delivers to the patient a 1875 mg or 2000 mg fixed dose of atezolizumab. In some embodiments, the subcutaneous administration device is a syringe pump.

In an additional aspect, the present disclosure provides a method of treating cancer in a subject in need thereof comprising administering to the subject a therapeutically effective amount of any of the liquid pharmaceutical formulations comprising an anti-TIGIT monoclonal antibody and an anti-PD-L1 monoclonal antibody disclosed herein.

In a further aspect, the present disclosure provides a method of treating cancer in a subject in need thereof comprising administering to the subject a therapeutically effective amount of any of the liquid pharmaceutical formulations comprising an anti-TIGIT monoclonal antibody disclosed herein and administering to the subject a therapeutically effective amount of an anti-PD-1 monoclonal antibody, or an anti-PD-L1 monoclonal antibody. In some embodiments, the anti-PD-1 monoclonal antibody, or anti-PD-L1 monoclonal antibody, and the liquid pharmaceutical formulation comprising an anti-TIGIT monoclonal antibody are administered simultaneously. In some embodiments, the anti-PD-1 monoclonal antibody, or anti-PD-L1 monoclonal antibody, and the liquid pharmaceutical formulation comprising an anti-TIGIT monoclonal antibody are mixed 24 hours or less prior to administration to the subject. In some embodiments, the anti-PD-1 monoclonal antibody, or anti-PD-L1 monoclonal antibody, and the liquid pharmaceutical formulation comprising an anti-TIGIT monoclonal antibody are mixed during administration to the subject.

In some embodiments of any of the methods of treating cancer, the method comprises administering an anti-PD-1 antibody. In some embodiments, the anti-PD-1 antibody is selected from the group consisting of lambrolizumab (MK-3475), nivolumab (MDX-1106), pembrolizumab, cemiplimab, and dostarlimab.

In some embodiments, the method comprises administering an anti-PD-L1 monoclonal antibody. In some embodiments, the anti-PD-L1 monoclonal antibody is selected from the group consisting of atezolizumab (MPDL3280A), durvalumab (MEDI4736), avelumab, and MDX-1105. In some embodiments, the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15. In some embodiments, the heavy chain variable region (VH) of the anti-PD-L1 monoclonal antibody comprises the amino acid sequence of SEQ ID NO: 16. In some embodiments, the light chain variable region (VL) of the anti-PD-L1 monoclonal antibody comprises the amino acid sequence of SEQ ID NO: 17. In some embodiments, the heavy chain variable region of the anti-PD-L1 monoclonal antibody comprises the amino acid sequence of SEQ ID NO: 16 and the light chain variable region of the anti-PD-L1 monoclonal antibody comprises the amino acid sequence of SEQ ID NO: 17. In some embodiments, the heavy chain of the anti-PD-L1 monoclonal antibody comprises the amino acid sequence of SEQ ID NO: 21. In some embodiments, the heavy chain of the anti-PD-L1 monoclonal antibody comprises the amino acid sequence of SEQ ID NO: 22. In some embodiments, the light chain of the anti-PD-L1 monoclonal antibody comprises the amino acid sequence of SEQ ID NO: 23. In some embodiments, the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23. In some embodiments, the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23.

In some embodiments, the anti-PD-L1 monoclonal antibody or anti-PD-1 monoclonal antibody is an IgG antibody. In some embodiments, the anti-PD-L1 monoclonal antibody or anti-PD-1 monoclonal antibody is an IgG1 or an IgG4 antibody. In some embodiments, the anti-PD-L1 monoclonal antibody or anti-PD-1 monoclonal antibody is a full-length antibody. In some embodiments, the anti-PD-L1 monoclonal antibody or anti-PD-1 monoclonal antibody is a full-length IgG antibody. In some embodiments, the anti-PD-L1 monoclonal antibody or anti-PD-1 monoclonal antibody is a full-length IgG1 antibody. In some embodiments, the anti-PD-L1 monoclonal antibody or anti-PD-1 monoclonal antibody is a full-length human IgG1 antibody. In some embodiments, the anti-PD-L1 monoclonal antibody or anti-PD-1 monoclonal antibody is a full-length humanized IgG1 antibody. The anti-PD-L1 monoclonal antibody or anti-PD-1 monoclonal antibody may be an antibody fragment. In some embodiments, the anti-PD-L1 monoclonal antibody or anti-PD-1 monoclonal antibody is a Fab, a Fab′, a F(ab′)₂, a Fv, or a scFv fragment. In some embodiments, the anti-PD-L1 monoclonal antibody or anti-PD-1 monoclonal antibody is a human antibody. In some embodiments, the anti-PD-L1 monoclonal antibody or anti-PD-1 monoclonal antibody is a humanized antibody.

In some embodiments, the liquid pharmaceutical formulation is administered intravenously. In some embodiments, the liquid pharmaceutical formulation is administered subcutaneously.

In some embodiments, the cancer is selected from the group consisting of a lung cancer, a non-small cell lung cancer, a renal cell cancer, a urothelial cancer, a ureter cancer, a urethral cancer, a colorectal cancer, a colon cancer, a rectal cancer, a kidney cancer, a sarcoma, an ovarian cancer, a breast cancer, a cervical cancer, a fallopian tube cancer, an endometrial cancer, a uterine cancer, a pancreatic cancer, a gastric carcinoma, a bladder cancer, an esophageal cancer, a mesothelioma, a melanoma, a head and neck cancer, a thyroid cancer, a sarcoma, a prostate cancer, a penile cancer, a glioblastoma, a thymic carcinoma, an esophageal carcinoma, a nasopharyngeal cancer, a mesothelioma, a liver cancer, a biliary tract cancer, a HPV-positive cancer, a leukemia, a lymphoma, a brain cancer, a neuroendocrine cancer, a myeloma, a mycosis fungoides, a Merkel cell cancer, a hematologic malignancy, a deficient mismatch repair (dMMR) cancer, and a microsatellite instability-high (MSI-H) cancer.

In some embodiments, the cancer is selected from the group consisting of a bladder cancer, a muscle-invasive bladder cancer, a urothelial carcinoma, a ureter cancer, a urethral cancer, a ureter urothelial carcinoma, a urethral urothelial carcinoma, a renal cancer, a renal pelvis cancer, a renal cell carcinoma, a clear-cell renal carcinoma, a rectal cancer, a colon cancer, a colorectal cancer, a sarcoma, an osteosarcoma, a leiomyosarcoma, a pleomorphic sarcoma, a myxofibrosarcoma, a liposarcoma, a chondrosarcoma, a lung cancer, a non-small cell lung cancer, a fallopian tube cancer, a peritoneal carcinoma, an esophageal cancer, an esophageal squamous cell carcinoma, a mesothelioma, a pleural mesothelioma, a peritoneal mesothelioma, an ovarian cancer, a cervical cancer, a cervical adenosquamous carcinoma, a breast cancer, a triple-negative breast cancer, a HER2-positive breast cancer, a HER2-negative breast cancer, an estrogen receptor-positive breast cancer, a progesterone receptor-positive breast cancer, a luminal B breast cancer, a lymphoma, a T-cell lymphoma, a B-cell lymphoma, a nasal-type lymphoma, non-Hodgkin's lymphoma, a follicular lymphoma, a penile carcinoma, a prostate cancer, a castration-resistant prostate cancer, an endometrial cancer, a uterine cancer, a myeloma, a multiple myeloma, a head and neck cancer, a prostate cancer, a melanoma, a cutaneous melanoma, a Merkel cell carcinoma, a pancreatic cancer, hepatocellular carcinoma, gastric cancer, a gastroesophageal junction adenocarcinoma, a glioblastoma, a glioblastoma multiforme, a mycosis fungoides, an HPV-positive cancer, a HPV-related cervical carcinoma, a HPV-related anal squamous cell carcinoma, a HPV-related penile squamous cell carcinoma, a HPV-related vulvar squamous cell carcinoma, a vulvar cancer, a vaginal cancer, an anal cancer, an oropharyngeal cancer, an oropharyngeal squamous cell carcinoma, a leukemia, an acute myeloid leukemia, a bone cancer, a solitary bone plasmacytoma, a squamous cell carcinoma, a cutaneous squamous cell carcinoma, a thyroid cancer, a microsatellite stability/proficient mismatch repair (MSS/pMMR) metastatic colorectal cancer, a deficient mismatch repair (dMMR) cancer, a microsatellite instability-high (MSI-H) cancer, a nasal-type extranodal NK/T-Cell lymphoma, a neuroendocrine cancer, a biliary tract cancer, a cholangiocarcinoma, and an intrahepatic cholangiocarcinoma.

In some embodiments, the cancer is selected from the group consisting of a Merkel cell carcinoma, a urothelial carcinoma, a renal cell carcinoma, non-small cell lung cancer, a breast cancer, a triple-negative breast cancer, a hepatocellular carcinoma, a melanoma, a Hodgkin's lymphoma, a head and neck cancer, a colorectal cancer, a gastric cancer, a cervical cancer, a primary mediastinal large B-cell lymphoma, a cutaneous squamous-cell carcinoma, a basal cell carcinoma, a bladder cancer, an endometrial cancer, an esophageal cancer, a malignant pleural mesothelioma, a tumor mutational burden (TMB)-high cancer, a deficient mismatch repair (dMMR) cancer, and a microsatellite instability-high (MSI-H) cancer.

In some embodiments, the cancer is selected from the group consisting of a lung cancer, a non-small cell lung cancer, a bronchogenic carcinoma, a breast cancer, a triple-negative breast cancer, an estrogen receptor-positive breast cancer, a HER2-positive breast cancer, a lobular metastatic breast cancer, a ductal breast carcinoma, a cervical cancer, a fallopian tube cancer, a fallopian tube serous adenocarcinoma, an ovarian cancer, an ovarian endometrioid tumor, an ovarian serous adenocarcinoma, an ovarian seromucinous carcinoma, a uterine cancer, an endometrial cancer, a skin cancer, a melanoma, a cutaneous melanoma, a Merkel cell carcinoma, a head and neck cancer, squamous cell carcinoma of head and neck, a hematologic malignancy, a leukemia, a myeloid leukemia, an acute myeloid leukemia, a chronic lymphocytic leukemia, a myelomonocytic leukemia, a thyroid cancer, thyroid gland carcinoma, a thymic carcinoma, a neuroendocrine cancer, a pheochromocytoma, a glioma, a glioblastoma multiforme, a paraganglioma, a lymphoma, a B-cell lymphoma, a Hodgkin lymphoma, a B-cell non-Hodgkin lymphoma, a non-Hodgkin's lymphoma, a cutaneous T-cell lymphoma, a diffuse large B-cell lymphoma, a follicular lymphoma, a marginal zone lymphoma, a pancreatic cancer, a pancreatic ductal adenocarcinoma, a rectal cancer, a colon cancer, a colorectal cancer, a urinary tract cancer, a genitourinary cancer, a mesothelioma, a pleural mesothelioma, a peritoneal mesothelioma, a sarcoma, a chondrosarcoma, a clear cell sarcoma, a liposarcoma, a myxoid/round cell liposarcoma, a synovial sarcoma, an alveolar soft part sarcoma, a gliosarcoma, a uterine carcinosarcoma, a kidney cancer, a non-clear cell kidney cancer, a renal cell carcinoma, a bladder cancer, a urothelial carcinoma, a muscle-invasive bladder cancer, a non-muscle invasive bladder cancer, a HER2-positive bladder cancer, a gallbladder carcinoma, a gastric cancer, an esophageal cancer, an esophageal squamous cell carcinoma, a gastrointestinal cancer, a gastroesophageal cancer, a gastroesophageal junction cancer, a HER2-positive gastric cancer, a primary peritoneal cancer, a cutaneous squamous cell carcinoma, a prostate cancer, a prostate adenocarcinoma, a castration-resistant prostate cancer, a urogenital cancer, a ureter urothelial carcinoma, a renal pelvis urothelial carcinoma, a urethral urothelial carcinoma, an appendix carcinoma, a penile cancer, an anal canal cancer, a hepatocellular carcinoma, a hepatobiliary cancer, an unresectable liver and intrahepatic bile duct carcinoma, a biliary tract cancer, a cholangiocarcinoma, an intrahepatic cholangiocarcinoma, an extrahepatic cholangiocarcinoma, an HPV-related cancer, an HPV-related anal squamous cell carcinoma, an HPV-related cervical squamous cell carcinoma, an HPV-related penile squamous cell carcinoma, an HPV-related vulvar squamous cell carcinoma, a nasopharynx carcinoma, a nasopharyngeal carcinoma, a laryngeal squamous cell carcinoma, a hypopharyngeal squamous cell carcinoma, an oral cavity squamous cell carcinoma, and a mycosis fungoides.

In some embodiments, the cancer is selected from the group consisting of urothelial carcinoma, non-small cell lung cancer (NSCLC), breast cancer, triple-negative breast cancer, hepatocellular carcinoma and melanoma.

In some embodiments, the cancer is selected from the group consisting of a multiple myeloma, a cervical cancer, an esophageal cancer, an esophageal squamous cell carcinoma, a lung cancer, a non-small cell lung cancer, a glioblastoma, an endometrial cancer, an ovarian cancer, a squamous cell cancer, a head and neck cancer.

In some embodiments, the cancer is selected from the group consisting of a cervical cancer, a squamous cell carcinoma of head and neck, a head and neck cancer, a non-small cell lung cancer, a non-squamous non-small cell lung cancer, an esophageal squamous cell carcinoma, an esophageal cancer, a breast cancer, a triple-negative breast cancer, a gastric cancer, a gastroesophageal junction adenocarcinoma, a multiple myeloma, a non-Hodgkin lymphoma, a B-cell lymphoma, a liver cancer, a bladder cancer, a urothelial carcinoma, a pancreatic cancer, and a pancreatic adenocarcinoma.

In some embodiments, the cancer is a solid tumor. In some embodiments, the cancer is a hematological cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B demonstrate Polysorbate 20 (PS20) degradation as measured by Fatty Acid Mass Spectrometry (FAMS). FIG. 1A shows a table of fatty acid generation (e.g., lauric acid and myristic acid) as measured in ng/ml/week at 25° C. and 40° C. FIG. 1B is a graph showing rate of degradation of PS20 at 25° C. using FAMS Comparison. aTIGIT refers to α-TIGIT.

FIGS. 2A-2D provide graphs showing the concentration of anti-TIGIT monoclonal antibody (tiragolumab) over time at different temperatures (−20° C. (FIG. 2A), 5° C. (FIG. 2B), 25° C. (FIG. 2C) and 40° C. (FIG. 2D)) as measured by evaporative light scattering detector (ELSD). “Solid” refers to the striped symbols and is used for Formulations with PS20. “Hollow” refers to the unfilled symbols and is used for Formulations with PX188.

FIGS. 3A.I, 3A.II, 3A.III, 3B.I, 3B.II, 3B.III, 3C.I, 3C.II, 3C.III, 3D.I, 3D.II, 3D.III, 3E.I, 3E.II, 3E.III, 3F.I, 3F.II, 3F.III, 3G.I, 3G.II, 3G.III, 3H.I, 3H.II, and 3H.III provide graphs that show stability of anti-TIGIT monoclonal antibody (tiragolumab) by measurement of aggregate formation and low molecular weight species (LMWS) over time or formation of charged isomers in acidic or basic conditions. FIG. 3A.I (Aggregate), FIG. 3A.II (Main), and FIG. 3A.III (low molecular weight species (LMWS)): Aggregates and LMWS were measured at 5° C. using size exclusion chromatography. FIG. 3B.I (Acidic), FIG. 3B.II (Main), and FIG. 3B.III (Basic): Charged isomers were measured at 5° C. using imaged capillary isoelectric focusing (ICIEF). FIG. 3C.I (Aggregate), FIG. 3C.II (Main), and FIG. 3C.III (LMWS): Aggregates and LMWS were measured at −20° C. using size exclusion chromatography. FIG. 3D.I (Acidic), FIG. 3D.II (Main), and FIG. 3D.III (Basic): Charged isomers were measured at −20° C. using ICIEF. FIG. 3E.I (Aggregate), FIG. 3E.II (Main), and FIG. 3E.III (LMWS): Aggregates and LMWS were measured at 25° C. using size exclusion chromatography. FIG. 3F.I (Acidic), FIG. 3F.II (Main), FIG. 3F.III (Basic): Charged isomers were measured at 25° C. using ICIEF. FIG. 3G.I (Aggregate), FIG. 3G.II (Main), and FIG. 3G.III (LMWS): Aggregates and LMWS were measured at 40° C. using size exclusion chromatography. FIG. 3H.I (Acidic); FIG. 3H.II (Main); FIG. 3H.III (Basic): Charged isomers were measured at 40° C. using ICIEF.

FIG. 4.I and FIG. 4.II provide graphs that show stability of anti-TIGIT monoclonal antibody (tiragolumab) by measurement of high molecular weight species (HMWS) and low molecular weight species (LMWS) used size exclusion chromatography (SEC) (FIG. 4.II), or formation of charged isomers in acidic or basic conditions using imaged capillary isoelectric focusing (ICIEF) (FIG. 4.I) based on different protein concentrations, pH, surfactant concentration, histidine acetate concentration, sucrose concentration, and surfactant type (e.g., PS20 or PX188).

FIG. 5 provides a graph showing the stability of an anti-TIGIT monoclonal antibody formulation (60 mg/mL anti-TIGIT monoclonal antibody (tiragolumab), 20 mM histidine acetate, 120 mM sucrose, pH 5.5 in 25 cc 316L Mini-Can) after freezing and thawing 7× as measured by SEC Main Peak in percentage.

FIG. 6 provides size-exclusion chromatography (SEC) for a high concentration tiragolumab formulation over one year at −20° C. storage. The high concentration formulation (“PH3 DS;” 60 mg/mL tiragolumab in 20 mM HisOAc, 240 mM sucrose, 10 mM methionine, 0.04% PS20 and pH 5.5) was evaluated at TO and following storage at −20° C. for 91 days, 9 months, and 12 months. “HisOAc”=histidine acetate and “RS”=Rep. Stability (400 L run).

FIG. 7 provides capillary electrophoresis (CE) for a high concentration tiragolumab formulation over one year at −20° C. storage. The high concentration formulation (“PH3 DS;” 60 mg/mL tiragolumab in 20 mM HisOAc, 240 mM sucrose, 10 mM methionine, 0.04% PS20 and pH 5.5) was evaluated at T0 and following storage at −20° C. for 91 days, 9 months, and 12 months. Minor differences in one of the LMW peaks may be due to assay variability. “HisOAc”=histidine histidine acetate and “RS”=Rep. Stability (400 L run).

FIGS. 8A-8D demonstrate the stability of tiragolumab drug product (DP; 60 mg/ml tiragolumab, 20 mM histidine acetate, 240 mM sucrose, 10 mM methionine, 0.4 mg/ml polysorbate 20, and pH 5.5) with different infusion systems. FIG. 8A provides the stability for Setup 1: PVC Bag, PVC Set, PC, and PEU Infusion Aids; PES I-line filter. FIG. 8B provides the stability for Setup 2: PO Bag, PE Set, PC and PTFE Infusion Aids, PES In-line Filter. FIG. 8C provides the stability for Setup 3: PE Bag, PBD Set, PC and PUR Infusion Aids, PSU In-line Filter. FIG. 8D provides the stability for Setup 4: PP Bag, PUR Set, PC and FEP Infusion Aids, PES In-line Filter. FIGS. 8A to 8D use the follow abbreviations: CL=clear; CO=colorless; FEP=fluorinated ethylene; HMW=high molecular weight; IE-HPLC=ion-exchange high-performance liquid chromatography; LIQ=liquid; LMW=low molecular weight; NT=not tested; PBD=polybutadiene; PC=polycarbonate; PE=polyethylene; PES=polyethersulfone; PEU=polyetherurethane; PFFP=practically free from particles; PO=polyolefin; PP=polypropylene; PSU=polysulfone; PTFE=polytetrafluoroethylene; PUR=polyurethane; PVC=polyvinyl chloride; SE-UPLC=size-exclusion ultra-high-performance liquid chromatography; UV=ultraviolet.

^{a} Protein content by UV was used for the drug product and high-dose samples. SE-UHPLC using a standard curve was used to determine protein concentration.

FIGS. 9A-9C provide the stability of the dose solution for co-infusion of tiragolumab drug product and TECENTRIQ® (atezolizumab) drug product with different infusion systems. FIG. 9A provides the stability for Setup 1: PVC Bag, PVC Set, PC and PEU Infusion Aids, PES In-line Filter. FIG. 9B provides the stability for Setup 2: PO Bag, PE Set, PC and PTFE Infusion Aids, PES In-line Filter. FIG. 9C provides the stability for Setup 3: PO Bag, PBD Set, PC and PUR Infusion Aids, PSU In-line Filter. FIGS. 9A to 9C use the follow abbreviations: CL=clear; CO=colorless; FEP=fluorinated ethylene; HMW=high molecular weight; IE-HPLC=ion-exchange high-performance liquid chromatography; LIQ=liquid; LMW=low molecular weight; N/A=not applicable; NT=not tested; PBD=polybutadiene; PC=polycarbonate; PE=polyethylene; PES=polyethersulfone; PEU=polyetherurethane; PFFP=practically free from particles; PO=polyolefin; PP=polypropylene; PSU=polysulfone; PTFE=polytetrafluoroethylene; PUR=polyurethane; PVC=polyvinyl chloride; SE-UPLC=size-exclusion ultra-high-performance liquid chromatography; UV=ultraviolet.

^{a} A hydrophilic interaction chromatography (HILIC) assay was developed to measure the protein content of tiragolumab and TECENTRIQ® (atezolizumab).
^{b} An alternate IE-HPLC method was developed to enable quantification of the charge variants of both tiragolumab and TECENTRIQ® (atezolizumab).

FIG. 10 provides a graph showing the viscosity of various concentrations of tiragolumab in different buffers and storage conditions. “HisOAc”=histidine acetate; “ArgSucc”-arginine succinate; and “Expon.”=exponential curve fit.

FIG. 11 provides measurements of the stability of various tiragolumab formulations (Formulation 1-160 mg/mL tiragolumab in 20 mM HisOAc, 240 mM sucrose, 10 mM methionine, 0.06% PS20 and pH 5.5; and Formulation 2-176 mg/mL tiragolumab in 30 mM HisOAc, 180 mM sucrose, 5 mM methionine, 0.08% PS20 and pH 5.5) after various freeze-thaw cycles in various formulations.

FIGS. 12A and 12B provides size-exclusion chromatography (SEC) for a high concentration tiragolumab formulation (FIG. 12A) and a very high concentration tiragolumab formulation (FIG. 12B). The high concentration formulation (“60 mg/ml tira;” 60 mg/ml tiragolumab in 20 mM HisOAc, 240 mM sucrose, 10 mM methionine, 0.04% PS20 and pH 5.5) was evaluated following storage at 40° C. for 7 days, 14, days, 30 days and 60 days, and the very high concentration formulation (“160 mg/ml tira;” 160 mg/mL tiragolumab in 20 mM HisOAc, 240 mM sucrose, 10 mM methionine, 0.06% PS20 and pH 5.5) was evaluated at TO and following storage at 40° C. for 2 weeks, 3 weeks, and 1 month.

FIGS. 13A and 13B provides ion-exchange chromatography (IEC) for a high concentration tiragolumab formulation (FIG. 13A) and a very high concentration tiragolumab formulation (FIG. 13B). The high concentration formulation (“60 mg/ml tira;” 60 mg/mL tiragolumab in 20 mM HisOAc, 240 mM sucrose, 10 mM methionine, 0.04% PS20 and pH 5.5) was evaluated at TO and following storage at 40° C. for 14 days and 30 days, and the very high concentration formulation (“160 ml/mg tira;” 160 mg/mL tiragolumab in 20 mM HisOAc, 240 mM sucrose, 10 mM methionine, 0.06% PS20 and pH 5.5) was evaluated at TO and following storage at 40° C. for 2 weeks, 3 weeks, and 1 month.

FIG. 14 provides a graph showing hyaluronidase activity following 25° C. storage over time (90 days) in a formulation comprising a pH of 5.2 or a pH of 5.5.

FIGS. 15A and 15B provide graphs showing the percentage of the high molecular weight fraction (HMWF; FIG. 15A) and low molecular weight fraction (LMWF; FIG. 15B) over time as measured by size exclusion chromatography of tiragolumab (Tira), atezolizumab (Atezo), or a combination of tiragolumab and atezolizumab at varying pH (e.g., 5.4, 5.8 and 6.2).

FIGS. 16A and 16B provide graphs showing the percentage of the tiragolumab (Tira; FIG. 16A) main peak and atezolizumab (Atezo; FIG. 16B) main peak over time as measured by ion exchange chromatography at pH 5.4, 5.8 and 6.2.

FIGS. 17A and 17B provide graphs showing the percentage of the low molecular weight forms (LMWF; “pre-peaks;” FIG. 17A-Sum of Pre-Peak Change Over Time) and high molecular weight forms (HMWF; FIG. 17B-Sum of HMWF Change Over Time) over time as measured by CE-SDS of tiragolumab (Tira), atezolizumab (Atezo), or a combination of tiragolumab and atezolizumab at varying pH (e.g., 5.4, 5.8 and 6.2).

FIG. 18 provides a treatment protocol using the formulations of this disclosure. FIG. 18 uses the following abbreviations: Atezo=atezolizumab; ECOG=Eastern Cooperative Oncology Group; IV=intravenous; PD=progressive disease; PK=pharmacokinetics; PS=Performance Status; Q3W=every 3 weeks; SC=subcutaneous; TBD=to be determined; Tira=tiragolumab.

^{a} Single dose of tiragolumab SC and atezolizumab SC co-mix (in the abdomen).
^{b} Three cycles of tiragolumab SC and atezolizumab SC co-mix Q3W (in the thigh).
^{c} Three cycles of tiragolumab SC and atezolizumab SC co-mix Q3W (in the abdomen).

FIG. 19 provides a treatment protocol using one of the formulations disclosed herein (40 mg/mL tiragolumab, 80 mg/mL atezolizumab, 20 mM histidine acetate, 240 mM sucrose, 0.06% (w/v) polysorbate 20, and a pH of 5.8). FIG. 19 uses the following abbreviations: CPI=checkpoint inhibitor; EAC=esophageal adenocarcinoma; ECOG PS-Eastern Cooperative Oncology Group Performance Status; ESCC=esophageal squamous cell carcinoma; FDC=fixed-dose combination; GEJ=gastroesophageal junction cancer; HCC=hepatocellular carcinoma; IMC=Internal Monitoring Committee; IV=intravenous; NSCLC=non-small cell lung cancer; PD=progressive disease; PD-L1=programmed death ligand-1; Q3W=every 3 weeks; RCC=renal cell cancer; SCCHN=squamous cell carcinoma of the head and neck; UBC=urothelial bladder; mets=metastases; and *=Enrollment will focus on subjects who have EAC, ESCC, GEJ, HCC, melanoma, NSCLC, RCC, SCCHN, and UBC. Additional tumor types may be added as the PD-L1 cutoffs become available.

FIG. 20 provides a treatment protocol using the formulations of this disclosure. FIG. 20 uses the following abbreviations: Atezo=atezolizumab; ECOG=Eastern Cooperative Oncology Group; IV=intravenous; PD=progressive disease; PK=pharmacokinetic; PS=Performance Status; Q3W=every 3 weeks; SC=subcutaneous; TBD=to be determined; Tira=tiragolumab

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The term “herein” means the entire disclosure.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the dependent (including multiple dependent) claims.

Any publications, patents and published patent applications referred to in this disclosure are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that, unless otherwise indicated or the context clearly indicates otherwise, the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) in particular embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Also, recitations of numerical values are not limited to specifically the recited value, but also include numbers that the skilled artisan would normally round to that value in the context of significant digits. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present disclosure. The materials, methods, and examples are illustrative only and not intended to be limiting.

### Definitions

“Administering” or “administration of” a substance, a compound, an agent, or a composition to a subject, as used herein, refers to the contact of that substance, compound, agent, or composition to the subject or a cell, tissue, organ or bodily fluid of the subject. Such administration can be carried out using one of a variety of methods known to those skilled in the art. For example, a substance, compound, agent, or composition can be administered orally or parenterally, such as by injection. In some embodiments, a substance, compound, agent or composition is administered subcutaneously. In some embodiments, a substance, compound, agent or composition is administered intravenously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject.

As used herein, the term “antibody” or “Ab” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

The terms “anti-TIGIT antibody,” “anti-TIGIT monoclonal antibody,” and “an antibody that specifically binds to TIGIT” are used interchangeably herein and refer to an antibody that is capable of binding TIGIT with sufficient affinity such that the antibody is useful as a diagnostic and/or therapeutic agent in targeting TIGIT. In some embodiments, the extent of binding of an anti-TIGIT antibody to an unrelated, non-TIGIT protein is less than about 10% of the binding of the antibody to TIGIT as measured, e.g., by a radioimmunoassay (RIA). In certain embodiments, an antibody that binds to TIGIT has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-TIGIT antibody binds to an epitope of TIGIT that is conserved among TIGIT from different species or an epitope on TIGIT that allows for cross-species reactivity, such as an epitope comprising amino acid residues Ser78, Ser80, and Lys82.

The terms “anti-PD-L1 antibody,” “anti-PD-L1 monoclonal antibody,” and “an antibody that specifically binds to PD-L1” are used interchangeably herein and refer to an antibody that is capable of binding PD-L1 with sufficient affinity such that the antibody is useful as a diagnostic and/or therapeutic agent in targeting PD-L1. In some embodiments, the extent of binding of an anti-PD-L1 antibody to an unrelated, non-PD-L1 protein is less than about 10% of the binding of the antibody to PD-L1 as measured, e.g., by a radioimmunoassay (RIA). In certain embodiments, an antibody that binds to PD-L1 has a dissociation constant (Kd) of ≤ 1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-PD-L1 antibody binds to an epitope of PD-L1 that is conserved among PD-L1 from different species or an epitope on PD-L1 that allows for cross-species reactivity.

The term “antibody fragment,” as used herein, refers to a portion of an intact antibody, preferably the antigen binding and/or the variable region of the intact antibody. Examples of antibody fragments include Fab, Fab′, F(ab′)₂, Fv fragments; diabodies; linear antibodies (see U.S. Pat. No. 5,641,870, Example 2; Zapata et al., Protein Eng. 8 (10): 1057-1062 [1995]); single-chain antibody molecules (such as scFv molecules) and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments with each having antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. The Fc fragment comprises the carboxy-terminal portions of both H chains held together by disulfides. The effector functions of antibodies are determined by sequences in the Fc region, the region which is also recognized by Fc receptors (FcR) found on certain types of cells. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

The term “buffer,” as used herein, refers to a reagent that can resist pH change upon the addition of an acid or base to maintain a relatively stable pH in a solution. Non-limiting examples of buffers include histidine, arginine, acetate, citrate, succinate, gluconate, phosphate, or combinations thereof. Other non-limiting examples include histidine, acetate, histidine acetate, histidine hydrochloride, histidine acetate and arginine, citrate, citric acid, sodium acetate, sodium citrate, arginine succinate, phosphate, disodium phosphate dihydrate, and sodium dihydrogen phosphate dihydrate or combinations thereof.

The term “atezolizumab,” as used herein, refers to anti-PD-L1 monoclonal antagonist antibody having the International Nonproprietary Names for Pharmaceutical Substances (INN) List 112 (WHO Drug Information, Vol. 28, No. 4, 2014, p. 488), or the CAS Registry Number 1380723-44-3.

The term “cancer,” as used herein, refers to a disease caused by an uncontrolled division of abnormal cells in a part of the body. The cancer may be locally advanced or metastatic. In some instances, the cancer is locally advanced. In some instances, the cancer is metastatic. In some instances, the cancer is recurrent. In some instances, the cancer may be unresectable (e.g., unresectable locally advanced or metastatic cancer).

The term “chimeric,” as used herein, antibody refers to an antibody in which a portion of the heavy and/or light chain is derived from a particular source or species, while the remainder of the heavy and/or light chain is derived from a different source or species.

The terms “full-length antibody” “intact antibody” and “whole antibody” are used interchangeably herein and refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. It is known in the art that during antibody expression C-terminal clipping of the antibody by carboxypeptidases occurs. Such clipped antibodies are considered to be in substantially intact form and, thus, full-length antibodies, despite the removal of one or more C-terminal amino acid residues. In some cases, the intact antibody may have one or more effector functions. In some embodiments, the intact antibody retains all effector functions. Optionally, one or more effector functions of the antibody may have been modified or eliminated.

As used herein, the term “effector function” refers to a biochemical event that results from the interaction of an antibody Fc region with an Fc receptor or another effector molecule (e.g., Fc receptor-Like (FcRL) molecules, complement component Clq, and Tripartite motif-containing protein 21 (TRIM21)). Effector functions include, but are not limited to, antibody dependent cell-mediated cytotoxicity (ADCC), antibody dependent cell-mediated phagocytosis (ADCP) and complement-dependent cellular cytotoxicity (CDC). The term “ADCC” or “antibody dependent cell-mediated cytotoxicity,” as used herein, refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. The term “ADCP” or “antibody dependent cell-mediated phagocytosis,” as used herein, refers to the cell-mediated reaction wherein nonspecific cytotoxic cells that express FcγRs recognize bound antibody on a target cell and subsequently cause phagocytosis of the target cell. The term “CDC” or “complement-dependent cellular cytotoxicity,” as used herein, refers to an effector function which leads to the activation of the classical complement pathway, which is triggered by the binding of an antibody to an antigen on the target cell, which activates a series of cascades containing complement-related protein groups in blood.

The term “human antibody” as used herein refers to an antibody that possesses an amino-acid sequence corresponding to that of an antibody produced by a human and/or has been made using any of the techniques known in the art for making human antibodies. A “human antibody” specifically excludes a humanized antibody comprising non-human antigen-binding residues. Human antibodies can be produced using various techniques known in the art, including phage-display libraries, mouse hybridoma, transgenic animals (e.g., mice) and single B cell technique. See, e.g., Lu et al, J. Biomed. Sci., 27:1 (2020); Hoogenboom and Winter, J. Mol. Biol., 227:381 (1991); Marks et al., J. Mol. Biol., 222:581 (1991), each of which is incorporated by reference herein in its entirety. Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce human antibodies in response to antigenic challenge, e.g., immunized xenomice (see, e.g., U.S. Pat. Nos. 6,075,181 and 6,150,584 regarding XENOMOUSE™ technology). Other transgenic animals for producing human antibodies are also known in the art, including, e.g., the HuMAb mouse, the UntiMAb mouse, the Transchromo mouse, the VelocImmune mice, the OmniRat, the OmniMouse, the Harbour Mouse, the Kymouse, the MeMo mouse, the AlivaMab mouse, etc. See, e.g., Brüggemann et al., Arch. Immunol. Ther. Exp. (Warsz.) 2015, vol. 63 (2): 101-108. Additional techniques are also known the art. See also, for example, Li et al., Proc. Natl. Acad. Sci. USA, 103:3557-3562 (2006) regarding human antibodies generated via a human B-cell hybridoma technology.

“Humanized” antibodies, as used herein, refer to chimeric antibodies that contain both human and non-human antibody sequences. Typically, humanized antibodies comprise minimal sequence derived from the non-human immunoglobulin. For example, humanized antibodies include human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, certain framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody, optionally, will also comprise at least a portion of an immunoglobulin constant region (Fc), typically that of a human immunoglobulin. For further details, see Jones et al, Nature 321:522-525 (1986); Riechmann et al, Nature 332:323-329 (1988); and Presta, Curr. Op. Struct. Biol. 2:593-596 (1992). See also, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

The term “hyaluronidase,” as used herein, refers to an enzyme that catalyzes the degradation of hyaluronic acid (also referred to as hyaluronan). Hyaluronidase transiently hydrolyzes hyaluronic acid, which is a component of the subcutaneous matrix, and reduces the viscosity of the extracellular matrix of the hypodermis to improve delivery of subcutaneously administered drugs in the systemic circulation. In some embodiments, hyaluronidase is a recombinant human hyaluronidase. In some embodiments, recombinant human hyaluronidase is administered subcutaneously.

The term “hypervariable region,” “HVR,” or “HV,” as used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3, in particular, is believed to play a unique role in conferring fine specificity to antibodies. See, e.g., Xu et ah, Immunity 13:37-45 (2000); Johnson and Wu, Methods in Molecular Biology 248:1-25 (Lo, ed., Human Press, Totowa, NJ, 2003). For example, naturally occurring camelid antibodies consisting of only a heavy chain are functional and stable in the absence of light chain. See, e.g., Hamers-Casterman et al, Nature 363:446-448 (1993); Sheriff et al, Nature Struct. Biol. 3:733-736 (1996).

A number of HVR delineations are used in the art and are encompassed herein. The Kabat Complementarity Determining Regions (CDRs) are based on sequence variability and are one of the most commonly used definitions (Kabat et al., Sequences of Proteins of Immunological Interest, 5th Ed. Public Health Service, National Institutes of Health, Bethesda, MD. (1991)). Chothia refers, instead, to the location of the structural loops (Chothia and Lesk, J. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures. The IMGT numbering system was created by taking into account the high conservation of the structure of the V domain and by integrating the knowledge acquired by the analysis of multiple sources: alignment of more than 5000 sequences, literature data on the framework (FR) and complementarity determining regions (CDR), structural data from X-ray diffraction studies and characterization of the CDR hypervariable loops. The residues from each of these HVRs are noted below in Table 1.

TABLE 1

| Loop | Kabat | AbM | Chothia | Contact | IMGT |
|---|---|---|---|---|---|
| L1 | L24- | L24- | L26- | L30- | L27- |
| | L34 | L34 | L32 | L36 | L32 |
| L2 | L50- | L50- | L50- | L46- | L50- |
| | L56 | L56 | L52 | L55 | L51 |
| L3 | L89- | L89- | L91- | L89- | L89- |
| | L97 | L97 | L96 | L96 | L97 |
| H1 | H31- | H26- | H26- | H30- | H26- |
| Kabat Numbering) | H35B | H35B | H32 | H35B | H35B |
| H1 | H31- | H26- | H26- | H30- | H26- |
| (Chothia Numbering) | H35 | H35 | H32 | H35 | H33 |
| H2 | H50- | H50- | H53- | H47- | H51- |
| | H65 | H58 | H55 | H58 | H56 |
| H3 | H95- | H95- | H96- | H93- | H93- |
| | H102 | H102 | H101 | H101 | H102 |

Unless otherwise indicated, the HVRs are determined according to Kabat et al., supra. HVRs may comprise “extended HVRs” as follows: residues 24-36 or 24-34 (L1), residues 46-56 or 50-56 (L2) and residues 89-97 or 89-96 (L3) in the VL and residues 26-35 (H1), residues 50-65 or 49-65 (H2) and residues 93-102, 94-102, or 95-102 (H3) in the VH, each according to Kabat numbering.

The term “monoclonal antibody,” as used herein, refers to an antibody obtained from a single clone of cells or a cell line that produce a population of substantially homogeneous antibodies, i.e., the individual antibodies produced by the cells are identical except for possible naturally occurring mutations that may be present in minor amounts. Monoclonal antibodies are highly specific and are directed against a single antigen. Furthermore, in contrast to polyclonal antibody preparations that typically include different antibodies directed against different determinants (epitopes), each monoclonal antibody is directed against a single determinant on the antigen. Monoclonal antibodies may be human, humanized or chimeric antibodies.

The term “stabilizer,” as used herein, refers to a reagent that reduces or minimizes oxidation of a composition. Non-limiting examples of a stabilizer include methionine, glycine, alanine, proline, taurine, betaine, octopine, glutamate, sarcosine, γ-aminobutyric acid, and trimethylamine N-oxide.

The terms “subject” and “patient” are used interchangeably herein and refer to a human in need of treatment. Accordingly, the term “subject” or “patient,” as used herein, means a human patient or subject to which the compositions of the disclosure may be administered. In some embodiments, the subject is in need of treatment of cancer.

The term “surfactant,” as used herein, refers to a reagent that lowers the surface tension between two liquids, between a gas and a liquid, or between a liquid and a solid. In some embodiments, the surfactant prevents the loss of protein due to surface adsorption. In some embodiments, the surfactant minimizes the potential formation of soluble aggregates and/or insoluble proteinaceous particles. Surfactants may be ionic, non-ionic, zwitterionic or a combination thereof. Non-limiting examples of surfactants include a polysorbate (e.g., Polysorbate 20 and Polysorbate 80), a poloxamer (e.g., Poloxamer 188), triton, octyl glucoside, polyethyl glycol, myristamidopropyl-dimethylamine, palmidopropyl-dimethylamine, isostearamidopropyl-dimethylamine, polypropyl glycol, copolymers of ethylene, copolymers of propylene glycol, sodium dodecyl sulfate, sodium laurel sulfate, lauryl-sulfobetaine, myristyl-sulfobetaine, linoleyl-sulfobetaine, stearyl-sulfobetaine, lauroamidopropyl-betaine, cocamidopropyl-betaine, linoleamidopropyl-betaine, myristamidopropyl-betaine, palmidopropyl-betaine, isostearamidopropyl-betaine, sodium methyl cocoyl-taurate, sodium methyl oleyl-taurate, and combinations thereof.

The term “effective amount,” as used herein, refers to at least the minimum amount of a substance, compound, agent or composition, such as an antibody, required to affect a measurable improvement of a particular disorder. A therapeutically effective amount herein may vary according to factors such as the disease state, age, sex, and weight of the patient, and the ability of the substance, compound, agent or composition, such as an antibody, to elicit a desired response in the individual. The appropriate amount and dosage regimen can be determined using routine skill in the art. A therapeutically effective amount is also one in which any toxic or detrimental effects of the treatment are outweighed by the therapeutically beneficial effects. A beneficial or desired result include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, a therapeutically effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; relieving to some extent one or more of the symptoms associated with the disorder and/or maintaining remission. A therapeutically effective amount can be administered in one or more administrations. For purposes of this disclosure, a therapeutically effective amount of drug, compound, or pharmaceutical composition is an amount sufficient to accomplish therapeutic treatment either directly or indirectly. As is understood in the clinical context, a therapeutically effective amount of a drug, compound, or pharmaceutical composition may or may not be achieved in conjunction with another drug, compound, or pharmaceutical composition. Thus, a “therapeutically effective amount” may be considered in the context of administering one or more therapeutic agents, and a single agent may be considered to be given in a therapeutically effective amount if, in conjunction with one or more other agents, a desirable result may be or is achieved.

The term “tonicity agent,” as used herein, refers to a reagent that affects the osmotic pressure gradient of a composition. In some embodiments, the tonicity agent is added to a composition to achieve isotonicity, wherein the osmotic pressure of a composition is the same as compared to reference composition. In some embodiments, the composition may be isotonic with the blood or other bodily fluid of the subject. The tonicity agent may be a salt. In some embodiments, the tonicity agent is a polyol, such as a sugar or a sugar alcohol. Non-limiting examples of tonicity agents include fructose, mannose, maltose, lactose, arabinose, xylose, ribose, rhamnose, galactose, glucose, sucrose, trehalose, sorbose, melezitose, raffinose, mannitol, xylitol, erythritol, threitol, sorbitol, glycerol, sodium chloride and potassium chloride.

The term “treatment” (and variations thereof such as “treat” or “treating”), as used herein, refers to clinical intervention designed to alter the natural course of the individual or cell being treated during the course of clinical pathology. Desirable effects of treatment include decreasing the rate of disease progression, ameliorating or palliating the disease state, preventing recurrence of cancer, preventing metastasis, and remission or improved prognosis. For example, an individual suffering from cancer is successfully “treated” if one or more symptoms associated with the cancer are mitigated or eliminated, including, but are not limited to, reducing the proliferation of (or destroying) cancerous cells, decreasing symptoms resulting from the cancer, increasing the quality of life of those suffering from the cancer, decreasing the dose of other medications required to treat the cancer, delaying the progression of the cancer, and/or prolonging survival of individuals.

The term “variable region” or “variable domain,” as used herein, refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) While a VH domain is typically paired with a VL domain, a single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al, J. Immunol. 150:880-887 (1993); Clarkson et al, Nature 352:624-628 (1991).

The term “vial” as used herein refers to a small container that stores a pharmaceutical formulation. In some embodiments, the vial is stoppered with a chlorobutyl elastomer stopper. In some embodiments, the vial is glass. In some embodiments, the vial is plastic.

### Overview

The present disclosure relates to anti-TIGIT monoclonal antibody formulations, articles of manufacture, and methods of treatment that are suitable for co-administration or co-formulation with anti-PD-L1 monoclonal antibodies to reduce a patient's treatment time and, therefore, increase patient compliance. Therapeutic proteins, such as therapeutic antibodies, are large and have complex surface chemistries. Accordingly, different therapeutic proteins, such as therapeutic antibodies, have different interactions with the components of a pharmaceutical formulation, such that a formulation that confers stability to one therapeutic antibody may not confer stability to another therapeutic antibody. Indeed, it is known in the art that the hydrophobicity of an antibody's CDR loops is a key determinant of the propensity of the antibody to aggregate. See, e.g., Perchiacca et al., Annu. Rev. Chem. Biomol. Eng. 3:263-286, 2012. Antibodies that bind to different antigens have different CDR residues and, therefore, different surface chemistries. As a result, different antibodies often require different formulation components to confer stability and/or bioavailability. The present disclosure provides anti-TIGIT monoclonal antibody pharmaceutical formulations that may either be co-administered with an anti-PD-L1 monoclonal antibody or include an anti-PD-L1 monoclonal antibody without negatively affecting the stability and/or bioavailability of either antibody. Such formulations are unexpected in view of the state of the art. The present disclosure also provides stable, high-concentration anti-TIGIT formulations, which reduce the administration time from hours to minutes, thereby increasing patient convenience and compliance. The present disclosure also provides stable, high-concentration anti-TIGIT formulations suitable for subcutaneous administration, which reduce the administration time from hours to minutes, thereby increasing patient convenience and compliance.

### Pharmaceutical Formulations

A first aspect of the present disclosure provides a liquid pharmaceutical formulation comprising (a) an anti-TIGIT monoclonal antibody; (b) a buffer; (c) a tonicity agent; and (d) a surfactant, wherein the liquid pharmaceutical formulation has a pH of about 5.4 to about 6.2. In some embodiments, the liquid pharmaceutical formulation is suitable for co-administration with an anti-PD-L1 monoclonal antibody.

Another aspect of the present disclosure provides a liquid pharmaceutical formulation comprising (a) an anti-TIGIT monoclonal antibody; (b) an anti-PD-L1 monoclonal antibody; (c) a buffer; (d) a tonicity agent; and (e) a surfactant, wherein the liquid pharmaceutical formulation has a pH of about 5.4 to about 6.2.

In some embodiments, the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments, the anti-TIGIT monoclonal antibody comprises a heavy chain variable region comprising the amino acid sequence of SEQ ID NO: 7. In some embodiments, the anti-TIGIT monoclonal antibody comprises a light chain variable region comprising the amino acid sequence of SEQ ID NO: 9. In some embodiments, the anti-TIGIT monoclonal antibody comprises a heavy chain variable region comprising the amino acid sequence of SEQ ID NO: 7 and a light chain variable region comprising the amino acid sequence of SEQ ID NO: 9. In some embodiments of any of the above aspects, the heavy chain of the anti-TIGIT monoclonal antibody comprises the amino acid sequence of SEQ ID NO: 18. In some embodiments of any of the above aspects, the heavy chain of the anti-TIGIT monoclonal antibody comprises the amino acid sequence of SEQ ID NO: 19. In some embodiments of any of the above aspects, the heavy chain of the anti-TIGIT monoclonal antibody comprises the amino acid sequence of SEQ ID NO: 24. In some embodiments of any of the above aspects, the heavy chain of the anti-TIGIT monoclonal antibody comprises the amino acid sequence of SEQ ID NO: 25. In some embodiments of any of the above aspects, the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 18 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 19 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 24 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 25 and the light chain of the anti-TIGIT monoclonal antibody comprises the amino acid sequence of SEQ ID NO: 20. In some embodiments, the anti-TIGIT monoclonal antibody is tiragolumab. In some embodiments, the anti-TIGIT monoclonal antibody is tiragolumab. Tiragolumab is described in WHO Drug Information (International Nonproprietary Names for Pharmaceutical Substances), Proposed INN: List 117, Vol. 31, No. 2, published Jun. 9, 2017 (see p. 343). In some embodiments, tiragolumab has the CAS Registry Number 1918185-84-8.

In some embodiments, the anti-TIGIT monoclonal antibody is an IgG antibody. The anti-TIGIT monoclonal antibody may be an IgG1 antibody, an IgG2 antibody, an IgG3 antibody, or an IgG4 antibody. In some embodiments, the anti-TIGIT monoclonal antibody is an IgG1 or an IgG4 antibody. In some embodiments, the anti-TIGIT monoclonal antibody is an IgG1 antibody. In some embodiments, the anti-TIGIT monoclonal antibody is a wild-type IgG1 antibody. In some embodiments, the anti-TIGIT monoclonal antibody comprises a human IgG1 Fc region that comprises one or more amino acid modifications. In some embodiments, the anti-TIGIT monoclonal antibody is an IgG4 antibody. In some embodiments, the anti-TIGIT monoclonal antibody is a wild-type IgG4 antibody. In some embodiments, the anti-TIGIT monoclonal antibody comprises a human IgG4 Fc region that comprises one or more amino acid modifications. In some embodiments, the anti-TIGIT monoclonal antibody is an antagonist antibodyIn some cases, the anti-TIGIT monoclonal antibody may have one or more effector functions. In some embodiments, the anti-TIGIT monoclonal antibody retains all effector functions. Optionally, one or more effector functions of the anti-TIGIT monoclonal antibody may have been modified or eliminated.

In some embodiments, the anti-TIGIT monoclonal antibody is a human antibody. In some embodiments, the anti-TIGIT monoclonal antibody is a humanized antibody. In some embodiments, the anti-TIGIT monoclonal antibody is a full-length antibody. In some embodiments, the anti-TIGIT monoclonal antibody is a full-length IgG antibody. In some embodiments, the anti-TIGIT monoclonal antibody is a full-length IgG1 antibody. In some embodiments, the anti-TIGIT monoclonal antibody is a full-length human IgG1 antibody. The anti-TIGIT monoclonal antibody may be an antibody fragment. In some embodiments, the anti-TIGIT monoclonal antibody is a Fab, a Fab′, a F(ab′)₂, a Fv, or a scFv fragment. In some embodiments, the anti-TIGIT monoclonal antibody is a Fab fragment. In some embodiments, the anti-TIGIT monoclonal antibody is a Fab′ fragment. In some embodiments, the anti-TIGIT monoclonal antibody is a F(ab′)₂, fragment. In some embodiments, the anti-TIGIT monoclonal antibody is a Fv fragment. In some embodiments, the anti-TIGIT monoclonal antibody is a scFv fragment. In some embodiments, the anti-TIGIT monoclonal antibody is a diabody. In some embodiments, the anti-TIGIT monoclonal antibody is a linear antibody. In some embodiments, the anti-TIGIT monoclonal antibody is a single-chain antibody molecule. In some embodiments, the anti-TIGIT monoclonal antibody is a multispecific antibody, e.g. formed from antibody fragments.

In some embodiments, the concentration of the anti-TIGIT monoclonal antibody is about 20 mg/mL to about 75 mg/mL. In some embodiments, the concentration of the anti-TIGIT monoclonal antibody is about 25 mg/mL to about 75 mg/mL. In some embodiments, the concentration of the anti-TIGIT monoclonal antibody is about 30 mg/mL to about 75 mg/mL. In some embodiments, the concentration of the anti-TIGIT monoclonal antibody is about 35 mg/mL to about 75 mg/mL. In some embodiments, the concentration of the anti-TIGIT monoclonal antibody is about 40 mg/mL to about 75 mg/mL. In some embodiments, the concentration of the anti-TIGIT monoclonal antibody is about 45 mg/mL to about 75 mg/mL. In some embodiments, the concentration of the anti-TIGIT monoclonal antibody is about 50 mg/mL to about 75 mg/mL. In some embodiments, the concentration of the anti-TIGIT monoclonal antibody is about 55 mg/mL to about 75 mg/mL. In some embodiments, the concentration of the anti-TIGIT monoclonal antibody is about 60 mg/mL to about 75 mg/mL. In some embodiments, the concentration of the anti-TIGIT monoclonal antibody is about 65 mg/mL to about 75 mg/mL. In some embodiments, the concentration of the anti-TIGIT monoclonal antibody is about 70 mg/mL to about 75 mg/mL. In some embodiments, the concentration of the anti-TIGIT monoclonal antibody is about 20 mg/mL to about 70 mg/mL. In some embodiments, the concentration of the anti-TIGIT monoclonal antibody is about 20 mg/mL to about 65 mg/mL. In some embodiments, the concentration of the anti-TIGIT monoclonal antibody is about 20 mg/mL to about 60 mg/mL. In some embodiments, the concentration of the anti-TIGIT monoclonal antibody is about 20 mg/mL to about 55 mg/mL. In some embodiments, the concentration of the anti-TIGIT monoclonal antibody is about 20 mg/mL to about 50 mg/mL. In some embodiments, the concentration of the anti-TIGIT monoclonal antibody is about 20 mg/mL to about 45 mg/mL. In some embodiments, the concentration of the anti-TIGIT monoclonal antibody is about 20 mg/mL to about 40 mg/mL. In some embodiments, the concentration of the anti-TIGIT monoclonal antibody is about 20 mg/mL to about 35 mg/mL. In some embodiments, the concentration of the anti-TIGIT monoclonal antibody is about 20 mg/mL to about 30 mg/mL. In some embodiments, the concentration of the anti-TIGIT monoclonal antibody is about 20 mg/mL to about 25 mg/mL. In some embodiments, the concentration of the anti-TIGIT monoclonal antibody is about 25 mg/mL to about 45 mg/mL. In some embodiments, the concentration of the anti-TIGIT monoclonal antibody is about 30 mg/mL to about 45 mg/mL. In some embodiments, the concentration of the anti-TIGIT monoclonal antibody is about 35 mg/mL to about 45 mg/mL. In some embodiments, the concentration of the anti-TIGIT monoclonal antibody is about 45 mg/mL to about 75 mg/mL. In some embodiments, the concentration of the anti-TIGIT monoclonal antibody is about 50 mg/mL to about 70 mg/mL. In some embodiments, the concentration of the anti-TIGIT monoclonal antibody is about 55 mg/mL to about 65 mg/mL. In some embodiments, the concentration of the anti-TIGIT monoclonal antibody is about 60 mg/mL to about 65 mg/mL. In some embodiments, the concentration of the anti-TIGIT monoclonal antibody is about 55 mg/mL to about 60 mg/mL.

In some embodiments, the concentration of the anti-TIGIT monoclonal antibody is about 20 mg/mL to about 180 mg/mL. In some embodiments, the concentration of the anti-TIGIT monoclonal antibody is about 20 mg/mL to about 160 mg/mL. In some embodiments, the concentration of the anti-TIGIT monoclonal antibody is about 20 mg/mL to about 140 mg/mL. In some embodiments, the concentration of the anti-TIGIT monoclonal antibody is about 20 mg/mL to about 120 mg/mL. In some embodiments, the concentration of the anti-TIGIT monoclonal antibody is about 20 mg/mL to about 100 mg/mL. In some embodiments, the concentration of the anti-TIGIT monoclonal antibody is about 20 mg/mL to about 80 mg/mL. In some embodiments, the concentration of the anti-TIGIT monoclonal antibody is about 20 mg/mL to about 60 mg/mL. In some embodiments, the concentration of the anti-TIGIT monoclonal antibody is about 20 mg/mL to about 40 mg/mL. In some embodiments, the concentration of the anti-TIGIT monoclonal antibody is about 40 mg/mL to about 180 mg/mL. In some embodiments, the concentration of the anti-TIGIT monoclonal antibody is about 60 mg/mL to about 180 mg/mL. In some embodiments, the concentration of the anti-TIGIT monoclonal antibody is about 80 mg/mL to about 180 mg/mL. In some embodiments, the concentration of the anti-TIGIT monoclonal antibody is about 100 mg/mL to about 180 mg/mL. In some embodiments, the concentration of the anti-TIGIT monoclonal antibody is about 120 mg/mL to about 180 mg/mL. In some embodiments, the concentration of the anti-TIGIT monoclonal antibody is about 140 mg/mL to about 180 mg/mL. In some embodiments, the concentration of the anti-TIGIT monoclonal antibody is about 160 mg/mL to about 180 mg/mL. In some embodiments, the concentration of the anti-TIGIT monoclonal antibody is about 30 mg/mL to about 50 mg/mL. In some embodiments, the concentration of the anti-TIGIT monoclonal antibody is about 40 mg/mL to about 160 mg/mL. In some embodiments, the concentration of the anti-TIGIT monoclonal antibody is about 40 mg/mL to about 140 mg/mL. In some embodiments, the concentration of the anti-TIGIT monoclonal antibody is about 40 mg/mL to about 120 mg/mL. In some embodiments, the concentration of the anti-TIGIT monoclonal antibody is about 40 mg/mL to about 100 mg/mL. In some embodiments, the concentration of the anti-TIGIT monoclonal antibody is about 40 mg/mL to about 80 mg/mL. In some embodiments, the concentration of the anti-TIGIT monoclonal antibody is about 40 mg/mL to about 60 mg/mL. In some embodiments, the concentration of the anti-TIGIT monoclonal antibody is about 50 mg/mL to about 70 mg/mL. In some embodiments, the concentration of the anti-TIGIT monoclonal antibody is about 150 mg/mL to about 170 mg/mL.

In some embodiments of any of the above formulations, the concentration of the anti-TIGIT monoclonal antibody is about 18 mg/mL to about 50 mg/mL. In some embodiments, the concentration of the anti-TIGIT monoclonal antibody is about 19 mg/mL to about 50 mg/mL. In some embodiments, the concentration of the anti-TIGIT monoclonal antibody is about 20 mg/mL to about 50 mg/mL. In some embodiments, the concentration of the anti-TIGIT monoclonal antibody is about 21 mg/mL to about 50 mg/mL. In some embodiments, the concentration of the anti-TIGIT monoclonal antibody is about 22 mg/mL to about 50 mg/mL. In some embodiments, the concentration of the anti-TIGIT monoclonal antibody is about 23 mg/mL to about 50 mg/mL. In some embodiments, the concentration of the anti-TIGIT monoclonal antibody is about 24 mg/mL to about 50 mg/mL. In some embodiments, the concentration of the anti-TIGIT monoclonal antibody is about 25 mg/mL to about 50 mg/mL. In some embodiments, the concentration of the anti-TIGIT monoclonal antibody is about 26 mg/mL to about 50 mg/mL. In some embodiments, the concentration of the anti-TIGIT monoclonal antibody is about 27 mg/mL to about 50 mg/mL. In some embodiments, the concentration of the anti-TIGIT monoclonal antibody is about 28 mg/mL to about 50 mg/mL. In some embodiments, the concentration of the anti-TIGIT monoclonal antibody is about 29 mg/mL to about 50 mg/mL. In some embodiments, the concentration of the anti-TIGIT monoclonal antibody is about 30 mg/mL to about 50 mg/mL. In some embodiments, the concentration of the anti-TIGIT monoclonal antibody is about 31 mg/mL to about 50 mg/mL. In some embodiments, the concentration of the anti-TIGIT monoclonal antibody is about 32 mg/mL to about 50 mg/mL. In some embodiments, the concentration of the anti-TIGIT monoclonal antibody is about 33 mg/mL to about 50 mg/mL. In some embodiments, the concentration of the anti-TIGIT monoclonal antibody is about 34 mg/ml to about 50 mg/mL. In some embodiments, the concentration of the anti-TIGIT monoclonal antibody is about 35 mg/mL to about 50 mg/mL. In some embodiments, the concentration of the anti-TIGIT monoclonal antibody is about 36 mg/mL to about 50 mg/mL. In some embodiments, the concentration of the anti-TIGIT monoclonal antibody is about 37 mg/mL to about 50 mg/mL. In some embodiments, the concentration of the anti-TIGIT monoclonal antibody is about 38 mg/mL to about 50 mg/mL. In some embodiments, the concentration of the anti-TIGIT monoclonal antibody is about 39 mg/mL to about 50 mg/mL. In some embodiments, the concentration of the anti-TIGIT monoclonal antibody is about 40 mg/mL to about 50 mg/mL.

In some embodiments, the concentration of the anti-TIGIT monoclonal antibody is about 20 mg/mL. In some embodiments, the concentration of the anti-TIGIT monoclonal antibody is about 25 mg/mL. In some embodiments, the concentration of the anti-TIGIT monoclonal antibody is about 30 mg/mL. In some embodiments, the concentration of the anti-TIGIT monoclonal antibody is about 35 mg/mL. In particular embodiments, the concentration of the anti-TIGIT monoclonal antibody is about 40 mg/mL. In some embodiments, the concentration of the anti-TIGIT monoclonal antibody is about 45 mg/mL. In some embodiments, the concentration of the anti-TIGIT monoclonal antibody is about 50 mg/mL. In some embodiments, the concentration of the anti-TIGIT monoclonal antibody is about 55 mg/mL. In some embodiments, the concentration of the anti-TIGIT monoclonal antibody is about 60 mg/mL. In some embodiments, the concentration of the anti-TIGIT monoclonal antibody is about 65 mg/mL. In some embodiments, the concentration of the anti-TIGIT monoclonal antibody is about 70 mg/mL. In some embodiments, the concentration of the anti-TIGIT monoclonal antibody is about 75 mg/mL. In some embodiments, the concentration of the anti-TIGIT monoclonal antibody is about 80 mg/mL. In some embodiments, the concentration of the anti-TIGIT monoclonal antibody is about 85 mg/mL. In some embodiments, the concentration of the anti-TIGIT monoclonal antibody is about 90 mg/mL. In some embodiments, the concentration of the anti-TIGIT monoclonal antibody is about 95 mg/mL. In some embodiments, the concentration of the anti-TIGIT monoclonal antibody is about 100 mg/mL. In some embodiments, the concentration of the anti-TIGIT monoclonal antibody is about 105 mg/mL. In some embodiments, the concentration of the anti-TIGIT monoclonal antibody is about 110 mg/mL. In some embodiments, the concentration of the anti-TIGIT monoclonal antibody is about 115 mg/mL. In some embodiments, the concentration of the anti-TIGIT monoclonal antibody is about 120 mg/mL. In some embodiments, the concentration of the anti-TIGIT monoclonal antibody is about 125 mg/mL. In some embodiments, the concentration of the anti-TIGIT monoclonal antibody is about 130 mg/mL. In some embodiments, the concentration of the anti-TIGIT monoclonal antibody is about 135 mg/mL. In some embodiments, the concentration of the anti-TIGIT monoclonal antibody is about 140 mg/mL. In some embodiments, the concentration of the anti-TIGIT monoclonal antibody is about 145 mg/mL. In some embodiments, the concentration of the anti-TIGIT monoclonal antibody is about 150 mg/mL. In some embodiments, the concentration of the anti-TIGIT monoclonal antibody is about 155 mg/mL. In particular embodiments, the concentration of the anti-TIGIT monoclonal antibody is about 160 mg/mL. In some embodiments, the concentration of the anti-TIGIT monoclonal antibody is about 165 mg/mL. In some embodiments, the concentration of the anti-TIGIT monoclonal antibody is about 170 mg/mL. In particular embodiments, the concentration of the anti-TIGIT monoclonal antibody is about 175 mg/mL. In some embodiments, the concentration of the anti-TIGIT monoclonal antibody is about 180 mg/mL. In some embodiments, the concentration of the anti-TIGIT monoclonal antibody is about 185 mg/mL. In some embodiments, the concentration of the anti-TIGIT monoclonal antibody is about 190 mg/mL. In some embodiments, the concentration of the anti-TIGIT monoclonal antibody is about 195 mg/mL. In some embodiments, the concentration of the anti-TIGIT monoclonal antibody is about 200 mg/mL.

In some embodiments of any of the above formulations, the concentration of the anti-TIGIT monoclonal antibody is about 31 mg/mL to about 49 mg/mL. In some embodiments, the concentration of the anti-TIGIT monoclonal antibody is about 32 mg/mL to about 48 mg/mL. In some embodiments, the concentration of the anti-TIGIT monoclonal antibody is about 33 mg/ml to about 47 mg/mL. In some embodiments, the concentration of the anti-TIGIT monoclonal antibody is about 34 mg/mL to about 46 mg/mL. In some embodiments, the concentration of the anti-TIGIT monoclonal antibody is about 35 mg/mL to about 45 mg/mL. In some embodiments, the concentration of the anti-TIGIT monoclonal antibody is about 36 mg/mL to about 44 mg/mL. In some embodiments, the concentration of the anti-TIGIT monoclonal antibody is about 37 mg/mL to about 43 mg/mL. In some embodiments, the concentration of the anti-TIGIT monoclonal antibody is about 38 mg/mL to about 42 mg/mL. In some embodiments, the concentration of the anti-TIGIT monoclonal antibody is about 39 mg/mL to about 41 mg/mL.

In some embodiments, the concentration of the anti-TIGIT monoclonal antibody is about 18 mg/mL. In some embodiments, the concentration of the anti-TIGIT monoclonal antibody is about 20 mg/mL. In some embodiments, the concentration of the anti-TIGIT monoclonal antibody is about 23 mg/mL. In some embodiments, the concentration of the anti-TIGIT monoclonal antibody is about 25 mg/mL. In some embodiments, the concentration of the anti-TIGIT monoclonal antibody is about 30 mg/mL. In some embodiments, the concentration of the anti-TIGIT monoclonal antibody is about 32 mg/mL. In some embodiments, the concentration of the anti-TIGIT monoclonal antibody is about 35 mg/mL. In some embodiments, the concentration of the anti-TIGIT monoclonal antibody is about 37 mg/mL. In particular embodiments, the concentration of the anti-TIGIT monoclonal antibody is about 40 mg/mL. In some embodiments, the concentration of the anti-TIGIT monoclonal antibody is about 43 mg/mL. In some embodiments, the concentration of the anti-TIGIT monoclonal antibody is about 45 mg/mL. In some embodiments, the concentration of the anti-TIGIT monoclonal antibody is about 48 mg/mL. In some embodiments, the concentration of the anti-TIGIT monoclonal antibody is about 50 mg/mL. In some embodiments, the concentration of the anti-TIGIT monoclonal antibody is about 53 mg/mL. In some embodiments, the concentration of the anti-TIGIT monoclonal antibody is about 55 mg/mL. In some embodiments, the concentration of the anti-TIGIT monoclonal antibody is about 60 mg/mL. In some embodiments, the concentration of the anti-TIGIT monoclonal antibody is about 65 mg/mL. In some embodiments, the concentration of the anti-TIGIT monoclonal antibody is about 70 mg/mL. In some embodiments, the concentration of the anti-TIGIT monoclonal antibody is about 75 mg/mL.

In some embodiments, the concentration of the anti-TIGIT monoclonal antibody is 20 mg/mL to 75 mg/mL. In some embodiments, the concentration of the anti-TIGIT monoclonal antibody is 25 mg/mL to 75 mg/mL. In some embodiments, the concentration of the anti-TIGIT monoclonal antibody is 30 mg/mL to 75 mg/mL. In some embodiments, the concentration of the anti-TIGIT monoclonal antibody is 35 mg/mL to 75 mg/mL. In some embodiments, the concentration of the anti-TIGIT monoclonal antibody is 40 mg/mL to 75 mg/mL. In some embodiments, the concentration of the anti-TIGIT monoclonal antibody is 45 mg/mL to 75 mg/mL. In some embodiments, the concentration of the anti-TIGIT monoclonal antibody is 50 mg/mL to 75 mg/mL. In some embodiments, the concentration of the anti-TIGIT monoclonal antibody is 55 mg/mL to 75 mg/mL. In some embodiments, the concentration of the anti-TIGIT monoclonal antibody is 60 mg/mL to 75 mg/mL. In some embodiments, the concentration of the anti-TIGIT monoclonal antibody is 65 mg/mL to 75 mg/mL. In some embodiments, the concentration of the anti-TIGIT monoclonal antibody is 70 mg/mL to 75 mg/mL. In some embodiments, the concentration of the anti-TIGIT monoclonal antibody is 20 mg/mL to 70 mg/mL. In some embodiments, the concentration of the anti-TIGIT monoclonal antibody is 20 mg/mL to 65 mg/mL. In some embodiments, the concentration of the anti-TIGIT monoclonal antibody is 20 mg/mL to 60 mg/mL. In some embodiments, the concentration of the anti-TIGIT monoclonal antibody is 20 mg/mL to 55 mg/mL. In some embodiments, the concentration of the anti-TIGIT monoclonal antibody is 20 mg/mL to 50 mg/mL. In some embodiments, the concentration of the anti-TIGIT monoclonal antibody is 20 mg/mL to 45 mg/mL. In some embodiments, the concentration of the anti-TIGIT monoclonal antibody is 20 mg/mL to 40 mg/mL. In some embodiments, the concentration of the anti-TIGIT monoclonal antibody is 20 mg/mL to 35 mg/mL. In some embodiments, the concentration of the anti-TIGIT monoclonal antibody is 20 mg/mL to 30 mg/mL. In some embodiments, the concentration of the anti-TIGIT monoclonal antibody is 20 mg/mL to 25 mg/mL. In some embodiments, the concentration of the anti-TIGIT monoclonal antibody is 25 mg/mL to 45 mg/mL. In some embodiments, the concentration of the anti-TIGIT monoclonal antibody is 30 mg/mL to 45 mg/mL. In some embodiments, the concentration of the anti-TIGIT monoclonal antibody is 35 mg/mL to 45 mg/mL. In some embodiments, the concentration of the anti-TIGIT monoclonal antibody is 45 mg/mL to 75 mg/mL. In some embodiments, the concentration of the anti-TIGIT monoclonal antibody is 50 mg/mL to 70 mg/mL. In some embodiments, the concentration of the anti-TIGIT monoclonal antibody is 55 mg/mL to 65 mg/mL. In some embodiments, the concentration of the anti-TIGIT monoclonal antibody is 60 mg/mL to 65 mg/mL. In some embodiments, the concentration of the anti-TIGIT monoclonal antibody is 55 mg/mL to 60 mg/mL.

In some embodiments, the concentration of the anti-TIGIT monoclonal antibody is 20 mg/mL to 180 mg/mL. In some embodiments, the concentration of the anti-TIGIT monoclonal antibody is 20 mg/mL to 160 mg/mL. In some embodiments, the concentration of the anti-TIGIT monoclonal antibody is 20 mg/mL to 140 mg/mL. In some embodiments, the concentration of the anti-TIGIT monoclonal antibody is 20 mg/mL to 120 mg/mL. In some embodiments, the concentration of the anti-TIGIT monoclonal antibody is 20 mg/mL to 100 mg/mL. In some embodiments, the concentration of the anti-TIGIT monoclonal antibody is 20 mg/mL to 80 mg/mL. In some embodiments, the concentration of the anti-TIGIT monoclonal antibody is 20 mg/mL to 60 mg/mL. In some embodiments, the concentration of the anti-TIGIT monoclonal antibody is 20 mg/mL to 40 mg/mL. In some embodiments, the concentration of the anti-TIGIT monoclonal antibody is 40 mg/mL to 180 mg/mL. In some embodiments, the concentration of the anti-TIGIT monoclonal antibody is 60 mg/mL to 180 mg/mL. In some embodiments, the concentration of the anti-TIGIT monoclonal antibody is 80 mg/mL to 180 mg/mL. In some embodiments, the concentration of the anti-TIGIT monoclonal antibody is 100 mg/mL to 180 mg/mL. In some embodiments, the concentration of the anti-TIGIT monoclonal antibody is 120 mg/mL to 180 mg/mL. In some embodiments, the concentration of the anti-TIGIT monoclonal antibody is 140 mg/mL to 180 mg/mL. In some embodiments, the concentration of the anti-TIGIT monoclonal antibody is 160 mg/mL to 180 mg/mL. In some embodiments, the concentration of the anti-TIGIT monoclonal antibody is 30 mg/mL to 50 mg/mL. In some embodiments, the concentration of the anti-TIGIT monoclonal antibody is 40 mg/mL to 160 mg/mL. In some embodiments, the concentration of the anti-TIGIT monoclonal antibody is 40 mg/mL to 140 mg/mL. In some embodiments, the concentration of the anti-TIGIT monoclonal antibody is 40 mg/mL to 120 mg/mL. In some embodiments, the concentration of the anti-TIGIT monoclonal antibody is 40 mg/mL to 100 mg/mL. In some embodiments, the concentration of the anti-TIGIT monoclonal antibody is 40 mg/mL to 80 mg/mL. In some embodiments, the concentration of the anti-TIGIT monoclonal antibody is 40 mg/mL to 60 mg/mL. In some embodiments, the concentration of the anti-TIGIT monoclonal antibody is 50 mg/mL to 70 mg/mL. In some embodiments, the concentration of the anti-TIGIT monoclonal antibody is 150 mg/mL to 170 mg/mL.

In some embodiments of any of the above formulations, the concentration of the anti-TIGIT monoclonal antibody is 18 mg/mL to 50 mg/mL. In some embodiments, the concentration of the anti-TIGIT monoclonal antibody is 19 mg/mL to 50 mg/mL. In some embodiments, the concentration of the anti-TIGIT monoclonal antibody is 20 mg/mL to 50 mg/mL. In some embodiments, the concentration of the anti-TIGIT monoclonal antibody is 21 mg/mL to 50 mg/mL. In some embodiments, the concentration of the anti-TIGIT monoclonal antibody is 22 mg/mL to 50 mg/mL. In some embodiments, the concentration of the anti-TIGIT monoclonal antibody is 23 mg/mL to 50 mg/mL. In some embodiments, the concentration of the anti-TIGIT monoclonal antibody is 24 mg/mL to 50 mg/mL. In some embodiments, the concentration of the anti-TIGIT monoclonal antibody is 25 mg/mL to 50 mg/mL. In some embodiments, the concentration of the anti-TIGIT monoclonal antibody is 26 mg/mL to 50 mg/mL. In some embodiments, the concentration of the anti-TIGIT monoclonal antibody is 27 mg/mL to 50 mg/mL. In some embodiments, the concentration of the anti-TIGIT monoclonal antibody is 28 mg/mL to 50 mg/mL. In some embodiments, the concentration of the anti-TIGIT monoclonal antibody is 29 mg/mL to 50 mg/mL. In some embodiments, the concentration of the anti-TIGIT monoclonal antibody is 30 mg/mL to 50 mg/mL. In some embodiments, the concentration of the anti-TIGIT monoclonal antibody is 31 mg/mL to 50 mg/mL. In some embodiments, the concentration of the anti-TIGIT monoclonal antibody is 32 mg/mL to 50 mg/mL. In some embodiments, the concentration of the anti-TIGIT monoclonal antibody is about 33 mg/mL to 50 mg/mL. In some embodiments, the concentration of the anti-TIGIT monoclonal antibody is 34 mg/mL to 50 mg/mL. In some embodiments, the concentration of the anti-TIGIT monoclonal antibody is 35 mg/mL to 50 mg/mL. In some embodiments, the concentration of the anti-TIGIT monoclonal antibody is 36 mg/mL to 50 mg/mL. In some embodiments, the concentration of the anti-TIGIT monoclonal antibody is 37 mg/mL to 50 mg/mL. In some embodiments, the concentration of the anti-TIGIT monoclonal antibody is 38 mg/mL to 50 mg/mL. In some embodiments, the concentration of the anti-TIGIT monoclonal antibody is 39 mg/mL to 50 mg/mL. In some embodiments, the concentration of the anti-TIGIT monoclonal antibody is 40 mg/mL to 50 mg/mL.

In some embodiments of any of the above formulations, the concentration of the anti-TIGIT monoclonal antibody is 31 mg/mL to 49 mg/mL. In some embodiments, the concentration of the anti-TIGIT monoclonal antibody is 32 mg/mL to 48 mg/mL. In some embodiments, the concentration of the anti-TIGIT monoclonal antibody is 33 mg/mL to 47 mg/mL. In some embodiments, the concentration of the anti-TIGIT monoclonal antibody is 34 mg/mL to 46 mg/mL. In some embodiments, the concentration of the anti-TIGIT monoclonal antibody is 35 mg/mL to 45 mg/mL. In some embodiments, the concentration of the anti-TIGIT monoclonal antibody is 36 mg/mL to 44 mg/mL. In some embodiments, the concentration of the anti-TIGIT monoclonal antibody is 37 mg/mL to 43 mg/mL. In some embodiments, the concentration of the anti-TIGIT monoclonal antibody is 38 mg/mL to 42 mg/mL. In some embodiments, the concentration of the anti-TIGIT monoclonal antibody is 39 mg/mL to 41 mg/mL.

In some embodiments, the concentration of the anti-TIGIT monoclonal antibody is 18 mg/mL. In some embodiments, the concentration of the anti-TIGIT monoclonal antibody is 20 mg/mL. In some embodiments, the concentration of the anti-TIGIT monoclonal antibody is 23 mg/mL. In some embodiments, the concentration of the anti-TIGIT monoclonal antibody is 25 mg/mL. In some embodiments, the concentration of the anti-TIGIT monoclonal antibody is 28 mg/mL. In some embodiments, the concentration of the anti-TIGIT monoclonal antibody is 30 mg/mL. In some embodiments, the concentration of the anti-TIGIT monoclonal antibody is 32 mg/mL. In some embodiments, the concentration of the anti-TIGIT monoclonal antibody is 35 mg/mL. In some embodiments, the concentration of the anti-TIGIT monoclonal antibody is 37 mg/mL. In particular embodiments, the concentration of the anti-TIGIT monoclonal antibody is 40 mg/mL. In some embodiments, the concentration of the anti-TIGIT monoclonal antibody is 43 mg/mL. In some embodiments, the concentration of the anti-TIGIT monoclonal antibody is 45 mg/mL. In some embodiments, the concentration of the anti-TIGIT monoclonal antibody is 48 mg/mL. In some embodiments, the concentration of the anti-TIGIT monoclonal antibody is 50 mg/mL. In some embodiments, the concentration of the anti-TIGIT monoclonal antibody is 53 mg/mL. In some embodiments, the concentration of the anti-TIGIT monoclonal antibody is 55 mg/mL. In some embodiments, the concentration of the anti-TIGIT monoclonal antibody is 60 mg/mL. In some embodiments, the concentration of the anti-TIGIT monoclonal antibody is 65 mg/mL. In some embodiments, the concentration of the anti-TIGIT monoclonal antibody is 70 mg/mL. In some embodiments, the concentration of the anti-TIGIT monoclonal antibody is 75 mg/mL.

In some embodiments, the concentration of the anti-TIGIT monoclonal antibody is 20 mg/mL. In some embodiments, the concentration of the anti-TIGIT monoclonal antibody is 25 mg/mL. In some embodiments, the concentration of the anti-TIGIT monoclonal antibody is 30 mg/mL. In some embodiments, the concentration of the anti-TIGIT monoclonal antibody is 35 mg/mL. In particular embodiments, the concentration of the anti-TIGIT monoclonal antibody is 40 mg/mL. In some embodiments, the concentration of the anti-TIGIT monoclonal antibody is 45 mg/mL. In some embodiments, the concentration of the anti-TIGIT monoclonal antibody is 50 mg/mL. In some embodiments, the concentration of the anti-TIGIT monoclonal antibody is 55 mg/mL. In some embodiments, the concentration of the anti-TIGIT monoclonal antibody is 60 mg/mL. In some embodiments, the concentration of the anti-TIGIT monoclonal antibody is 65 mg/mL. In some embodiments, the concentration of the anti-TIGIT monoclonal antibody is 70 mg/mL. In some embodiments, the concentration of the anti-TIGIT monoclonal antibody is 75 mg/mL. In some embodiments, the concentration of the anti-TIGIT monoclonal antibody is 80 mg/mL. In some embodiments, the concentration of the anti-TIGIT monoclonal antibody is 85 mg/mL. In some embodiments, the concentration of the anti-TIGIT monoclonal antibody is 90 mg/mL. In some embodiments, the concentration of the anti-TIGIT monoclonal antibody is 95 mg/mL. In some embodiments, the concentration of the anti-TIGIT monoclonal antibody is 100 mg/mL. In some embodiments, the concentration of the anti-TIGIT monoclonal antibody is 105 mg/mL. In some embodiments, the concentration of the anti-TIGIT monoclonal antibody is 110 mg/mL. In some embodiments, the concentration of the anti-TIGIT monoclonal antibody is 115 mg/mL. In some embodiments, the concentration of the anti-TIGIT monoclonal antibody is 120 mg/mL. In some embodiments, the concentration of the anti-TIGIT monoclonal antibody is 125 mg/mL. In some embodiments, the concentration of the anti-TIGIT monoclonal antibody is 130 mg/mL. In some embodiments, the concentration of the anti-TIGIT monoclonal antibody is 135 mg/mL. In some embodiments, the concentration of the anti-TIGIT monoclonal antibody is 140 mg/mL. In some embodiments, the concentration of the anti-TIGIT monoclonal antibody is 145 mg/mL. In some embodiments, the concentration of the anti-TIGIT monoclonal antibody is 150 mg/mL. In some embodiments, the concentration of the anti-TIGIT monoclonal antibody is 155 mg/mL. In particular embodiments, the concentration of the anti-TIGIT monoclonal antibody is 160 mg/mL. In some embodiments, the concentration of the anti-TIGIT monoclonal antibody is 165 mg/mL. In some embodiments, the concentration of the anti-TIGIT monoclonal antibody is 170 mg/mL. In some embodiments, the concentration of the anti-TIGIT monoclonal antibody is 175 mg/mL. In particular embodiments, the concentration of the anti-TIGIT monoclonal antibody is 176 mg/mL. In some embodiments, the concentration of the anti-TIGIT monoclonal antibody is 180 mg/mL. In some embodiments, the concentration of the anti-TIGIT monoclonal antibody is 185 mg/mL. In some embodiments, the concentration of the anti-TIGIT monoclonal antibody is 190 mg/mL. In some embodiments, the concentration of the anti-TIGIT monoclonal antibody is 195 mg/mL. In some embodiments, the concentration of the anti-TIGIT monoclonal antibody is 200 mg/mL.

In some embodiments, the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments, the anti-PD-L1 monoclonal antibody comprises a heavy chain variable region comprising the amino acid sequence of SEQ ID NO: 16. In some embodiments, the anti-PD-L1 monoclonal antibody comprises a light chain variable region comprising the amino acid sequence of SEQ ID NO: 17. In some embodiments, the anti-PD-L1 monoclonal antibody comprises a heavy chain variable region comprising the amino acid sequence of SEQ ID NO: 16 and a light chain variable region comprising the amino acid sequence of SEQ ID NO: 17. In some embodiments of any of the above aspects, the heavy chain of the anti-PD-L1 monoclonal antibody comprises the amino acid sequence of SEQ ID NO: 21. In some embodiments of any of the above aspects, the heavy chain of the anti-PD-L1 monoclonal antibody comprises the amino acid sequence of SEQ ID NO: 22. In some embodiments of any of the above aspects, the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23. In some embodiments, the anti-PD-L1 monoclonal monoclonal antibody is atezolizumab. In some embodiments, the anti-PD-L1 monoclonal antibody is atezolizumab.

In some embodiments, the anti-PD-L1 monoclonal antibody is an IgG antibody. The anti-PD-L1 monoclonal antibody may be an IgG1 antibody, an IgG2 antibody, an IgG3 antibody, or an IgG4 antibody. In some embodiments, the anti-PD-L1 monoclonal antibody is an IgG1 or an IgG4 antibody. In some embodiments, the anti-PD-L1 monoclonal antibody is an IgG1 antibody. In some embodiments, the anti-PD-L1 monoclonal antibody is an IgG4 antibody. In some embodiments, the anti-PD-L1 monoclonal antibody is an antagonist antibody. In some cases, the anti-PD-L1 monoclonal antibody may have one or more effector functions. In some embodiments, the anti-PD-L1 monoclonal antibody retains all effector functions. Optionally, one or more effector functions of the anti-PD-L1 monoclonal antibody may have been modified or eliminated.

In some embodiments, the anti-PD-L1 monoclonal antibody is a human antibody. In some embodiments, the anti-PD-L1 monoclonal antibody is a humanized antibody. In some embodiments, the anti-PD-L1 monoclonal antibody is a full-length antibody. In some embodiments, the anti-PD-L1 monoclonal antibody is a full-length IgG antibody. In some embodiments, the anti-PD-L1 monoclonal antibody is a full-length IgG1 antibody. In some embodiments, the anti-PD-L1 monoclonal antibody is a full-length human IgG1 antibody. In some embodiments, the anti-PD-L1 monoclonal antibody is a full-length humanized IgG1 antibody. The anti-PD-L1 monoclonal antibody may be an antibody fragment. In some embodiments, the anti-PD-L1 monoclonal antibody is a Fab, a Fab′, a F(ab′)₂, a Fv, or a scFv fragment. In some embodiments, the anti-PD-L1 monoclonal antibody is a Fab fragment. In some embodiments, the anti-PD-L1 monoclonal antibody is a Fab′ fragment. In some embodiments, the anti-PD-L1 monoclonal antibody is a F(ab′)₂, fragment. In some embodiments, the anti-PD-L1 monoclonal antibody is a Fv fragment. In some embodiments, the anti-PD-L1 monoclonal antibody is a scFv fragment. In some embodiments, the anti-PD-L1 monoclonal antibody is a diabody. In some embodiments, the anti-PD-L1 monoclonal antibody is a linear antibody. In some embodiments, the anti-PD-L1 monoclonal antibody is a single chain antibody molecule. In some embodiments, the anti-PD-L1 monoclonal antibody is a multispecific antibody, e.g. formed from antibody fragments. In some embodiments, the anti-PD-L1 monoclonal antibody is atezolizumab, marketed as TECENTRIQ®. Atezolizumab is described in WHO Drug Information (International Nonproprietary Names for Pharmaceutical Substances), Proposed INN: List 112, Vol. 28, No. 4, 2014 (see page 488). In some embodiments, atezolizumab has the CAS Registry Number 1380723-44-3.

In some embodiments, the concentration of the anti-PD-L1 monoclonal antibody is about 60 mg/mL to about 100 mg/mL. In some embodiments, the concentration of the anti-PD-L1 monoclonal antibody is about 60 mg/mL to about 90 mg/mL. In some embodiments, the concentration of the anti-PD-L1 monoclonal antibody is about 60 mg/mL to about 80 mg/mL. In some embodiments, the concentration of the anti-PD-L1 monoclonal antibody is about 60 mg/mL to about 70 mg/mL. In some embodiments, the concentration of the anti-PD-L1 monoclonal antibody is about 70 mg/mL to about 100 mg/mL. In some embodiments, the concentration of the anti-PD-L1 monoclonal antibody is about 80 mg/mL to about 100 mg/mL. In some embodiments, the concentration of the anti-PD-L1 monoclonal antibody is about 90 mg/mL to about 100 mg/mL. In some embodiments, the concentration of the anti-PD-L1 monoclonal antibody is about 70 mg/mL to about 90 mg/mL. In some embodiments, the concentration of the anti-PD-L1 monoclonal antibody is about 70 mg/mL to about 80 mg/mL. In some embodiments, the concentration of the anti-PD-L1 monoclonal antibody is about 65 mg/mL to about 95 mg/mL. In some embodiments, the concentration of the anti-PD-L1 monoclonal antibody is about 75 mg/mL to about 85 mg/mL.

In some embodiments, the concentration of the anti-PD-L1 monoclonal antibody is about 60 mg/mL. In some embodiments, the concentration of the anti-PD-L1 monoclonal antibody is about 65 mg/mL. In some embodiments, the concentration of the anti-PD-L1 monoclonal antibody is about 70 mg/mL. In some embodiments, the concentration of the anti-PD-L1 monoclonal antibody is about 75 mg/mL. In particular embodiments, the concentration of the anti-PD-L1 monoclonal antibody is about 80 mg/mL. In some embodiments, the concentration of the anti-PD-L1 monoclonal antibody is about 85 mg/mL. In some embodiments, the concentration of the anti-PD-L1 monoclonal antibody is about 90 mg/mL. In some embodiments, the concentration of the anti-PD-L1 monoclonal antibody is about 95 mg/mL. In some embodiments, the concentration of the anti-PD-L1 monoclonal antibody is about 100 mg/mL.

In some embodiments, the concentration of the anti-PD-L1 monoclonal antibody is 60 mg/mL to 100 mg/mL. In some embodiments, the concentration of the anti-PD-L1 monoclonal antibody is 60 mg/mL to 90 mg/mL. In some embodiments, the concentration of the anti-PD-L1 monoclonal antibody is 60 mg/mL to 80 mg/mL. In some embodiments, the concentration of the anti-PD-L1 monoclonal antibody is 60 mg/mL to 70 mg/mL. In some embodiments, the concentration of the anti-PD-L1 monoclonal antibody is 70 mg/mL to 100 mg/mL. In some embodiments, the concentration of the anti-PD-L1 monoclonal antibody is 80 mg/mL to 100 mg/mL. In some embodiments, the concentration of the anti-PD-L1 monoclonal antibody is 90 mg/mL to 100 mg/mL. In some embodiments, the concentration of the anti-PD-L1 monoclonal antibody is 70 mg/mL to 90 mg/mL. In some embodiments, the concentration of the anti-PD-L1 monoclonal antibody is 70 mg/mL to 80 mg/mL. In some embodiments, the concentration of the anti-PD-L1 monoclonal antibody is 65 mg/mL to 95 mg/mL. In some embodiments, the concentration of the anti-PD-L1 monoclonal antibody is 75 mg/mL to 85 mg/mL.

In some embodiments, the concentration of the anti-PD-L1 monoclonal antibody is 60 mg/mL. In some embodiments, the concentration of the anti-PD-L1 monoclonal antibody is 65 mg/mL. In some embodiments, the concentration of the anti-PD-L1 monoclonal antibody is 70 mg/mL. In some embodiments, the concentration of the anti-PD-L1 monoclonal antibody is 75 mg/mL. In particular embodiments, the concentration of the anti-PD-L1 monoclonal antibody is 80 mg/mL. In some embodiments, the concentration of the anti-PD-L1 monoclonal antibody is 85 mg/mL. In some embodiments, the concentration of the anti-PD-L1 monoclonal antibody is 90 mg/mL. In some embodiments, the concentration of the anti-PD-L1 monoclonal antibody is 95 mg/mL. In some embodiments, the concentration of the anti-PD-L1 monoclonal antibody is 100 mg/mL.

The liquid pharmaceutical formation of the disclosure comprises a buffer. In some embodiments, the buffer is an acidic salt. In some embodiments, the buffer comprises histidine, arginine, acetate, citrate, succinate, gluconate, phosphate, or a combination thereof. In some embodiments, the buffer comprises histidine, arginine, acetate, citrate, succinate, gluconate, phosphate, or an acidic salt form thereof. In some embodiments, the buffer is selected from the group consisting of histidine, acetate, histidine acetate, histidine hydrochloride, histidine acetate and arginine, citrate, citric acid, sodium acetate, sodium citrate, arginine succinate, phosphate, disodium phosphate dihydrate, and sodium dihydrogen phosphate dihydrate. In some embodiments, the buffer is histidine. In some embodiments, the buffer is acetate. In some embodiments, the buffer is histidine acetate. In some embodiments, the buffer is histidine hydrochloride. In some embodiments, the buffer is histidine acetate and arginine. In some embodiments, the buffer is citrate. In some embodiments, the buffer is citric acid. In some embodiments, the buffer is sodium acetate. In some embodiments, the buffer is sodium citrate. In some embodiments, the buffer is arginine succinate. In some embodiments, the buffer is phosphate. In some embodiments, the buffer is disodium phosphate dihydrate. In some embodiments, the buffer is sodium dihydrogen phosphate dihydrate.

The skilled artisan would recognize that, depending on the other components and the pH of the composition, a buffer that is added as a salt form may be converted (or partially converted) to the acid form (and vice versa) and, therefore, exist in the composition as either the salt form, the acid form, or a mixture of both. For example, if sodium citrate is added to a composition, in solution, it may remain in citrate form, be converted to citric acid, or be present as a mixture of citrate and citric acid forms. Similarly, if citric acid is added to a composition, in solution, it may remain in citric acid form, be converted to a citrate salt, or be present as a mixture of the citric acid and citrate forms. Accordingly, when determining the concentration of the buffer in a composition, the skilled artisan would consider both the salt and acid forms in determining the buffer concentration. For example, if a composition includes a certain concentration of a citrate salt, the skilled artisan would include both the citrate salt and the citric acid in the composition when determining the buffer concentration.

In some embodiments, the concentration of the buffer is about 10 mM to about 30 mM. In some embodiments, the concentration of the buffer is about 15 mM to about 25 mM. In some embodiments, the concentration of the buffer is about 12 mM to about 28 mM. In some embodiments, the concentration of the buffer is about 10 mM. In some embodiments, the concentration of the buffer is about 15 mM. In some embodiments, the concentration of the buffer is about 20 mM. In some embodiments, the concentration of the buffer is about 25 mM. In some embodiments, the concentration of the buffer is about 30 mM.

In some embodiments, the concentration of the buffer is 10 mM to 30 mM. In some embodiments, the concentration of the buffer is 15 mM to 25 mM. In some embodiments, the concentration of the buffer is 12 mM to 28 mM. In some embodiments, the concentration of the buffer is 10 mM. In some embodiments, the concentration of the buffer is 15 mM. In some embodiments, the concentration of the buffer is 20 mM. In some embodiments, the concentration of the buffer is 25 mM. In some embodiments, the concentration of the buffer is 30 mM.

The liquid pharmaceutical formulation of the disclosure comprises a tonicity agent. In some embodiments, the tonicity agent is a salt or a polyol. In some embodiments, the tonicity agent is a salt. In some embodiments, the tonicity agent is a polyol. In some embodiments, the polyol is a sugar or a sugar alcohol. In some embodiments, the polyol is a sugar. In some embodiments, the polyol is a sugar alcohol. In some embodiments, the tonicity agent is a sugar alcohol selected from the group consisting of mannitol, xylitol, erythritol, threitol, sorbitol and glycerol. In some embodiments, the sugar alcohol is mannitol. In some embodiments, the sugar alcohol is xylitol. In some embodiments, the sugar alcohol is erythritol. In some embodiments, the sugar alcohol is threitol. In some embodiments, the sugar alcohol is sorbitol. In some embodiments, the sugar alcohol is glycerol.

In some embodiments, the tonicity agent is a reducing sugar. In some embodiments, the reducing sugar is selected from the group consisting of fructose, mannose, maltose, lactose, arabinose, xylose, ribose, rhamnose, galactose and glucose. In some embodiments, the reducing sugar is fructose. In some embodiments, the reducing sugar is mannose. In some embodiments, the reducing sugar is maltose. In some embodiments, the reducing sugar is lactose. In some embodiments, the reducing sugar is arabinose. In some embodiments, the reducing sugar is xylose. In some embodiments, the reducing sugar is ribose. In some embodiments, the reducing sugar is rhamnose. In some embodiments, the reducing sugar is galactose. In some embodiments, the reducing sugar is glucose.

In some embodiments, the tonicity agent is a non-reducing sugar. In some embodiments, the non-reducing sugar is selected from the group consisting of sucrose, trehalose, sorbose, melezitose and raffinose. In some embodiments, the non-reducing sugar is sucrose. In some embodiments, the non-reducing sugar is trehalose. In some embodiments, the non-reducing sugar is sorbose. In some embodiments, the non-reducing sugar is melezitose. In some embodiments, the non-reducing sugar is raffinose. In particular embodiments, the tonicity agent is sucrose.

In some embodiments, the tonicity agent is a salt. In some embodiments, the salt is sodium chloride or potassium chloride. In some embodiments, the salt is sodium chloride. In some embodiments, the salt is potassium chloride.

In some embodiments, the tonicity agent is selected from the group consisting of sodium chloride, mannitol, sucrose, glucose, glycerol, and potassium chloride. In some embodiments, the tonicity agent is sodium chloride. In some embodiments, the tonicity agent is mannitol. In some embodiments, the tonicity agent is sucrose. In some embodiments, the tonicity agent is glucose. In some embodiments, the tonicity agent is glycerol. In some embodiments, the tonicity agent is potassium chloride.

In some embodiments, the concentration of the tonicity agent is about 100 mM to about 300 mM. In some embodiments, the concentration of the tonicity agent is about 120 mM to about 280 mM. In some embodiments, the concentration of the tonicity agent is about 140 mM to about 280 mM. In some embodiments, the concentration of the tonicity agent is about 160 mM to about 280 mM. In some embodiments, the concentration of the tonicity agent is about 180 mM to about 280 mM. In some embodiments, the concentration of the tonicity agent is about 200 mM to about 280 mM. In some embodiments, the concentration of the tonicity agent is about 220 mM to about 280 mM. In some embodiments, the concentration of the tonicity agent is about 240 mM to about 280 mM. In some embodiments, the concentration of the tonicity agent is about 260 mM to about 280 mM. In some embodiments, the concentration of the tonicity agent is about 220 mM to about 260 mM. In some embodiments, the concentration of the tonicity agent is about 230 mM to about 250 mM. In some embodiments, the concentration of the tonicity agent is about 200 mM to about 260 mM. In some embodiments, the concentration of the tonicity agent is about 180 mM to about 300 mM. In some embodiments, the concentration of the tonicity agent is about 200 mM to about 300 mM. In some embodiments, the concentration of the tonicity agent is about 220 mM to about 300 mM. In some embodiments, the concentration of the tonicity agent is about 240 mM to about 300 mM. In some embodiments, the concentration of the tonicity agent is about 250 mM to about 300 mM.

In some embodiments, the concentration of the tonicity agent is about 100 mM. In some embodiments, the concentration of the tonicity agent is about 110 mM. In some embodiments, the concentration of the tonicity agent is about 120 mM. In some embodiments, the concentration of the tonicity agent is about 130 mM. In some embodiments, the concentration of the tonicity agent is about 140 mM. In some embodiments, the concentration of the tonicity agent is about 150 mM. In some embodiments, the concentration of the tonicity agent is about 160 mM. In some embodiments, the concentration of the tonicity agent is about 170 mM. In some embodiments, the concentration of the tonicity agent is about 180 mM. In some embodiments, the concentration of the tonicity agent is about 190 mM. In some embodiments, the concentration of the tonicity agent is about 200 mM. In some embodiments, the concentration of the tonicity agent is about 210 mM. In some embodiments, the concentration of the tonicity agent is about 220 mM. In some embodiments, the concentration of the tonicity agent is about 230 mM. In particular embodiments, the concentration of the tonicity agent is about 240 mM. In some embodiments, the concentration of the tonicity agent is about 250 mM. In some embodiments, the concentration of the tonicity agent is about 260 mM. In some embodiments, the concentration of the tonicity agent is about 270 mM. In some embodiments, the concentration of the tonicity agent is about 280 mM. In some embodiments, the concentration of the tonicity agent is about 290 mM. In some embodiments, the concentration of the tonicity agent is about 300 mM.

In some embodiments, the concentration of the tonicity agent is 100 mM to 300 mM. In some embodiments, the concentration of the tonicity agent is 120 mM to 280 mM. In some embodiments, the concentration of the tonicity agent is 140 mM to 280 mM. In some embodiments, the concentration of the tonicity agent is 160 mM to 280 mM. In some embodiments, the concentration of the tonicity agent is 180 mM to 280 mM. In some embodiments, the concentration of the tonicity agent is 200 mM to 280 mM. In some embodiments, the concentration of the tonicity agent is 220 mM to 280 mM. In some embodiments, the concentration of the tonicity agent is 240 mM to 280 mM. In some embodiments, the concentration of the tonicity agent is 260 mM to 280 mM. In some embodiments, the concentration of the tonicity agent is 220 mM to 260 mM. In some embodiments, the concentration of the tonicity agent is 230 mM to 250 mM. In some embodiments, the concentration of the tonicity agent is 200 mM to 260 mM. In some embodiments, the concentration of the tonicity agent is 180 mM to 300 mM. In some embodiments, the concentration of the tonicity agent is 200 mM to 300 mM. In some embodiments, the concentration of the tonicity agent is 220 mM to 300 mM. In some embodiments, the concentration of the tonicity agent is 240 mM to 300 mM. In some embodiments, the concentration of the tonicity agent is 250 mM to 300 mM.

In some embodiments, the concentration of the tonicity agent is 100 mM. In some embodiments, the concentration of the tonicity agent is 110 mM. In some embodiments, the concentration of the tonicity agent is 120 mM. In some embodiments, the concentration of the tonicity agent is 130 mM. In some embodiments, the concentration of the tonicity agent is 140 mM. In some embodiments, the concentration of the tonicity agent is 150 mM. In some embodiments, the concentration of the tonicity agent is 160 mM. In some embodiments, the concentration of the tonicity agent is 170 mM. In some embodiments, the concentration of the tonicity agent is 180 mM. In some embodiments, the concentration of the tonicity agent is 190 mM. In some embodiments, the concentration of the tonicity agent is 200 mM. In some embodiments, the concentration of the tonicity agent is 210 mM. In some embodiments, the concentration of the tonicity agent is 220 mM. In some embodiments, the concentration of the tonicity agent is 230 mM. In particular embodiments, the concentration of the tonicity agent is 240 mM. In some embodiments, the concentration of the tonicity agent is 250 mM. In some embodiments, the concentration of the tonicity agent is 260 mM. In some embodiments, the concentration of the tonicity agent is 270 mM. In some embodiments, the concentration of the tonicity agent is 280 mM. In some embodiments, the concentration of the tonicity agent is 290 mM. In some embodiments, the concentration of the tonicity agent is 300 mM.

The liquid pharmaceutical formation comprises a surfactant. In some embodiments, the surfactant is a non-ionic surfactant, an ionic surfactant, a zwitterionic surfactant or a combination thereof. In some embodiments, the surfactant is a non-ionic surfactant. In some embodiments, the surfactant is an ionic surfactant. In some embodiments, the surfactant is a zwitterionic surfactant.

In some embodiments, the surfactant is a non-ionic surfactant selected from the group consisting of a polysorbate, a poloxamer, triton, octyl glucoside, myristamidopropyl-dimethylamine, palmidopropyl-dimethylamine, isostearamidopropyl-dimethylamine, polyethyl glycol, polypropyl glycol, copolymers of ethylene, copolymers of propylene glycol, and combinations thereof. In some embodiments, the surfactant is a polysorbate. Polysorbates are amphipathic, nonionic surfactants derived from ethoxylated sorbitan or isosorbide (a derivative of sorbitol) esterified with fatty acids. The polysorbate may be Polysorbate 20 or Polysorbate 80. In some embodiments, the polysorbate is Polysorbate 20. In some embodiments, the polysorbate is Polysorbate 80. In some embodiments, the surfactant is a poloxamer. Poloxamers are block co-polymers of polyoxyethylene and polyoxypropylene and include poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, poloxamer 105 Benzoate, and poloxamer 182 Dibenzoate. In some embodiments, the poloxamer is poloxamer 188 or 407. In some embodiments, the poloxamer is poloxamer 188. In some embodiments, the surfactant is triton. In some embodiments, the surfactant is octyl glucoside. In some embodiments, the surfactant is myristamidopropyl-dimethylamine. In some embodiments, the surfactant is palmidopropyl-dimethylamine. In some embodiments, the surfactant is isostearamidopropyl-dimethylamine. In some embodiments, the surfactant is polyethyl glycol. In some embodiments, the surfactant is polypropyl glycol. In some embodiments, the surfactant is copolymers of ethylene. In some embodiments, the surfactant is copolymers of propylene glycol.

In some embodiments, the surfactant is selected from the group consisting of Polysorbate 20, Polysorbate 80, Poloxamer 188, and combinations thereof. In particular embodiments, the surfactant is Polysorbate 20. In some embodiments, the surfactant is Polysorbate 80. In some embodiments, the surfactant is Poloxamer 188.

In some embodiments, the surfactant is a zwitterionic surfactant selected from the group consisting of sodium dodecyl sulfate, sodium laurel sulfate, lauryl-sulfobetaine, myristyl-sulfobetaine, linoleyl-sulfobetaine, stearyl-sulfobetaine, lauroamidopropyl-betaine, cocamidopropyl-betaine, linoleamidopropyl-betaine, myristamidopropyl-betaine, palmidopropyl-betaine, isostearamidopropyl-betaine, sodium methyl cocoyl-taurate, sodium methyl oleyl-taurate, and combinations thereof. In some embodiments, the surfactant is sodium dodecyl sulfate. In some embodiments, the surfactant is sodium laurel sulfate. In some embodiments, the surfactant is lauryl-sulfobetaine. In some embodiments, the surfactant is myristyl-sulfobetaine. In some embodiments, the surfactant is linoleyl-sulfobetaine. In some embodiments, the surfactant is stearyl-sulfobetaine. In some embodiments, the surfactant is lauroamidopropyl-betaine. In some embodiments, the surfactant is cocamidopropyl-betaine. In some embodiments, the surfactant is linoleamidopropyl-betaine. In some embodiments, the surfactant is myristamidopropyl-betaine. In some embodiments, the surfactant is palmidopropyl-betaine. In some embodiments, the surfactant is isostearamidopropyl-betaine. In some embodiments, the surfactant is sodium methyl cocoyl-taurate. In some embodiments, the surfactant is sodium methyl oleyl-taurate.

In some embodiments, the concentration of the surfactant is about 0.1 mg/mL to about 1 mg/mL. In some embodiments, the concentration of the surfactant is about 0.2 mg/mL to about 1 mg/mL. In some embodiments, the concentration of the surfactant is about 0.3 mg/mL to about 1 mg/mL. In some embodiments, the concentration of the surfactant is about 0.4 mg/ml to about 1 mg/mL. In some embodiments, the concentration of the surfactant is about 0.5 mg/mL to about 1 mg/mL. In some embodiments, the concentration of the surfactant is about 0.6 mg/mL to about 1 mg/mL. In some embodiments, the concentration of the surfactant is about 0.7 mg/mL to about 1 mg/mL. In some embodiments, the concentration of the surfactant is about 0.8 mg/mL to about 1 mg/mL. In some embodiments, the concentration of the surfactant is about 0.9 mg/mL to about 1 mg/mL. In some embodiments, the concentration of the surfactant is about 0.1 mg/mL to about 0.9 mg/mL. In some embodiments, the concentration of the surfactant is about 0.1 mg/mL to about 0.8 mg/mL. In some embodiments, the concentration of the surfactant is about 0.1 mg/mL to about 0.7 mg/mL. In some embodiments, the concentration of the surfactant is about 0.1 mg/mL to about 0.6 mg/mL. In some embodiments, the concentration of the surfactant is about 0.1 mg/mL to about 0.5 mg/mL. In some embodiments, the concentration of the surfactant is about 0.1 mg/mL to about 0.4 mg/mL. In some embodiments, the concentration of the surfactant is about 0.1 mg/mL to about 0.3 mg/mL. In some embodiments, the concentration of the surfactant is about 0.1 mg/mL to about 0.2 mg/mL. In some embodiments, the concentration of the surfactant is about 0.2 mg/mL to about 0.9 mg/mL. In some embodiments, the concentration of the surfactant is about 0.2 mg/mL to about 0.8 mg/mL. In some embodiments, the concentration of the surfactant is about 0.2 mg/mL to about 0.7 mg/mL. In some embodiments, the concentration of the surfactant is about 0.2 mg/mL to about 0.6 mg/mL. In some embodiments, the concentration of the surfactant is about 0.2 mg/mL to about 0.5 mg/mL. In some embodiments, the concentration of the surfactant is about 0.2 mg/mL to about 0.4 mg/mL. In some embodiments, the concentration of the surfactant is about 0.2 mg/mL to about 0.3 mg/mL. In some embodiments, the concentration of the surfactant is about 0.3 mg/mL to about 0.4 mg/mL. In some embodiments, the concentration of the surfactant is about 0.3 mg/mL to about 0.5 mg/mL. In some embodiments, the concentration of the surfactant is about 0.3 mg/mL to about 0.6 mg/mL. In some embodiments, the concentration of the surfactant is about 0.3 mg/mL to about 0.7 mg/mL. In some embodiments, the concentration of the surfactant is about 0.3 mg/mL to about 0.8 mg/mL. In some embodiments, the concentration of the surfactant is about 0.3 mg/mL to about 0.9 mg/mL. In some embodiments, the concentration of the surfactant is about 0.4 mg/mL to about 0.9 mg/mL. In some embodiments, the concentration of the surfactant is about 0.4 mg/ml to about 0.8 mg/mL. In some embodiments, the concentration of the surfactant is about 0.4 mg/mL to about 0.7 mg/mL. In some embodiments, the concentration of the surfactant is about 0.4 mg/mL to about 0.6 mg/mL. In some embodiments, the concentration of the surfactant is about 0.4 mg/mL to about 0.5 mg/mL. In some embodiments, the concentration of the surfactant is about 0.5 mg/mL to about 0.6 mg/mL. In some embodiments, the concentration of the surfactant is about 0.5 mg/mL to about 0.7 mg/mL. In some embodiments, the concentration of the surfactant is about 0.5 mg/mL to about 0.8 mg/mL. In some embodiments, the concentration of the surfactant is about 0.5 mg/mL to about 0.9 mg/mL. In some embodiments, the concentration of the surfactant is about 0.6 mg/mL to about 0.7 mg/mL. In some embodiments, the concentration of the surfactant is about 0.6 mg/mL to about 0.8 mg/mL. In some embodiments, the concentration of the surfactant is about 0.6 mg/mL to about 0.9 mg/mL. In some embodiments, the concentration of the surfactant is about 0.7 mg/mL to about 0.8 mg/mL. In some embodiments, the concentration of the surfactant is about 0.7 mg/mL to about 0.9 mg/mL. In some embodiments, the concentration of the surfactant is about 0.8 mg/mL to about 0.9 mg/mL.

In some embodiments, the concentration of the surfactant is about 0.1 mg/mL. In some embodiments, the concentration of the surfactant is about 0.2 mg/mL. In some embodiments, the concentration of the surfactant is about 0.3 mg/mL. In some embodiments, the concentration of the surfactant is about 0.4 mg/mL. In some embodiments, the concentration of the surfactant is about 0.5 mg/mL. In particular embodiments, the concentration of the surfactant is about 0.6 mg/mL. In some embodiments, the concentration of the surfactant is about 0.7 mg/mL. In particular embodiments, the concentration of the surfactant is about 0.8 mg/mL. In some embodiments, the concentration of the surfactant is about 0.9 mg/mL. In some embodiments, the concentration of the surfactant is about 1 mg/mL.

In some embodiments, the concentration of the surfactant is 0.1 mg/mL to 1 mg/mL. In some embodiments, the concentration of the surfactant is 0.2 mg/mL to 1 mg/mL. In some embodiments, the concentration of the surfactant is 0.3 mg/mL to 1 mg/mL. In some embodiments, the concentration of the surfactant is 0.4 mg/mL to 1 mg/mL. In some embodiments, the concentration of the surfactant is 0.5 mg/mL to 1 mg/mL. In some embodiments, the concentration of the surfactant is 0.6 mg/mL to 1 mg/mL. In some embodiments, the concentration of the surfactant is 0.7 mg/mL to 1 mg/mL. In some embodiments, the concentration of the surfactant is 0.8 mg/mL to 1 mg/mL. In some embodiments, the concentration of the surfactant is 0.9 mg/mL to 1 mg/mL. In some embodiments, the concentration of the surfactant is 0.1 mg/mL to 0.9 mg/mL. In some embodiments, the concentration of the surfactant is 0.1 mg/mL to 0.8 mg/mL. In some embodiments, the concentration of the surfactant is 0.1 mg/mL to 0.7 mg/mL. In some embodiments, the concentration of the surfactant is 0.1 mg/mL to 0.6 mg/mL. In some embodiments, the concentration of the surfactant is 0.1 mg/mL to 0.5 mg/mL. In some embodiments, the concentration of the surfactant is 0.1 mg/mL to 0.4 mg/mL. In some embodiments, the concentration of the surfactant is 0.1 mg/mL to 0.3 mg/mL. In some embodiments, the concentration of the surfactant is 0.1 mg/mL to 0.2 mg/mL. In some embodiments, the concentration of the surfactant is 0.2 mg/mL to 0.9 mg/mL. In some embodiments, the concentration of the surfactant is 0.2 mg/mL to 0.8 mg/mL. In some embodiments, the concentration of the surfactant is 0.2 mg/mL to 0.7 mg/mL. In some embodiments, the concentration of the surfactant is 0.2 mg/mL to 0.6 mg/mL. In some embodiments, the concentration of the surfactant is 0.2 mg/mL to 0.5 mg/mL. In some embodiments, the concentration of the surfactant is 0.2 mg/mL to 0.4 mg/mL. In some embodiments, the concentration of the surfactant is 0.2 mg/mL to 0.3 mg/mL. In some embodiments, the concentration of the surfactant is 0.3 mg/mL to 0.4 mg/mL. In some embodiments, the concentration of the surfactant is 0.3 mg/mL to 0.5 mg/mL. In some embodiments, the concentration of the surfactant is 0.3 mg/mL to 0.6 mg/mL. In some embodiments, the concentration of the surfactant is 0.3 mg/mL to 0.7 mg/mL. In some embodiments, the concentration of the surfactant is 0.3 mg/mL to 0.8 mg/mL. In some embodiments, the concentration of the surfactant is 0.3 mg/mL to 0.9 mg/mL. In some embodiments, the concentration of the surfactant is 0.4 mg/mL to 0.9 mg/mL. In some embodiments, the concentration of the surfactant is 0.4 mg/mL to 0.8 mg/mL. In some embodiments, the concentration of the surfactant is 0.4 mg/mL to 0.7 mg/mL. In some embodiments, the concentration of the surfactant is 0.4 mg/mL to 0.6 mg/mL. In some embodiments, the concentration of the surfactant is 0.4 mg/mL to 0.5 mg/mL. In some embodiments, the concentration of the surfactant is 0.5 mg/mL to 0.6 mg/mL. In some embodiments, the concentration of the surfactant is 0.5 mg/mL to 0.7 mg/mL. In some embodiments, the concentration of the surfactant is 0.5 mg/mL to 0.8 mg/mL. In some embodiments, the concentration of the surfactant is 0.5 mg/mL to 0.9 mg/mL. In some embodiments, the concentration of the surfactant is 0.6 mg/mL to 0.7 mg/mL. In some embodiments, the concentration of the surfactant is 0.6 mg/mL to 0.8 mg/mL. In some embodiments, the concentration of the surfactant is 0.6 mg/mL to 0.9 mg/mL. In some embodiments, the concentration of the surfactant is 0.7 mg/mL to 0.8 mg/mL. In some embodiments, the concentration of the surfactant is 0.7 mg/mL to 0.9 mg/mL. In some embodiments, the concentration of the surfactant is 0.8 mg/mL to 0.9 mg/mL.

In some embodiments, the concentration of the surfactant is 0.1 mg/mL. In some embodiments, the concentration of the surfactant is 0.2 mg/mL. In some embodiments, the concentration of the surfactant is 0.3 mg/mL. In some embodiments, the concentration of the surfactant is 0.4 mg/mL. In some embodiments, the concentration of the surfactant is 0.5 mg/mL. In particular embodiments, the concentration of the surfactant is 0.6 mg/mL. In some embodiments, the concentration of the surfactant is 0.7 mg/mL. In particular embodiments, the concentration of the surfactant is 0.8 mg/mL. In some embodiments, the concentration of the surfactant is 0.9 mg/mL. In some embodiments, the concentration of the surfactant is 1 mg/mL.

In some embodiments, the liquid pharmaceutical formulation further comprises a stabilizer. In some embodiments, the stabilizer is selected from the group consisting of methionine, glycine, alanine, proline, taurine, betaine, octopine, glutamate, sarcosine, γ-aminobutyric acid, and trimethylamine N-oxide. In particular embodiments, the stabilizer is methionine. In some embodiments, the stabilizer is glycine. In some embodiments, the stabilizer is alanine. In some embodiments, the stabilizer is proline. In some embodiments, the stabilizer is taurine. In some embodiments, the stabilizer is betaine. In some embodiments, the stabilizer is octopine. In some embodiments, the stabilizer is glutamate. In some embodiments, the stabilizer is sarcosine. In some embodiments, the stabilizer is γ-aminobutyric acid. In some embodiments, the stabilizer is trimethylamine N-oxide.

In some embodiments, the concentration of the stabilizer is about 5 mM to about 20 mM. In some embodiments, the concentration of the stabilizer is about 5 mM to about 15 mM. In some embodiments, the concentration of the stabilizer is about 8 mM to about 12 mM. In some embodiments, the concentration of the stabilizer is about 9 mM to about 11 mM. In some embodiments, the concentration of the stabilizer is about 5 mM. In some embodiments, the concentration of the stabilizer is about 6 mM. In some embodiments, the concentration of the stabilizer is about 7 mM. In some embodiments, the concentration of the stabilizer is about 8 mM. In some embodiments, the concentration of the stabilizer is about 9 mM. In particular embodiments, the concentration of the stabilizer is about 10 mM. In some embodiments, the concentration of the stabilizer is about 11 mM. In some embodiments, the concentration of the stabilizer is about 12 mM. In some embodiments, the concentration of the stabilizer is about 13 mM. In some embodiments, the concentration of the stabilizer is about 14 mM. In some embodiments, the concentration of the stabilizer is about 15 mM. In some embodiments, the concentration of the stabilizer is about 16 mM. In some embodiments, the concentration of the stabilizer is about 17 mM. In some embodiments, the concentration of the stabilizer is about 18 mM. In some embodiments, the concentration of the stabilizer is about 19 mM. In some embodiments, the concentration of the stabilizer is about 20 mM.

In some embodiments, the concentration of the stabilizer is 0 mM to 15 mM. In some embodiments, the concentration of the stabilizer is 5 mM to 20 mM. In some embodiments, the concentration of the stabilizer is 5 mM to 15 mM. In some embodiments, the concentration of the stabilizer is 8 mM to 12 mM. In some embodiments, the concentration of the stabilizer is 9 mM to 11 mM. In some embodiments, the concentration of the stabilizer is 5 mM. In some embodiments, the concentration of the stabilizer is 6 mM. In some embodiments, the concentration of the stabilizer is 7 mM. In some embodiments, the concentration of the stabilizer is 8 mM. In some embodiments, the concentration of the stabilizer is 9 mM. In particular embodiments, the concentration of the stabilizer is 10 mM. In some embodiments, the concentration of the stabilizer is 11 mM. In some embodiments, the concentration of the stabilizer is 12 mM. In some embodiments, the concentration of the stabilizer is 13 mM. In some embodiments, the concentration of the stabilizer is 14 mM. In some embodiments, the concentration of the stabilizer is 15 mM. In some embodiments, the concentration of the stabilizer is 16 mM. In some embodiments, the concentration of the stabilizer is 17 mM. In some embodiments, the concentration of the stabilizer is 18 mM. In some embodiments, the concentration of the stabilizer is 19 mM. In some embodiments, the concentration of the stabilizer is 20 mM.

In some embodiments, the pH of the liquid pharmaceutical formulation is about 4.0 to about 7.0. In some embodiments, the pH of the liquid pharmaceutical formulation is about 4.5 to about 7.0. In some embodiments, the pH of the liquid pharmaceutical formulation is about 5.0 to about 7.0. In some embodiments, the pH of the liquid pharmaceutical formulation is about 5.5 to about 7.0. In some embodiments, the pH of the liquid pharmaceutical formulation is about 6.0 to about 7.0. In some embodiments, the pH of the liquid pharmaceutical formulation is about 6.5 to about 7.0. In some embodiments, the pH of the liquid pharmaceutical formulation is about 4.0 to about 6.5. In some embodiments, the pH of the liquid pharmaceutical formulation is about 4.0 to about 6.0. In some embodiments, the pH of the liquid pharmaceutical formulation is about 4.0 to about 5.5. In some embodiments, the pH of the liquid pharmaceutical formulation is about 4.0 to about 5.0. In some embodiments, the pH of the liquid pharmaceutical formulation is about 4.0 to about 4.5. In some embodiments, the pH of the liquid pharmaceutical formulation is about 4.5 to about 6.5. In some embodiments, the pH of the liquid pharmaceutical formulation is about 4.5 to about 6.0. In some embodiments, the pH of the liquid pharmaceutical formulation is about 4.5 to about 5.5. In some embodiments, the pH of the liquid pharmaceutical formulation is about 4.5 to about 5.0. In some embodiments, the pH of the liquid pharmaceutical formulation is about 5.0 to about 6.5. In some embodiments, the pH of the liquid pharmaceutical formulation is about 5.0 to about 6.0. In some embodiments, the pH of the liquid pharmaceutical formulation is about 5.0 to about 5.5. In some embodiments, the pH of the liquid pharmaceutical formulation is about 5.2 to about 5.8. In some embodiments, the pH of the liquid pharmaceutical formulation is about 5.2 to about 6.1. In some embodiments, the pH of the liquid pharmaceutical formulation is about 5.4 to about 5.5. In some embodiments, the pH of the liquid pharmaceutical formulation is about 5.5 to about 6.5. In some embodiments, the pH of the liquid pharmaceutical formulation is about 5.5 to about 6.0. In some embodiments, the pH of the liquid pharmaceutical formulation is about 5.5 to about 6.1. In some embodiments, the pH of the liquid pharmaceutical formulation is about 5.7 to about 5.9. In some embodiments, the pH of the liquid pharmaceutical formulation is about 6.0 to about 6.5.

In some embodiments, the pH of the liquid pharmaceutical formulation is about 4.0. In some embodiments, the pH of the liquid pharmaceutical formulation is about 4.1. In some embodiments, the pH of the liquid pharmaceutical formulation is about 4.2. In some embodiments, the pH of the liquid pharmaceutical formulation is about 4.3. In some embodiments, the pH of the liquid pharmaceutical formulation is about 4.4. In some embodiments, the pH of the liquid pharmaceutical formulation is about 4.5. In some embodiments, the pH of the liquid pharmaceutical formulation is about 4.6. In some embodiments, the pH of the liquid pharmaceutical formulation is about 4.7. In some embodiments, the pH of the liquid pharmaceutical formulation is about 4.8. In some embodiments, the pH of the liquid pharmaceutical formulation is about 4.9. In some embodiments, the pH of the liquid pharmaceutical formulation is about 5.0. In some embodiments, the pH of the liquid pharmaceutical formulation is about 5.1. In some embodiments, the pH of the liquid pharmaceutical formulation is about 5.2. In some embodiments, the pH of the liquid pharmaceutical formulation is about 5.3. In some embodiments, the pH of the liquid pharmaceutical formulation is about 5.4. In particular embodiments, the pH of the liquid pharmaceutical formulation is about 5.5. In some embodiments, the pH of the liquid pharmaceutical formulation is about 5.6. In some embodiments, the pH of the liquid pharmaceutical formulation is about 5.7. In particular embodiments, the pH of the liquid pharmaceutical formulation is about 5.8. In some embodiments, the pH of the liquid pharmaceutical formulation is about 5.9. In some embodiments, the pH of the liquid pharmaceutical formulation is about 6.0. In some embodiments, the pH of the liquid pharmaceutical formulation is about 6.2. In some embodiments, the pH of the liquid pharmaceutical formulation is about 6.4. In some embodiments, the pH of the liquid pharmaceutical formulation is about 6.5. In some embodiments, the pH of the liquid pharmaceutical formulation is about 6.6. In some embodiments, the pH of the liquid pharmaceutical formulation is about 6.7. In some embodiments, the pH of the liquid pharmaceutical formulation is about 6.8. In some embodiments, the pH of the liquid pharmaceutical formulation is about 6.9. In some embodiments, the pH of the liquid pharmaceutical formulation is about 7.0.

In some embodiments, the pH of the liquid pharmaceutical formulation is 4.0 to 7.0. In some embodiments, the pH of the liquid pharmaceutical formulation is 4.5 to 7.0. In some embodiments, the pH of the liquid pharmaceutical formulation is 5.0 to 7.0. In some embodiments, the pH of the liquid pharmaceutical formulation is 5.5 to 7.0. In some embodiments, the pH of the liquid pharmaceutical formulation is 6.0 to 7.0. In some embodiments, the pH of the liquid pharmaceutical formulation is 6.5 to 7.0. In some embodiments, the pH of the liquid pharmaceutical formulation is 4.0 to 6.5. In some embodiments, the pH of the liquid pharmaceutical formulation is 4.0 to 6.0. In some embodiments, the pH of the liquid pharmaceutical formulation is 4.0 to 5.5. In some embodiments, the pH of the liquid pharmaceutical formulation is 4.0 to 5.0. In some embodiments, the pH of the liquid pharmaceutical formulation is 4.0 to 4.5. In some embodiments, the pH of the liquid pharmaceutical formulation is 4.5 to 6.5. In some embodiments, the pH of the liquid pharmaceutical formulation is 4.5 to 6.0. In some embodiments, the pH of the liquid pharmaceutical formulation is 4.5 to 5.5. In some embodiments, the pH of the liquid pharmaceutical formulation is 4.5 to 5.0. In some embodiments, the pH of the liquid pharmaceutical formulation is 5.0 to 6.5. In some embodiments, the pH of the liquid pharmaceutical formulation is 5.0 to 6.0. In some embodiments, the pH of the liquid pharmaceutical formulation is 5.0 to 5.5. In some embodiments, the pH of the liquid pharmaceutical formulation is 5.2 to 5.8. In some embodiments, the pH of the liquid pharmaceutical formulation is 5.2 to 6.1. In some embodiments, the pH of the liquid pharmaceutical formulation is 5.4 to 5.5. In some embodiments, the pH of the liquid pharmaceutical formulation is 5.5 to 6.5. In some embodiments, the pH of the liquid pharmaceutical formulation is 5.5 to 6.0. In some embodiments, the pH of the liquid pharmaceutical formulation is 5.5 to 6.1. In some embodiments, the pH of the liquid pharmaceutical formulation is 5.7 to 5.9. In some embodiments, the pH of the liquid pharmaceutical formulation is 6.0 to 6.5.

In some embodiments, the pH of the liquid pharmaceutical formulation is 4.0. In some embodiments, the pH of the liquid pharmaceutical formulation is 4.1. In some embodiments, the pH of the liquid pharmaceutical formulation is 4.2. In some embodiments, the pH of the liquid pharmaceutical formulation is 4.3. In some embodiments, the pH of the liquid pharmaceutical formulation is 4.4. In some embodiments, the pH of the liquid pharmaceutical formulation is 4.5. In some embodiments, the pH of the liquid pharmaceutical formulation is 4.6. In some embodiments, the pH of the liquid pharmaceutical formulation is 4.7. In some embodiments, the pH of the liquid pharmaceutical formulation is 4.8. In some embodiments, the pH of the liquid pharmaceutical formulation is 4.9. In some embodiments, the pH of the liquid pharmaceutical formulation is 5.0. In some embodiments, the pH of the liquid pharmaceutical formulation is 5.1. In some embodiments, the pH of the liquid pharmaceutical formulation is 5.2. In some embodiments, the pH of the liquid pharmaceutical formulation is 5.3. In some embodiments, the pH of the liquid pharmaceutical formulation is 5.4. In particular embodiments, the pH of the liquid pharmaceutical formulation is 5.5. In some embodiments, the pH of the liquid pharmaceutical formulation is 5.6. In some embodiments, the pH of the liquid pharmaceutical formulation is 5.7. In particular embodiments, the pH of the liquid pharmaceutical formulation is 5.8. In some embodiments, the pH of the liquid pharmaceutical formulation is 5.9. In some embodiments, the pH of the liquid pharmaceutical formulation is 6.0. In some embodiments, the pH of the liquid pharmaceutical formulation is 6.2. In some embodiments, the pH of the liquid pharmaceutical formulation is 6.4. In some embodiments, the pH of the liquid pharmaceutical formulation is 6.5. In some embodiments, the pH of the liquid pharmaceutical formulation is 6.6. In some embodiments, the pH of the liquid pharmaceutical formulation is 6.7. In some embodiments, the pH of the liquid pharmaceutical formulation is 6.8. In some embodiments, the pH of the liquid pharmaceutical formulation is 6.9. In some embodiments, the pH of the liquid pharmaceutical formulation is 7.0.

In some embodiments, the anti-TIGIT monoclonal antibody and the anti-PD-L1 monoclonal antibody are in a ratio of about 1:2. For example, if the anti-TIGIT monoclonal antibody is present at a concentration of about 40 mg/ml, then the anti-PD-L1 monoclonal antibody is present at a concentration of about 80 mg/ml. Similarly, if the anti-TIGIT monoclonal antibody is present at a concentration of 40 mg/ml, then the anti-PD-L1 monoclonal antibody is present at a concentration of 80 mg/ml.

In some embodiments, the liquid pharmaceutical formulation further comprises a hyaluronidase. In some embodiments, the hyaluronidase is a recombinant human hyaluronidase. In some embodiments, the hyaluronidase is a human soluble PH20 hyaluronidase glycoprotein. In particular embodiments, the hyaluronidase is rHuPH20.

In some embodiments, the concentration of the hyaluronidase is about 1000 U/mL to about 7000 U/mL. In some embodiments, the concentration of the hyaluronidase is about 1000 U/mL to about 6000 U/mL. In some embodiments, the concentration of the hyaluronidase is about 1000 U/mL to about 5000 U/mL. In some embodiments, the concentration of the hyaluronidase is about 1000 U/mL to about 4000 U/mL. In some embodiments, the concentration of the hyaluronidase is about 1000 U/mL to about 3000 U/mL. In some embodiments, the concentration of the hyaluronidase is about 1000 U/mL to about 2000 U/mL. In some embodiments, the concentration of the hyaluronidase is about 1000 U/mL to about 1500 U/mL. In some embodiments, the concentration of the hyaluronidase is about 1500 U/mL to about 3000 U/mL. In some embodiments, the concentration of the hyaluronidase is about 1500 U/mL to about 2500 U/mL. In some embodiments, the concentration of the hyaluronidase is about 1500 U/mL to about 2000 U/mL. In some embodiments, the concentration of the hyaluronidase is about 1800 U/mL to about 2200 U/mL.

In some embodiments, the concentration of the hyaluronidase is about 1000 U/mL. In some embodiments, the concentration of the hyaluronidase is about 1500 U/mL. In particular embodiments, the concentration of the hyaluronidase is about 2000 U/mL. In some embodiments, the concentration of the hyaluronidase is about 2500 U/mL. In some embodiments, the concentration of the hyaluronidase is about 3000 U/mL. In some embodiments, the concentration of the hyaluronidase is about 3500 U/mL. In some embodiments, the concentration of the hyaluronidase is about 4000 U/mL. In some embodiments, the concentration of the hyaluronidase is about 4500 U/mL. In some embodiments, the concentration of the hyaluronidase is about 5000 U/mL. In some embodiments, the concentration of the hyaluronidase is about 5500 U/mL. In some embodiments, the concentration of the hyaluronidase is about 6000 U/mL. In some embodiments, the concentration of the hyaluronidase is about 6500 U/mL. In some embodiments, the concentration of the hyaluronidase is about 7000 U/mL.

In some embodiments, the concentration of the hyaluronidase is 1000 U/mL to 7000 U/mL. In some embodiments, the concentration of the hyaluronidase is 1000 U/mL to 6000 U/mL. In some embodiments, the concentration of the hyaluronidase is 1000 U/mL to 5000 U/mL. In some embodiments, the concentration of the hyaluronidase is 1000 U/mL to 4000 U/mL. In some embodiments, the concentration of the hyaluronidase is 1000 U/mL to 3000 U/mL. In some embodiments, the concentration of the hyaluronidase is 1000 U/mL to 2000 U/mL. In some embodiments, the concentration of the hyaluronidase is 1000 U/mL to 1500 U/mL. In some embodiments, the concentration of the hyaluronidase is 1500 U/mL to 3000 U/mL. In some embodiments, the concentration of the hyaluronidase is 1500 U/mL to 2500 U/mL. In some embodiments, the concentration of the hyaluronidase is 1500 U/mL to 2000 U/mL. In some embodiments, the concentration of the hyaluronidase is 1800 U/mL to 2200 U/mL.

In some embodiments, the concentration of the hyaluronidase is 1000 U/mL. In some embodiments, the concentration of the hyaluronidase is 1500 U/mL. In particular embodiments, the concentration of the hyaluronidase is 2000 U/mL. In some embodiments, the concentration of the hyaluronidase is 2500 U/mL. In some embodiments, the concentration of the hyaluronidase is 3000 U/mL. In some embodiments, the concentration of the hyaluronidase is 3500 U/mL. In some embodiments, the concentration of the hyaluronidase is 4000 U/mL. In some embodiments, the concentration of the hyaluronidase is 4500 U/mL. In some embodiments, the concentration of the hyaluronidase is 5000 U/mL. In some embodiments, the concentration of the hyaluronidase is 5500 U/mL. In some embodiments, the concentration of the hyaluronidase is 6000 U/mL. In some embodiments, the concentration of the hyaluronidase is 6500 U/mL. In some embodiments, the concentration of the hyaluronidase is 7000 U/mL.

In some embodiments, the liquid pharmaceutical formulation comprises 50 mg/mL to 70 mg/mL anti-TIGIT monoclonal antibody, 15 mM to 25 mM buffer, 230 to 250 mM tonicity agent, 5 mM to 15 mM stabilizer, 0.3 mg/mL to 0.5 mg/mL surfactant, and pH of 5.4 to 5.6. In some embodiments, the liquid pharmaceutical formulation comprises 50 mg/mL to 70 mg/mL anti-TIGIT monoclonal antibody, 15 mM to 25 mM histidine acetate, 230 mM to 250 mM sucrose, 5 mM to 15 mM methionine, 0.3 mg/mL to 0.5 mg/mL Polysorbate 20, and pH of 5.4 to 5.6. In some embodiments, the liquid pharmaceutical formulation comprises 55 mg/mL to 65 mg/mL anti-TIGIT monoclonal antibody, 18 mM to 22 mM buffer, 235 mM to 245 mM tonicity agent, 8 mM to 12 mM stabilizer, 0.35 mg/mL to 0.45 mg/mL surfactant, and pH of 5.45 to 5.55. In some embodiments, the liquid pharmaceutical formulation comprises 55 mg/mL to 65 mg/mL anti-TIGIT monoclonal antibody, 18 mM to 22 mM histidine acetate, 235 mM to 245 mM sucrose, 8 mM to 12 mM methionine, 0.35 mg/mL to 0.45 mg/mL Polysorbate 20, and pH of 5.45 to 5.55.

In some embodiments, the liquid pharmaceutical formulation comprises about 50 to about 70 mg/mL anti-TIGIT monoclonal antibody, about 15 mM to about 25 mM buffer, about 230 mM to about 250 mM tonicity agent, about 5 mM to about 15 mM stabilizer, about 0.3 mg/mL to about 0.5 mg/mL surfactant, and pH of about 5.4 to about 5.6. In some embodiments, the liquid pharmaceutical formulation comprises about 50 mg/mL to about 70 mg/mL anti-TIGIT monoclonal antibody, about 15 mM to about 25 mM histidine acetate, about 230 mM to about 250 mM sucrose, about 5 mM to about 15 mM methionine, about 0.3 mg/mL to about 0.5 mg/mL Polysorbate 20, and pH of about 5.4 to 5.6. In some embodiments, the liquid pharmaceutical formulation comprises about 55 mg/mL to about 65 mg/mL anti-TIGIT monoclonal antibody, about 18 mM to about 22 mM buffer, about 235 mM to about 245 mM tonicity agent, about 8 mM to about 12 mM stabilizer, about 0.35 mg/mL to about 0.45 mg/ml surfactant, and pH of about 5.45 to about 5.55. In some embodiments, the liquid pharmaceutical formulation comprises about 55 mg/mL to about 65 mg/mL anti-TIGIT monoclonal antibody, about 18 mM to about 22 mM histidine acetate, about 235 mM to about 245 mM sucrose, about 8 mM to about 12 mM methionine, about 0.35 mg/mL to about 0.45 mg/mL Polysorbate 20, and pH of about 5.45 to about 5.55.

In some embodiments, the liquid pharmaceutical formulation comprises 60 mg/mL anti-TIGIT monoclonal antibody, 20 mM buffer, 240 mM tonicity agent, 10 mM stabilizer, 0.4 mg/mL surfactant, and pH 5.5. In some embodiments, the liquid pharmaceutical formulation comprises 60 mg/mL anti-TIGIT monoclonal antibody, 20 mM histidine acetate, 240 mM sucrose, 10 mM methionine, 0.4 mg/mL Polysorbate 20, and pH 5.5. It is within the skill in the art to convert mg/ml into % (w/v). For example, 0.4 mg/ml Polysorbate 20 is equivalent to 0.04% (w/v) Polysorbate 20.

In some embodiments, the liquid pharmaceutical formulation comprises about 60 mg/mL anti-TIGIT monoclonal antibody, about 20 mM buffer, about 240 mM tonicity agent, about 10 mM stabilizer, about 0.4 mg/mL surfactant, and pH of about 5.5. In some embodiments, the liquid pharmaceutical formulation comprises about 60 mg/mL anti-TIGIT monoclonal antibody, about 20 mM histidine acetate, about 240 mM sucrose, about 10 mM methionine, about 0.4 mg/mL Polysorbate 20, and pH of about 5.5.

In some embodiments, the liquid pharmaceutical formulation comprises 150 to 170 mg/mL anti-TIGIT monoclonal antibody, 15 to 25 mM buffer, 230 to 250 mM tonicity agent, 5 to 15 mM stabilizer, 0.5 to 0.7 mg/mL surfactant, and pH of 5.4 to 5.6. In some embodiments, the liquid pharmaceutical formulation comprises 150 to 170 mg/mL anti-TIGIT monoclonal antibody, 15 to 25 mM histidine acetate, 230 to 250 mM sucrose, 5 to 15 mM methionine, 0.5 to 0.7 mg/mL Polysorbate 20, and pH of 5.4 to 5.6. In some embodiments, the liquid pharmaceutical formulation comprises 155 to 165 mg/mL anti-TIGIT monoclonal antibody, 18 to 22 mM buffer, 235 to 245 mM tonicity agent, 8 to 12 mM stabilizer, 0.55 to 0.65 mg/mL surfactant, and pH of 5.45 to 5.55. In some embodiments, the liquid pharmaceutical formulation comprises 155 to 165 mg/mL anti-TIGIT monoclonal antibody, 18 to 22 mM histidine acetate, 235 to 245 mM sucrose, 8 to 12 mM methionine, 0.55 to 0.65 mg/mL Polysorbate 20, and pH of 5.45 to 5.55.

In some embodiments, the liquid pharmaceutical formulation comprises about 150 to about 170 mg/mL anti-TIGIT monoclonal antibody, about 15 to about 25 mM buffer, about 230 to about 250 mM tonicity agent, about 5 to about 15 mM stabilizer, about 0.5 to about 0.7 mg/mL surfactant, and pH of about 5.4 to about 5.6. In some embodiments, the liquid pharmaceutical formulation comprises about 150 to about 170 mg/mL anti-TIGIT monoclonal antibody, about 15 to about 25 mM histidine acetate, about 230 to about 250 mM sucrose, about 5 to about 15 mM methionine, about 0.5 to about 0.7 mg/mL Polysorbate 20, and pH of about 5.4 to about 5.6. In some embodiments, the liquid pharmaceutical formulation comprises about 155 to about 165 mg/mL anti-TIGIT monoclonal antibody, about 18 to about 22 mM buffer, about 235 to about 245 mM tonicity agent, about 8 to about 12 mM stabilizer, about 0.55 to about 0.65 mg/mL surfactant, and pH of about 5.45 to about 5.55. In some embodiments, the liquid pharmaceutical formulation comprises about 155 to about 165 mg/mL anti-TIGIT monoclonal antibody, about 18 to about 22 mM histidine acetate, about 235 to about 245 mM sucrose, about 8 to about 12 mM methionine, about 0.55 to about 0.65 mg/mL Polysorbate 20, and pH of about 5.45 to about 5.55.

In some embodiments, the liquid pharmaceutical formulation comprises 160 mg/mL anti-TIGIT monoclonal antibody, 20 mM buffer, 240 mM tonicity agent, 10 mM stabilizer, 0.6 mg/mL surfactant, and pH 5.5. In some embodiments, the liquid pharmaceutical formulation comprises 160 mg/mL anti-TIGIT monoclonal antibody, 20 mM histidine acetate, 240 mM sucrose, 10 mM methionine, 0.6 mg/mL Polysorbate 20, and pH 5.5. In some embodiments, the liquid pharmaceutical formulation comprises 176 mg/mL anti-TIGIT monoclonal antibody, 30 mM histidine acetate, 180 mM sucrose, 5 mM methionine, 0.8 mg/mL Polysorbate 20, and pH 5.5.

In some embodiments, the liquid pharmaceutical formulation comprises about 160 mg/mL anti-TIGIT monoclonal antibody, about 20 mM buffer, about 240 mM tonicity agent, about 10 mM stabilizer, about 0.6 mg/mL surfactant, and pH of about 5.5. In some embodiments, the liquid pharmaceutical formulation comprises about 160 mg/mL anti-TIGIT monoclonal antibody, about 20 mM histidine acetate, about 240 mM sucrose, about 10 mM methionine, about 0.6 mg/mL Polysorbate 20, and pH of about 5.5. In some embodiments, the liquid pharmaceutical formulation comprises about 176 mg/mL anti-TIGIT monoclonal antibody, about 30 mM histidine acetate, about 180 mM sucrose, about 5 mM methionine, about 0.8 mg/mL Polysorbate 20, and pH of about 5.5.

In some embodiments, the liquid pharmaceutical formulation comprises 150 to 170 mg/mL anti-TIGIT monoclonal antibody, 1000 to 3000 U/mL hyaluronidase, 15 to 25 mM buffer, 230 to 250 mM tonicity agent, 5 to 15 mM stabilizer, 0.5 to 0.7 mg/mL surfactant, and pH of 5.4 to 5.6. In some embodiments, the liquid pharmaceutical formulation comprises 150 to 170 mg/mL anti-TIGIT monoclonal antibody, 1000 to 3000 U/mL hyaluronidase, 15 to 25 mM histidine acetate, 230 to 250 mM sucrose, 5 to 15 mM methionine, 0.5 to 0.7 mg/mL Polysorbate 20, and pH of 5.4 to 5.6. In some embodiments, the liquid pharmaceutical formulation comprises 155 to 165 mg/mL anti-TIGIT monoclonal antibody, 1500 to 2500 U/mL hyaluronidase, 18 to 22 mM buffer, 235 to 245 mM tonicity agent, 8 to 12 mM stabilizer, 0.55 to 0.65 mg/mL surfactant, and pH of 5.45 to 5.55. In some embodiments, the liquid pharmaceutical formulation comprises 155 to 165 mg/mL anti-TIGIT monoclonal antibody, 1500 to 2500 U/mL hyaluronidase, 18 to 22 mM histidine acetate, 235 to 245 mM sucrose, 8 to 12 mM methionine, 0.55 to 0.65 mg/mL Polysorbate 20, and pH of 5.45 to 5.55.

In some embodiments, the liquid pharmaceutical formulation comprises about 150 to about 170 mg/mL anti-TIGIT monoclonal antibody, about 1000 to about 3000 U/mL hyaluronidase, about 15 to about 25 mM buffer, about 230 to about 250 mM tonicity agent, about 5 to about 15 mM stabilizer, about 0.5 to about 0.7 mg/mL surfactant, and pH of about 5.4 to about 5.6. In some embodiments, the liquid pharmaceutical formulation comprises about 150 to about 170 mg/mL anti-TIGIT monoclonal antibody, about 1000 to about 3000 U/mL hyaluronidase, about 15 to about 25 mM histidine acetate, about 230 to about 250 mM sucrose, about 5 to about 15 mM methionine, about 0.5 to about 0.7 mg/mL Polysorbate 20, and pH of about 5.4 to about 5.6. In some embodiments, the liquid pharmaceutical formulation comprises about 155 to about 165 mg/mL anti-TIGIT monoclonal antibody, about 1500 to about 2500 U/mL hyaluronidase, about 18 to about 22 mM buffer, about 235 to about 245 mM tonicity agent, about 8 to about 12 mM stabilizer, about 0.55 to about 0.65 mg/mL surfactant, and pH of about 5.45 to about 5.55. In some embodiments, the liquid pharmaceutical formulation comprises about 155 to about 165 mg/mL anti-TIGIT monoclonal antibody, about 1500 to about 2500 U/mL hyaluronidase, about 18 to about 22 mM histidine acetate, about 235 to about 245 mM sucrose, about 8 to about 12 mM methionine, about 0.55 to about 0.65 mg/mL Polysorbate 20, and pH of about 5.45 to about 5.55.

In some embodiments, the liquid pharmaceutical formulation comprises 160 mg/mL anti-TIGIT monoclonal antibody, 2000 U/mL hyaluronidase, 20 mM buffer, 240 mM tonicity agent, 10 mM stabilizer, 0.6 mg/mL surfactant, and pH 5.5. In some embodiments, the liquid pharmaceutical formulation comprises 160 mg/mL anti-TIGIT monoclonal antibody, 2000 U/mL hyaluronidase, 20 mM histidine acetate, 240 mM sucrose, 10 mM methionine, 0.6 mg/mL Polysorbate 20, and pH 5.5.

In some embodiments, the liquid pharmaceutical formulation comprises about 160 mg/mL anti-TIGIT monoclonal antibody, about 2000 U/mL hyaluronidase, about 20 mM buffer, about 240 mM tonicity agent, about 10 mM stabilizer, about 0.6 mg/mL surfactant, and pH of about 5.5. In some embodiments, the liquid pharmaceutical formulation comprises about 160 mg/mL anti-TIGIT monoclonal antibody, about 2000 U/mL hyaluronidase, about 20 mM histidine acetate, about 240 mM sucrose, about 10 mM methionine, about 0.6 mg/mL Polysorbate 20, and pH of about 5.5.

In some embodiments, the liquid pharmaceutical formulation comprises 30 to 50 mg/mL anti-TIGIT monoclonal antibody, 70 mg/mL to 90 mg/mL anti-PD-L1 monoclonal antibody, 15 mM to 25 mM buffer, 230 mM to 250 mM tonicity agent, 5 mM to 15 mM stabilizer, 0.5 mg/mL to 0.7 mg/mL surfactant, and pH of 5.7 to 5.9. In some embodiments, the liquid pharmaceutical formulation comprises 30 mg/mL to 50 mg/mL anti-TIGIT monoclonal antibody, 70 mg/mL to 90 mg/mL anti-PD-L1 monoclonal antibody, 15 mM to 25 mM histidine acetate, 230 mM to 250 mM sucrose, 5 mM to 15 mM methionine, 0.5 mg/mL to 0.7 mg/mL Polysorbate 20, and pH of 5.7 to 5.9. In some embodiments, the liquid pharmaceutical formulation comprises 35 mg/mL to 45 mg/mL anti-TIGIT monoclonal antibody, 75 mg/mL to 85 mg/mL anti-PD-L1 monoclonal antibody, 18 mM to 22 mM buffer, 235 mM to 245 mM tonicity agent, 8 mM to 12 mM stabilizer, 0.55 mg/mL to 0.65 mg/mL surfactant, and pH of 5.75 to 5.85. In some embodiments, the liquid pharmaceutical formulation comprises 35 mg/mL to 45 mg/mL anti-TIGIT monoclonal antibody, 75 mg/mL to 85 mg/mL anti-PD-L1 monoclonal antibody, 18 mM to 22 mM histidine acetate, 235 mM to 245 mM sucrose, 8 mM to 12 mM methionine, 0.55 mg/mL to 0.65 mg/mL Polysorbate 20, and pH of 5.75 to 5.85.

In some embodiments, the liquid pharmaceutical formulation comprises about 30 mg/mL to about 50 mg/mL anti-TIGIT monoclonal antibody, about 70 mg/mL to about 90 mg/mL anti-PD-L1 monoclonal antibody, about 15 mM to about 25 mM buffer, about 230 mM to about 250 mM tonicity agent, about 5 mM to about 15 mM stabilizer, about 0.5 mg/mL to about 0.7 mg/mL surfactant, and pH of about 5.7 to about 5.9. In some embodiments, the liquid pharmaceutical formulation comprises about 30 mg/mL to about 50 mg/mL anti-TIGIT monoclonal antibody, about 70 mg/mL to about 90 mg/mL anti-PD-L1 monoclonal antibody, about 15 mM to about 25 mM histidine acetate, about 230 mM to about 250 mM sucrose, about 5 mM to about 15 mM methionine, about 0.5 mg/mL to about 0.7 mg/mL Polysorbate 20, and pH of about 5.7 to about 5.9. In some embodiments, the liquid pharmaceutical formulation comprises about 35 mg/mL to about 45 mg/mL anti-TIGIT monoclonal antibody, about 75 mg/mL to about 85 mg/mL anti-PD-L1 monoclonal antibody, about 18 mM to about 22 mM buffer, about 235 mM to about 245 mM tonicity agent, about 8 mM to about 12 mM stabilizer, about 0.55 mg/mL to about 0.65 mg/mL surfactant, and pH of about 5.75 to about 5.85. In some embodiments, the liquid pharmaceutical formulation comprises about 35 mg/mL to about 45 mg/mL anti-TIGIT monoclonal antibody, about 75 mg/mL to about 85 mg/mL anti-PD-L1 monoclonal antibody, about 18 mM to about 22 mM histidine acetate, about 235 mM to about 245 mM sucrose, about 8 mM to about 12 mM methionine, about 0.55 mg/mL to about 0.65 mg/mL Polysorbate 20, and pH of about 5.75 to about 5.85.

In some embodiments, the liquid pharmaceutical formulation comprises 40 mg/mL anti-TIGIT monoclonal antibody, 80 mg/mL anti-PD-L1 monoclonal antibody, 20 mM buffer, 240 mM tonicity agent, 10 mM stabilizer, 0.6 mg/mL surfactant, and pH 5.8. In some embodiments, the liquid pharmaceutical formulation comprises 40 mg/mL anti-TIGIT monoclonal antibody, 80 mg/mL anti-PD-L1 monoclonal antibody, 20 mM histidine acetate, 240 mM sucrose, 10 mM methionine, 0.6 mg/mL Polysorbate 20, and pH 5.8.

In some embodiments, the liquid pharmaceutical formulation comprises about 40 mg/mL anti-TIGIT monoclonal antibody, about 80 mg/mL anti-PD-L1 monoclonal antibody, about 20 mM buffer, about 240 mM tonicity agent, about 10 mM stabilizer, about 0.6 mg/ml surfactant, and pH of about 5.8. In some embodiments, the liquid pharmaceutical formulation comprises about 40 mg/mL anti-TIGIT monoclonal antibody, about 80 mg/mL anti-PD-L1 monoclonal antibody, about 20 mM histidine acetate, about 240 mM sucrose, about 10 mM methionine, about 0.6 mg/mL Polysorbate 20, and pH of about 5.8.

In some embodiments, the liquid pharmaceutical formulation is formulated to be administered intravenously or subcutaneously. In some embodiments, the liquid pharmaceutical formulation is formulated to be administered intravenously. In some embodiments, the liquid pharmaceutical formulation is formulated to be administered subcutaneously.

In some embodiments, the anti-TIGIT monoclonal antibody in the formulation is not subject to prior lyophilization. In some embodiments, the anti-PD-L1 monoclonal antibody in the formulation is not subject to prior lyophilization.

A further aspect of the present disclosure provides liquid pharmaceutical formulations comprising an anti-TIGIT monoclonal antibody and an anti-PD-L1 monoclonal antibody. In some embodiments, the liquid pharmaceutical formulation comprises: (a) 18 mg/mL to 176 mg/mL of an anti-TIGIT monoclonal antibody; (b) 54 mg/mL to 137.5 mg/mL of an anti-PD-L1 monoclonal antibody; (c) 5 mM to 30 mM of a histidine buffer; (d) 120 mM to 320 mM of sucrose; and (e) 0.02% (w/v) to 0.08% (w/v) polysorbate 20, wherein the liquid pharmaceutical formulation is characterized by a pH of about 5.2-6.1; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments liquid pharmaceutical formulation comprises 18 mg/mL to 176 mg/mL of an anti-TIGIT monoclonal antibody, 54 mg/mL to 137.5 mg/mL of an anti-PD-L1 monoclonal antibody, 5 mM to 30 mM of a histidine buffer, 120 mM to 320 mM of sucrose, 0.02% (w/v) to 0.08% (w/v) polysorbate 20, and a pH of about 5.2-6.1. In some embodiments, the liquid pharmaceutical formulation comprises 36 mg/mL to 44 mg/mL of the anti-TIGIT monoclonal antibody, 72 mg/mL to 88 mg/mL of the anti-PD-L1 monoclonal antibody, 15 mM to 25 mM of the histidine buffer, 200 mM to 280 mM of sucrose, 0.04% (w/v) to 0.08% (w/v) polysorbate 20, and pH of about 5.5-6.1. In some embodiments, the liquid pharmaceutical formulation comprises 40 mg/mL of the anti-TIGIT monoclonal antibody, 80 mg/mL of the anti-PD-L1 monoclonal antibody, 20 mM of the histidine buffer, 240 mM of sucrose, 0.06% (w/v) of polysorbate 20, and pH of about 5.5. In some embodiments, the liquid pharmaceutical formulation comprises 40 mg/mL of the anti-TIGIT monoclonal antibody, 80 mg/mL of the anti-PD-L1 monoclonal antibody, 20 mM of the histidine buffer, 240 mM of sucrose, 0.06% (w/v) of polysorbate 20, and pH of about 5.6. In some embodiments, the liquid pharmaceutical formulation comprises 40 mg/mL of the anti-TIGIT monoclonal antibody, 80 mg/mL of the anti-PD-L1 monoclonal antibody, 20 mM of the histidine buffer, 240 mM of sucrose, 0.06% (w/v) of polysorbate 20, and pH of about 5.7. In some embodiments, the liquid pharmaceutical formulation comprises 40 mg/mL of the anti-TIGIT monoclonal antibody, 80 mg/mL of the anti-PD-L1 monoclonal antibody, 20 mM of the histidine buffer, 240 mM of sucrose, 0.06% (w/v) of polysorbate 20, and pH of about 5.8. In some embodiments liquid pharmaceutical formulation comprises about 18 mg/mL to about 176 mg/mL of an anti-TIGIT monoclonal antibody, about 54 mg/mL to about 137.5 mg/mL of an anti-PD-L1 monoclonal antibody, about 5 mM to about 30 mM of a histidine buffer, about 120 mM to about 320 mM of sucrose, about 0.02% (w/v) to about 0.08% (w/v) polysorbate 20, and a pH of about 5.2-6.1. In some embodiments, the liquid pharmaceutical formulation comprises about 36 mg/mL to about 44 mg/mL of the anti-TIGIT monoclonal antibody, about 72 mg/mL to about 88 mg/mL of the anti-PD-L1 monoclonal antibody, about 15 mM to about 25 mM of the histidine buffer, about 200 mM to about 280 mM of sucrose, about 0.04% (w/v) to about 0.08% (w/v) polysorbate 20, and pH of about 5.5-6.1. In some embodiments, the liquid pharmaceutical formulation comprises about 40 mg/ml of the anti-TIGIT monoclonal antibody, about 80 mg/mL of the anti-PD-L1 monoclonal antibody, about 20 mM of the histidine buffer, about 240 mM of sucrose, about 0.06% (w/v) of polysorbate 20, and pH of about 5.8.

Another aspect of the present disclosure provides liquid pharmaceutical formulations comprising an anti-TIGIT monoclonal antibody and an anti-PD-L1 monoclonal antibody. In some embodiments, the liquid pharmaceutical formulation comprises: (a) 18 mg/mL to 75 mg/mL of an anti-TIGIT monoclonal antibody; (b) 54 mg/mL to 137.5 mg/mL of an anti-PD-L1 monoclonal antibody; (c) 12 mM to 28 mM of a histidine buffer; (d) 100 mM to 300 mM of sucrose; and (e) 0.02% (w/v) to 0.08% (w/v) polysorbate 20, wherein the liquid pharmaceutical formulation is characterized by a pH of about 5.4-6.2; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments, the liquid pharmaceutical formulation comprises (a) 30 mg/mL to 50 mg/mL anti-TIGIT monoclonal antibody; (b) 70 mg/mL to 90 mg/mL anti-PD-L1 monoclonal antibody; (c) 15 mM to 25 mM histidine acetate; (d) 200 mM to 280 mM sucrose; and (3) 0.04% (w/v) to 0.08% (w/v) Polysorbate 20, wherein the liquid pharmaceutical formulation is characterized by a pH of 5.6 to 6.0. In some embodiments, the liquid pharmaceutical formulation comprises (a) 36 mg/mL to 44 mg/mL anti-TIGIT monoclonal antibody; (b) 72 mg/mL to 88 mg/mL anti-PD-L1 monoclonal antibody; (c) 18 mM to 22 mM histidine acetate; (d) 220 mM to 260 mM sucrose; and (3) 0.05% (w/v) to 0.07% (w/v) Polysorbate 20, wherein the liquid pharmaceutical formulation is characterized by a pH of 5.7 to 5.9.

Another aspect of the present disclosure provides liquid pharmaceutical formulations comprising an anti-TIGIT monoclonal antibody and suitable for co-administration with an anti-PD-L1 monoclonal antibody. In some embodiments, the anti-PD-L1 monoclonal antibody, is an anti-PD-L1 monoclonal antibody disclosed supra. In some embodiments, the liquid pharmaceutical formulation comprises: (a) 18 mg/mL to 176 mg/mL of an anti-TIGIT monoclonal antibody; (b) 5 mM to 30 mM of histidine acetate; (c) 100 mM to 320 mM of sucrose; and (d) 0.01% (w/v) to 0.08% (w/v) polysorbate 20, wherein the liquid pharmaceutical formulation is characterized by a pH of about 5.0-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6. In some embodiments, the liquid pharmaceutical formulation comprises: (a) about 18 mg/mL to about 176 mg/mL of an anti-TIGIT monoclonal antibody; (b) about 5 mM to about 30 mM of histidine acetate; (c) about 100 mM to about 320 mM of sucrose; and (d) about 0.01% (w/v) to about 0.08% (w/v) polysorbate 20, wherein the liquid pharmaceutical formulation is characterized by a pH of about 5.0-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments the formulation comprises: (a) 144 mg/mL to 176 mg/mL of the anti-TIGIT monoclonal antibody; (b) 5 mM to 25 mM of the histidine buffer; (c) 180 mM to 320 mM of sucrose; and (d) 0.05% (w/v) to 0.08% (w/v) polysorbate 20, wherein the liquid pharmaceutical formulation is characterized by a pH of about 5.2-5.8. In some embodiments, the formulation comprises: (a) 160 mg/mL of the anti-TIGIT monoclonal antibody; (b) 20 mM of the histidine buffer; (c) 240 mM of sucrose; and (d) 0.06% (w/v) of polysorbate 20, wherein the liquid pharmaceutical formulation is characterized by a pH of about 5.5. In some embodiments the formulation comprises: (a) about 144 mg/mL to about 176 mg/mL of the anti-TIGIT monoclonal antibody; (b) 5 mM to 25 mM of the histidine buffer; (c) about 180 mM to about 320 mM of sucrose; and (d) about 0.05% (w/v) to about 0.08% (w/v) polysorbate 20, wherein the liquid pharmaceutical formulation is characterized by a pH of about 5.2-5.8. In some embodiments, the formulation comprises: (a) about 160 mg/mL of the anti-TIGIT monoclonal antibody; (b) about 20 mM of the histidine buffer; (c) about 240 mM of sucrose; and (d) about 0.06% (w/v) of polysorbate 20, wherein the liquid pharmaceutical formulation is characterized by a pH of about 5.5.

An additional aspect of the present disclosure provides liquid pharmaceutical formulations suitable for subcutaneous injection and comprising an anti-TIGIT monoclonal antibody. In some embodiments the liquid pharmaceutical formulation comprises: (a) 18 mg/ml to 176 mg/mL of an anti-TIGIT monoclonal antibody; (b) 500 U/mL to 2600 U/mL of a hyaluronidase; (c) 5 mM to 30 mM of a histidine buffer; (d) 180 mM to 320 mM of sucrose; and (e) 0.03% (w/v) to 0.08% (w/v) polysorbate 20, wherein the liquid pharmaceutical formulation is characterized by a pH of about 5.2-6.0; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments, the liquid pharmaceutical formulation comprises 18 mg/mL to 176 mg/mL of an anti-TIGIT monoclonal antibody, (b) 500 U/mL to 2600 U/mL of a hyaluronidase, 5 mM to 30 mM of a histidine buffer, 180 mM to 320 mM of sucrose, 0.03% (w/v) to 0.08% (w/v) polysorbate 20, and pH of about 5.2-6.0. In some embodiments, the liquid pharmaceutical formulation comprises 144 mg/mL to 176 mg/mL of an anti-TIGIT monoclonal antibody, (b) 1400 U/mL to 2600 U/mL of a hyaluronidase, 5 mM to 25 mM of a histidine buffer, 180 mM to 320 mM of sucrose, 0.04% (w/v) to 0.08% (w/v) polysorbate 20, and pH of about 5.2-5.8. In some embodiments, the liquid pharmaceutical formulation comprises 160 mg/mL of an anti-TIGIT monoclonal antibody, (b) 2000 U/mL of a hyaluronidase, 20 mM of a histidine buffer, 240 mM of sucrose, 0.06% (w/v) polysorbate 20, and pH of about 5.5. In some embodiments, the liquid pharmaceutical formulation comprises 144 mg/mL to 176 mg/mL of the anti-TIGIT monoclonal antibody, 5 mM to 25 mM of the histidine buffer, 180 mM to 320 mM of sucrose, 0.05% (w/v) to 0.08% (w/v) polysorbate 20, pH of about 5.2-5.8. In some embodiments, the liquid pharmaceutical formulation comprises 160 mg/mL of the anti-TIGIT monoclonal antibody, 20 mM of the histidine buffer, 240 mM of sucrose, 0.06% (w/v) of polysorbate 20, and pH of about 5.5. In some embodiments, the liquid pharmaceutical formulation comprises about 18 mg/mL to about 176 mg/mL of an anti-TIGIT monoclonal antibody (b) about 500 U/mL to about 2600 U/mL of a hyaluronidase, about 5 mM to about 30 mM of a histidine buffer, about 180 mM to about 320 mM of sucrose, about 0.03% (w/v) to about 0.08% (w/v) polysorbate 20, and pH of about 5.2-6.0. In some embodiments, the liquid pharmaceutical formulation comprises about 144 mg/mL to about 176 mg/mL of the anti-TIGIT monoclonal antibody, about 5 mM to about 25 mM of the histidine buffer, about 180 mM to about 320 mM of sucrose, about 0.05% (w/v) to about 0.08% (w/v) polysorbate 20, pH of about 5.2-5.8. In some embodiments, the liquid pharmaceutical formulation comprises about 160 mg/mL of the anti-TIGIT monoclonal antibody, about 20 mM of the histidine buffer, about 240 mM of sucrose, about 0.06% (w/v) of polysorbate 20, and pH of about 5.5.

In some embodiments, the hyaluronidase is a recombinant human hyaluronidase. In some embodiments, the hyaluronidase is a human soluble PH20 hyaluronidase glycoprotein, such as rHuPH20. In some embodiments, the hyaluronidase is a human soluble PH20 hyaluronidase glycoprotein. In particular embodiments, the hyaluronidase is rHuPH20.

In another aspect, the present disclosure provides liquid pharmaceutical formulations suitable for subcutaneous injection and comprising an anti-TIGIT monoclonal antibody, an anti-PD-L1 monoclonal antibody, and hyaluronidase. In some embodiments, the liquid pharmaceutical formulation comprises: (a) 30 mg/mL to 60 mg/mL of an anti-TIGIT monoclonal antibody; (b) 60 mg/mL to 120 mg/mL of an anti-PD-L1 monoclonal antibody; (c) 500 U/mL to 2600 U/mL hyaluronidase; (d) 5 mM to 30 mM of a histidine buffer; (e) 180 mM to 320 mM of sucrose; and (f) 0.03% (w/v) to 0.08% (w/v) polysorbate 20, wherein the liquid pharmaceutical formulation is characterized by a pH of about 5.2-6.1; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments, the formulation comprises: (a) 30 mg/mL to 60 mg/mL of the anti-TIGIT monoclonal antibody; (b) 60 mg/mL to 120 mg/mL of the anti-PD-L1 monoclonal antibody; (c) 1000 U/mL to 3000 U/mL hyaluronidase; (d) 15 mM to 25 mM of the histidine buffer; (e) 200 mM to 280 mM of sucrose; and (f) 0.04% (w/v) to 0.08% (w/v) polysorbate 20, wherein the liquid pharmaceutical formulation is characterized by a pH of about 5.2-6.1. In some embodiments, the formulation comprises: (a) 35 mg/mL to 45 mg/mL of the anti-TIGIT monoclonal antibody; (b) 70 mg/mL to 90 mg/mL of the anti-PD-L1 monoclonal antibody; (c) 1500 U/mL to 2500 U/mL hyaluronidase; (d) 18 mM to 22 mM of the histidine buffer; (3) 220 mM to 260 mM of sucrose; and (f) 0.05% (w/v) to 0.07% (w/v) polysorbate 20, wherein the liquid pharmaceutical formulation is characterized by a pH of about 5.5-5.8. In some embodiments, the formulation comprises: (a) 40 mg/mL to 50 mg/mL of the anti-TIGIT monoclonal antibody; (b) 80 mg/mL to 100 mg/mL of the anti-PD-L1 monoclonal antibody; (c) 1500 U/mL to 2500 U/mL hyaluronidase; (d) 18 mM to 22 mM of the histidine buffer; (e) 220 mM to 260 mM of sucrose; and (f) 0.05% (w/v) to 0.07% (w/v) polysorbate 20, wherein the liquid pharmaceutical formulation is characterized by a pH of about 5.5-5.8.

In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 30 mg/mL of the anti-TIGIT monoclonal antibody; (b) 60 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 35 mg/mL of the anti-TIGIT monoclonal antibody; (b) 70 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 40 mg/mL of the anti-TIGIT monoclonal antibody; (b) 80 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 45 mg/mL of the anti-TIGIT monoclonal antibody; (b) 90 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 50 mg/mL of the anti-TIGIT monoclonal antibody; (b) 100 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 55 mg/mL of the anti-TIGIT monoclonal antibody; (b) 110 mg/mL of the anti-PD-L1 monoclonal antibody; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments of any of the above formulations, the histidine buffer is histidine acetate.

In some embodiments of any of the above formulations, the heavy chain variable region of the anti-TIGIT monoclonal antibody comprises the amino acid sequence of SEQ ID NO: 7. In some embodiments, the heavy chain variable region of the anti-TIGIT monoclonal antibody comprises the amino acid sequence of SEQ ID NO: 8. In some embodiments, the light chain variable region of the anti-TIGIT monoclonal antibody comprises the amino acid sequence of SEQ ID NO: 9. In some embodiments, the heavy chain variable region of the anti-TIGIT monoclonal antibody comprises the amino acid sequence of SEQ ID NO: 7 and the light chain variable region of the anti-TIGIT monoclonal antibody comprises the amino acid sequence of 9. In some embodiments, the heavy chain variable region of the anti-TIGIT monoclonal antibody comprises the amino acid sequence of SEQ ID NO: 8 and the light chain variable region of the anti-TIGIT monoclonal antibody comprises the amino acid sequence of 9.

In some embodiments of any of the above formulations, the anti-TIGIT monoclonal antibody is an IgG antibody. In some embodiments, the anti-TIGIT monoclonal antibody is an IgG1 or an IgG4 antibody. In some embodiments, the anti-TIGIT monoclonal antibody is an IgG1 antibody. In some embodiments, the anti-TIGIT monoclonal antibody is an IgG4 antibody. In some embodiments, the anti-TIGIT monoclonal antibody is a full-length antibody. In some embodiments, the anti-TIGIT monoclonal antibody is a human antibody. In some embodiments, the anti-TIGIT monoclonal antibody is a humanized antibody. In some embodiments, the anti-TIGIT monoclonal antibody is a full-length IgG antibody. In some embodiments, the anti-TIGIT monoclonal antibody is a full-length IgG1 antibody. In some embodiments, the anti-TIGIT monoclonal antibody is a full-length human IgG1 antibody. In some embodiments, the anti-TIGIT monoclonal antibody is a full-length humanized IgG1 antibody. The anti-TIGIT monoclonal antibody may be an antibody fragment. In some embodiments, the anti-TIGIT monoclonal antibody is a Fab, a Fab′, a F(ab′)₂, a Fv, or a scFv fragment. In some embodiments, the anti-TIGIT monoclonal antibody is a Fab fragment. In some embodiments, the anti-TIGIT monoclonal antibody is a Fab′ fragment. In some embodiments, the anti-TIGIT monoclonal antibody is a F(ab′)₂, fragment. In some embodiments, the anti-TIGIT monoclonal antibody is a Fv fragment. In some embodiments, the anti-TIGIT monoclonal antibody is a scFv fragment. In some embodiments, the anti-TIGIT monoclonal antibody is a diabody. In some embodiments, the anti-TIGIT monoclonal antibody is a linear antibody. In some embodiments, the anti-TIGIT monoclonal antibody is a single-chain antibody molecule. In some embodiments, the anti-TIGIT monoclonal antibody is a multispecific antibody, e.g. formed from antibody fragments.

In some embodiments of any of the above formulations, the anti-TIGIT monoclonal antibody inhibits or blocks the interaction of CD226 with TIGIT.

In some embodiments of any of the above formulations, the heavy chain variable region of the anti-PD-L1 monoclonal antibody comprises the amino acid sequence of SEQ ID NO: 16. In some embodiments, the light chain variable region of the anti-PD-L1 monoclonal antibody comprises the amino acid sequence of SEQ ID NO: 17. In some embodiments, the heavy chain variable region of the anti-PD-L1 monoclonal antibody comprises the amino acid sequence of SEQ ID NO: 16 and the light chain variable region of the anti-PD-L1 monoclonal antibody comprises the amino acid sequence of SEQ ID NO: 17. In some embodiments, the anti-PD-L1 monoclonal antibody is atezolizumab, marketed as TECENTRIQ®. Atezolizumab is described in WHO Drug Information (International Nonproprietary Names for Pharmaceutical Substances), Proposed INN: List 112, Vol. 28, No. 4, 2014 (see page 488). In some embodiments, atezolizumab has the CAS Registry Number 1380723-44-3.

In some embodiments of any of the above formulations, the anti-PD-L1 monoclonal antibody is an IgG antibody. In some embodiments, the anti-PD-L1 monoclonal antibody is an IgG1 or an IgG4 antibody. In some embodiments, the anti-PD-L1 monoclonal antibody is an IgG1 antibody. In some embodiments, the anti-PD-L1 monoclonal antibody is an IgG4 antibody. In some embodiments, the anti-PD-L1 monoclonal antibody is a full-length antibody. In some embodiments, the anti-PD-L1 monoclonal antibody is a human antibody. In some embodiments, the anti-PD-L1 monoclonal antibody is a humanized antibody. In some embodiments, the anti-PD-L1 monoclonal antibody is a full-length IgG antibody. In some embodiments, the anti-PD-L1 monoclonal antibody is a full-length IgG1 antibody. In some embodiments, the anti-PD-L1 monoclonal antibody is a full-length human IgG1 antibody. In some embodiments, the anti-PD-L1 monoclonal antibody is a full-length humanized IgG1 antibody.

In some embodiments of any of the above formulations, the formulation further comprises a stabilizer. In some embodiments, the stabilizer is selected from the group consisting of methionine, glycine, alanine, proline, taurine, betaine, octopine, glutamate, sarcosine, γ-aminobutyric acid, and trimethylamine N-oxide. In particular embodiments, the stabilizer is methionine. In some embodiments, the stabilizer is glycine. In some embodiments, the stabilizer is alanine. In some embodiments, the stabilizer is proline. In some embodiments, the stabilizer is taurine. In some embodiments, the stabilizer is betaine. In some embodiments, the stabilizer is octopine. In some embodiments, the stabilizer is glutamate. In some embodiments, the stabilizer is sarcosine. In some embodiments, the stabilizer is γ-aminobutyric acid. In some embodiments, the stabilizer is trimethylamine N-oxide. In some embodiments, the concentration of the stabilizer is about 0 mM to about 15 mM. In some embodiments, the concentration of the stabilizer is about 5 mM to about 15 mM. In particular embodiments, the concentration of the stabilizer is about 10 mM. In some embodiments, the concentration of the stabilizer is 0 mM to about 15 mM. In some embodiments, the concentration of the stabilizer is 5 mM to 15 mM. In particular embodiments, the concentration of the stabilizer is 10 mM.

In some embodiments of any of the above formulations, the concentration of the anti-TIGIT monoclonal antibody is 18 mg/mL to 176 mg/mL. In some embodiments, the concentration of the anti-TIGIT monoclonal antibody is 18 mg/mL to 166 mg/mL. In some embodiments, the concentration of the anti-TIGIT monoclonal antibody is 18 mg/mL to 160 mg/mL. In some embodiments, the concentration of the anti-TIGIT monoclonal antibody is 18 mg/mL to 156 mg/mL. In some embodiments, the concentration of the anti-TIGIT monoclonal antibody is 18 mg/mL to 156 mg/mL. In some embodiments, the concentration of the anti-TIGIT monoclonal antibody is 18 mg/mL to 146 mg/mL. In some embodiments, the concentration of the anti-TIGIT monoclonal antibody is 18 mg/mL to 136 mg/mL. In some embodiments, the concentration of the anti-TIGIT monoclonal antibody is 18 mg/mL to 126 mg/mL. In some embodiments, the concentration of the anti-TIGIT monoclonal antibody is 18 mg/mL to 116 mg/mL. In some embodiments, the concentration of the anti-TIGIT monoclonal antibody is 18 mg/mL to 106 mg/mL. In some embodiments, the concentration of the anti-TIGIT monoclonal antibody is 18 mg/mL to 96 mg/mL. In some embodiments, the concentration of the anti-TIGIT monoclonal antibody is 18 mg/mL to 86 mg/mL. In some embodiments, the concentration of the anti-TIGIT monoclonal antibody is 18 mg/mL to 76 mg/mL. In some embodiments, the concentration of the anti-TIGIT monoclonal antibody is 18 mg/mL to 66 mg/mL. In some embodiments, the concentration of the anti-TIGIT monoclonal antibody is 18 mg/mL to 56 mg/mL. In some embodiments, the concentration of the anti-TIGIT monoclonal antibody is 18 mg/mL to 46 mg/mL. In some embodiments, the concentration of the anti-TIGIT monoclonal antibody is 26 mg/mL to 54 mg/mL. In some embodiments, the concentration of the anti-TIGIT monoclonal antibody is 31 mg/mL to 49 mg/mL. In some embodiments, the concentration of the anti-TIGIT monoclonal antibody is 36 mg/mL to 44 mg/mL. In some embodiments, the concentration of the anti-TIGIT monoclonal antibody is 124 mg/mL to 196 mg/mL. In some embodiments, the concentration of the anti-TIGIT monoclonal antibody is 134 mg/mL to 186 mg/mL. In some embodiments, the concentration of the anti-TIGIT monoclonal antibody is 139 mg/mL to 181 mg/mL. In some embodiments, the concentration of the anti-TIGIT monoclonal antibody is 144 mg/mL to 176 mg/mL.

In some embodiments of any of the above formulations, the concentration of the anti-TIGIT monoclonal antibody is 20 mg/mL to 180 mg/mL. In some embodiments, the concentration of the anti-TIGIT monoclonal antibody is 20 mg/mL to 170 mg/mL. In some embodiments, the concentration of the anti-TIGIT monoclonal antibody is 20 mg/mL to 160 mg/mL. In some embodiments, the concentration of the anti-TIGIT monoclonal antibody is 20 mg/mL to 155 mg/mL. In some embodiments, the concentration of the anti-TIGIT monoclonal antibody is 20 mg/mL to 150 mg/mL. In some embodiments, the concentration of the anti-TIGIT monoclonal antibody is 20 mg/mL to 145 mg/mL. In some embodiments, the concentration of the anti-TIGIT monoclonal antibody is 20 mg/mL to 140 mg/mL. In some embodiments, the concentration of the anti-TIGIT monoclonal antibody is 20 mg/mL to 135 mg/mL. In some embodiments, the concentration of the anti-TIGIT monoclonal antibody is 20 mg/mL to 130 mg/mL. In some embodiments, the concentration of the anti-TIGIT monoclonal antibody is 20 mg/mL to 125 mg/mL. In some embodiments, the concentration of the anti-TIGIT monoclonal antibody is 20 mg/mL to 120 mg/mL. In some embodiments, the concentration of the anti-TIGIT monoclonal antibody is 20 mg/mL to 115 mg/mL. In some embodiments, the concentration of the anti-TIGIT monoclonal antibody is 20 mg/mL to 110 mg/mL. In some embodiments, the concentration of the anti-TIGIT monoclonal antibody is 20 mg/mL to 105 mg/mL. In some embodiments, the concentration of the anti-TIGIT monoclonal antibody is 20 mg/mL to 100 mg/mL. In some embodiments, the concentration of the anti-TIGIT monoclonal antibody is 20 mg/mL to 95 mg/mL. In some embodiments, the concentration of the anti-TIGIT monoclonal antibody is 20 mg/mL to 90 mg/mL. In some embodiments, the concentration of the anti-TIGIT monoclonal antibody is 20 mg/mL to 85 mg/mL. In some embodiments, the concentration of the anti-TIGIT monoclonal antibody is 20 mg/mL to 80 mg/mL. In some embodiments, the concentration of the anti-TIGIT monoclonal antibody is 20 mg/mL to 75 mg/mL. In some embodiments, the concentration of the anti-TIGIT monoclonal antibody is 20 mg/mL to 70 mg/mL. In some embodiments, the concentration of the anti-TIGIT monoclonal antibody is 20 mg/mL to 65 mg/mL. In some embodiments, the concentration of the anti-TIGIT monoclonal antibody is 20 mg/mL to 60 mg/mL. In some embodiments, the concentration of the anti-TIGIT monoclonal antibody is 20 mg/mL to 55 mg/mL. In some embodiments, the concentration of the anti-TIGIT monoclonal antibody is 20 mg/mL to 50 mg/mL. In some embodiments, the concentration of the anti-TIGIT monoclonal antibody is 20 mg/mL to 45 mg/mL. In some embodiments, the concentration of the anti-TIGIT monoclonal antibody is 20 mg/mL to 40 mg/mL. In some embodiments, the concentration of the anti-TIGIT monoclonal antibody is 25 mg/mL to 55 mg/mL. In some embodiments, the concentration of the anti-TIGIT monoclonal antibody is 30 mg/mL to 50 mg/mL. In some embodiments, the concentration of the anti-TIGIT monoclonal antibody is 35 mg/mL to 45 mg/mL. In some embodiments, the concentration of the anti-TIGIT monoclonal antibody is 125 mg/mL to 200 mg/mL. In some embodiments, the concentration of the anti-TIGIT monoclonal antibody is 135 mg/mL to 185 mg/mL. In some embodiments, the concentration of the anti-TIGIT monoclonal antibody is 140 mg/mL to 180 mg/mL. In some embodiments, the concentration of the anti-TIGIT monoclonal antibody is 145 mg/mL to 175 mg/mL. In some embodiments, the concentration of the anti-TIGIT monoclonal antibody is 150 mg/mL to 170 mg/mL. In some embodiments, the concentration of the anti-TIGIT monoclonal antibody is 155 mg/mL to 165 mg/mL.

In some embodiments of any of the above formulations, the concentration of the anti-TIGIT monoclonal antibody is about 18 mg/mL to about 176 mg/mL. In some embodiments, the concentration of the anti-TIGIT monoclonal antibody is about 18 mg/mL to about 166 mg/mL. In some embodiments, the concentration of the anti-TIGIT monoclonal antibody is about 18 mg/mL to about 160 mg/mL. In some embodiments, the concentration of the anti-TIGIT monoclonal antibody is about 18 mg/mL to about 156 mg/mL. In some embodiments, the concentration of the anti-TIGIT monoclonal antibody is about 18 mg/mL to about 156 mg/mL. In some embodiments, the concentration of the anti-TIGIT monoclonal antibody is about 18 mg/mL to about 146 mg/mL. In some embodiments, the concentration of the anti-TIGIT monoclonal antibody is about 18 mg/mL to about 136 mg/mL. In some embodiments, the concentration of the anti-TIGIT monoclonal antibody is about 18 mg/mL to about 126 mg/mL. In some embodiments, the concentration of the anti-TIGIT monoclonal antibody is about 18 mg/mL to about 116 mg/mL. In some embodiments, the concentration of the anti-TIGIT monoclonal antibody is about 18 mg/mL to about 106 mg/mL. In some embodiments, the concentration of the anti-TIGIT monoclonal antibody is about 18 mg/mL to about 96 mg/mL. In some embodiments, the concentration of the anti-TIGIT monoclonal antibody is about 18 mg/mL to about 86 mg/mL. In some embodiments, the concentration of the anti-TIGIT monoclonal antibody is about 18 mg/mL to about 76 mg/mL. In some embodiments, the concentration of the anti-TIGIT monoclonal antibody is about 18 mg/mL to about 66 mg/mL. In some embodiments, the concentration of the anti-TIGIT monoclonal antibody is about 18 mg/mL to about 56 mg/mL. In some embodiments, the concentration of the anti-TIGIT monoclonal antibody is about 18 mg/mL to about 46 mg/mL. In some embodiments, the concentration of the anti-TIGIT monoclonal antibody is about 26 mg/mL to about 54 mg/mL. In some embodiments, the concentration of the anti-TIGIT monoclonal antibody is about 31 mg/mL to about 49 mg/mL. In some embodiments, the concentration of the anti-TIGIT monoclonal antibody is about 36 mg/ml to about 44 mg/mL. In some embodiments, the concentration of the anti-TIGIT monoclonal antibody is about 124 mg/mL to about 196 mg/mL. In some embodiments, the concentration of the anti-TIGIT monoclonal antibody is about 134 mg/mL to about 186 mg/mL. In some embodiments, the concentration of the anti-TIGIT monoclonal antibody is about 139 mg/mL to about 181 mg/mL. In some embodiments, the concentration of the anti-TIGIT monoclonal antibody is about 144 mg/mL to about 176 mg/mL.

In some embodiments of any of the above formulations, the concentration of the anti-TIGIT monoclonal antibody is about 20 mg/mL to about 180 mg/mL. In some embodiments, the concentration of the anti-TIGIT monoclonal antibody is about 20 mg/mL to about 170 mg/mL. In some embodiments, the concentration of the anti-TIGIT monoclonal antibody is about 20 mg/mL to about 160 mg/mL. In some embodiments, the concentration of the anti-TIGIT monoclonal antibody is about 20 mg/mL to about 155 mg/mL. In some embodiments, the concentration of the anti-TIGIT monoclonal antibody is about 20 mg/mL to about 150 mg/mL. In some embodiments, the concentration of the anti-TIGIT monoclonal antibody is about 20 mg/mL to about 145 mg/mL. In some embodiments, the concentration of the anti-TIGIT monoclonal antibody is about 20 mg/mL to about 140 mg/mL. In some embodiments, the concentration of the anti-TIGIT monoclonal antibody is about 20 mg/mL to about 135 mg/mL. In some embodiments, the concentration of the anti-TIGIT monoclonal antibody is about 20 mg/mL to about 130 mg/mL. In some embodiments, the concentration of the anti-TIGIT monoclonal antibody is about 20 mg/mL to about 125 mg/mL. In some embodiments, the concentration of the anti-TIGIT monoclonal antibody is about 20 mg/mL to about 120 mg/mL. In some embodiments, the concentration of the anti-TIGIT monoclonal antibody is about 20 mg/mL to about 115 mg/mL. In some embodiments, the concentration of the anti-TIGIT monoclonal antibody is about 20 mg/mL to about 110 mg/mL. In some embodiments, the concentration of the anti-TIGIT monoclonal antibody is about 20 mg/mL to about 105 mg/mL. In some embodiments, the concentration of the anti-TIGIT monoclonal antibody is about 20 mg/mL to about 100 mg/mL. In some embodiments, the concentration of the anti-TIGIT monoclonal antibody is about 20 mg/mL to about 95 mg/mL. In some embodiments, the concentration of the anti-TIGIT monoclonal antibody is about 20 mg/mL to about 90 mg/mL. In some embodiments, the concentration of the anti-TIGIT monoclonal antibody is about 20 mg/mL to about 85 mg/mL. In some embodiments, the concentration of the anti-TIGIT monoclonal antibody is about 20 mg/mL to about 80 mg/mL. In some embodiments, the concentration of the anti-TIGIT monoclonal antibody is about 20 mg/mL to about 75 mg/mL. In some embodiments, the concentration of the anti-TIGIT monoclonal antibody is about 20 mg/mL to about 70 mg/mL. In some embodiments, the concentration of the anti-TIGIT monoclonal antibody is about 20 mg/mL to about 65 mg/mL. In some embodiments, the concentration of the anti-TIGIT monoclonal antibody is about 20 mg/mL to about 60 mg/mL. In some embodiments, the concentration of the anti-TIGIT monoclonal antibody is about 20 mg/mL to about 55 mg/mL. In some embodiments, the concentration of the anti-TIGIT monoclonal antibody is about 20 mg/mL to about 50 mg/mL. In some embodiments, the concentration of the anti-TIGIT monoclonal antibody is about 20 mg/mL to about 45 mg/mL. In some embodiments, the concentration of the anti-TIGIT monoclonal antibody is about 20 mg/mL to about 40 mg/mL. In some embodiments, the concentration of the anti-TIGIT monoclonal antibody is about 25 mg/mL to about 55 mg/mL. In some embodiments, the concentration of the anti-TIGIT monoclonal antibody is about 30 mg/mL to about 50 mg/mL. In some embodiments, the concentration of the anti-TIGIT monoclonal antibody is about 35 mg/mL to about 45 mg/mL. In some embodiments, the concentration of the anti-TIGIT monoclonal antibody is about 125 mg/mL to about 200 mg/mL. In some embodiments, the concentration of the anti-TIGIT monoclonal antibody is about 135 mg/mL to about 185 mg/mL. In some embodiments, the concentration of the anti-TIGIT monoclonal antibody is about 140 mg/mL to about 180 mg/mL. In some embodiments, the concentration of the anti-TIGIT monoclonal antibody is about 145 mg/mL to about 175 mg/mL. In some embodiments, the concentration of the anti-TIGIT monoclonal antibody is about 150 mg/mL to about 170 mg/mL. In some embodiments, the concentration of the anti-TIGIT monoclonal antibody is about 155 mg/mL to about 165 mg/mL.

In some embodiments of any of the above formulations, the concentration of the anti-TIGIT monoclonal antibody is about 18 mg/mL. In some embodiments, the concentration of the anti-TIGIT monoclonal antibody is about 23 mg/mL. In some embodiments, the concentration of the anti-TIGIT monoclonal antibody is about 28 mg/mL. In some embodiments, the concentration of the anti-TIGIT monoclonal antibody is about 32 mg/mL. In some embodiments, the concentration of the anti-TIGIT monoclonal antibody is about 37 mg/mL. In some embodiments, the concentration of the anti-TIGIT monoclonal antibody is about 40 mg/mL. In some embodiments, the concentration of the anti-TIGIT monoclonal antibody is about 43 mg/mL. In some embodiments, the concentration of the anti-TIGIT monoclonal antibody is about 48 mg/mL. In some embodiments, the concentration of the anti-TIGIT monoclonal antibody is about 53 mg/mL. In some embodiments, the concentration of the anti-TIGIT monoclonal antibody is about 58 mg/mL. In some embodiments, the concentration of the anti-TIGIT monoclonal antibody is about 63 mg/mL. In some embodiments, the concentration of the anti-TIGIT monoclonal antibody is about 68 mg/mL. In some embodiments, the concentration of the anti-TIGIT monoclonal antibody is about 73 mg/mL. In some embodiments, the concentration of the anti-TIGIT monoclonal antibody is about 78 mg/mL. In some embodiments, the concentration of the anti-TIGIT monoclonal antibody is about 83 mg/mL. In some embodiments, the concentration of the anti-TIGIT monoclonal antibody is about 88 mg/mL. In some embodiments, the concentration of the anti-TIGIT monoclonal antibody is about 93 mg/mL. In some embodiments, the concentration of the anti-TIGIT monoclonal antibody is about 98 mg/mL. In some embodiments, the concentration of the anti-TIGIT monoclonal antibody is about 103 mg/mL. In some embodiments, the concentration of the anti-TIGIT monoclonal antibody is about 108 mg/mL. In some embodiments, the concentration of the anti-TIGIT monoclonal antibody is about 113 mg/mL. In some embodiments, the concentration of the anti-TIGIT monoclonal antibody is about 118 mg/mL. In some embodiments, the concentration of the anti-TIGIT monoclonal antibody is about 123 mg/mL. In some embodiments, the concentration of the anti-TIGIT monoclonal antibody is about 128 mg/mL. In some embodiments, the concentration of the anti-TIGIT monoclonal antibody is about 133 mg/mL. In some embodiments, the concentration of the anti-TIGIT monoclonal antibody is about 138 mg/mL. In some embodiments, the concentration of the anti-TIGIT monoclonal antibody is about 143 mg/mL. In some embodiments, the concentration of the anti-TIGIT monoclonal antibody is about 148 mg/mL. In some embodiments, the concentration of the anti-TIGIT monoclonal antibody is about 153 mg/mL. In some embodiments, the concentration of the anti-TIGIT monoclonal antibody is about 158 mg/mL. In some embodiments, the concentration of the anti-TIGIT monoclonal antibody is about 160 mg/mL. In some embodiments, the concentration of the anti-TIGIT monoclonal antibody is about 163 mg/mL. In some embodiments, the concentration of the anti-TIGIT monoclonal antibody is about 168 mg/mL. In some embodiments, the concentration of the anti-TIGIT monoclonal antibody is about 173 mg/mL. In some embodiments, the concentration of the anti-TIGIT monoclonal antibody is about 176 mg/mL. In some embodiments, the concentration of the anti-TIGIT monoclonal antibody is about 178 mg/mL. In some embodiments, the concentration of the anti-TIGIT monoclonal antibody is about 183 mg/mL. In some embodiments, the concentration of the anti-TIGIT monoclonal antibody is about 188 mg/mL.

In some embodiments of any of the above formulations, the concentration of the anti-TIGIT monoclonal antibody is about 20 mg/mL. In some embodiments, the concentration of the anti-TIGIT monoclonal antibody is about 25 mg/mL. In some embodiments, the concentration of the anti-TIGIT monoclonal antibody is about 30 mg/mL. In some embodiments, the concentration of the anti-TIGIT monoclonal antibody is about 35 mg/mL. In particular embodiments, the concentration of the anti-TIGIT monoclonal antibody is about 40 mg/mL. In some embodiments, the concentration of the anti-TIGIT monoclonal antibody is about 45 mg/mL. In some embodiments, the concentration of the anti-TIGIT monoclonal antibody is about 50 mg/mL. In some embodiments, the concentration of the anti-TIGIT monoclonal antibody is about 55 mg/mL. In some embodiments, the concentration of the anti-TIGIT monoclonal antibody is about 60 mg/mL. In some embodiments, the concentration of the anti-TIGIT monoclonal antibody is about 65 mg/mL. In some embodiments, the concentration of the anti-TIGIT monoclonal antibody is about 70 mg/mL. In some embodiments, the concentration of the anti-TIGIT monoclonal antibody is about 75 mg/mL. In some embodiments, the concentration of the anti-TIGIT monoclonal antibody is about 80 mg/mL. In some embodiments, the concentration of the anti-TIGIT monoclonal antibody is about 85 mg/mL. In some embodiments, the concentration of the anti-TIGIT monoclonal antibody is about 90 mg/mL. In some embodiments, the concentration of the anti-TIGIT monoclonal antibody is about 95 mg/mL. In some embodiments, the concentration of the anti-TIGIT monoclonal antibody is about 100 mg/mL. In some embodiments, the concentration of the anti-TIGIT monoclonal antibody is about 105 mg/mL. In some embodiments, the concentration of the anti-TIGIT monoclonal antibody is about 110 mg/mL. In some embodiments, the concentration of the anti-TIGIT monoclonal antibody is about 115 mg/mL. In some embodiments, the concentration of the anti-TIGIT monoclonal antibody is about 120 mg/mL. In some embodiments, the concentration of the anti-TIGIT monoclonal antibody is about 125 mg/mL. In some embodiments, the concentration of the anti-TIGIT monoclonal antibody is about 130 mg/mL. In some embodiments, the concentration of the anti-TIGIT monoclonal antibody is about 135 mg/mL. In some embodiments, the concentration of the anti-TIGIT monoclonal antibody is about 140 mg/mL. In some embodiments, the concentration of the anti-TIGIT monoclonal antibody is about 145 mg/mL. In some embodiments, the concentration of the anti-TIGIT monoclonal antibody is about 150 mg/mL. In some embodiments, the concentration of the anti-TIGIT monoclonal antibody is about 155 mg/mL. In particular embodiments, the concentration of the anti-TIGIT monoclonal antibody is about 160 mg/mL. In some embodiments, the concentration of the anti-TIGIT monoclonal antibody is about 165 mg/mL. In some embodiments, the concentration of the anti-TIGIT monoclonal antibody is about 170 mg/mL. In some embodiments, the concentration of the anti-TIGIT monoclonal antibody is about 175 mg/mL. In some embodiments, the concentration of the anti-TIGIT monoclonal antibody is about 180 mg/mL. In some embodiments, the concentration of the anti-TIGIT monoclonal antibody is about 185 mg/mL. In some embodiments, the concentration of the anti-TIGIT monoclonal antibody is about 190 mg/mL. In some embodiments, the concentration of the anti-TIGIT monoclonal antibody is about 195 mg/mL. In some embodiments, the concentration of the anti-TIGIT monoclonal antibody is about 200 mg/mL.

In some embodiments of any of the above formulations, the concentration of the anti-TIGIT monoclonal antibody is 18 mg/mL. In some embodiments, the concentration of the anti-TIGIT monoclonal antibody is 23 mg/mL. In some embodiments, the concentration of the anti-TIGIT monoclonal antibody is 28 mg/mL. In some embodiments, the concentration of the anti-TIGIT monoclonal antibody is 32 mg/mL. In some embodiments, the concentration of the anti-TIGIT monoclonal antibody is 37 mg/mL. In some embodiments, the concentration of the anti-TIGIT monoclonal antibody is 40 mg/mL. In some embodiments, the concentration of the anti-TIGIT monoclonal antibody is 43 mg/mL. In some embodiments, the concentration of the anti-TIGIT monoclonal antibody is 48 mg/mL. In some embodiments, the concentration of the anti-TIGIT monoclonal antibody is 53 mg/mL. In some embodiments, the concentration of the anti-TIGIT monoclonal antibody is 58 mg/mL. In some embodiments, the concentration of the anti-TIGIT monoclonal antibody is 63 mg/mL. In some embodiments, the concentration of the anti-TIGIT monoclonal antibody is 68 mg/mL. In some embodiments, the concentration of the anti-TIGIT monoclonal antibody is 73 mg/mL. In some embodiments, the concentration of the anti-TIGIT monoclonal antibody is 78 mg/mL. In some embodiments, the concentration of the anti-TIGIT monoclonal antibody is 83 mg/mL. In some embodiments, the concentration of the anti-TIGIT monoclonal antibody is 88 mg/mL. In some embodiments, the concentration of the anti-TIGIT monoclonal antibody is 93 mg/mL. In some embodiments, the concentration of the anti-TIGIT monoclonal antibody is 98 mg/mL. In some embodiments, the concentration of the anti-TIGIT monoclonal antibody is 103 mg/mL. In some embodiments, the concentration of the anti-TIGIT monoclonal antibody is 108 mg/mL. In some embodiments, the concentration of the anti-TIGIT monoclonal antibody is 113 mg/mL. In some embodiments, the concentration of the anti-TIGIT monoclonal antibody is 118 mg/mL. In some embodiments, the concentration of the anti-TIGIT monoclonal antibody is 123 mg/mL. In some embodiments, the concentration of the anti-TIGIT monoclonal antibody is 128 mg/mL. In some embodiments, the concentration of the anti-TIGIT monoclonal antibody is 133 mg/mL. In some embodiments, the concentration of the anti-TIGIT monoclonal antibody is 138 mg/mL. In some embodiments, the concentration of the anti-TIGIT monoclonal antibody is 143 mg/mL. In some embodiments, the concentration of the anti-TIGIT monoclonal antibody is 148 mg/mL. In some embodiments, the concentration of the anti-TIGIT monoclonal antibody is 153 mg/mL. In some embodiments, the concentration of the anti-TIGIT monoclonal antibody is 158 mg/mL. In some embodiments, the concentration of the anti-TIGIT monoclonal antibody is 160 mg/mL. In some embodiments, the concentration of the anti-TIGIT monoclonal antibody is 163 mg/mL. In some embodiments, the concentration of the anti-TIGIT monoclonal antibody is 168 mg/mL. In some embodiments, the concentration of the anti-TIGIT monoclonal antibody is 173 mg/mL. In some embodiments, the concentration of the anti-TIGIT monoclonal antibody is 176 mg/mL. In some embodiments, the concentration of the anti-TIGIT monoclonal antibody is 178 mg/mL. In some embodiments, the concentration of the anti-TIGIT monoclonal antibody is 183 mg/mL. In some embodiments, the concentration of the anti-TIGIT monoclonal antibody is 188 mg/mL.

In some embodiments of any of the above formulations, the concentration of the anti-TIGIT monoclonal antibody is 20 mg/mL. In some embodiments, the concentration of the anti-TIGIT monoclonal antibody is 25 mg/mL. In some embodiments, the concentration of the anti-TIGIT monoclonal antibody is 30 mg/mL. In some embodiments, the concentration of the anti-TIGIT monoclonal antibody is 35 mg/mL. In particular embodiments, the concentration of the anti-TIGIT monoclonal antibody is 40 mg/mL. In some embodiments, the concentration of the anti-TIGIT monoclonal antibody is 45 mg/mL. In some embodiments, the concentration of the anti-TIGIT monoclonal antibody is 50 mg/mL. In some embodiments, the concentration of the anti-TIGIT monoclonal antibody is 55 mg/mL. In some embodiments, the concentration of the anti-TIGIT monoclonal antibody is 60 mg/mL. In some embodiments, the concentration of the anti-TIGIT monoclonal antibody is 65 mg/mL. In some embodiments, the concentration of the anti-TIGIT monoclonal antibody is 70 mg/mL. In some embodiments, the concentration of the anti-TIGIT monoclonal antibody is 75 mg/mL. In some embodiments, the concentration of the anti-TIGIT monoclonal antibody is 80 mg/mL. In some embodiments, the concentration of the anti-TIGIT monoclonal antibody is 85 mg/mL. In some embodiments, the concentration of the anti-TIGIT monoclonal antibody is 90 mg/mL. In some embodiments, the concentration of the anti-TIGIT monoclonal antibody is 95 mg/mL. In some embodiments, the concentration of the anti-TIGIT monoclonal antibody is 100 mg/mL. In some embodiments, the concentration of the anti-TIGIT monoclonal antibody is 105 mg/mL. In some embodiments, the concentration of the anti-TIGIT monoclonal antibody is 110 mg/mL. In some embodiments, the concentration of the anti-TIGIT monoclonal antibody is 115 mg/mL. In some embodiments, the concentration of the anti-TIGIT monoclonal antibody is 120 mg/mL. In some embodiments, the concentration of the anti-TIGIT monoclonal antibody is 125 mg/mL. In some embodiments, the concentration of the anti-TIGIT monoclonal antibody is 130 mg/mL. In some embodiments, the concentration of the anti-TIGIT monoclonal antibody is 135 mg/mL. In some embodiments, the concentration of the anti-TIGIT monoclonal antibody is 140 mg/mL. In some embodiments, the concentration of the anti-TIGIT monoclonal antibody is 145 mg/mL. In some embodiments, the concentration of the anti-TIGIT monoclonal antibody is 150 mg/mL. In some embodiments, the concentration of the anti-TIGIT monoclonal antibody is 155 mg/mL. In particular embodiments, the concentration of the anti-TIGIT monoclonal antibody is 160 mg/mL. In some embodiments, the concentration of the anti-TIGIT monoclonal antibody is 165 mg/mL. In some embodiments, the concentration of the anti-TIGIT monoclonal antibody is 170 mg/mL. In some embodiments, the concentration of the anti-TIGIT monoclonal antibody is 175 mg/mL. In some embodiments, the concentration of the anti-TIGIT monoclonal antibody is 180 mg/mL. In some embodiments, the concentration of the anti-TIGIT monoclonal antibody is 185 mg/mL. In some embodiments, the concentration of the anti-TIGIT monoclonal antibody is 190 mg/mL. In some embodiments, the concentration of the anti-TIGIT monoclonal antibody is 195 mg/mL. In some embodiments, the concentration of the anti-TIGIT monoclonal antibody is 200 mg/mL.

In some embodiments of any of the above formulations, the concentration of the anti-TIGIT monoclonal antibody is about 18 mg/mL. In some embodiments, the concentration of the anti-TIGIT monoclonal antibody is about 23 mg/mL. In some embodiments, the concentration of the anti-TIGIT monoclonal antibody is about 28 mg/mL. In some embodiments, the concentration of the anti-TIGIT monoclonal antibody is about 32 mg/mL. In some embodiments, the concentration of the anti-TIGIT monoclonal antibody is about 37 mg/mL. In some embodiments, the concentration of the anti-TIGIT monoclonal antibody is about 40 mg/mL. In some embodiments, the concentration of the anti-TIGIT monoclonal antibody is about 43 mg/mL. In some embodiments, the concentration of the anti-TIGIT monoclonal antibody is about 48 mg/mL. In some embodiments, the concentration of the anti-TIGIT monoclonal antibody is about 53 mg/mL. In some embodiments, the concentration of the anti-TIGIT monoclonal antibody is about 58 mg/mL. In some embodiments, the concentration of the anti-TIGIT monoclonal antibody is about 63 mg/mL. In some embodiments, the concentration of the anti-TIGIT monoclonal antibody is about 68 mg/mL. In some embodiments, the concentration of the anti-TIGIT monoclonal antibody is about 73 mg/mL. In some embodiments, the concentration of the anti-TIGIT monoclonal antibody is about 78 mg/mL. In some embodiments, the concentration of the anti-TIGIT monoclonal antibody is about 83 mg/mL. In some embodiments, the concentration of the anti-TIGIT monoclonal antibody is about 88 mg/mL. In some embodiments, the concentration of the anti-TIGIT monoclonal antibody is about 93 mg/mL. In some embodiments, the concentration of the anti-TIGIT monoclonal antibody is about 98 mg/mL. In some embodiments, the concentration of the anti-TIGIT monoclonal antibody is about 103 mg/mL. In some embodiments, the concentration of the anti-TIGIT monoclonal antibody is about 108 mg/mL. In some embodiments, the concentration of the anti-TIGIT monoclonal antibody is about 113 mg/mL. In some embodiments, the concentration of the anti-TIGIT monoclonal antibody is about 118 mg/mL. In some embodiments, the concentration of the anti-TIGIT monoclonal antibody is about 123 mg/mL. In some embodiments, the concentration of the anti-TIGIT monoclonal antibody is about 128 mg/mL. In some embodiments, the concentration of the anti-TIGIT monoclonal antibody is about 133 mg/mL. In some embodiments, the concentration of the anti-TIGIT monoclonal antibody is about 138 mg/mL. In some embodiments, the concentration of the anti-TIGIT monoclonal antibody is about 143 mg/mL. In some embodiments, the concentration of the anti-TIGIT monoclonal antibody is about 148 mg/mL. In some embodiments, the concentration of the anti-TIGIT monoclonal antibody is about 153 mg/mL. In some embodiments, the concentration of the anti-TIGIT monoclonal antibody is about 158 mg/mL. In some embodiments, the concentration of the anti-TIGIT monoclonal antibody is about 160 mg/mL. In some embodiments, the concentration of the anti-TIGIT monoclonal antibody is about 163 mg/mL. In some embodiments, the concentration of the anti-TIGIT monoclonal antibody is about 168 mg/mL. In some embodiments, the concentration of the anti-TIGIT monoclonal antibody is about 173 mg/mL. In some embodiments, the concentration of the anti-TIGIT monoclonal antibody is about 176 mg/mL. In some embodiments, the concentration of the anti-TIGIT monoclonal antibody is about 178 mg/mL. In some embodiments, the concentration of the anti-TIGIT monoclonal antibody is about 183 mg/mL. In some embodiments, the concentration of the anti-TIGIT monoclonal antibody is about 188 mg/mL.

In some embodiments of any of the above formulations, the concentration of the anti-TIGIT monoclonal antibody is about 20 mg/mL. In some embodiments, the concentration of the anti-TIGIT monoclonal antibody is about 25 mg/mL. In some embodiments, the concentration of the anti-TIGIT monoclonal antibody is about 30 mg/mL. In some embodiments, the concentration of the anti-TIGIT monoclonal antibody is about 35 mg/mL. In some embodiments, the concentration of the anti-TIGIT monoclonal antibody is about 40 mg/mL. In some embodiments, the concentration of the anti-TIGIT monoclonal antibody is about 45 mg/mL. In some embodiments, the concentration of the anti-TIGIT monoclonal antibody is about 50 mg/mL. In some embodiments, the concentration of the anti-TIGIT monoclonal antibody is about 55 mg/mL. In some embodiments, the concentration of the anti-TIGIT monoclonal antibody is about 60 mg/mL. In some embodiments, the concentration of the anti-TIGIT monoclonal antibody is about 65 mg/mL. In some embodiments, the concentration of the anti-TIGIT monoclonal antibody is about 70 mg/mL. In some embodiments, the concentration of the anti-TIGIT monoclonal antibody is about 75 mg/mL. In some embodiments, the concentration of the anti-TIGIT monoclonal antibody is about 80 mg/mL. In some embodiments, the concentration of the anti-TIGIT monoclonal antibody is about 85 mg/mL. In some embodiments, the concentration of the anti-TIGIT monoclonal antibody is about 90 mg/mL. In some embodiments, the concentration of the anti-TIGIT monoclonal antibody is about 95 mg/mL. In some embodiments, the concentration of the anti-TIGIT monoclonal antibody is about 100 mg/mL. In some embodiments, the concentration of the anti-TIGIT monoclonal antibody is about 105 mg/mL. In some embodiments, the concentration of the anti-TIGIT monoclonal antibody is about 110 mg/mL. In some embodiments, the concentration of the anti-TIGIT monoclonal antibody is about 115 mg/mL. In some embodiments, the concentration of the anti-TIGIT monoclonal antibody is about 120 mg/mL. In some embodiments, the concentration of the anti-TIGIT monoclonal antibody is about 125 mg/mL. In some embodiments, the concentration of the anti-TIGIT monoclonal antibody is about 130 mg/mL. In some embodiments, the concentration of the anti-TIGIT monoclonal antibody is about 135 mg/mL. In some embodiments, the concentration of the anti-TIGIT monoclonal antibody is about 140 mg/mL. In some embodiments, the concentration of the anti-TIGIT monoclonal antibody is about 145 mg/mL. In some embodiments, the concentration of the anti-TIGIT monoclonal antibody is about 150 mg/mL. In some embodiments, the concentration of the anti-TIGIT monoclonal antibody is about 155 mg/mL. In some embodiments, the concentration of the anti-TIGIT monoclonal antibody is about 165 mg/mL. In some embodiments, the concentration of the anti-TIGIT monoclonal antibody is about 170 mg/mL. In some embodiments, the concentration of the anti-TIGIT monoclonal antibody is about 175 mg/mL. In some embodiments, the concentration of the anti-TIGIT monoclonal antibody is about 180 mg/mL. In some embodiments, the concentration of the anti-TIGIT monoclonal antibody is about 185 mg/mL. In some embodiments, the concentration of the anti-TIGIT monoclonal antibody is about 190 mg/mL. In some embodiments, the concentration of the anti-TIGIT monoclonal antibody is about 195 mg/mL. In some embodiments, the concentration of the anti-TIGIT monoclonal antibody is about 200 mg/mL.

In some embodiments, the formulation comprises: (a) 30 mg/mL of tiragolumab; (b) 60 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 30 mg/mL of tiragolumab; (b) 60 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 30 mg/mL of tiragolumab; (b) 60 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 30 mg/mL of tiragolumab; (b) 60 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 30 mg/mL of tiragolumab; (b) 60 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 35 mg/mL of tiragolumab; (b) 70 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 35 mg/mL of tiragolumab; (b) 70 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 35 mg/mL of tiragolumab; (b) 70 mg/mL of the atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 35 mg/mL of tiragolumab; (b) 70 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 35 mg/mL of tiragolumab; (b) 70 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 40 mg/mL of tiragolumab; (b) 80 mg/mL of atezolizumab; (c) 20 mM of the histidine buffer; (d) 240 mM of sucrose; and (e) 0.06% (w/v) of polysorbate 20, wherein the liquid pharmaceutical formulation is characterized by a pH of about 5.5-5.8. In some embodiments, the formulation comprises: (a) 40 mg/ml of tiragolumab; (b) 80 mg/mL of atezolizumab; (c) 20 mM of the histidine buffer; (d) 240 mM of sucrose; and (e) 0.06% (w/v) of polysorbate 20, wherein the liquid pharmaceutical formulation is characterized by a pH of about 5.5. In some embodiments, the formulation comprises: (a) 40 mg/mL of tiragolumab; (b) 80 mg/mL of atezolizumab; (c) 20 mM of the histidine buffer; (d) 240 mM of sucrose; and (e) 0.06% (w/v) of polysorbate 20, wherein the liquid pharmaceutical formulation is characterized by a pH of about 5.6. In some embodiments, the formulation comprises: (a) 40 mg/mL of tiragolumab; (b) 80 mg/mL of atezolizumab; (c) 20 mM of the histidine buffer; (d) 240 mM of sucrose; and (e) 0.06% (w/v) of polysorbate 20, wherein the liquid pharmaceutical formulation is characterized by a pH of about 5.7. In some embodiments, the formulation comprises: (a) 40 mg/mL of tiragolumab; (b) 80 mg/mL of atezolizumab; (c) 20 mM of the histidine buffer; (d) 240 mM of sucrose; and (e) 0.06% (w/v) of polysorbate 20, wherein the liquid pharmaceutical formulation is characterized by a pH of about 5.8.

In some embodiments, the formulation comprises: (a) 45 mg/mL of tiragolumab; (b) 90 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 45 mg/mL of tiragolumab; (b) 90 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 45 mg/mL of tiragolumab; (b) 90 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 45 mg/mL of tiragolumab; (b) 90 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 45 mg/mL of tiragolumab; (b) 90 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 50 mg/mL of tiragolumab; (b) 100 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 50 mg/mL of tiragolumab; (b) 100 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 50 mg/mL of tiragolumab; (b) 100 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 50 mg/mL of tiragolumab; (b) 100 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 50 mg/ml of tiragolumab; (b) 100 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments, the formulation comprises: (a) 55 mg/mL of tiragolumab; (b) 110 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5-5.8. In some embodiments, the formulation comprises: (a) 55 mg/mL of tiragolumab; (b) 110 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.5. In some embodiments, the formulation comprises: (a) 55 mg/mL of tiragolumab; (b) 110 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.6. In some embodiments, the formulation comprises: (a) 55 mg/mL of tiragolumab; (b) 110 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.7. In some embodiments, the formulation comprises: (a) 55 mg/mL of tiragolumab; (b) 110 mg/mL of atezolizumab; (c) 2000 U/mL hyaluronidase; (d) 20 mM of the histidine buffer; (e) 240 mM of sucrose; and (f) 0.06% (w/v) of polysorbate 20, wherein the liquid pharmaceutical formulation is characterized by a pH of about 5.8.

In some embodiments the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.2-5.8. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.2. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.3. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.4. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.5. In some embodiments, the formulation comprises: (a) 160 mg/ml of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.6. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.7. In some embodiments, the formulation comprises: (a) 160 mg/mL of tiragolumab; (b) 20 mM of the histidine buffer; (c) 240 mM of sucrose; and (d) 0.06% (w/v) of polysorbate 20, wherein the liquid pharmaceutical formulation is characterized by a pH of about 5.8.

In some embodiments the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.2-5.8. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.2. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.3. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.4. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.5. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.6. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.7. In some embodiments, the formulation comprises: (a) 160 mg/mL of tiragolumab; (b) 2000 U/mL of the hyaluronidase; (c) 20 mM of the histidine buffer; (d) 240 mM of sucrose; and (e) 0.06% (w/v) of polysorbate 20, wherein the liquid pharmaceutical formulation is characterized by a pH of about 5.8.

In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.2-5.8. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.2. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.3. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.4. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.5. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.6. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.7. In some embodiments the formulation comprises: (a) 60 mg/mL of tiragolumab; (b) 20 mM of the histidine buffer; (c) 240 mM of sucrose; and (d) 0.04% (w/v) of polysorbate 20, wherein the liquid pharmaceutical formulation is characterized by a pH of about 5.8.

### Articles of Manufacture

A further aspect of the disclosure provides an article of manufacture comprising any of the liquid pharmaceutical formulations disclosed herein. In some embodiments, the article of manufacture comprises a liquid pharmaceutical formulation comprising (a) an anti-TIGIT monoclonal antibody; (b) a buffer; (c) a tonicity agent; and (d) a surfactant, wherein the liquid pharmaceutical formulation is characterized by a pH of about 4.0 to about 7.0. In some embodiments, the article of manufacture comprises a liquid pharmaceutical formulation comprising (a) an anti-TIGIT monoclonal antibody (b) an anti-PD-L1 monoclonal antibody; (c) a buffer; (d) a tonicity agent; and (e) a surfactant, wherein the liquid pharmaceutical formulation is characterized by a pH of about 4.0 to about 7.0.

In some embodiments, the article of manufacture is a vial. In some embodiments, the vial is a 10 cc vial. In some embodiments, the vial is a 15 cc vial. In some embodiments, the vial is a 20 cc vial. In some embodiments, the vial is a 25 cc vial. In some embodiments, the vial is a 30 cc vial. In some embodiments, the vial is a 35 cc vial. In some embodiments, the vial is a 40 cc vial. In some embodiments, the vial is a 45 cc vial. In some embodiments, the vial is a 50 cc vial. In some embodiments, the vial is a glass vial. In some embodiments, the vial is a plastic vial.

In some embodiments, the vial is stoppered with a chlorobutyl elastomer stopper. In some embodiments, the stopper is a D21-7S stopper. Without being bound by theory, the D21-7S stopper leads to reduced particle formation in the liquid pharmaceutical formulation. In some embodiments, the D21-7S stopper has a thinner stopper septum.

In some embodiments, the article of manufacture is a pre-filled syringe. In some embodiments, the pre-filled syringe is a 10 cc pre-filled syringe. In some embodiments, the pre-filled syringe is a 15 cc pre-filled syringe. In some embodiments, the pre-filled syringe is a 20 cc pre-filled syringe. In some embodiments, the pre-filled syringe is a 25 cc pre-filled syringe. In some embodiments, the pre-filled syringe is a 30 cc pre-filled syringe. In some embodiments, the pre-filled syringe is a 35 cc pre-filled syringe. In some embodiments, the pre-filled syringe is a 40 cc pre-filled syringe. In some embodiments, the pre-filled syringe is a 45 cc pre-filled syringe. In some embodiments, the pre-filled syringe is a 50 cc pre-filled syringe.

In some embodiments, the article comprises about 3 mL to about 30 mL of the liquid pharmaceutical formulation. In some embodiments, the article comprises about 3 mL to about 25 mL of the liquid pharmaceutical formulation. In some embodiments, the article comprises about 3 mL to about 20 mL of the liquid pharmaceutical formulation. In some embodiments, the article comprises about 4 mL to about 25 mL of the liquid pharmaceutical formulation. In some embodiments, the article comprises about 4 mL to about 22 mL of the liquid pharmaceutical formulation. In some embodiments, the article comprises about 4 mL to about 20 mL of the liquid pharmaceutical formulation. In some embodiments, the article comprises about 4 mL to about 18 mL of the liquid pharmaceutical formulation. In some embodiments, the article comprises about 4 mL to about 16 mL of the liquid pharmaceutical formulation. In some embodiments, the article comprises about 4 mL to about 14 mL of the liquid pharmaceutical formulation. In some embodiments, the article comprises about 4 mL to about 12 mL of the liquid pharmaceutical formulation. In some embodiments, the article comprises about 4 mL to about 10 mL of the liquid pharmaceutical formulation. In some embodiments, the article comprises about 4 mL to about 8 mL of the liquid pharmaceutical formulation. In some embodiments, the article comprises about 5 mL to about 7 mL of the liquid pharmaceutical formulation. In some embodiments, the article comprises about 5 mL to about 8 mL of the liquid pharmaceutical formulation. In some embodiments, the article comprises about 5 mL to about 10 mL of the liquid pharmaceutical formulation. In some embodiments, the article comprises about 5 mL to about 21 mL of the liquid pharmaceutical formulation. In some embodiments, the article comprises about 5.5 mL to about 7.5 mL of the liquid pharmaceutical formulation. In some embodiments, the article comprises about 6 mL to about 8 mL of the liquid pharmaceutical formulation. In some embodiments, the article comprises about 6 mL to about 10 mL of the liquid pharmaceutical formulation. In some embodiments, the article comprises about 6 mL to about 12 mL of the liquid pharmaceutical formulation. In some embodiments, the article comprises about 8 mL to about 12 mL of the liquid pharmaceutical formulation. In some embodiments, the article comprises about 9 mL to about 11 mL of the liquid pharmaceutical formulation. In some embodiments, the article comprises about 15 mL to about 30 mL of the liquid pharmaceutical formulation. In some embodiments, the article comprises about 18 mL to about 30 mL of the liquid pharmaceutical formulation. In some embodiments, the article comprises about 18 mL to about 28 mL of the liquid pharmaceutical formulation. In some embodiments, the article comprises about 18 mL to about 26 mL of the liquid pharmaceutical formulation. In some embodiments, the article comprises about 18 mL to about 24 mL of the liquid pharmaceutical formulation. In some embodiments, the article comprises about 19 mL to about 23 mL of the liquid pharmaceutical formulation. In some embodiments, the article comprises about 20 mL to about 22 mL of the liquid pharmaceutical formulation.

In some embodiments, the article comprises about 3 mL of the liquid pharmaceutical formulation. In some embodiments, the article comprises about 3.5 mL of the liquid pharmaceutical formulation. In some embodiments, the article comprises about 4 mL of the liquid pharmaceutical formulation. In some embodiments, the article comprises about 4.5 mL of the liquid pharmaceutical formulation. In some embodiments, the article comprises about 5 mL of the liquid pharmaceutical formulation. In some embodiments, the article comprises about 5.5 mL of the liquid pharmaceutical formulation. In some embodiments, the article comprises about 6 mL of the liquid pharmaceutical formulation. In some embodiments, the article comprises about 6.5 mL of the liquid pharmaceutical formulation. In some embodiments, the article comprises about 7 mL of the liquid pharmaceutical formulation. In some embodiments, the article comprises about 7.5 mL of the liquid pharmaceutical formulation. In some embodiments, the article comprises about 8 mL of the liquid pharmaceutical formulation. In some embodiments, the article comprises about 8.5 mL of the liquid pharmaceutical formulation. In some embodiments, the article comprises about 9 mL of the liquid pharmaceutical formulation. In some embodiments, the article comprises about 9.5 mL of the liquid pharmaceutical formulation. In some embodiments, the article comprises about 10 mL of the liquid pharmaceutical formulation. In some embodiments, the article comprises about 10.5 mL of the liquid pharmaceutical formulation. In some embodiments, the article comprises about 11 mL of the liquid pharmaceutical formulation. In some embodiments, the article comprises about 11.5 mL of the liquid pharmaceutical formulation. In some embodiments, the article comprises about 12 mL of the liquid pharmaceutical formulation. In some embodiments, the article comprises about 12.5 mL of the liquid pharmaceutical formulation. In some embodiments, the article comprises about 13 mL of the liquid pharmaceutical formulation. In some embodiments, the article comprises about 13.5 mL of the liquid pharmaceutical formulation. In some embodiments, the article comprises about 14 mL of the liquid pharmaceutical formulation. In some embodiments, the article comprises about 14.5 mL of the liquid pharmaceutical formulation. In some embodiments, the article comprises about 15 mL of the liquid pharmaceutical formulation. In some embodiments, the article comprises about 15.5 mL of the liquid pharmaceutical formulation. In some embodiments, the article comprises about 16 mL of the liquid pharmaceutical formulation. In some embodiments, the article comprises about 16.5 mL of the liquid pharmaceutical formulation. In some embodiments, the article comprises about 17 mL of the liquid pharmaceutical formulation. In some embodiments, the article comprises about 17.5 mL of the liquid pharmaceutical formulation. In some embodiments, the article comprises about 18 mL of the liquid pharmaceutical formulation. In some embodiments, the article comprises about 18.5 mL of the liquid pharmaceutical formulation. In some embodiments, the article comprises about 19 mL of the liquid pharmaceutical formulation. In some embodiments, the article comprises about 19.5 mL of the liquid pharmaceutical formulation. In some embodiments, the article comprises about 20 mL of the liquid pharmaceutical formulation. In some embodiments, the article comprises about 21 mL of the liquid pharmaceutical formulation. In some embodiments, the article comprises about 22 mL of the liquid pharmaceutical formulation. In some embodiments, the article comprises about 23 mL of the liquid pharmaceutical formulation. In some embodiments, the article comprises about 24 mL of the liquid pharmaceutical formulation. In some embodiments, the article comprises about 25 mL of the liquid pharmaceutical formulation. In some embodiments, the article comprises about 26 mL of the liquid pharmaceutical formulation. In some embodiments, the article comprises about 27 mL of the liquid pharmaceutical formulation. In some embodiments, the article comprises about 28 mL of the liquid pharmaceutical formulation. In some embodiments, the article comprises about 29 mL of the liquid pharmaceutical formulation. In some embodiments, the article comprises about 30 mL of the liquid pharmaceutical formulation.

In some embodiments, the article comprises 3 mL to 30 mL of the liquid pharmaceutical formulation. In some embodiments, the article comprises 3 mL to 25 mL of the liquid pharmaceutical formulation. In some embodiments, the article comprises 3 mL to 20 mL of the liquid pharmaceutical formulation. In some embodiments, the article comprises 4 mL to 25 mL of the liquid pharmaceutical formulation. In some embodiments, the article comprises 4 mL to 22 mL of the liquid pharmaceutical formulation. In some embodiments, the article comprises 4 mL to 20 mL of the liquid pharmaceutical formulation. In some embodiments, the article comprises 4 mL to 18 mL of the liquid pharmaceutical formulation. In some embodiments, the article comprises 4 mL to 16 mL of the liquid pharmaceutical formulation. In some embodiments, the article comprises 4 mL to 14 mL of the liquid pharmaceutical formulation. In some embodiments, the article comprises 4 mL to 12 mL of the liquid pharmaceutical formulation. In some embodiments, the article comprises 4 mL to 10 mL of the liquid pharmaceutical formulation. In some embodiments, the article comprises 4 mL to 8 mL of the liquid pharmaceutical formulation. In some embodiments, the article comprises 5 mL to 7 mL of the liquid pharmaceutical formulation. In some embodiments, the article comprises 5 mL to 8 mL of the liquid pharmaceutical formulation. In some embodiments, the article comprises 5 mL to 10 mL of the liquid pharmaceutical formulation. In some embodiments, the article comprises 5 mL to 21 mL of the liquid pharmaceutical formulation. In some embodiments, the article comprises 5.5 mL to 7.5 mL of the liquid pharmaceutical formulation. In some embodiments, the article comprises 6 mL to 8 mL of the liquid pharmaceutical formulation. In some embodiments, the article comprises 6 mL to 10 mL of the liquid pharmaceutical formulation. In some embodiments, the article comprises 6 mL to 12 mL of the liquid pharmaceutical formulation. In some embodiments, the article comprises 8 mL to 12 mL of the liquid pharmaceutical formulation. In some embodiments, the article comprises 9 mL to 11 mL of the liquid pharmaceutical formulation. In some embodiments, the article comprises 15 mL to 30 mL of the liquid pharmaceutical formulation. In some embodiments, the article comprises 18 mL to 30 mL of the liquid pharmaceutical formulation. In some embodiments, the article comprises 18 mL to 28 mL of the liquid pharmaceutical formulation. In some embodiments, the article comprises 18 mL to 26 mL of the liquid pharmaceutical formulation. In some embodiments, the article comprises 18 mL to 24 mL of the liquid pharmaceutical formulation. In some embodiments, the article comprises 19 mL to 23 mL of the liquid pharmaceutical formulation. In some embodiments, the article comprises 20 mL to 22 mL of the liquid pharmaceutical formulation.

In some embodiments, the article comprises 3 mL of the liquid pharmaceutical formulation. In some embodiments, the article comprises 3.5 mL of the liquid pharmaceutical formulation. In some embodiments, the article comprises 4 mL of the liquid pharmaceutical formulation. In some embodiments, the article comprises 4.5 mL of the liquid pharmaceutical formulation. In some embodiments, the article comprises 5 mL of the liquid pharmaceutical formulation. In some embodiments, the article comprises 5.5 mL of the liquid pharmaceutical formulation. In some embodiments, the article comprises 6 mL of the liquid pharmaceutical formulation. In some embodiments, the article comprises 6.5 mL of the liquid pharmaceutical formulation. In some embodiments, the article comprises 7 mL of the liquid pharmaceutical formulation. In some embodiments, the article comprises 7.5 mL of the liquid pharmaceutical formulation. In some embodiments, the article comprises 8 mL of the liquid pharmaceutical formulation. In some embodiments, the article comprises 8.5 mL of the liquid pharmaceutical formulation. In some embodiments, the article comprises 9 mL of the liquid pharmaceutical formulation. In some embodiments, the article comprises 9.5 mL of the liquid pharmaceutical formulation. In some embodiments, the article comprises 10 mL of the liquid pharmaceutical formulation. In some embodiments, the article comprises 10.5 mL of the liquid pharmaceutical formulation. In some embodiments, the article comprises 11 mL of the liquid pharmaceutical formulation. In some embodiments, the article comprises 11.5 mL of the liquid pharmaceutical formulation. In some embodiments, the article comprises 12 mL of the liquid pharmaceutical formulation. In some embodiments, the article comprises 12.5 mL of the liquid pharmaceutical formulation. In some embodiments, the article comprises 13 mL of the liquid pharmaceutical formulation. In some embodiments, the article comprises 13.5 mL of the liquid pharmaceutical formulation. In some embodiments, the article comprises 14 mL of the liquid pharmaceutical formulation. In some embodiments, the article comprises 14.5 mL of the liquid pharmaceutical formulation. In some embodiments, the article comprises 15 mL of the liquid pharmaceutical formulation. In some embodiments, the article comprises 15.5 mL of the liquid pharmaceutical formulation. In some embodiments, the article comprises 16 mL of the liquid pharmaceutical formulation. In some embodiments, the article comprises 16.5 mL of the liquid pharmaceutical formulation. In some embodiments, the article comprises 17 mL of the liquid pharmaceutical formulation. In some embodiments, the article comprises 17.5 mL of the liquid pharmaceutical formulation. In some embodiments, the article comprises 18 mL of the liquid pharmaceutical formulation. In some embodiments, the article comprises 18.5 mL of the liquid pharmaceutical formulation. In some embodiments, the article comprises 19 mL of the liquid pharmaceutical formulation. In some embodiments, the article comprises 19.5 mL of the liquid pharmaceutical formulation. In some embodiments, the article comprises 20 mL of the liquid pharmaceutical formulation. In some embodiments, the article comprises 21 mL of the liquid pharmaceutical formulation. In some embodiments, the article comprises 22 mL of the liquid pharmaceutical formulation. In some embodiments, the article comprises 23 mL of the liquid pharmaceutical formulation. In some embodiments, the article comprises 24 mL of the liquid pharmaceutical formulation. In some embodiments, the article comprises 25 mL of the liquid pharmaceutical formulation. In some embodiments, the article comprises 26 mL of the liquid pharmaceutical formulation. In some embodiments, the article comprises 27 mL of the liquid pharmaceutical formulation. In some embodiments, the article comprises 28 mL of the liquid pharmaceutical formulation. In some embodiments, the article comprises 29 mL of the liquid pharmaceutical formulation. In some embodiments, the article comprises 30 mL of the liquid pharmaceutical formulation.

In some embodiments, the concentration of the anti-TIGIT monoclonal antibody in the article is about 160 mg/mL. In some embodiments, the concentration of the anti-TIGIT monoclonal antibody in the article is about 150 mg/mL. In some embodiments, the concentration of the anti-TIGIT monoclonal antibody in the article is about 140 mg/mL. In some embodiments, the concentration of the anti-TIGIT monoclonal antibody in the article is about 130 mg/mL. In some embodiments, the concentration of the anti-TIGIT monoclonal antibody in the article is about 120 mg/mL. In some embodiments, the concentration of the anti-TIGIT monoclonal antibody in the article is about 110 mg/mL. In some embodiments, the concentration of the anti-TIGIT monoclonal antibody in the article is about 100 mg/mL. In some embodiments, the concentration of the anti-TIGIT monoclonal antibody in the article is about 90 mg/mL. In some embodiments, the concentration of the anti-TIGIT monoclonal antibody in the article is about 80 mg/mL. In some embodiments, the concentration of the anti-TIGIT monoclonal antibody in the article is about 70 mg/mL. In some embodiments, the concentration of the anti-TIGIT monoclonal antibody in the article is about 60 mg/mL. In some embodiments, the concentration of the anti-TIGIT monoclonal antibody in the article is about 50 mg/mL. In some embodiments, the concentration of the anti-TIGIT monoclonal antibody in the article is about 45 mg/mL. In some embodiments, the concentration of the anti-TIGIT monoclonal antibody in the article is about 40 mg/mL. In some embodiments, the concentration of the anti-TIGIT monoclonal antibody in the article is about 35 mg/mL. In some embodiments, the concentration of the anti-TIGIT monoclonal antibody in the article is about 30 mg/mL. In some embodiments, the concentration of the anti-TIGIT monoclonal antibody in the article is about 25 mg/mL. In some embodiments, the concentration of the anti-TIGIT monoclonal antibody in the article is about 20 mg/mL.

In some embodiments, the concentration of the anti-TIGIT monoclonal antibody in the article is 160 mg/mL. In some embodiments, the concentration of the anti-TIGIT monoclonal antibody in the article is 150 mg/mL. In some embodiments, the concentration of the anti-TIGIT monoclonal antibody in the article is 140 mg/mL. In some embodiments, the concentration of the anti-TIGIT monoclonal antibody in the article is 130 mg/mL. In some embodiments, the concentration of the anti-TIGIT monoclonal antibody in the article is 120 mg/mL. In some embodiments, the concentration of the anti-TIGIT monoclonal antibody in the article is 110 mg/mL. In some embodiments, the concentration of the anti-TIGIT monoclonal antibody in the article is 100 mg/mL. In some embodiments, the concentration of the anti-TIGIT monoclonal antibody in the article is 90 mg/mL. In some embodiments, the concentration of the anti-TIGIT monoclonal antibody in the article is 80 mg/mL. In some embodiments, the concentration of the anti-TIGIT monoclonal antibody in the article is 70 mg/mL. In some embodiments, the concentration of the anti-TIGIT monoclonal antibody in the article is 60 mg/mL. In some embodiments, the concentration of the anti-TIGIT monoclonal antibody in the article is 50 mg/mL. In some embodiments, the concentration of the anti-TIGIT monoclonal antibody in the article is 45 mg/mL. In some embodiments, the concentration of the anti-TIGIT monoclonal antibody in the article is 40 mg/mL. In some embodiments, the concentration of the anti-TIGIT monoclonal antibody in the article is 35 mg/mL. In some embodiments, the concentration of the anti-TIGIT monoclonal antibody in the article is 30 mg/mL. In some embodiments, the concentration of the anti-TIGIT monoclonal antibody in the article is 25 mg/mL. In some embodiments, the concentration of the anti-TIGIT monoclonal antibody in the article is 20 mg/mL.

An additional aspect of this disclosure provides an article of manufacture comprising a formulation comprising 18 mg/mL to 176 mg/mL of an anti-TIGIT monoclonal antibody, and characterized by a pH of about 5.2-6.0. In some embodiments, an article of manufacture comprises a formulation comprising 144 mg/mL to 176 mg/mL of an anti-TIGIT monoclonal antibody, and characterized by a pH of about 5.2-5.8. In some embodiments, an article of manufacture comprises a formulation comprising 160 mg/mL of an anti-TIGIT monoclonal antibody, and characterized by a pH of about 5.5. In some embodiments, an article of manufacture comprising a formulation comprising 18 mg/mL to 176 mg/mL of an anti-TIGIT monoclonal antibody. In some embodiments, an article of manufacture comprises a formulation comprising 144 mg/mL to 176 mg/mL of an anti-TIGIT monoclonal antibody. In some embodiments, an article of manufacture comprises a formulation comprising 160 mg/mL of an anti-TIGIT monoclonal antibody.

Another aspect of the present disclosure provides an article of manufacture comprising a formulation suitable for subcutaneous injection comprising 18 mg/mL to 176 mg/mL of an anti-TIGIT monoclonal antibody, and 500 U/mL to 2600 U/mL of a hyaluronidase, wherein the formulation is characterized by a pH of about 5.2-6.0. In some embodiments, the formulation suitable for subcutaneous injection comprises 144 mg/mL to 176 mg/mL of an anti-TIGIT monoclonal antibody, and 1400 U/mL to 2600 U/mL of a hyaluronidase and is characterized by a pH of about 5.2-5.8. In some embodiments, an article of manufacture of this disclosure is suitable for subcutaneous injection comprises 160 mg/mL of an anti-TIGIT monoclonal antibody, and 2000 U/mL of a hyaluronidase, wherein the article of manufacture is characterized by a pH of about 5.5. In some embodiments, the formulation suitable for subcutaneous injection comprises 18 mg/mL to 176 mg/mL of an anti-TIGIT monoclonal antibody, and 500 U/mL to 2600 U/mL of a hyaluronidase. In some embodiments, the formulation suitable for subcutaneous injection comprises 144 mg/mL to 176 mg/mL of an anti-TIGIT monoclonal antibody, and 1400 U/mL to 2600 U/mL of a hyaluronidase. In some embodiments, an article of manufacture of this disclosure is suitable for subcutaneous injection comprises 160 mg/mL of an anti-TIGIT monoclonal antibody, and 2000 U/mL of a hyaluronidase.

A further aspect of the present disclosure provides an article of manufacture comprising a formulation comprising 18 mg/mL to 176 mg/mL of an anti-TIGIT monoclonal antibody, and 54 mg/mL to 137.5 mg/mL of an anti-PD-L1 monoclonal antibody, wherein the formulation characterized by a pH of about 5.2-6.1. In some embodiments, the article of manufacture comprises a formulation comprising 36 mg/mL to 44 mg/mL of an anti-TIGIT monoclonal antibody, and 72 mg/mL to 88 mg/mL of an anti-PD-L1 monoclonal antibody, wherein the formulation is characterized by a pH of about 5.5-6.1. In some embodiments, the article of manufacture comprises a formulation comprising 30 mg/mL to 60 mg/mL of an anti-TIGIT monoclonal antibody, and 70 mg/mL to 110 mg/mL of an anti-PD-L1 monoclonal antibody, wherein the formulation is characterized by a pH of about 5.2-6.1. In some embodiments, the article of manufacture comprises a formulation comprising 35 mg/mL to 50 mg/mL of an anti-TIGIT monoclonal antibody, and 70 mg/mL to 100 mg/mL of an anti-PD-L1 monoclonal antibody, wherein the formulation is characterized by a pH of about 5.5-5.8. In some embodiments, the article of manufacture comprises a formulation comprising 18 mg/mL to 176 mg/mL of an anti-TIGIT monoclonal antibody, and 54 mg/mL to 137.5 mg/mL of an anti-PD-L1 monoclonal antibody. In some embodiments, the article of manufacture comprises a formulation comprising 36 mg/mL to 44 mg/mL of an anti-TIGIT monoclonal antibody, and 72 mg/mL to 88 mg/mL of an anti-PD-L1 monoclonal antibody. In some embodiments, the article of manufacture comprises a formulation comprising 30 mg/mL to 60 mg/mL of an anti-TIGIT monoclonal antibody, and 70 mg/mL to 110 mg/mL of an anti-PD-L1 monoclonal antibody. In some embodiments, the article of manufacture comprises a formulation comprising 35 mg/mL to 50 mg/mL of an anti-TIGIT monoclonal antibody, and 70 mg/mL to 100 mg/mL of an anti-PD-L1 monoclonal antibody.

In some embodiments, the article of manufacture comprises a formulation comprising 40 mg/mL of an anti-TIGIT monoclonal antibody, and 80 mg/mL of an anti-PD-L1 monoclonal antibody. In some embodiments, the article of manufacture comprises a formulation comprising 40 mg/mL of an anti-TIGIT monoclonal antibody, and 80 mg/mL of an anti-PD-L1 monoclonal antibody, wherein the formulation is characterized by a pH of about 5.5. In some embodiments, the article of manufacture comprises a formulation comprising 40 mg/mL of an anti-TIGIT monoclonal antibody, and 80 mg/mL of an anti-PD-L1 monoclonal antibody, wherein the formulation is characterized by a pH of about 5.6. In some embodiments, the article of manufacture comprises a formulation comprising 40 mg/mL of an anti-TIGIT monoclonal antibody, and 80 mg/mL of an anti-PD-L1 monoclonal antibody, wherein the formulation is characterized by a pH of about 5.7. In some embodiments, the article of manufacture comprises a formulation comprising 40 mg/mL of an anti-TIGIT monoclonal antibody, and 80 mg/mL of an anti-PD-L1 monoclonal antibody, wherein the formulation is characterized by a pH of about 5.8. In some embodiments, the article of manufacture comprises a formulation comprising 45 mg/mL of an anti-TIGIT monoclonal antibody, and 90 mg/mL of an anti-PD-L1 monoclonal antibody. In some embodiments, the article of manufacture comprises a formulation comprising 45 mg/mL of an anti-TIGIT monoclonal antibody, and 90 mg/mL of an anti-PD-L1 monoclonal antibody, wherein the formulation is characterized by a pH of about 5.5. In some embodiments, the article of manufacture comprises a formulation comprising 45 mg/mL of an anti-TIGIT monoclonal antibody, and 90 mg/mL of an anti-PD-L1 monoclonal antibody, wherein the formulation is characterized by a pH of about 5.6. In some embodiments, the article of manufacture comprises a formulation comprising 45 mg/mL of an anti-TIGIT monoclonal antibody, and 90 mg/mL of an anti-PD-L1 monoclonal antibody, wherein the formulation is characterized by a pH of about 5.7. In some embodiments, the article of manufacture comprises a formulation comprising 45 mg/mL of an anti-TIGIT monoclonal antibody, and 90 mg/mL of an anti-PD-L1 monoclonal antibody, wherein the formulation is characterized by a pH of about 5.8.

An additional aspect of the present disclosure provides an article of manufacture comprising a formulation that is suitable for subcutaneous injection, comprises 30 mg/mL to 60 mg/mL of an anti-TIGIT monoclonal antibody, 60 mg/mL to 120 mg/mL of an anti-PD-L1 monoclonal antibody, and 500 U/mL to 2600 U/mL of a hyaluronidase and is characterized by a pH of about 5.2-6.1. In some embodiments, the formulation suitable for subcutaneous injection comprises 35 mg/mL to 50 mg/mL of an anti-TIGIT monoclonal antibody, 70 mg/mL to 100 mg/mL of an anti-PD-L1 monoclonal antibody, and 1400 U/mL to 2600 U/mL of a hyaluronidase and is characterized by a pH of about 5.5-5.8. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase and is characterized by a pH of about 5.5-5.8. In some embodiments, the formulation suitable for subcutaneous injection comprises 30 mg/mL to 60 mg/mL of an anti-TIGIT monoclonal antibody, 60 mg/mL to 120 mg/mL of an anti-PD-L1 monoclonal antibody, and 500 U/mL to 2600 U/mL of a hyaluronidase. In some embodiments, the formulation suitable for subcutaneous injection comprises 35 mg/mL to 50 mg/mL of an anti-TIGIT monoclonal antibody, 70 mg/mL to 100 mg/mL of an anti-PD-L1 monoclonal antibody, and 1400 U/mL to 2600 U/mL of a hyaluronidase. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase and is characterized by a pH of about 5.5. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase and is characterized by a pH of about 5.6. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase and is characterized by a pH of about 5.7. In some embodiments, the formulation suitable for subcutaneous injection comprises 40 mg/mL of an anti-TIGIT monoclonal antibody, 80 mg/mL of an anti-PD-L1 monoclonal antibody, and 2000 U/mL of a hyaluronidase and is characterized by a pH of about 5.8. In some embodiments, the formulation suitable for subcutaneous injection comprises 45 mg/mL of an anti-TIGIT monoclonal antibody, 90 mg/mL of an anti-PD-L1 monoclonal antibody, and 2000 U/mL of a hyaluronidase. In some embodiments, the formulation suitable for subcutaneous injection comprises 45 mg/mL of an anti-TIGIT monoclonal antibody, 90 mg/mL of an anti-PD-L1 monoclonal antibody, and 2000 U/mL of a hyaluronidase and is characterized by a pH of about 5.5-5.8. In some embodiments, the formulation suitable for subcutaneous injection comprises 45 mg/mL of an anti-TIGIT monoclonal antibody, 90 mg/mL of an anti-PD-L1 monoclonal antibody, and 2000 U/mL of a hyaluronidase and is characterized by a pH of about 5.6. In some embodiments, formulation suitable for subcutaneous injection comprises 45 mg/mL of an anti-TIGIT monoclonal antibody, 90 mg/mL of an anti-PD-L1 monoclonal antibody, and 2000 U/mL of a hyaluronidase and is characterized by a pH of about 5.7. In some embodiments, the formulation suitable for subcutaneous injection comprises 45 mg/mL of an anti-TIGIT monoclonal antibody, 90 mg/mL of an anti-PD-L1 monoclonal antibody, and 2000 U/mL of a hyaluronidase and is characterized by a pH of about 5.8.

Another aspect of the present disclosure provides an article of manufacture comprising a subcutaneous administration device, which contains and delivers to a patient a 880 mg fixed dose of an anti-TIGIT monoclonal antibody. In some embodiments, the subcutaneous administration device further delivers to the patient a 1875 mg or 2000 mg fixed dose of an anti-PD-L1 monoclonal antibody. In some embodiments, the subcutaneous administration device is a syringe pump.

A further aspect of the present disclosure provides an article of manufacture comprising a formulation suitable for subcutaneous injection comprising 880 mg of an anti-TIGIT monoclonal antibody and hyaluronidase. In some embodiments, the hyaluronidase is a recombinant human hyaluronidase. In some embodiments, the hyaluronidase is a human soluble PH20 hyaluronidase glycoprotein, such as rHuPH20. In some embodiments, the concentration of the hyaluronidase is 500 U/mL to 2600 U/mL. In some embodiments, the concentration of the hyaluronidase is 1400 U/mL to 2600 U/mL. In some embodiments, the concentration of the hyaluronidase is 2000 U/mL.

In another aspect, the present disclosure provides an article of manufacture comprising a formulation suitable for subcutaneous injection comprising: (a) 880 mg of an anti-TIGIT monoclonal antibody, (b) 1875 mg or 2000 mg of an anti-PD-L1 monoclonal antibody, (c) 5 mM to 30 mM of a histidine buffer, (d) 180 mM to 320 mM of sucrose, (e) 0.03% (w/v) to 0.08% (w/v) polysorbate 20, (f) 500 U/mL to 2600 U/mL hyaluronidase, pH of about 5.2-6.1. In some embodiments, the article of manufacture comprises a formulation comprising: (a) 880 mg of an anti-TIGIT monoclonal antibody, (b) 1875 mg or 2000 mg of an anti-PD-L1 monoclonal antibody, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of an anti-TIGIT monoclonal antibody, (b) 1875 mg or 2000 mg of an anti-PD-L1 monoclonal antibody, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of an anti-TIGIT monoclonal antibody, (b) 1875 mg of an anti-PD-L1 monoclonal antibody, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of an anti-TIGIT monoclonal antibody, (b) 2000 mg of an anti-PD-L1 monoclonal antibody, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8.

In some embodiments of any of the above aspects, the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6. In some embodiments of any of the above aspects, the anti-TIGIT monoclonal antibody comprises a VH comprising the amino acid sequence of SEQ ID NO: 7. In some embodiments of any of the above aspects, the anti-TIGIT monoclonal antibody comprises a VH comprising the amino acid sequence of SEQ ID NO: 8. In some embodiments of any of the above aspects, the anti-TIGIT monoclonal antibody comprises a VL comprising the amino acid sequence of SEQ ID NO: 9. In some embodiments of any of the above aspects, the anti-TIGIT monoclonal antibody comprises a VH comprising the amino acid sequence of SEQ ID NO: 7 and a VL comprising the amino acid sequence of SEQ ID NO: 9. In some embodiments of any of the above aspects, the heavy chain of the anti-TIGIT monoclonal antibody comprises the amino acid sequence of SEQ ID NO: 18. In some embodiments of any of the above aspects, the heavy chain of the anti-TIGIT monoclonal antibody comprises the amino acid sequence of SEQ ID NO: 19. In some embodiments of any of the above aspects, the heavy chain of the anti-TIGIT monoclonal antibody comprises the amino acid sequence of SEQ ID NO: 24. In some embodiments of any of the above aspects, the heavy chain of the anti-TIGIT monoclonal antibody comprises the amino acid sequence of SEQ ID NO: 25. In some embodiments of any of the above aspects, the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 18 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 19 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 24 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 25 and the light chain of the anti-TIGIT monoclonal antibody comprises the amino acid sequence of SEQ ID NO: 20. In some embodiments of any of the above aspects, the anti-TIGIT monoclonal antibody is a full-length antibody. In some embodiments of any of the above aspects, the anti-TIGIT monoclonal antibody is a full-length IgG antibody. In some embodiments of any of the above aspects, the anti-TIGIT monoclonal antibody is a full-length IgG1 antibody. In some embodiments of any of the above aspects, the anti-TIGIT monoclonal antibody is a full-length human IgG1 antibody. In some embodiments of any of the above aspects, the anti-TIGIT monoclonal antibody is a full-length humanized IgG1 antibody. In some embodiments of any of the above aspects, the anti-TIGIT monoclonal antibody retains one or more effector functions. In some embodiments of any of the above aspects, the anti-TIGIT monoclonal antibody retains all effector functions. In some embodiments of any of the above aspects, one or more effector functions of the anti-TIGIT monoclonal antibody may have been modified or eliminated. In some embodiments of any of the above aspects, the anti-TIGIT monoclonal antibody is tiragolumab. In some embodiments of any of the above aspects, the anti-TIGIT monoclonal antibody is tiragolumab. Tiragolumab is described in WHO Drug Information (International Nonproprietary Names for Pharmaceutical Substances), Proposed INN: List 117, Vol. 31, No. 2, published Jun. 9, 2017 (see p. 343). In some embodiments, tiragolumab has the CAS Registry Number 1918185-84-8. The anti-TIGIT monoclonal antibody may be an antibody fragment. In some embodiments, the anti-TIGIT monoclonal antibody is a Fab, Fab′, F(ab′)₂, a Fv or a scFv fragment. In some embodiments, the anti-TIGIT monoclonal antibody is a Fab fragment. In some embodiments, the anti-TIGIT monoclonal antibody is a Fab′ fragment. In some embodiments, the anti-TIGIT monoclonal antibody is a F(ab′)₂, fragment. In some embodiments, the anti-TIGIT monoclonal antibody is a Fv fragment. In some embodiments, the anti-TIGIT monoclonal antibody is a scFv fragment. In some embodiments, the anti-TIGIT monoclonal antibody is a diabody. In some embodiments, the anti-TIGIT monoclonal antibody is a linear antibody. In some embodiments, the anti-TIGIT monoclonal antibody is a single-chain antibody molecule. In some embodiments, the anti-TIGIT monoclonal antibody is a multispecific antibody, e.g. formed from antibody fragments.

In some embodiments of any of the above aspects, the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15. In some embodiments of any of the above aspects, the anti-PD-L1 monoclonal antibody comprises a VH comprising the amino acid sequence of SEQ ID NO: 16. In some embodiments of any of the above aspects, the anti-PD-L1 monoclonal antibody comprises a VH comprising the amino acid sequence of SEQ ID NO: 16. In some embodiments of any of the above aspects, the anti-PD-L1 monoclonal antibody comprises a VL comprising the amino acid sequence of SEQ ID NO: 17. In some embodiments of any of the above aspects, the anti-PD-L1 monoclonal antibody comprises a VH comprising the amino acid sequence of SEQ ID NO: 16 and a VL comprising the amino acid sequence of SEQ ID NO: 17. In some embodiments of any of the above aspects, the heavy chain of the anti-PD-L1 monoclonal antibody comprises the amino acid sequence of SEQ ID NO: 21. In some embodiments of any of the above aspects, the heavy chain of the anti-PD-L1 monoclonal antibody comprises the amino acid sequence of SEQ ID NO: 22. In some embodiments of any of the above aspects, the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23. In some embodiments of any of the above aspects, the anti-PD-L1 monoclonal antibody is a full-length antibody. In some embodiments, the anti-PD-L1 monoclonal antibody is a full-length IgG antibody. In some embodiments, the anti-PD-L1 monoclonal antibody is a full-length IgG1 antibody. In some embodiments, the anti-PD-L1 monoclonal antibody is a full-length human IgG1 antibody. In some embodiments, the anti-PD-L1 monoclonal antibody is a full-length humanized IgG1 antibody. In some embodiments of any of the above aspects, the anti-PD-L1 monoclonal antibody retains one or more effector functions. In some embodiments of any of the above aspects, the anti-PD-L1 monoclonal antibody retains all effector functions. In some embodiments of any of the above aspects, one or more effector functions of the anti-PD-L1 monoclonal antibody may have been modified or eliminated. In some embodiments, the anti-PD-L1 monoclonal antibody is atezolizumab. In some embodiments, the anti-PD-L1 monoclonal antibody is atezolizumab. In some embodiments, the anti-PD-L1 monoclonal antibody is atezolizumab, marketed as TECENTRIQ™. Atezolizumab is described in WHO Drug Information (International Nonproprietary Names for Pharmaceutical Substances), Proposed INN: List 112, Vol. 28, No. 4, 2014 (see page 488). In some embodiments, atezolizumab has the CAS Registry Number 1380723-44-3.

An additional aspect of the present disclosure provides an article of manufacture comprising a subcutaneous administration device, which contains and delivers to a patient a 880 mg fixed dose of tiragolumab. In some embodiments, the subcutaneous administration device further delivers to the patient a 1875 mg or 2000 mg fixed dose of atezolizumab. In some embodiments, the subcutaneous administration device is a syringe pump.

A further aspect of the present disclosure provides an article of manufacture comprising a formulation suitable for subcutaneous injection comprising 880 mg of tiragolumab monoclonal antibody and hyaluronidase. In some embodiments, the hyaluronidase is a recombinant human hyaluronidase. In some embodiments, the hyaluronidase is a human soluble PH20 hyaluronidase glycoprotein, such as rHuPH20. In some embodiments, the concentration of the hyaluronidase is 500 U/mL to 2600 U/mL. In some embodiments, the concentration of the hyaluronidase is 1400 U/mL to 2600 U/mL. In some embodiments, the concentration of the hyaluronidase is 2000 U/mL.

In another aspect, the present disclosure provides an article of manufacture comprising a formulation suitable for subcutaneous injection comprising: (a) 880 mg of tiragolumab, (b) 1875 mg or 2000 mg of atezolizumab, (c) 5 mM to 30 mM of a histidine buffer, (d) 180 mM to 320 mM of sucrose, (e) 0.03% (w/v) to 0.08% (w/v) polysorbate 20, (f) 500 U/mL to 2600 U/mL hyaluronidase, pH of about 5.2-6.1. In some embodiments, the article of manufacture comprises a formulation comprising: (a) 880 mg of tiragolumab, (b) 1875 mg or 2000 mg of atezolizumab, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of tiragolumab, (b) 1875 mg or 2000 mg of atezolizumab, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of tiragolumab, (b) 1875 mg of atezolizumab, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8. In some embodiments, the article of manufacture comprises a formulation suitable for subcutaneous injection comprising: (a) 880 mg of tiragolumab, (b) 2000 mg of atezolizumab, (c) 15 mM to 25 mM of a histidine buffer, (d) 200 mM to 280 mM of sucrose, (e) 0.04% (w/v) to 0.08% (w/v) polysorbate 20, (f) 1400 U/mL to 2600 U/mL hyaluronidase, pH of about 5.5-5.8.

In some embodiments of any of the above aspects, the article of manufacture comprises a subcutaneous administration device. In some embodiments, the subcutaneous administration device is selected from the group consisting of a syringe, a syringe pump, an injection device, an infusion pump, an injector pen, a needleless device, an autoinjector, and a subcutaneous patch delivery system. In some embodiments, the subcutaneous administration device is a syringe. In some embodiments, the subcutaneous administration device is a syringe pump. In some embodiments, the subcutaneous administration device is an injection device. In some embodiments, the subcutaneous administration device is an infusion pump. In some embodiments, the subcutaneous administration device is an injector pen. In some embodiments, the subcutaneous administration device is a needleless device. In some embodiments, the subcutaneous administration device is an autoinjector. In some embodiments, the subcutaneous administration device is a subcutaneous patch delivery system. In some embodiments, the subcutaneous administration device is a pre-filled syringe.

In some embodiments of any of the above aspects, the formulation contained in the article of manufacture is a formulation of this disclosure. In some embodiments of any of the above aspects, the article of manufacture contains a formulation of this disclosure.

### Methods of Treating Cancer

An additional aspect of the disclosure provides a method of treating cancer in a subject in need thereof comprising administering to the subject a therapeutically effective amount of any of the liquid pharmaceutical formulations disclosed herein. In some embodiments, the method comprises administering a therapeutically effective amount of a liquid pharmaceutical formulation comprising (a) an anti-TIGIT monoclonal antibody; (b) a buffer; (c) a tonicity agent; and (d) a surfactant, wherein the liquid pharmaceutical formulation is characterized by a pH of about 4.0 to about 7.0. In some embodiments, the method comprises administering a therapeutically effective amount of a liquid pharmaceutical formulation comprising (a) an anti-TIGIT monoclonal antibody; (b) an anti-PD-L1 monoclonal antibody; (c) a buffer; (d) a tonicity agent; and (e) a surfactant, wherein the liquid pharmaceutical formulation is characterized by a pH of about 4.0 to about 7.0.

An additional aspect of the disclosure provides use of a therapeutically effective amount of any of the liquid pharmaceutical formulations disclosed herein in the manufacture of a medicament for treating cancer in a subject in need thereof.

In some embodiments, the liquid pharmaceutical formulation comprising (a) an anti-TIGIT monoclonal antibody; (b) a buffer; (c) a tonicity agent; and (d) a surfactant, wherein the liquid pharmaceutical formulation is characterized by a pH of about 4.0 to about 7.0. In some embodiments, the liquid pharmaceutical formulation comprising (a) an anti-TIGIT monoclonal antibody; (b) an anti-PD-L1 monoclonal antibody; (c) a buffer; (d) a tonicity agent; and (e) a surfactant, wherein the liquid pharmaceutical formulation is characterized by a pH of about 4.0 to about 7.0.

A further aspect of the disclosure provides a therapeutically effective amount of any of the liquid pharmaceutical formulations disclosed herein for use in treating cancer in a subject in need thereof.

In some embodiments, the liquid pharmaceutical formulation for use comprises a liquid pharmaceutical formulation comprising (a) an anti-TIGIT monoclonal antibody; (b) a buffer; (c) a tonicity agent; and (d) a surfactant, wherein the liquid pharmaceutical formulation is characterized by a pH of about 4.0 to about 7.0. In some embodiments, the liquid pharmaceutical formulations for use comprise a liquid pharmaceutical formulation comprising (a) an anti-TIGIT monoclonal antibody; (b) an anti-PD-L1 monoclonal antibody; (c) a buffer; (d) a tonicity agent; and (e) a surfactant, wherein the liquid pharmaceutical formulation is characterized by a pH of about 4.0 to about 7.0.

The liquid pharmaceutical formulation may be administered parenterally. In some embodiments, the liquid pharmaceutical formulation is administered by injection. In some embodiments, the liquid pharmaceutical formulation is administered intravenously or subcutaneously. In some embodiments, the liquid pharmaceutical formulation is administered intravenously. In some embodiments, the liquid pharmaceutical formulation is administered subcutaneously.

An additional aspect of the disclosure provides a method of treating cancer in a subject in need thereof comprising administering to the subject an anti-TIGIT monoclonal antibody at a fixed dose of 880 mg.

Another aspect of the disclosure provides a method of treating cancer in a subject in need thereof comprising administering to the subject tiragolumab at a dose of 880 mg.

In another aspect, the disclosure provides a method for treating cancer in a subject in need thereof comprising subcutaneously administering to the subject a fixed dose of 880 mg of an anti-TIGIT monoclonal antibody. In another aspect, the disclosure provides a method for treating cancer in a subject in need thereof comprising subcutaneously administering to the human subject a fixed dose of 880 mg of an anti-TIGIT monoclonal antibody and a 1800 mg or 2000 mg fixed dose of an anti-PD-L1 monoclonal antibody.

In another aspect, the disclosure provides a method for treating cancer in a subject in need thereof comprising subcutaneously administering to the subject a fixed dose of 880 mg of tiragolumab. In another aspect, the disclosure provides a method for treating cancer in a subject in need thereof comprising subcutaneously administering to the human subject a fixed dose of 880 mg of tiragolumab and a 1800 mg or 2000 mg fixed dose of atezolizumab.

In some embodiments of any of the above aspects, the anti-TIGIT monoclonal antibody may be administered parenterally. In some embodiments, the anti-TIGIT monoclonal antibody is administered by injection. In some embodiments, the anti-TIGIT monoclonal antibody is administered intravenously or subcutaneously. In some embodiments, the anti-TIGIT monoclonal antibody is administered intravenously. In some embodiments, the anti-TIGIT monoclonal antibody is administered subcutaneously.

In some embodiments of any of the above aspects, the anti-PD-L1 monoclonal antibody may be administered parenterally. In some embodiments, the anti-PD-L1 monoclonal antibody is administered by injection. In some embodiments, the anti-PD-L1 monoclonal antibody is administered intravenously or subcutaneously. In some embodiments, the anti-PD-L1 monoclonal antibody is administered intravenously. In some embodiments, the anti-PD-L1 monoclonal antibody is administered subcutaneously.

In some embodiments, the anti-TIGIT monoclonal antibody and the anti-PD-L1 monoclonal antibody are co-mixed. In some embodiments, the anti-TIGIT monoclonal antibody and the anti-PD-L1 monoclonal antibody are coformulated.

In some embodiments of any of the above aspects, the subject is human. In some embodiments of any of the above aspects, the subject has not received prior checkpoint inhibitor treatment (i.e. is CPI-Naive). In some embodiments of any of the above aspects, the subject has not received prior treatment with an anti-PD-L1 monoclonal antibody, an anti-PD-1 antibody, an anti-CTL1-4 antibody, or an anti-TIGIT monoclonal antibody. In some embodiments of any of the above aspects, the subject has not received prior treatment with an anti-PD-L1 monoclonal antibody. In some embodiments of any of the above aspects, the subject has not received prior treatment with an anti-PD-1 antibody. In some embodiments of any of the above aspects, the subject has not received prior treatment with an anti-CTL1-4 antibody. In some embodiments of any of the above aspects, the subject has not received prior treatment with an anti-TIGIT monoclonal antibody. In some embodiments of any of the above aspects, the subject is cancer immunotherapy (CIT) naive (i.e. is CIT-Naive).

In some embodiments of any of the above aspects, the cancer is selected from the group consisting of a lung cancer, a non-small cell lung cancer, a renal cell cancer, a urothelial cancer, a ureter cancer, a urethral cancer, a colorectal cancer, a colon cancer, a rectal cancer, a kidney cancer, a sarcoma, an ovarian cancer, a breast cancer, a cervical cancer, a fallopian tube cancer, an endometrial cancer, a uterine cancer, a pancreatic cancer, a gastric carcinoma, a bladder cancer, an esophageal cancer, a mesothelioma, a melanoma, a head and neck cancer, a thyroid cancer, a sarcoma, a prostate cancer, a penile cancer, a glioblastoma, a thymic carcinoma, an esophageal carcinoma, a nasopharyngeal cancer, a mesothelioma, a liver cancer, a biliary tract cancer, a HPV-positive cancer, a leukemia, a lymphoma, a brain cancer, a neuroendocrine cancer, a myeloma, a mycosis fungoides, a Merkel cell cancer, a hematologic malignancy, a deficient mismatch repair (dMMR) cancer, and a microsatellite instability-high (MSI-H) cancer. In some embodiments of any of the above aspects, the cancer is a lung cancer. In some embodiments of any of the above aspects, the cancer is a non-small cell lung cancer. In some embodiments of any of the above aspects, the cancer is a renal cell cancer. In some embodiments of any of the above aspects, the cancer is a urothelial cancer. In some embodiments of any of the above aspects, the cancer is a ureter cancer. In some embodiments, the cancer is a urethral cancer. In some embodiments of any of the above aspects, the cancer is a colorectal cancer. In some embodiments of any of the above aspects, the cancer is a colon cancer. In some embodiments of any of the above aspects, the cancer is a rectal cancer. In some embodiments of any of the above aspects, the cancer is a kidney cancer. In some embodiments of any of the above aspects, the cancer is a sarcoma. In some embodiments of any of the above aspects, the cancer is an ovarian cancer. In some embodiments of any of the above aspects, the cancer is a breast cancer. In some embodiments of any of the above aspects, the cancer is a cervical cancer. In some embodiments of any of the above aspects, the cancer is a fallopian tube cancer. In some embodiments of any of the above aspects, the cancer is an endometrial cancer. In some embodiments of any of the above aspects, the cancer is a uterine cancer. In some embodiments of any of the above aspects, the cancer is a pancreatic cancer. In some embodiments of any of the above aspects, the cancer is a gastric carcinoma. In some embodiments of any of the above aspects, the cancer is a bladder cancer. In some embodiments of any of the above aspects, the cancer is an esophageal cancer. In some embodiments of any of the above aspects, the cancer is a mesothelioma. In some embodiments of any of the above aspects, the cancer is a melanoma. In some embodiments of any of the above aspects, the cancer is a head and neck cancer. In some embodiments of any of the above aspects, the cancer is a thyroid cancer. In some embodiments of any of the above aspects, the cancer is a sarcoma. In some embodiments of any of the above aspects, the cancer is a prostate cancer. In some embodiments of any of the above aspects, the cancer is a penile cancer. In some embodiments of any of the above aspects, the cancer is a glioblastoma. In some embodiments of any of the above aspects, the cancer is a thymic carcinoma. In some embodiments of any of the above aspects, the cancer is an esophageal carcinoma. In some embodiments of any of the above aspects, the cancer is a nasopharyngeal cancer. In some embodiments of any of the above aspects, the cancer is a mesothelioma. In some embodiments of any of the above aspects, the cancer is a liver cancer. In some embodiments of any of the above aspects, the cancer is a biliary tract cancer. In some embodiments of any of the above aspects, the cancer is a HPV-positive cancer. In some embodiments of any of the above aspects, the cancer is a leukemia. In some embodiments of any of the above aspects, the cancer is a lymphoma. In some embodiments of any of the above aspects, the cancer is a brain cancer. In some embodiments of any of the above aspects, the cancer is a neuroendocrine cancer. In some embodiments of any of the above aspects, the cancer is a myeloma. In some embodiments of any of the above aspects, the cancer is a mycosis fungoides. In some embodiments of any of the above aspects, the cancer is a Merkel cell cancer. In some embodiments of any of the above aspects, the cancer is a hematologic malignancy. In some embodiments of any of the above aspects, the cancer is a deficient mismatch repair (dMMR) cancer. In some embodiments of any of the above aspects, the cancer is a microsatellite instability-high (MSI-H) cancer.

In some embodiments of any of the above aspects, the cancer is selected from the group consisting of a bladder cancer, a muscle-invasive bladder cancer, a urothelial carcinoma, a ureter cancer, a urethral cancer, a ureter urothelial carcinoma, a urethral urothelial carcinoma, a renal cancer, a renal pelvis cancer, a renal cell carcinoma, a clear-cell renal carcinoma, a rectal cancer, a colon cancer, a colorectal cancer, a sarcoma, an osteosarcoma, a leiomyosarcoma, a pleomorphic sarcoma, a myxofibrosarcoma, a liposarcoma, a chondrosarcoma, a lung cancer, a non-small cell lung cancer, a fallopian tube cancer, a peritoneal carcinoma, an esophageal cancer, an esophageal squamous cell carcinoma, a mesothelioma, a pleural mesothelioma, a peritoneal mesothelioma, an ovarian cancer, a cervical cancer, a cervical adenosquamous carcinoma, a breast cancer, a triple-negative breast cancer, a HER2-positive breast cancer, a HER2-negative breast cancer, an estrogen receptor-positive breast cancer, a progesterone receptor-positive breast cancer, a luminal B breast cancer, a lymphoma, a T-cell lymphoma, a B-cell lymphoma, a nasal-type lymphoma, non-Hodgkin's lymphoma, a follicular lymphoma, a penile carcinoma, a prostate cancer, a castration-resistant prostate cancer, an endometrial cancer, a uterine cancer, a myeloma, a multiple myeloma, a head and neck cancer, a prostate cancer, a melanoma, a cutaneous melanoma, a Merkel cell carcinoma, a pancreatic cancer, hepatocellular carcinoma, gastric cancer, a gastroesophageal junction adenocarcinoma, a glioblastoma, a glioblastoma multiforme, a mycosis fungoides, an HPV-positive cancer, a HPV-related cervical carcinoma, a HPV-related anal squamous cell carcinoma, a HPV-related penile squamous cell carcinoma, a HPV-related vulvar squamous cell carcinoma, a vulvar cancer, a vaginal cancer, an anal cancer, an oropharyngeal cancer, an oropharyngeal squamous cell carcinoma, a leukemia, an acute myeloid leukemia, a bone cancer, a solitary bone plasmacytoma, a squamous cell carcinoma, a cutaneous squamous cell carcinoma, a thyroid cancer, a microsatellite stability/proficient mismatch repair (MSS/pMMR) metastatic colorectal cancer, a deficient mismatch repair (dMMR) cancer, a microsatellite instability-high (MSI-H) cancer, a nasal-type extranodal NK/T-Cell lymphoma, a neuroendocrine cancer, a biliary tract cancer, a cholangiocarcinoma, and an intrahepatic cholangiocarcinoma.

In some embodiments of any of the above aspects, the breast cancer is a triple-negative breast cancer, a HER2-positive breast cancer, a HER2-negative breast cancer, an estrogen receptor-positive breast cancer, a progesterone receptor-positive breast cancer, or a luminal B breast cancer.

In some embodiments of any of the above aspects, the lymphoma is a T-cell lymphoma, a B-cell lymphoma, a nasal-type lymphoma, non-Hodgkin's lymphoma, or a follicular lymphoma.

In some embodiments of any of the above aspects, the cancer is selected from the group consisting of a Merkel cell carcinoma, a urothelial carcinoma, a renal cell carcinoma, non-small cell lung cancer, a breast cancer, a triple-negative breast cancer, a hepatocellular carcinoma, a melanoma, a Hodgkin's lymphoma, a head and neck cancer, a colorectal cancer, a gastric cancer, a cervical cancer, a primary mediastinal large B-cell lymphoma, a cutaneous squamous-cell carcinoma, a basal cell carcinoma, a bladder cancer, an endometrial cancer, an esophageal cancer, a malignant pleural mesothelioma, a tumor mutational burden (TMB)-high cancer, a deficient mismatch repair (dMMR) cancer, and a microsatellite instability-high (MSI-H) cancer.

In some embodiments of any of the above aspects, the cancer is selected from the group consisting of a lung cancer, a non-small cell lung cancer, a bronchogenic carcinoma, a breast cancer, a triple-negative breast cancer, an estrogen receptor-positive breast cancer, a HER2-positive breast cancer, a lobular metastatic breast cancer, a ductal breast carcinoma, a cervical cancer, a fallopian tube cancer, a fallopian tube serous adenocarcinoma, an ovarian cancer, an ovarian endometrioid tumor, an ovarian serous adenocarcinoma, an ovarian seromucinous carcinoma, a uterine cancer, an endometrial cancer, a skin cancer, a melanoma, a cutaneous melanoma, a Merkel cell carcinoma, a head and neck cancer, squamous cell carcinoma of head and neck, a hematologic malignancy, a leukemia, a myeloid leukemia, an acute myeloid leukemia, a chronic lymphocytic leukemia, a myelomonocytic leukemia, a thyroid cancer, thyroid gland carcinoma, a thymic carcinoma, a neuroendocrine cancer, a pheochromocytoma, a glioma, a glioblastoma multiforme, a paraganglioma, a lymphoma, a B-cell lymphoma, a Hodgkin lymphoma, a B-cell non-Hodgkin lymphoma, a non-Hodgkin's lymphoma, a cutaneous T-cell lymphoma, a diffuse large B-cell lymphoma, a follicular lymphoma, a marginal zone lymphoma, a pancreatic cancer, a pancreatic ductal adenocarcinoma, a rectal cancer, a colon cancer, a colorectal cancer, a urinary tract cancer, a genitourinary cancer, a mesothelioma, a pleural mesothelioma, a peritoneal mesothelioma, a sarcoma, a chondrosarcoma, a clear cell sarcoma, a liposarcoma, a myxoid/round cell liposarcoma, a synovial sarcoma, an alveolar soft part sarcoma, a gliosarcoma, a uterine carcinosarcoma, a kidney cancer, a non-clear cell kidney cancer, a renal cell carcinoma, a bladder cancer, a urothelial carcinoma, a muscle-invasive bladder cancer, a non-muscle invasive bladder cancer, a HER2-positive bladder cancer, a gallbladder carcinoma, a gastric cancer, an esophageal cancer, an esophageal squamous cell carcinoma, a gastrointestinal cancer, a gastroesophageal cancer, a gastroesophageal junction cancer, a HER2-positive gastric cancer, a primary peritoneal cancer, a cutaneous squamous cell carcinoma, a prostate cancer, a prostate adenocarcinoma, a castration-resistant prostate cancer, a urogenital cancer, a ureter urothelial carcinoma, a renal pelvis urothelial carcinoma, a urethral urothelial carcinoma, an appendix carcinoma, a penile cancer, an anal canal cancer, a hepatocellular carcinoma, a hepatobiliary cancer, an unresectable liver and intrahepatic bile duct carcinoma, a biliary tract cancer, a cholangiocarcinoma, an intrahepatic cholangiocarcinoma, an extrahepatic cholangiocarcinoma, an HPV-related cancer, an HPV-related anal squamous cell carcinoma, an HPV-related cervical squamous cell carcinoma, an HPV-related penile squamous cell carcinoma, an HPV-related vulvar squamous cell carcinoma, a nasopharynx carcinoma, a nasopharyngeal carcinoma, a laryngeal squamous cell carcinoma, a hypopharyngeal squamous cell carcinoma, an oral cavity squamous cell carcinoma, and a mycosis fungoides.

In some embodiments of any of the above aspects, the caner is selected from the group consisting of urothelial carcinoma, non-small cell lung cancer (NSCLC), breast cancer, triple-negative breast cancer, hepatocellular carcinoma and melanoma.

In some embodiments of any of the above aspects, the cancer is selected from the group consisting of a multiple myeloma, a cervical cancer, an esophageal cancer, an esophageal squamous cell carcinoma, a lung cancer, a non-small cell lung cancer, a glioblastoma, an endometrial cancer, an ovarian cancer, a squamous cell cancer, a head and neck cancer.

In some embodiments of any of the above aspects, the cancer is selected from the group consisting of a cervical cancer, a squamous cell carcinoma of head and neck, a head and neck cancer, a non-small cell lung cancer, a non-squamous non-small cell lung cancer, an esophageal squamous cell carcinoma, an esophageal cancer, a breast cancer, a triple-negative breast cancer, a gastric cancer, a gastroesophageal junction adenocarcinoma, a multiple myeloma, a non-Hodgkin lymphoma, a B-cell lymphoma, a liver cancer, a bladder cancer, a urothelial carcinoma, a pancreatic cancer, and a pancreatic adenocarcinoma.

In some embodiments of any of the above aspects, the cancer is a solid tumor. In some embodiments of any of the above aspects, the solid tumor is PD-L1 positive. In some embodiments of any of the above aspects, the solid tumor is a histologically-confirmed PD-L1 solid tumor. In some embodiments of any of the above aspects, the solid tumor is locally advanced, recurrent, or metastatic.

In some embodiments of any of the above aspects, the cancer is a hematological cancer.

In some embodiments of any of the above aspects, the method comprises administering to the subject a therapeutically effective amount of a liquid pharmaceutical formulation comprising (a) an anti-TIGIT monoclonal antibody; (b) a buffer; (c) a tonicity agent; and (d) a surfactant, wherein the liquid pharmaceutical formulation is characterized by a pH of about 4.0 to about 7.0, and administering to the subject a therapeutically effective amount of an anti-PD-1 or an anti-PD-L1 monoclonal antibody.

In some embodiments of any of the above aspects, the method comprises administering to the subject a therapeutically effective amount of a liquid pharmaceutical formulation comprising an anti-TIGIT monoclonal antibody, and administering to the subject a therapeutically effective amount of an anti-PD-1 or an anti-PD-L1 monoclonal antibody.

In some embodiments of any of the above aspects, the liquid pharmaceutical formulation and the anti-PD-1 or the anti-PD-L1 monoclonal antibody are administered simultaneously. In some embodiments of any of the above aspects, the liquid pharmaceutical formulation and the anti-PD-1 or the anti-PD-L1 monoclonal antibody are administered separately.

In some embodiments of any of the above aspects, the liquid pharmaceutical formulation and the anti-PD-1 or the anti-PD-L1 monoclonal antibody are mixed 24 hours or less prior to administration to the subject. In some embodiments of any of the above aspects, the liquid pharmaceutical formulation and the anti-PD-1 or the anti-PD-L1 monoclonal antibody are mixed 23 hours or less prior to administration to the subject. In some embodiments of any of the above aspects, the liquid pharmaceutical formulation and the anti-PD-1 or the anti-PD-L1 monoclonal antibody are mixed 22 hours or less prior to administration to the subject. In some embodiments of any of the above aspects, the liquid pharmaceutical formulation and the anti-PD-1 or the anti-PD-L1 monoclonal antibody are mixed 21 hours or less prior to administration to the subject. In some embodiments of any of the above aspects, the liquid pharmaceutical formulation and the anti-PD-1 or the anti-PD-L1 monoclonal antibody are mixed 20 hours or less prior to administration to the subject. In some embodiments of any of the above aspects, the liquid pharmaceutical formulation and the anti-PD-1 or the anti-PD-L1 monoclonal antibody are mixed 19 hours or less prior to administration to the subject. In some embodiments of any of the above aspects, the liquid pharmaceutical formulation and the anti-PD-1 or the anti-PD-L1 monoclonal antibody are mixed 18 hours or less prior to administration to the subject. In some embodiments of any of the above aspects, the liquid pharmaceutical formulation and the anti-PD-1 or the anti-PD-L1 monoclonal antibody are mixed 17 hours or less prior to administration to the subject. In some embodiments of any of the above aspects, the liquid pharmaceutical formulation and the anti-PD-1 or the anti-PD-L1 monoclonal antibody are mixed 16 hours or less prior to administration to the subject. In some embodiments of any of the above aspects, the liquid pharmaceutical formulation and the anti-PD-1 or the anti-PD-L1 monoclonal antibody are mixed 15 hours or less prior to administration to the subject. In some embodiments of any of the above aspects, the liquid pharmaceutical formulation and the anti-PD-1 or the anti-PD-L1 monoclonal antibody are mixed 14 hours or less prior to administration to the subject. In some embodiments of any of the above aspects, the liquid pharmaceutical formulation and the anti-PD-1 or the anti-PD-L1 monoclonal antibody are mixed 13 hours or less prior to administration to the subject. In some embodiments of any of the above aspects, the liquid pharmaceutical formulation and the anti-PD-1 or the anti-PD-L1 monoclonal antibody are mixed 12 hours or less prior to administration to the subject. In some embodiments of any of the above aspects, the liquid pharmaceutical formulation and the anti-PD-1 or the anti-PD-L1 monoclonal antibody are mixed 11 hours or less prior to administration to the subject. In some embodiments of any of the above aspects, the liquid pharmaceutical formulation and the anti-PD-1 or the anti-PD-L1 monoclonal antibody are mixed 10 hours or less prior to administration to the subject. In some embodiments of any of the above aspects, the liquid pharmaceutical formulation and the anti-PD-1 or the anti-PD-L1 monoclonal antibody are mixed 9 hours or less prior to administration to the subject. In some embodiments of any of the above aspects, the liquid pharmaceutical formulation and the anti-PD-1 or the anti-PD-L1 monoclonal antibody are mixed 8 hours or less prior to administration to the subject. In some embodiments of any of the above aspects, the liquid pharmaceutical formulation and the anti-PD-1 or the anti-PD-L1 monoclonal antibody are mixed 7 hours or less prior to administration to the subject. In some embodiments of any of the above aspects, the liquid pharmaceutical formulation and the anti-PD-1 or the anti-PD-L1 monoclonal antibody are mixed 6 hours or less prior to administration to the subject. In some embodiments of any of the above aspects, the liquid pharmaceutical formulation and the anti-PD-1 or the anti-PD-L1 monoclonal antibody are mixed 5 hours or less prior to administration to the subject. In some embodiments of any of the above aspects, the liquid pharmaceutical formulation and the anti-PD-1 or the anti-PD-L1 monoclonal antibody are mixed 4 hours or less prior to administration to the subject. In some embodiments of any of the above aspects, the liquid pharmaceutical formulation and the anti-PD-1 or the anti-PD-L1 monoclonal antibody are mixed 3 hours or less prior to administration to the subject. In some embodiments of any of the above aspects, the liquid pharmaceutical formulation and the anti-PD-1 or the anti-PD-L1 monoclonal antibody are mixed 2 hours or less prior to administration to the subject. In some embodiments of any of the above aspects, the liquid pharmaceutical formulation and the anti-PD-1 or the anti-PD-L1 monoclonal antibody are mixed 1 hours or less prior to administration to the subject. In some embodiments of any of the above aspects, the liquid pharmaceutical formulation and the anti-PD-1 or the anti-PD-L1 monoclonal antibody are mixed 45 minutes or less prior to administration to the subject. In some embodiments of any of the above aspects, the liquid pharmaceutical formulation and the anti-PD-1 or the anti-PD-L1 monoclonal antibody are mixed 30 minutes or less prior to administration to the subject. In some embodiments of any of the above aspects, the liquid pharmaceutical formulation and the anti-PD-1 or the anti-PD-L1 monoclonal antibody are mixed 15 minutes or less prior to administration to the subject. In some embodiments of any of the above aspects, the liquid pharmaceutical formulation and the anti-PD-1 or the anti-PD-L1 monoclonal antibody are mixed 10 minutes or less prior to administration to the subject. In some embodiments of any of the above aspects, the liquid pharmaceutical formulation and the anti-PD-1 or the anti-PD-L1 monoclonal antibody are mixed 5 minutes or less prior to administration to the subject. In some embodiments of any of the above aspects, the liquid pharmaceutical formulation and the anti-PD-1 or the anti-PD-L1 monoclonal antibody are mixed 4 minutes or less prior to administration to the subject. In some embodiments of any of the above aspects, the liquid pharmaceutical formulation and the anti-PD-1 or the anti-PD-L1 monoclonal antibody are mixed 3 minutes or less prior to administration to the subject. In some embodiments of any of the above aspects, the liquid pharmaceutical formulation and the anti-PD-1 or the anti-PD-L1 monoclonal antibody are mixed 2 minutes or less prior to administration to the subject. In some embodiments of any of the above aspects, the liquid pharmaceutical formulation and the anti-PD-1 or the anti-PD-L1 monoclonal antibody are mixed 1 minutes or less prior to administration to the subject.

In some embodiments of any of the above aspects, the liquid pharmaceutical formulation and the anti-PD-1 or the anti-PD-L1 monoclonal antibody are mixed during administration to the subject.

In some embodiments of any of the above aspects, the liquid pharmaceutical formulation and the anti-PD-1 or the anti-PD-L1 monoclonal antibody are administered parenterally. In some embodiments of any of the above aspects, the liquid pharmaceutical formulation and the anti-PD-1 or the anti-PD-L1 monoclonal antibody are administered intravenously or subcutaneously. In some embodiments of any of the above aspects, the liquid pharmaceutical formulation and the anti-PD-1 or the anti-PD-L1 monoclonal antibody are administered intravenously. In some embodiments of any of the above aspects, liquid pharmaceutical formulation and the anti-PD-1 or the anti-PD-L1 monoclonal antibody are administered subcutaneously.

In any of the methods of treatment disclosed herein, the anti-TIGIT monoclonal antibody may comprise a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

In some embodiments of any of the above aspects, the anti-TIGIT monoclonal antibody comprises a heavy chain variable region comprising the amino acid sequence of SEQ ID NO: 7. In some embodiments of any of the above aspects, the anti-TIGIT monoclonal antibody comprises a light chain variable region comprising the amino acid sequence of SEQ ID NO: 9. In some embodiments of any of the above aspects, the anti-TIGIT monoclonal antibody comprises a heavy chain variable region comprising the amino acid sequence of SEQ ID NO: 7 and a light chain variable region comprising the amino acid sequence of SEQ ID NO: 9. In some embodiments of any of the above aspects, the heavy chain of the anti-TIGIT monoclonal antibody comprises the amino acid sequence of SEQ ID NO: 18. In some embodiments of any of the above aspects, the heavy chain of the anti-TIGIT monoclonal antibody comprises the amino acid sequence of SEQ ID NO: 19. In some embodiments of any of the above aspects, the heavy chain of the anti-TIGIT monoclonal antibody comprises the amino acid sequence of SEQ ID NO: 24. In some embodiments of any of the above aspects, the heavy chain of the anti-TIGIT monoclonal antibody comprises the amino acid sequence of SEQ ID NO: 25. In some embodiments of any of the above aspects, the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 18 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 19 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 24 and the light chain of the anti-TIGIT monoclonal antibody comprises the amino acid sequence of SEQ ID NO: 20. In some embodiments of any of the above aspects, the heavy chain of the anti-TIGIT monoclonal antibody comprises the amino acid sequence of SEQ ID NO: 25 and the light chain of the anti-TIGIT monoclonal antibody comprises the amino acid sequence of SEQ ID NO: 20. In some embodiments, the anti-TIGIT monoclonal antibody is tiragolumab. In some embodiments, the anti-TIGIT monoclonal antibody is tiragolumab.

In some embodiments of any of the above aspects, the anti-TIGIT monoclonal antibody is an IgG antibody. The anti-TIGIT monoclonal antibody may be an IgG1 antibody, an IgG2 antibody, an IgG3 antibody, or an IgG4 antibody. In some embodiments of any of the above aspects, the anti-TIGIT monoclonal antibody is an IgG1 or an IgG4 antibody. In some embodiments of any of the above aspects, the anti-TIGIT monoclonal antibody is an IgG1 antibody. In some embodiments of any of the above aspects, the anti-TIGIT monoclonal antibody is an IgG4 antibody. In some embodiments of any of the above aspects, the anti-TIGIT monoclonal antibody is an antagonist antibody. In some embodiments of any of the above aspects, the anti-TIGIT monoclonal antibody is a full-length antibody. In some embodiments of any of the above aspects, the anti-TIGIT monoclonal antibody is a full-length IgG antibody. In some embodiments of any of the above aspects, the anti-TIGIT monoclonal antibody is a full-length IgG1 antibody. In some embodiments of any of the above aspects, the anti-TIGIT monoclonal antibody is a full-length human IgG1 antibody. In some embodiments of any of the above aspects, the anti-TIGIT monoclonal antibody is a full-length humanized IgG1 antibody. In some cases, the anti-TIGIT monoclonal antibody may have one or more effector functions. In some embodiments of any of the above aspects, the anti-TIGIT monoclonal antibody retains all effector functions. Optionally, one or more effector functions of the anti-TIGIT monoclonal antibody may have been modified or eliminated.

In some embodiments of any of the above aspects, the anti-TIGIT monoclonal antibody is a human antibody. In some embodiments of any of the above aspects, the anti-TIGIT monoclonal antibody is a humanized antibody. The anti-TIGIT monoclonal antibody may be an antibody fragment. In some embodiments, the anti-TIGIT monoclonal antibody is a Fab, a Fab′, a F(ab′)₂, a Fv fragment, or a scFv fragment. In some embodiments, the anti-TIGIT monoclonal antibody is a Fab fragment. In some embodiments, the anti-TIGIT monoclonal antibody is a Fab′ fragment. In some embodiments, the anti-TIGIT monoclonal antibody is a F(ab′)₂, fragment. In some embodiments, the anti-TIGIT monoclonal antibody is a Fv fragment. In some embodiments, the anti-TIGIT monoclonal antibody is a scFv fragment. In some embodiments, the anti-TIGIT monoclonal antibody is a diabody. In some embodiments, the anti-TIGIT monoclonal antibody is a linear antibody. In some embodiments, the anti-TIGIT monoclonal antibody is a single-chain antibody molecule. In some embodiments, the anti-TIGIT monoclonal antibody is a multispecific antibody, e.g. formed from antibody fragments.

In any of the methods of treatment, uses, or formulations-for-use disclosed herein, the anti-PD-L1 monoclonal antibody may comprise a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

In some embodiments of any of the above aspects, the anti-PD-L1 monoclonal antibody comprises a heavy chain variable region comprising the amino acid sequence of SEQ ID NO: 16. In some embodiments of any of the above aspects, the anti-PD-L1 monoclonal antibody comprises a light chain variable region comprising the amino acid sequence of SEQ ID NO: 17. In some embodiments of any of the above aspects, the anti-PD-L1 monoclonal antibody comprises a heavy chain variable region comprising the amino acid sequence of SEQ ID NO: 16 and a light chain variable region comprising the amino acid sequence of SEQ ID NO: 17. In some embodiments of any of the above aspects, the heavy chain of the anti-PD-L1 monoclonal antibody comprises the amino acid sequence of SEQ ID NO: 21. In some embodiments of any of the above aspects, the heavy chain of the anti-PD-L1 monoclonal antibody comprises the amino acid sequence of SEQ ID NO: 22. In some embodiments of any of the above aspects, the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23. In some embodiments of any of the above aspects, the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23. In some embodiments of any of the above aspects, the anti-PD-L1 monoclonal antibody is atezolizumab. In some embodiments of any of the above aspects, the anti-PD-L1 monoclonal antibody is atezolizumab. In some embodiments of any of the above aspects, the anti-PD-L1 monoclonal antibody is atezolizumab, marketed as TECENTRIQ™. Atezolizumab is described in WHO Drug Information (International Nonproprietary Names for Pharmaceutical Substances), Proposed INN: List 112, Vol. 28, No. 4, 2014 (see page 488). In some embodiments of any of the above aspects, atezolizumab has the CAS Registry Number 1380723-44-3.

In some embodiments of any of the above aspects, the anti-PD-L1 monoclonal antibody is an IgG antibody. The anti-PD-L1 monoclonal antibody may be an IgG1 antibody, an IgG2 antibody, an IgG3 antibody, or an IgG4 antibody. In some embodiments of any of the above aspects, the anti-PD-L1 monoclonal antibody is an IgG1 or an IgG4 antibody. In some embodiments of any of the above aspects, the anti-PD-L1 monoclonal antibody is an IgG1 antibody. In some embodiments of any of the above aspects, the anti-PD-L1 monoclonal antibody is an IgG4 antibody. In some embodiments of any of the above aspects, the anti-PD-L1 monoclonal antibody is an antagonist antibody. In some embodiments of any of the above aspects, the anti-PD-L1 monoclonal antibody is a full-length antibody. In some embodiments, the anti-PD-L1 monoclonal antibody is a full-length IgG antibody. In some embodiments, the anti-PD-L1 monoclonal antibody is a full-length IgG1 antibody. In some embodiments, the anti-PD-L1 monoclonal antibody is a full-length human IgG1 antibody. In some embodiments, the anti-PD-L1 monoclonal antibody is a full-length humanized IgG1 antibody. In some cases, the anti-PD-L1 monoclonal antibody may have one or more effector functions. In some embodiments of any of the above aspects, the anti-PD-L1 monoclonal antibody retains all effector functions. Optionally, one or more effector functions of the anti-PD-L1 monoclonal antibody may have been modified or eliminated.

In some embodiments of any of the above aspects, the anti-PD-L1 monoclonal antibody is a human antibody. In some embodiments of any of the above aspects, the anti-PD-L1 monoclonal antibody is a humanized antibody. The anti-PD-L1 monoclonal antibody may be an antibody fragment. In some embodiments, the anti-PD-L1 monoclonal antibody is a Fab, a Fab′, a F(ab′)₂, a Fv, or a scFv fragment. In some embodiments, the anti-PD-L1 monoclonal antibody is a Fab fragment. In some embodiments, the anti-PD-L1 monoclonal antibody is a Fab′ fragment. In some embodiments, the anti-PD-L1 monoclonal antibody is a F(ab′)₂, fragment. In some embodiments, the anti-PD-L1 monoclonal antibody is a Fv fragment. In some embodiments, the anti-PD-L1 monoclonal antibody is a scFv fragment. In some embodiments, the anti-PD-L1 monoclonal antibody is a diabody. In some embodiments, the anti-PD-L1 monoclonal antibody is a linear antibody. In some embodiments, the anti-PD-L1 monoclonal antibody is a single chain antibody molecule. In some embodiments, the anti-PD-L1 monoclonal antibody is a multispecific antibody, e.g. formed from antibody fragments.

In some embodiments of any of the above aspects, the method of treating, use, or formulation-for-use comprises administering an anti-PD-1 antibody. In some embodiments of any of the above aspects, the anti-PD-1 antibody is selected from the group consisting of lambrolizumab (MK-3475), nivolumab (MDX-1106), pembrolizumab, cemiplimab, and dostarlimab. In some embodiments of any of the above aspects, the anti-PD-1 antibody is lambrolizumab (MK-3475). In some embodiments of any of the above aspects, the anti-PD-1 antibody is nivolumab (MDX-1106). In some embodiments of any of the above aspects, the anti-PD-1 antibody is pembrolizumab. In some embodiments of any of the above aspects, the anti-PD-1 antibody is cemiplimab. In some embodiments of any of the above aspects, the anti-PD-1 antibody is dostarlimab.

In some embodiments of any of the above aspects, the anti-PD-L1 monoclonal antibody is administered in a formulation comprising histidine acetate in a concentration of about 15 mM to about 25 mM, sucrose in a concentration of about 200 mM to about 280 mM, polysorbate in a concentration of about 0.04% (w/v) to about 0.08% (w/v), methionine in a concentration of about 5 mM to about 15 mM, and pH of about 5.3 to about 6.0. In some embodiments of any of the above aspects, the anti-PD-L1 monoclonal antibody is administered in a formulation comprising about 20 mM histidine acetate, about 240 mM sucrose, about 0.06% (w/v) polysorbate 20, about 10 mM methionine, and a pH of about 5.8. In some embodiments of any of the above aspects, the anti-PD-L1 monoclonal antibody is administered in a formulation comprising histidine acetate in a concentration of 15 mM to 25 mM, sucrose in a concentration of 200 mM to 280 mM, polysorbate in a concentration of 0.04% (w/v) to 0.08% (w/v), methionine in a concentration of 5 mM to 15 mM, and pH of 5.3 to 6.0. In some embodiments of any of the above aspects, the anti-PD-L1 monoclonal antibody is administered in a formulation comprising 20 mM histidine acetate, 240 mM sucrose, 0.06% (w/v) polysorbate 20, 10 mM methionine, and a pH of 5.8.

Specific anti-TIGIT monoclonal antibody and anti-PD-L1 monoclonal antibody sequences are provided in Table 2.

TABLE 2
Amino acid sequences.

| SEQ ID NO | Antibody | Ab Region | Amino acid sequence |
|---|---|---|---|
| 1 | Anti-TIGIT | HVR-H1 | SNSAAWN |
| 2 | Anti-TIGIT | HVR-H2 | KTYYRFKWYSDYAVSVKG |
| 3 | Anti-TIGIT | HVR-H3 | ESTTYDLLAGPFDY |
| 4 | Anti-TIGIT | HVR-L1 | KSSQTVLYSSNNKKYLA |
| 5 | Anti-TIGIT | HVR-L2 | WASTRES |
| 6 | Anti-TIGIT | HVR-L3 | QQYYSTPFT |
| 7 | Anti-TIGIT | Heavy chain | EVQLQQSGPGLVKPSQTLSLTCAISGDSV |
| | | variable | SSNSAAWNWIRQSPSRGLEWLGKTYYRF |
| | | region (VH) | KWYSDYAVSVKGRITINPDTSKNQFSLQL |
| | | | NSVTPEDTAVFYCTRESTTYDLLAGPFDY |
| | | | WGQGTLVTVSS |
| 8 | Anti-TIGIT | VH | QVQLQQSGPGLVKPSQTLSLTCAISGDSV |
| | | | SSNSAAWNWIRQSPSRGLEWLGKTYYRF |
| | | | KWYSDYAVSVKGRITINPDTSKNQFSLQL |
| | | | NSVTPEDTAVFYCTRESTTYDLLAGPFDY |
| | | | WGQGTLVTVSS |
| 9 | Anti-TIGIT | Light chain | DIVMTQSPDSLAVSLGERATINCKSSQTV |
| | | variable | LYSSNNKKYLAWYQQKPGQPPNLLIYW |
| | | region (VL) | ASTRESGVPDRFSGSGSGTDFTLTISSLQA |
| | | | EDVAVYYCQQYYSTPFTFGPGTKVEIK |
| 18 | Anti-TIGIT | Heavy chain | EVQLQQSGPGLVKPSQTLSLTCAISGDSV |
| | | (no C- | SSNSAAWNWIRQSPSRGLEWLGKTYYRF |
| | | terminal | KWYSDYAVSVKGRITINPDTSKNQFSLQL |
| | | lysine) | NSVTPEDTAVFYCTRESTTYDLLAGPFDY |
| | | | WGQGTLVTVSSASTKGPSVFPLAPSSKST |
| | | | SGGTAALGCLVKDYFPEPVTVSWNSGAL |
| | | | TSGVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPG |
| 19 | Anti-TIGIT | Heavy chain | EVQLQQSGPGLVKPSQTLSLTCAISGDSV |
| | | (with C- | SSNSAAWNWIRQSPSRGLEWLGKTYYRF |
| | | terminal | KWYSDYAVSVKGRITINPDTSKNQFSLQL |
| | | lysine) | NSVTPEDTAVFYCTRESTTYDLLAGPFDY |
| | | | WGQGTLVTVSSASTKGPSVFPLAPSSKST |
| | | | SGGTAALGCLVKDYFPEPVTVSWNSGAL |
| | | | TSGVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPGK |
| 24 | Anti-TIGIT | Heavy chain | QVQLQQSGPGLVKPSQTLSLTCAISGDSV |
| | | (E1Q; no C- | SSNSAAWNWIRQSPSRGLEWLGKTYYRF |
| | | terminal | KWYSDYAVSVKGRITINPDTSKNQFSLQL |
| | | lysine) | NSVTPEDTAVFYCTRESTTYDLLAGPFDY |
| | | | WGQGTLVTVSSASTKGPSVFPLAPSSKST |
| | | | SGGTAALGCLVKDYFPEPVTVSWNSGAL |
| | | | TSGVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPG |
| 25 | Anti-TIGIT | Heavy chain | QVQLQQSGPGLVKPSQTLSLTCAISGDSV |
| | | (E1Q; with C- | SSNSAAWNWIRQSPSRGLEWLGKTYYRF |
| | | terminal | KWYSDYAVSVKGRITINPDTSKNQFSLQL |
| | | lysine) | NSVTPEDTAVFYCTRESTTYDLLAGPFDY |
| | | | WGQGTLVTVSSASTKGPSVFPLAPSSKST |
| | | | SGGTAALGCLVKDYFPEPVTVSWNSGAL |
| | | | TSGVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPGK |
| 20 | Anti-TIGIT | Light chain | DIVMTQSPDSLAVSLGERATINCKSSQTV |
| | | | LYSSNNKKYLAWYQQKPGQPPNLLIYW |
| | | | ASTRESGVPDRFSGSGSGTDFTLTISSLQA |
| | | | EDVAVYYCQQYYSTPFTFGPGTKVEIKRT |
| | | | VAAPSVFIFPPSDEQLKSGTASVVCLLNN |
| | | | FYPREAKVQWKVDNALQSGNSQESVTE |
| | | | QDSKDSTYSLSSTLTLSKADYEKHKVYA |
| | | | CEVTHQGLSSPVTKSFNRGEC |
| 10 | Anti-PD-L1 | HVR-H1 | GFTFSDSWIH |
| 11 | Anti-PD-L1 | HVR-H2 | AWISPYGGSTYYADSVKG |
| 12 | Anti-PD-L1 | HVR-H3 | RHWPGGFDY |
| 13 | Anti-PD-L1 | HVR-L1 | RASQDVSTAVA |
| 14 | Anti-PD-L1 | HVR-L2 | SASFLYS |
| 15 | Anti-PD-L1 | HVR-L3 | QQYLYHPAT |
| 16 | Anti-PD-L1 | VH | EVQLVESGGGLVQPGGSLRLSCAASGFTF |
| | | | SDSWIHWVRQAPGKGLEWVAWISPYGG |
| | | | STYYADSVKGRFTISADTSKNTAYLQMN |
| | | | SLRAEDTAVYYCARRHWPGGFDYWGQG |
| | | | TLVTVSS |
| 17 | Anti-PD-L1 | VL | DIQMTQSPSSLSASVGDRVTITCRASQDV |
| | | | STAVAWYQQKPGKAPKLLIYSASFLYSG |
| | | | VPSRFSGSGSGTDFTLTISSLQPEDFATYY |
| | | | CQQYLYHPATFGQGTKVEIKR |
| 21 | Anti-PD-L1 | Heavy chain | EVQLVESGGGLVQPGGSLRLSCAASGFTF |
| | | (no C- | SDSWIHWVRQAPGKGLEWVAWISPYGG |
| | | terminal | STYYADSVKGRFTISADTSKNTAYLQMN |
| | | lysine) | SLRAEDTAVYYCARRHWPGGFDYWGQG |
| | | | TLVTVSSASTKGPSVFPLAPSSKSTSGGTA |
| | | | ALGCLVKDYFPEPVTVSWNSGALTSGVH |
| | | | TFPAVLQSSGLYSLSSVVTVPSSSLGTQTY |
| | | | ICNVNHKPSNTKVDKKVEPKSCDKTHTC |
| | | | PPCPAPELLGGPSVFLFPPKPKDTLMISRT |
| | | | PEVTCVVVDVSHEDPEVKFNWYVDGVE |
| | | | VHNAKTKPREEQYASTYRVVSVLTVLHQ |
| | | | DWLNGKEYKCKVSNKALPAPIEKTISKA |
| | | | KGQPREPQVYTLPPSREEMTKNQVSLTCL |
| | | | VKGFYPSDIAVEWESNGQPENNYKTTPP |
| | | | VLDSDGSFFLYSKLTVDKSRWQQGNVFS |
| | | | CSVMHEALHNHYTQKSLSLSPG |
| 22 | Anti-PD-L1 | Heavy chain | EVQLVESGGGLVQPGGSLRLSCAASGFTF |
| | | (with C- | SDSWIHWVRQAPGKGLEWVAWISPYGG |
| | | terminal | STYYADSVKGRFTISADTSKNTAYLQMN |
| | | lysine) | SLRAEDTAVYYCARRHWPGGFDYWGQG |
| | | | TLVTVSSASTKGPSVFPLAPSSKSTSGGTA |
| | | | ALGCLVKDYFPEPVTVSWNSGALTSGVH |
| | | | TFPAVLQSSGLYSLSSVVTVPSSSLGTQTY |
| | | | ICNVNHKPSNTKVDKKVEPKSCDKTHTC |
| | | | PPCPAPELLGGPSVFLFPPKPKDTLMISRT |
| | | | PEVTCVVVDVSHEDPEVKFNWYVDGVE |
| | | | VHNAKTKPREEQYASTYRVVSVLTVLHQ |
| | | | DWLNGKEYKCKVSNKALPAPIEKTISKA |
| | | | KGQPREPQVYTLPPSREEMTKNQVSLTCL |
| | | | VKGFYPSDIAVEWESNGQPENNYKTTPP |
| | | | VLDSDGSFFLYSKLTVDKSRWQQGNVFS |
| | | | CSVMHEALHNHYTQKSLSLSPGK |
| 23 | Anti-PD-L1 | Light chain | DIQMTQSPSSLSASVGDRVTITCRASQDV |
| | | | STAVAWYQQKPGKAPKLLIYSASFLYSG |
| | | | VPSRFSGSGSGTDFTLTISSLQPEDFATYY |
| | | | CQQYLYHPATFGQGTKVEIKRTVAAPSV |
| | | | FIFPPSDEQLKSGTASVVCLLNNFYPREA |
| | | | KVQWKVDNALQSGNSQESVTEQDSKDS |
| | | | TYSLSSTLTLSKADYEKHKVYACEVTHQ |
| | | | GLSSPVTKSFNRGEC |

It is known in the art that, during the expression and processing of antibodies in a cell culture, C-terminal clipping by carboxypeptidases in the culture medium occurs. In some instances, only the C-terminal lysine residue of the heavy chain is clipped. In other instances, additional residues may be clipped. The sequences provided herein are also intended to encompass variants that occur, e.g. by C-terminal clipping, as a result of the production process.

It is also known in the art that the C-terminal cleavage process is imprecise and that additional C-terminal residues are cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated.

In some embodiments, the anti-TIGIT monoclonal antibody is co-mixed with the anti-PD-L1 monoclonal antibody. In some embodiments, the anti-TIGIT monoclonal antibody is subcutaneously co-mixed with the anti-PD-L1 monoclonal antibody. In some embodiments, the anti-TIGIT monoclonal antibody is co-mixed with the anti-PD-L1 monoclonal antibody in the abdomen. In some embodiments, the anti-TIGIT monoclonal antibody is subcutaneously co-mixed with the anti-PD-L1 monoclonal antibody in the abdomen. In some embodiments, the anti-TIGIT monoclonal antibody is co-mixed with the anti-PD-L1 monoclonal antibody in the thigh. In some embodiments, the anti-TIGIT monoclonal antibody is subcutaneously co-mixed with the anti-PD-L1 monoclonal antibody in the thigh. In some embodiments, administration of the anti-PD-L1 monoclonal antibody is simultaneous with administration of the anti-TIGIT monoclonal antibody. In some embodiments, intravenous administration of the anti-PD-L1 monoclonal antibody is sequential to the administration of the anti-TIGIT monoclonal antibody. In some embodiments, administration of the anti-PD-L1 monoclonal antibody is prior to administration of the anti-TIGIT monoclonal antibody. In some embodiments, administration of the anti-PD-L1 monoclonal antibody is subsequent to administration of the anti-TIGIT monoclonal antibody.

In some embodiments, the anti-TIGIT monoclonal antibody is administered at a frequency selected from the group consisting of Q1W, Q2W, Q3W, Q4W, Q5W and Q6W. In some embodiments, the anti-TIGIT monoclonal antibody is administered at a frequency of Q3W in one or more cycles. In some embodiments, the anti-PD-L1 monoclonal antibody is administered at a frequency of Q3W in one or more cycles. In some embodiments, the anti-PD-L1 monoclonal antibody and anti-TIGIT monoclonal antibody are independently administered at a frequency of Q3W in one or more cycles.

In some embodiments, 880 mg anti-TIGIT monoclonal antibody is co-mixed or co-formulated with 1875 mg or 2000 mg anti-PD-L1 monoclonal antibody. In some embodiments, 880 mg anti-TIGIT monoclonal antibody is co-mixed with 2000 mg anti-PD-L1 monoclonal antibody. In some embodiments, 880 mg anti-TIGIT monoclonal antibody is co-mixed with 1875 mg anti-PD-L1 monoclonal antibody. In some embodiments, the co-mixture is administered subcutaneously. In some embodiments, the co-mixture is subcutaneously administered in the thigh. In some embodiments, the co-mixture is subcutaneously administered in the abdomen. In some embodiments, 880 mg anti-TIGIT monoclonal antibody is co-mixed with 2000 mg anti-PD-L1 monoclonal antibody and subcutaneously administered in the abdomen of a subject in need thereof. In some embodiments, 880 mg anti-TIGIT monoclonal antibody is co-mixed with 1875 mg anti-PD-L1 monoclonal antibody and subcutaneously administered in the thigh of a subject in need thereof.

In some embodiments, following the subcutaneous administration of the co-mixture, 1200 mg anti-PD-L1 monoclonal antibody and 600 mg anti-TIGIT monoclonal antibody are intravenously administered Q3W. In some embodiments, the intravenous Q3W administration of 1200 mg anti-PD-L1 monoclonal antibody and 600 mg anti-TIGIT monoclonal antibody begins in Cycle 2 (i.e. after one cycle of subcutaneous administration of the co-mixture). In some embodiments, the intravenous Q3W administration of 1200 mg anti-PD-L1 monoclonal antibody and 600 mg anti-TIGIT monoclonal antibody begins in Cycle 4 (i.e. after three cycles of subcutaneous administration of the co-mixture). In some embodiments, 1200 mg anti-PD-L1 monoclonal antibody and 600 mg anti-TIGIT monoclonal antibody are separately administered Q3W intravenously. In some embodiments, 2000 mg anti-PD-L1 monoclonal antibody and 880 mg anti-TIGIT monoclonal antibody are administered Q3W, e.g., as a coformulation described herein. In some embodiments, 2000 mg anti-PD-L1 monoclonal antibody and 880 mg anti-TIGIT monoclonal antibody are intravenously administered Q3W. In some embodiments, 2000 mg anti-PD-L1 monoclonal antibody and 880 mg anti-TIGIT monoclonal antibody are separately administered Q3W intravenously.

In some embodiments, the liquid pharmaceutical formulation of this disclosure is administered every three weeks (Q3W). In some embodiments, the liquid pharmaceutical formulation of this disclosure is administered at a frequency of Q3W subcutaneously. In some embodiments, the liquid pharmaceutical formulation of this disclosure is administered at a frequency of Q3W intravenously.

### EXEMPLARY EMBODIMENTS

Particular embodiments of the disclosure are set forth in the following numbered paragraphs:
1. A liquid pharmaceutical formulation comprising:
   (a) 18 mg/mL to 176 mg/mL of an anti-TIGIT monoclonal antibody;
   (b) 54 mg/mL to 137.5 mg/mL of an anti-PD-L1 monoclonal antibody;
   (c) 5 mM to 30 mM of a histidine buffer;
   (d) 120 mM to 320 mM of sucrose; and
   (e) 0.02% (w/v) to 0.08% (w/v) polysorbate 20,
wherein the liquid pharmaceutical formulation is characterized by a pH of about 5.2-6.1; wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.
2. The liquid pharmaceutical formulation of embodiment 1, wherein the formulation comprises:
   (a) 36 mg/mL to 44 mg/mL of the anti-TIGIT monoclonal antibody;
   (b) 72 mg/mL to 88 mg/mL of the anti-PD-L1 monoclonal antibody;
   (c) 15 mM to 25 mM of the histidine buffer;
   (d) 200 mM to 280 mM of sucrose; and
   (e) 0.04% (w/v) to 0.08% (w/v) polysorbate 20,
wherein the liquid pharmaceutical formulation is characterized by a pH of about 5.5-6.1.
3. The liquid pharmaceutical formulation of embodiment 1 or 2, wherein the formulation comprises:
   (a) 40 mg/mL of the anti-TIGIT monoclonal antibody;
   (b) 80 mg/mL of the anti-PD-L1 monoclonal antibody;
   (c) 20 mM of the histidine buffer;
   (d) 240 mM of sucrose; and
   (e) 0.06% (w/v) of polysorbate 20,
wherein the liquid pharmaceutical formulation is characterized by a pH of about 5.8.
4. A liquid pharmaceutical formulation comprising:
   (a) 18 mg/mL to 75 mg/mL of an anti-TIGIT monoclonal antibody;
   (b) 54 mg/mL to 137.5 mg/mL of an anti-PD-L1 monoclonal antibody;
   (c) 12 mM to 28 mM of a histidine buffer;
   (d) 100 mM to 300 mM of sucrose; and
   (e) 0.02% (w/v) to 0.08% (w/v) polysorbate 20,
wherein the liquid pharmaceutical formulation is characterized by a pH of about 5.4-6.2;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.
5. The liquid pharmaceutical formulation of embodiment 4, wherein the formulation comprises:
   (a) 30 mg/mL to 50 mg/mL of the anti-TIGIT monoclonal antibody;
   (b) 70 mg/mL to 90 mg/mL of the anti-PD-L1 monoclonal antibody;
   (c) 15 mM to 25 mM of the histidine buffer;
   (d) 200 mM to 280 mM of sucrose; and
   (e) 0.04% (w/v) to 0.08% (w/v) polysorbate 20, wherein the liquid pharmaceutical formulation is characterized by a pH of about 5.6-6.0.
6. The liquid pharmaceutical formulation of embodiment 4 or 5, wherein the formulation comprises:
   (a) 36 mg/mL to 44 mg/mL of the anti-TIGIT monoclonal antibody;
   (b) 72 mg/mL to 88 mg/mL of the anti-PD-L1 monoclonal antibody;
   (c) 18 mM to 22 mM of the histidine buffer;
   (d) 220 mM to 260 mM of sucrose; and
   (e) 0.05% (w/v) to 0.07% (w/v) of polysorbate 20, wherein the liquid pharmaceutical formulation is characterized by a pH of about 5.7 to 5.9.
7. The liquid pharmaceutical formulation of any one of embodiments 4-6, wherein the formulation comprises:
   (a) 40 mg/mL of the anti-TIGIT monoclonal antibody;
   (b) 80 mg/mL of the anti-PD-L1 monoclonal antibody;
   (c) 20 mM of the histidine buffer;
   (d) 240 mM of sucrose; and
   (e) 0.06% (w/v) of polysorbate 20,
wherein the liquid pharmaceutical formulation is characterized by a pH of about 5.8.
8. A liquid pharmaceutical formulation comprising:
   (a) 18 mg/mL to 176 mg/mL of an anti-TIGIT monoclonal antibody;
   (b) 500 U/mL to 2600 U/mL of a hyaluronidase;
   (c) 5 mM to 30 mM of a histidine buffer;
   (d) 180 mM to 320 mM of sucrose; and
   (e) 0.03% (w/v) to 0.08% (w/v) polysorbate 20,
wherein the liquid pharmaceutical formulation is characterized by a pH of about 5.2-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.
9. The liquid pharmaceutical formulation of embodiment 8, wherein the formulation comprises:
   (a) 144 mg/mL to 176 mg/mL of the anti-TIGIT monoclonal antibody;
   (b) 1400 U/mL to 2600 U/mL of the hyaluronidase;
   (c) 5 mM to 25 mM of the histidine buffer;
   (d) 180 mM to 320 mM of sucrose; and
   (e) 0.04% (w/v) to 0.08% (w/v) polysorbate 20,
wherein the liquid pharmaceutical formulation is characterized by a pH of about 5.2-5.8.
10. The liquid pharmaceutical formulation of embodiment 8 or 9, wherein the formulation comprises:
   (a) 160 mg/mL of the anti-TIGIT monoclonal antibody;
   (b) 2000 U/mL of the hyaluronidase;
   (c) 20 mM of the histidine buffer;
   (d) 240 mM of sucrose; and
   (e) 0.06% (w/v) of polysorbate 20,
wherein the liquid pharmaceutical formulation is characterized by a pH of about 5.5.
11. A liquid pharmaceutical formulation comprising:
   (a) 30 mg/mL to 60 mg/mL of an anti-TIGIT monoclonal antibody;
   (b) 60 mg/mL to 120 mg/mL of an anti-PD-L1 monoclonal antibody;
   (c) 500 U/mL to 2600 U/mL of a hyaluronidase;
   (d) 5 mM to 30 mM of a histidine buffer;
   (e) 180 mM to 320 mM of sucrose; and
   (f) 0.03% (w/v) to 0.08% (w/v) polysorbate 20,
wherein the liquid pharmaceutical formulation is characterized by a pH of about 5.2-6.1;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.
12. The liquid pharmaceutical formulation of embodiment 11, wherein the formulation comprises:
   (a) 35 mg/mL to 55 mg/mL of the anti-TIGIT monoclonal antibody;
   (b) 70 mg/mL to 110 mg/mL of the anti-PD-L1 monoclonal antibody;
   (c) 1400 U/mL to 2600 U/mL of the hyaluronidase;
   (d) 5 mM to 25 mM of the histidine buffer;
   (e) 180 mM to 320 mM of sucrose; and
   (f) 0.04% (w/v) to 0.08% (w/v) polysorbate 20,
wherein the liquid pharmaceutical formulation is characterized by a pH of about 5.5-5.8.
13. The liquid pharmaceutical formulation of embodiment 11 or 12, wherein the formulation comprises:
   (a) 30 mg/mL of the anti-TIGIT monoclonal antibody;
   (b) 60 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
14. The liquid pharmaceutical formulation of embodiment 11 or 12, wherein the formulation comprises:
   (a) 35 mg/mL of the anti-TIGIT monoclonal antibody;
   (b) 70 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
15. The liquid pharmaceutical formulation of embodiment 11 or 12, wherein the formulation comprises:
   (a) 40 mg/mL of the anti-TIGIT monoclonal antibody;
   (b) 80 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
16. The liquid pharmaceutical formulation of embodiment 11 or 12, wherein the formulation comprises:
   (a) 45 mg/mL of the anti-TIGIT monoclonal antibody;
   (b) 90 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
17. The liquid pharmaceutical formulation of embodiment 11 or 12, wherein the formulation comprises:
   (a) 50 mg/mL of the anti-TIGIT monoclonal antibody;
   (b) 100 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
18. The liquid pharmaceutical formulation of embodiment 11 or 12, wherein the formulation comprises:
   (a) 55 mg/mL of the anti-TIGIT monoclonal antibody;
   (b) 110 mg/mL of the anti-PD-L1 monoclonal antibody;
   (c) 2000 U/mL of the hyaluronidase;
   (d) 20 mM of the histidine buffer;
   (e) 240 mM of sucrose; and
   (f) 0.06% (w/v) polysorbate 20,
wherein the liquid pharmaceutical formulation is characterized by a pH of about 5.5-5.8.
19. A liquid pharmaceutical formulation comprising:
   (a) 18 mg/mL to 176 mg/mL of an anti-TIGIT monoclonal antibody;
   (b) 5 mM to 30 mM of histidine acetate;
   (c) 100 mM to 320 mM of sucrose; and
   (d) 0.01% (w/v) to 0.08% (w/v) polysorbate 20,
wherein the liquid pharmaceutical formulation is characterized by a pH of about 5.0-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.
20. The liquid pharmaceutical formulation of embodiment 19, wherein the formulation comprises:
   (a) 144 mg/mL to 176 mg/mL of the anti-TIGIT monoclonal antibody;
   (b) 5 mM to 25 mM of the histidine buffer;
   (c) 180 mM to 320 mM of sucrose; and
   (d) 0.05% (w/v) to 0.08% (w/v) polysorbate 20,
wherein the liquid pharmaceutical formulation is characterized by a pH of about 5.2-5.8.
21. The liquid pharmaceutical formulation of embodiment 19 or 20, wherein the formulation comprises:
   (a) 160 mg/mL of the anti-TIGIT monoclonal antibody;
   (b) 20 mM of the histidine buffer;
   (c) 240 mM of sucrose; and
   (d) 0.06% (w/v) of polysorbate 20,
wherein the liquid pharmaceutical formulation is characterized by a pH of about 5.5.
22. A liquid pharmaceutical formulation comprising:
   (a) 18 mg/mL to 75 mg/mL of an anti-TIGIT monoclonal antibody;
   (b) 5 mM to 30 mM of histidine acetate;
   (c) 100 mM to 320 mM of sucrose; and
   (d) 0.01% (w/v) to 0.08% (w/v) polysorbate 20,
wherein the liquid pharmaceutical formulation is characterized by a pH of about 5.0-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.
23. The liquid pharmaceutical formulation of embodiment 22, wherein the formulation comprises:
   (a) 50 mg/mL to 70 mg/mL of the anti-TIGIT monoclonal antibody;
   (b) 15 mM to 25 mM of the histidine buffer;
   (c) 200 mM to 280 mM of sucrose; and
   (d) 0.02% (w/v) to 0.06% (w/v) polysorbate 20,
wherein the liquid pharmaceutical formulation is characterized by a pH of about 5.2-5.8.
24. The liquid pharmaceutical formulation of embodiment 22 or 23, wherein the formulation comprises:
   (a) 54 mg/mL to 66 mg/mL of the anti-TIGIT monoclonal antibody;
   (b) 18 mM to 22 mM of the histidine buffer;
   (c) 220 mM to 260 mM of sucrose; and
   (d) 0.03% (w/v) to 0.05% (w/v) polysorbate 20,
wherein the liquid pharmaceutical formulation is characterized by a pH of about 5.4-5.6.
25. The liquid pharmaceutical formulation of any one of embodiments 22-24, wherein the formulation comprises:
   (a) 60 mg/mL of the anti-TIGIT monoclonal antibody;
   (b) 20 mM of the histidine buffer;
   (c) 240 mM of sucrose; and
   (d) 0.04% (w/v) of polysorbate 20,
wherein the liquid pharmaceutical formulation is characterized by a pH of about 5.5.
26. The liquid pharmaceutical formulation according to any one of embodiments 1-25, wherein the histidine buffer is histidine acetate.
27. The liquid pharmaceutical formulation according to any one of embodiments 1-26, wherein the heavy chain variable region of the anti-TIGIT monoclonal antibody comprises the amino acid sequence of SEQ ID NO: 7 or SEQ ID NO: 8.
28. The liquid pharmaceutical formulation according to any one of embodiments 1-27, wherein the light chain variable region of the anti-TIGIT monoclonal antibody comprises the amino acid sequence of SEQ ID NO: 9.
29. The liquid pharmaceutical formulation according to any one of embodiments 1-28, wherein the heavy chain variable region of the anti-TIGIT monoclonal antibody comprises the amino acid sequence of SEQ ID NO: 7 or SEQ ID NO: 8 and the light chain variable region of the anti-TIGIT monoclonal antibody comprises the amino acid sequence of SEQ ID NO: 9.
30. The liquid pharmaceutical formulation according to any one of embodiments 1-29, wherein the heavy chain of the anti-TIGIT monoclonal antibody comprises the amino acid sequence of SEQ ID NO: 18 or SEQ ID NO: 24.
31. The liquid pharmaceutical formulation according to any one of embodiments 1-29, wherein the heavy chain of the anti-TIGIT monoclonal antibody comprises the amino acid sequence of SEQ ID NO: 19 or SEQ ID NO: 25.
32. The liquid pharmaceutical formulation according to any one of embodiments 1-31, wherein the light chain of the anti-TIGIT monoclonal antibody comprises the amino acid sequence of SEQ ID NO: 20.
33. The liquid pharmaceutical formulation according to any one of embodiments 1-30, wherein the heavy chain of the anti-TIGIT monoclonal antibody comprises the amino acid sequence of SEQ ID NO: 18 or SEQ ID NO: 24, and the light chain of the anti-TIGIT monoclonal antibody comprises the amino acid sequence of SEQ ID NO: 20.
34. The liquid pharmaceutical formulation according to any one of embodiments 1-29 and 31, wherein the heavy chain of the anti-TIGIT monoclonal antibody comprises the amino acid sequence of SEQ ID NO: 19 or SEQ ID NO: 25, and the light chain of the anti-TIGIT monoclonal antibody comprises the amino acid sequence of SEQ ID NO: 20.
35. The liquid pharmaceutical formulation according to any one of embodiments 1-34, wherein the anti-TIGIT monoclonal antibody is an IgG antibody.
36. The liquid pharmaceutical formulation according to embodiment 35, wherein the anti-TIGIT monoclonal antibody is an IgG1 or an IgG4 antibody.
37. The liquid pharmaceutical formulation according to any one of embodiments 1-36, wherein the anti-TIGIT monoclonal antibody is a full-length antibody.
38. The liquid pharmaceutical formulation according to any one of embodiments 1-26 and 35-37, wherein the anti-TIGIT monoclonal antibody is a human antibody.
39. The liquid pharmaceutical formulation according to any one of embodiments 1-37, wherein the anti-TIGIT monoclonal antibody is a humanized antibody.
40. The liquid pharmaceutical formulation according to any one of embodiments 1-39, wherein the anti-TIGIT monoclonal antibody inhibits or blocks the interaction of CD226 with TIGIT.
41. The liquid pharmaceutical formulation according to any one of embodiments 1-26, wherein the anti-TIGIT monoclonal antibody is tiragolumab.
42. The liquid pharmaceutical formulation according to any one of embodiments 1-7 and 27-41, wherein the heavy chain variable region of the anti-PD-L1 monoclonal antibody comprises the amino acid sequence of SEQ ID NO: 16.
43. The liquid pharmaceutical formulation according to any one of embodiments 1-7 and 27-41, wherein the light chain variable region of the anti-PD-L1 monoclonal antibody comprises the amino acid sequence of SEQ ID NO: 17.
44. The liquid pharmaceutical formulation according to any one of embodiments 1-7 and 27-43, wherein the heavy chain variable region of the anti-PD-L1 monoclonal antibody comprises the amino acid sequence of SEQ ID NO: 16 and the light chain variable region of the anti-PD-L1 monoclonal antibody comprises the amino acid sequence of SEQ ID NO: 17.
45. The liquid pharmaceutical formulation according to any one of embodiments 1-7 and 27-44, wherein the heavy chain of the anti-PD-L1 monoclonal antibody comprises the amino acid sequence of SEQ ID NO: 21.
46. The liquid pharmaceutical formulation according to any one of embodiments 1-7 and 27-44, wherein the heavy chain of the anti-PD-L1 monoclonal antibody comprises the amino acid sequence of SEQ ID NO: 22.
47. The liquid pharmaceutical formulation according to any one of embodiments 1-7 and 27-46, wherein the light chain of the anti-PD-L1 monoclonal antibody comprises the amino acid sequence of SEQ ID NO: 23.
48. The liquid pharmaceutical formulation according to any one of embodiments 1-7 and 27-45 and 47, wherein the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23.
49. The liquid pharmaceutical formulation according to any one of embodiments 1-7 and 27-44 and 46-47, wherein the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23.
50. The liquid pharmaceutical formulation according to any one of embodiments 1-7 and 27-49, wherein the anti-PD-L1 monoclonal antibody is an IgG antibody.
51. The liquid pharmaceutical formulation according to embodiment 50, wherein the anti-PD-L1 monoclonal antibody is an IgG1 or an IgG4 antibody.
52. The liquid pharmaceutical formulation according to any one of embodiments 1-7 and 27-51, wherein the anti-PD-L1 monoclonal antibody is a full-length antibody.
53. The liquid pharmaceutical formulation according to any one of embodiments 1-7 and 27-41 and 50-52, wherein the anti-PD-L1 monoclonal antibody is a human antibody.
54. The liquid pharmaceutical formulation according to any one of embodiments 1-7 and 27-52, wherein the anti-PD-L1 monoclonal antibody is a humanized antibody.
55. The liquid pharmaceutical formulation according to any one of embodiments 1-7 and 27-41, wherein the anti-PD-L1 monoclonal antibody is atezolizumab.
56. The liquid pharmaceutical formulation according to any one of embodiments 1-55, wherein the formulation further comprises a stabilizer.
57. The liquid pharmaceutical formulation according to embodiment 56, wherein the stabilizer is selected from the group consisting of methionine, glycine, alanine, proline, taurine, betaine, octopine, glutamate, sarcosine, γ-aminobutyric acid, and trimethylamine N-oxide.
58. The liquid pharmaceutical formulation according to embodiment 57, wherein the stabilizer is methionine.
59. The liquid pharmaceutical formulation according to any one of embodiments 56-58, wherein the concentration of the stabilizer is about 5 mM to about 15 mM.
60. The liquid pharmaceutical formulation according to embodiment 59, wherein the concentration of the stabilizer is about 10 mM.
61. The liquid pharmaceutical formulation according to any one of embodiments 8-10 and 26-60, wherein the hyaluronidase is a recombinant human hyaluronidase.
62. The liquid pharmaceutical formulation according to embodiment 61, wherein the recombinant human hyaluronidase is a human soluble PH20 hyaluronidase glycoprotein, such as rHuPH20.
63. An article of manufacture comprising the liquid pharmaceutical formulation according to any one of embodiments 1-62.
64. The article of manufacture according to embodiment 63, wherein the article of manufacture is a vial.
65. The article of manufacture according to embodiment 64, wherein the vial is stoppered with a chlorobutyl elastomer stopper.
66. The article of manufacture according to embodiment 63, wherein the article of manufacture is a pre-filled syringe.
67. The article of manufacture according to embodiment 63, wherein the article of manufacture is a syringe pump.
68. The article of manufacture according to any one of embodiments 63-67, wherein the article comprises about 3 mL to about 30 mL of the liquid pharmaceutical formulation.
69. The article of manufacture according to embodiment 68, wherein the article comprises about 10 mL of the liquid pharmaceutical formulation.
70. The article of manufacture according to embodiment 68, wherein the article comprises about 7 mL of the liquid pharmaceutical formulations.
71. The article of manufacture according to embodiment 68, wherein the article comprises about 6.5 mL of the liquid pharmaceutical formulation.
72. The article of manufacture according to embodiment 68, wherein the article comprises about 21 mL of the liquid pharmaceutical formulation.
73. A method of treating cancer in a subject in need thereof comprising administering to the subject a therapeutically effective amount of the liquid pharmaceutical formulation according to any one of embodiments 1-62.
74. Use of the liquid pharmaceutical formulation according to any one of embodiments 1-62 in the manufacture of a medicament for treating cancer in a subject in need thereof.
75. The liquid pharmaceutical formulation according to any one of embodiments 1-62 for use in treating cancer in a subject in need thereof.
76. The method according to embodiment 73, the use according to embodiment 74 or the liquid pharmaceutical formulation according to embodiment 75, wherein the liquid pharmaceutical formulation is administered intravenously.
77. The method according to embodiment 73, the use according to embodiment 74 or the liquid pharmaceutical formulation according to embodiment 75, wherein the liquid pharmaceutical formulation is administered subcutaneously.
78. The method according to embodiment 76 or 77, the use according to embodiment 76 or 77 or the liquid pharmaceutical formulation according to embodiment 76 or 77, wherein the liquid pharmaceutical formulation is administered every three weeks (Q3W).
79. The method according to any one of embodiments 73 and 76-78, the use according to any one of embodiments 74 and 76-78 or the liquid pharmaceutical formulation according to any one of embodiments 75-78, wherein the cancer is selected from the group consisting of a lung cancer, a non-small cell lung cancer, a renal cell cancer, a urothelial cancer, a ureter cancer, a urethral cancer, a colorectal cancer, a colon cancer, a rectal cancer, a kidney cancer, a sarcoma, an ovarian cancer, a breast cancer, a cervical cancer, a fallopian tube cancer, an endometrial cancer, a uterine cancer, a pancreatic cancer, a gastric carcinoma, a bladder cancer, an esophageal cancer, a mesothelioma, a melanoma, a head and neck cancer, a thyroid cancer, a sarcoma, a prostate cancer, a penile cancer, a glioblastoma, a thymic carcinoma, an esophageal carcinoma, a nasopharyngeal cancer, a mesothelioma, a liver cancer, a biliary tract cancer, a HPV-positive cancer, a leukemia, a lymphoma, a brain cancer, a neuroendocrine cancer, a myeloma, a mycosis fungoides, a Merkel cell cancer, a hematologic malignancy, a deficient mismatch repair (dMMR) cancer, and a microsatellite instability-high (MSI-H) cancer.
80. The method according to any one of embodiments 73 and 76-78, the use according to any one of embodiments 74 and 76-78 or the liquid pharmaceutical formulation according to any one of embodiments 75-78, wherein the cancer is selected from the group consisting of a bladder cancer, a muscle-invasive bladder cancer, a urothelial carcinoma, a ureter cancer, a urethral cancer, a ureter urothelial carcinoma, a urethral urothelial carcinoma, a renal cancer, a renal pelvis cancer, a renal cell carcinoma, a clear-cell renal carcinoma, a rectal cancer, a colon cancer, a colorectal cancer, a sarcoma, an osteosarcoma, a leiomyosarcoma, a pleomorphic sarcoma, a myxofibrosarcoma, a liposarcoma, a chondrosarcoma, a lung cancer, a non-small cell lung cancer, a fallopian tube cancer, a peritoneal carcinoma, an esophageal cancer, an esophageal squamous cell carcinoma, a mesothelioma, a pleural mesothelioma, a peritoneal mesothelioma, an ovarian cancer, a cervical cancer, a cervical adenosquamous carcinoma, a breast cancer, a triple-negative breast cancer, a HER2-positive breast cancer, a HER2-negative breast cancer, an estrogen receptor-positive breast cancer, a progesterone receptor-positive breast cancer, a luminal B breast cancer, a lymphoma, a T-cell lymphoma, a B-cell lymphoma, a nasal-type lymphoma, non-Hodgkin's lymphoma, a follicular lymphoma, a penile carcinoma, a prostate cancer, a castration-resistant prostate cancer, an endometrial cancer, a uterine cancer, a myeloma, a multiple myeloma, a head and neck cancer, a prostate cancer, a melanoma, a cutaneous melanoma, a Merkel cell carcinoma, a pancreatic cancer, hepatocellular carcinoma, gastric cancer, a gastroesophageal junction adenocarcinoma, a glioblastoma, a glioblastoma multiforme, a mycosis fungoides, an HPV-positive cancer, a HPV-related cervical carcinoma, a HPV-related anal squamous cell carcinoma, a HPV-related penile squamous cell carcinoma, a HPV-related vulvar squamous cell carcinoma, a vulvar cancer, a vaginal cancer, an anal cancer, an oropharyngeal cancer, an oropharyngeal squamous cell carcinoma, a leukemia, an acute myeloid leukemia, a bone cancer, a solitary bone plasmacytoma, a squamous cell carcinoma, a cutaneous squamous cell carcinoma, a thyroid cancer, a microsatellite stability/proficient mismatch repair (MSS/pMMR) metastatic colorectal cancer, a deficient mismatch repair (dMMR) cancer, a microsatellite instability-high (MSI-H) cancer, a nasal-type extranodal NK/T-Cell lymphoma, a neuroendocrine cancer, a biliary tract cancer, a cholangiocarcinoma, and an intrahepatic cholangiocarcinoma.
81. The method according to any one of embodiments 73 and 76-78, the use according to any one of embodiments 74 and 76-78 or the liquid pharmaceutical formulation according to any one of embodiments 75-78, wherein the cancer is selected from the group consisting of a Merkel cell carcinoma, a urothelial carcinoma, a renal cell carcinoma, non-small cell lung cancer, a breast cancer, a triple-negative breast cancer, a hepatocellular carcinoma, a melanoma, a Hodgkin's lymphoma, a head and neck cancer, a colorectal cancer, a gastric cancer, a cervical cancer, a primary mediastinal large B-cell lymphoma, a cutaneous squamous-cell carcinoma, a basal cell carcinoma, a bladder cancer, an endometrial cancer, an esophageal cancer, a malignant pleural mesothelioma, a tumor mutational burden (TMB)-high cancer, a deficient mismatch repair (dMMR) cancer, and a microsatellite instability-high (MSI-H) cancer.
82. The method according to any one of embodiments 73 and 76-78, the use according to any one of embodiments 74 and 76-78 or the liquid pharmaceutical formulation according to any one of embodiments 75-78, wherein the cancer is selected from the group consisting of a lung cancer, a non-small cell lung cancer, a bronchogenic carcinoma, a breast cancer, a triple-negative breast cancer, an estrogen receptor-positive breast cancer, a HER2-positive breast cancer, a lobular metastatic breast cancer, a ductal breast carcinoma, a cervical cancer, a fallopian tube cancer, a fallopian tube serous adenocarcinoma, an ovarian cancer, an ovarian endometrioid tumor, an ovarian serous adenocarcinoma, an ovarian seromucinous carcinoma, a uterine cancer, an endometrial cancer, a skin cancer, a melanoma, a cutaneous melanoma, a Merkel cell carcinoma, a head and neck cancer, squamous cell carcinoma of head and neck, a hematologic malignancy, a leukemia, a myeloid leukemia, an acute myeloid leukemia, a chronic lymphocytic leukemia, a myelomonocytic leukemia, a thyroid cancer, thyroid gland carcinoma, a thymic carcinoma, a neuroendocrine cancer, a pheochromocytoma, a glioma, a glioblastoma multiforme, a paraganglioma, a lymphoma, a B-cell lymphoma, a Hodgkin lymphoma, a B-cell non-Hodgkin lymphoma, a non-Hodgkin's lymphoma, a cutaneous T-cell lymphoma, a diffuse large B-cell lymphoma, a follicular lymphoma, a marginal zone lymphoma, a pancreatic cancer, a pancreatic ductal adenocarcinoma, a rectal cancer, a colon cancer, a colorectal cancer, a urinary tract cancer, a genitourinary cancer, a mesothelioma, a pleural mesothelioma, a peritoneal mesothelioma, a sarcoma, a chondrosarcoma, a clear cell sarcoma, a liposarcoma, a myxoid/round cell liposarcoma, a synovial sarcoma, an alveolar soft part sarcoma, a gliosarcoma, a uterine carcinosarcoma, a kidney cancer, a non-clear cell kidney cancer, a renal cell carcinoma, a bladder cancer, a urothelial carcinoma, a muscle-invasive bladder cancer, a non-muscle invasive bladder cancer, a HER2-positive bladder cancer, a gallbladder carcinoma, a gastric cancer, an esophageal cancer, an esophageal squamous cell carcinoma, a gastrointestinal cancer, a gastroesophageal cancer, a gastroesophageal junction cancer, a HER2-positive gastric cancer, a primary peritoneal cancer, a cutaneous squamous cell carcinoma, a prostate cancer, a prostate adenocarcinoma, a castration-resistant prostate cancer, a urogenital cancer, a ureter urothelial carcinoma, a renal pelvis urothelial carcinoma, a urethral urothelial carcinoma, an appendix carcinoma, a penile cancer, an anal canal cancer, a hepatocellular carcinoma, a hepatobiliary cancer, an unresectable liver and intrahepatic bile duct carcinoma, a biliary tract cancer, a cholangiocarcinoma, an intrahepatic cholangiocarcinoma, an extrahepatic cholangiocarcinoma, an HPV-related cancer, an HPV-related anal squamous cell carcinoma, an HPV-related cervical squamous cell carcinoma, an HPV-related penile squamous cell carcinoma, an HPV-related vulvar squamous cell carcinoma, a nasopharynx carcinoma, a nasopharyngeal carcinoma, a laryngeal squamous cell carcinoma, a hypopharyngeal squamous cell carcinoma, an oral cavity squamous cell carcinoma, and a mycosis fungoides.
83. The method according to any one of embodiments 73 and 76-78, the use according to any one of embodiments 74 and 76-78 or the liquid pharmaceutical formulation according to any one of embodiments 75-78, wherein the cancer is selected from the group consisting of urothelial carcinoma, non-small cell lung cancer (NSCLC), breast cancer, triple-negative breast cancer, hepatocellular carcinoma and melanoma.
84. The method according to any one of embodiments 73 and 76-78, the use according to any one of embodiments 74 and 76-78 or the liquid pharmaceutical formulation according to any one of embodiments 75-78, wherein the cancer is selected from the group consisting of a multiple myeloma, a cervical cancer, an esophageal cancer, an esophageal squamous cell carcinoma, a lung cancer, a non-small cell lung cancer, a glioblastoma, an endometrial cancer, an ovarian cancer, a squamous cell cancer, a head and neck cancer.
85. The method according to any one of embodiments 73 and 76-78, the use according to any one of embodiments 74 and 76-78 or the liquid pharmaceutical formulation according to any one of embodiments 75-78, wherein the cancer is selected from the group consisting of a cervical cancer, a squamous cell carcinoma of head and neck, a head and neck cancer, a non-small cell lung cancer, a non-squamous non-small cell lung cancer, an esophageal squamous cell carcinoma, an esophageal cancer, a breast cancer, a triple-negative breast cancer, a gastric cancer, a gastroesophageal junction adenocarcinoma, a multiple myeloma, a non-Hodgkin lymphoma, a B-cell lymphoma, a liver cancer, a bladder cancer, a urothelial carcinoma, a pancreatic cancer, and a pancreatic adenocarcinoma.
86. The method according to any one of embodiments 73 and 76-78, the use according to any one of embodiments 74 and 76-78 or the liquid pharmaceutical formulation according to any one of embodiments 75-78, wherein the cancer is a solid tumor.
87. The method according to any one of embodiments 73 and 76-78, the use according to any one of embodiments 74 and 76-78 or the liquid pharmaceutical formulation according to any one of embodiments 75-78, wherein the cancer is a hematological cancer.
88. A method of treating cancer in a subject in need thereof comprising administering to the subject a therapeutically effective amount of the liquid pharmaceutical formulation according to any one of embodiments 8-25 and 26-62 to the extent they depend from any one of embodiments 8-25 and administering to the subject a therapeutically effective amount of an anti-PD-1 monoclonal antibody, or an anti-PD-L1 monoclonal antibody.
89. Use of the liquid pharmaceutical formulation according to any one of embodiments 8-25 and 26-62 to the extent they depend from any one of embodiments 8-25 and a therapeutically effective amount of an anti-PD-1 monoclonal antibody, or an anti-PD-L1 monoclonal antibody in the manufacture of a medicament for treating cancer in a subject in need thereof.
90. The liquid pharmaceutical formulation according to any one of embodiments 8-25 and 26-62 to the extent they depend from any one of embodiments 8-25 and a therapeutically effective amount of an anti-PD-1 monoclonal antibody, or an anti-PD-L1 monoclonal antibody for use in treating cancer in a subject in need thereof.
91. The method according to embodiment 88, the use according to embodiment 89 or the liquid pharmaceutical formulation according to embodiment 90, wherein the liquid pharmaceutical formulation and the anti-PD-1 monoclonal antibody, or anti-PD-L1 monoclonal antibody, are administered simultaneously.
92. The method according to embodiment 88 or 91, the use according to embodiment 89 or 91, or the liquid pharmaceutical formulation according to embodiment 90 or 91, wherein the liquid pharmaceutical formulation and the anti-PD-1 monoclonal antibody, or anti-PD-L1 monoclonal antibody, are mixed 24 hours or less prior to administration to the subject.
93. The method according to embodiment 92, the use according to embodiment 92 or the liquid pharmaceutical formulation according to embodiment 92, wherein the liquid pharmaceutical formulation and the anti-PD-1 monoclonal antibody, or anti-PD-L1 monoclonal antibody, are mixed during administration to the subject.
94. The method according to any one of any one of embodiments 88 and 91-93, the use according to any one of embodiments 89 and 91-93 or the liquid pharmaceutical formulation according to any one of embodiments 90-93, wherein the liquid pharmaceutical formulation is administered intravenously.
95. The method according to any one of any one of embodiments 88 and 91-93, the use according to any one of embodiments 89 and 91-93 or the liquid pharmaceutical formulation according to any one of embodiments 90-93, wherein the liquid pharmaceutical formulation is administered subcutaneously.
96. The method according to embodiment 94 or 95, the use according to embodiment 94 or 95 or the liquid pharmaceutical formulation according to embodiment 94 or 95, wherein the liquid pharmaceutical formulation is administered every three weeks (Q3W).
97. The method according to any one of embodiments 88 and 91-96, the use according to any one of embodiments 89 and 91-96 or the liquid pharmaceutical formulation according to any one of embodiments 90-96, wherein the cancer is selected from the group consisting of a lung cancer, a non-small cell lung cancer, a renal cell cancer, a urothelial cancer, a ureter cancer, a urethral cancer, a colorectal cancer, a colon cancer, a rectal cancer, a kidney cancer, a sarcoma, an ovarian cancer, a breast cancer, a cervical cancer, a fallopian tube cancer, an endometrial cancer, a uterine cancer, a pancreatic cancer, a gastric carcinoma, a bladder cancer, an esophageal cancer, a mesothelioma, a melanoma, a head and neck cancer, a thyroid cancer, a sarcoma, a prostate cancer, a penile cancer, a glioblastoma, a thymic carcinoma, an esophageal carcinoma, a nasopharyngeal cancer, a mesothelioma, a liver cancer, a biliary tract cancer, a HPV-positive cancer, a leukemia, a lymphoma, a brain cancer, a neuroendocrine cancer, a myeloma, a mycosis fungoides, a Merkel cell cancer, a hematologic malignancy, a deficient mismatch repair (dMMR) cancer, and a microsatellite instability-high (MSI-H) cancer.
98. The method according to any one of embodiments 88 and 91-96, the use according to any one of embodiments 89 and 91-96 or the liquid pharmaceutical formulation according to any one of embodiments 90-96, wherein the cancer is selected from the group consisting of a bladder cancer, a muscle-invasive bladder cancer, a urothelial carcinoma, a ureter cancer, a urethral cancer, a ureter urothelial carcinoma, a urethral urothelial carcinoma, a renal cancer, a renal pelvis cancer, a renal cell carcinoma, a clear-cell renal carcinoma, a rectal cancer, a colon cancer, a colorectal cancer, a sarcoma, an osteosarcoma, a leiomyosarcoma, a pleomorphic sarcoma, a myxofibrosarcoma, a liposarcoma, a chondrosarcoma, a lung cancer, a non-small cell lung cancer, a fallopian tube cancer, a peritoneal carcinoma, an esophageal cancer, an esophageal squamous cell carcinoma, a mesothelioma, a pleural mesothelioma, a peritoneal mesothelioma, an ovarian cancer, a cervical cancer, a cervical adenosquamous carcinoma, a breast cancer, a triple-negative breast cancer, a HER2-positive breast cancer, a HER2-negative breast cancer, an estrogen receptor-positive breast cancer, a progesterone receptor-positive breast cancer, a luminal B breast cancer, a lymphoma, a T-cell lymphoma, a B-cell lymphoma, a nasal-type lymphoma, non-Hodgkin's lymphoma, a follicular lymphoma, a penile carcinoma, a prostate cancer, a castration-resistant prostate cancer, an endometrial cancer, a uterine cancer, a myeloma, a multiple myeloma, a head and neck cancer, a prostate cancer, a melanoma, a cutaneous melanoma, a Merkel cell carcinoma, a pancreatic cancer, hepatocellular carcinoma, gastric cancer, a gastroesophageal junction adenocarcinoma, a glioblastoma, a glioblastoma multiforme, a mycosis fungoides, an HPV-positive cancer, a HPV-related cervical carcinoma, a HPV-related anal squamous cell carcinoma, a HPV-related penile squamous cell carcinoma, a HPV-related vulvar squamous cell carcinoma, a vulvar cancer, a vaginal cancer, an anal cancer, an oropharyngeal cancer, an oropharyngeal squamous cell carcinoma, a leukemia, an acute myeloid leukemia, a bone cancer, a solitary bone plasmacytoma, a squamous cell carcinoma, a cutaneous squamous cell carcinoma, a thyroid cancer, a microsatellite stability/proficient mismatch repair (MSS/pMMR) metastatic colorectal cancer, a deficient mismatch repair (dMMR) cancer, a microsatellite instability-high (MSI-H) cancer, a nasal-type extranodal NK/T-Cell lymphoma, a neuroendocrine cancer, a biliary tract cancer, a cholangiocarcinoma, and an intrahepatic cholangiocarcinoma.
99. The method according to any one of embodiments 88 and 91-96, the use according to any one of embodiments 89 and 91-96 or the liquid pharmaceutical formulation according to any one of embodiments 90-96, wherein the cancer is selected from the group consisting of a Merkel cell carcinoma, a urothelial carcinoma, a renal cell carcinoma, non-small cell lung cancer, a breast cancer, a triple-negative breast cancer, a hepatocellular carcinoma, a melanoma, a Hodgkin's lymphoma, a head and neck cancer, a colorectal cancer, a gastric cancer, a cervical cancer, a primary mediastinal large B-cell lymphoma, a cutaneous squamous-cell carcinoma, a basal cell carcinoma, a bladder cancer, an endometrial cancer, an esophageal cancer, a malignant pleural mesothelioma, a tumor mutational burden (TMB)-high cancer, a deficient mismatch repair (dMMR) cancer, and a microsatellite instability-high (MSI-H) cancer.
100. The method according to any one of embodiments 88 and 91-96, the use according to any one of embodiments 89 and 91-96 or the liquid pharmaceutical formulation according to any one of embodiments 90-96, wherein the cancer is selected from the group consisting of a lung cancer, a non-small cell lung cancer, a bronchogenic carcinoma, a breast cancer, a triple-negative breast cancer, an estrogen receptor-positive breast cancer, a HER2-positive breast cancer, a lobular metastatic breast cancer, a ductal breast carcinoma, a cervical cancer, a fallopian tube cancer, a fallopian tube serous adenocarcinoma, an ovarian cancer, an ovarian endometrioid tumor, an ovarian serous adenocarcinoma, an ovarian seromucinous carcinoma, a uterine cancer, an endometrial cancer, a skin cancer, a melanoma, a cutaneous melanoma, a Merkel cell carcinoma, a head and neck cancer, squamous cell carcinoma of head and neck, a hematologic malignancy, a leukemia, a myeloid leukemia, an acute myeloid leukemia, a chronic lymphocytic leukemia, a myelomonocytic leukemia, a thyroid cancer, thyroid gland carcinoma, a thymic carcinoma, a neuroendocrine cancer, a pheochromocytoma, a glioma, a glioblastoma multiforme, a paraganglioma, a lymphoma, a B-cell lymphoma, a Hodgkin lymphoma, a B-cell non-Hodgkin lymphoma, a non-Hodgkin's lymphoma, a cutaneous T-cell lymphoma, a diffuse large B-cell lymphoma, a follicular lymphoma, a marginal zone lymphoma, a pancreatic cancer, a pancreatic ductal adenocarcinoma, a rectal cancer, a colon cancer, a colorectal cancer, a urinary tract cancer, a genitourinary cancer, a mesothelioma, a pleural mesothelioma, a peritoneal mesothelioma, a sarcoma, a chondrosarcoma, a clear cell sarcoma, a liposarcoma, a myxoid/round cell liposarcoma, a synovial sarcoma, an alveolar soft part sarcoma, a gliosarcoma, a uterine carcinosarcoma, a kidney cancer, a non-clear cell kidney cancer, a renal cell carcinoma, a bladder cancer, a urothelial carcinoma, a muscle-invasive bladder cancer, a non-muscle invasive bladder cancer, a HER2-positive bladder cancer, a gallbladder carcinoma, a gastric cancer, an esophageal cancer, an esophageal squamous cell carcinoma, a gastrointestinal cancer, a gastroesophageal cancer, a gastroesophageal junction cancer, a HER2-positive gastric cancer, a primary peritoneal cancer, a cutaneous squamous cell carcinoma, a prostate cancer, a prostate adenocarcinoma, a castration-resistant prostate cancer, a urogenital cancer, a ureter urothelial carcinoma, a renal pelvis urothelial carcinoma, a urethral urothelial carcinoma, an appendix carcinoma, a penile cancer, an anal canal cancer, a hepatocellular carcinoma, a hepatobiliary cancer, an unresectable liver and intrahepatic bile duct carcinoma, a biliary tract cancer, a cholangiocarcinoma, an intrahepatic cholangiocarcinoma, an extrahepatic cholangiocarcinoma, an HPV-related cancer, an HPV-related anal squamous cell carcinoma, an HPV-related cervical squamous cell carcinoma, an HPV-related penile squamous cell carcinoma, an HPV-related vulvar squamous cell carcinoma, a nasopharynx carcinoma, a nasopharyngeal carcinoma, a laryngeal squamous cell carcinoma, a hypopharyngeal squamous cell carcinoma, an oral cavity squamous cell carcinoma, and a mycosis fungoides.
101. The method according to any one of embodiments 88 and 91-96, the use according to any one of embodiments 89 and 91-96 or the liquid pharmaceutical formulation according to any one of embodiments 90-96, wherein the cancer is selected from the group consisting of urothelial carcinoma, non-small cell lung cancer (NSCLC), breast cancer, triple-negative breast cancer, hepatocellular carcinoma and melanoma.
102. The method according to any one of embodiments 88 and 91-96, the use according to any one of embodiments 89 and 91-96 or the liquid pharmaceutical formulation according to any one of embodiments 90-96, wherein the cancer is selected from the group consisting of a multiple myeloma, a cervical cancer, an esophageal cancer, an esophageal squamous cell carcinoma, a lung cancer, a non-small cell lung cancer, a glioblastoma, an endometrial cancer, an ovarian cancer, a squamous cell cancer, a head and neck cancer.
103. The method according to any one of embodiments 88 and 91-96, the use according to any one of embodiments 89 and 91-96 or the liquid pharmaceutical formulation according to any one of embodiments 90-96, wherein the cancer is selected from the group consisting of a cervical cancer, a squamous cell carcinoma of head and neck, a head and neck cancer, a non-small cell lung cancer, a non-squamous non-small cell lung cancer, an esophageal squamous cell carcinoma, an esophageal cancer, a breast cancer, a triple-negative breast cancer, a gastric cancer, a gastroesophageal junction adenocarcinoma, a multiple myeloma, a non-Hodgkin lymphoma, a B-cell lymphoma, a liver cancer, a bladder cancer, a urothelial carcinoma, a pancreatic cancer, and a pancreatic adenocarcinoma.
104. The method according to any one of embodiments 88 and 91-96, the use according to any one of embodiments 89 and 91-96 or the liquid pharmaceutical formulation according to any one of embodiments 90-96, wherein the cancer is a solid tumor.
105. The method according to any one of embodiments 88 and 91-96, the use according to any one of embodiments 89 and 91-96 or the liquid pharmaceutical formulation according to any one of embodiments 90-96, wherein the cancer is a hematological cancer.
106. The method according to any one of embodiments 88 and 91-105, the use according to any one of embodiments 89 and 91-105 or the liquid pharmaceutical formulation according to any one of embodiments 90-105, wherein the method comprises administering an anti-PD-1 antibody.
107. The method according to any one of embodiments 88 and 91-105, the use according to any one of embodiments 89 and 91-105 or the liquid pharmaceutical formulation according to any one of embodiments 90-105, wherein the method comprises administering an anti-PD-L1 monoclonal antibody.
108. The method according to embodiment 107, the use according to embodiment 107 or the liquid pharmaceutical formulation according to embodiment 107, wherein the anti-PD-L1 monoclonal antibody is selected from the group consisting of atezolizumab (MPDL3280A), durvalumab (MEDI4736), avelumab, and MDX-1105.
109. The method according to embodiment 107, the use according to embodiment 107 or the liquid pharmaceutical formulation according to embodiment 107, wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.
110. The method according to embodiment 109, the use according to embodiment 109 or the liquid pharmaceutical formulation according to embodiment 109, wherein the heavy chain variable region of the anti-PD-L1 monoclonal antibody comprises the amino acid sequence of SEQ ID NO: 16.
111. The method according to embodiment 109 or 110, the use according to embodiment 109 or 110, or the liquid pharmaceutical formulation according to embodiment 109 or 110, wherein the light chain variable region of the anti-PD-L1 monoclonal antibody comprises the amino acid sequence of SEQ ID NO: 17.
112. The method according to any one of embodiments 109-111, the use according to any one of embodiments 109-111 or the liquid pharmaceutical formulation according to any one of embodiments 109-111, wherein the heavy chain variable region of the anti-PD-L1 monoclonal antibody comprises the amino acid sequence of SEQ ID NO: 16 and the light chain variable region of the anti-PD-L1 monoclonal antibody comprises the amino acid sequence of SEQ ID NO: 17.
113. The method according to any one of embodiments 88 and 91-112, the use according to any one of embodiments 89 and 91-112 or the liquid pharmaceutical formulation according to any one of embodiments 90-112, wherein the anti-PD-L1 monoclonal antibody or anti-PD-1 monoclonal antibody is an IgG antibody.
114. The method according to embodiment 113, the use according to embodiment 113 or the liquid pharmaceutical formulation according to embodiment 113, wherein the anti-PD-L1 monoclonal antibody or anti-PD-1 monoclonal antibody is an IgG1 or an IgG4 antibody.
115. The method according to any one of embodiments 88 and 91-114, the use according to any one of embodiments 89 and 91-114 or the liquid pharmaceutical formulation according to any one of embodiments 90-114, wherein the anti-PD-L1 monoclonal antibody or anti-PD-1 monoclonal antibody is a full-length antibody.
116. The method according to any one of embodiments 88, 91-107 and 113-115, the use according to any one of embodiments 89, 91-107 and 113-115 or the liquid pharmaceutical formulation according to any one of embodiments 90-107 and 113-115, wherein the anti-PD-L1 monoclonal antibody or anti-PD-1 monoclonal antibody is a human antibody.
117. The method according to any one of embodiments 88, 91-107 and 109-115, the use according to any one of embodiments 89, 91-107 and 109-115 or the liquid pharmaceutical formulation according to any one of embodiments 90-107 and 109-115, wherein the anti-PD-L1 monoclonal antibody or anti-PD-1 monoclonal antibody is a humanized antibody.
118. An article of manufacture comprising a formulation that comprises 18 mg/mL to 176 mg/mL of an anti-TIGIT monoclonal antibody.
119. The article of manufacture according to embodiment 118, wherein the formulation comprises 144 mg/mL to 176 mg/mL of the anti-TIGIT monoclonal antibody.
120. The article of manufacture according to embodiment 118 or 119, wherein the formulation comprises 160 mg/mL of the anti-TIGIT monoclonal antibody.
121. The article of manufacture according to any one of embodiments 118-120, wherein the article of manufacture further comprises a formulation comprising an anti-PD-L1 monoclonal antibody.
122. The article of manufacture according to embodiment 121, wherein the anti-PD-L1 monoclonal antibody is atezolizumab.
123. An article of manufacture comprising a formulation that comprises:
   (a) 30 mg/mL to 60 mg/mL of an anti-TIGIT monoclonal antibody; and
   (b) 60 mg/mL to 120 mg/mL of an anti-PD-L1 monoclonal antibody.
124. The article of manufacture according to embodiment 123, wherein the formulation comprises:
   (a) 35 mg/mL to 55 mg/mL of the anti-TIGIT monoclonal antibody; and
   (b) 70 mg/mL to 110 mg/mL of the anti-PD-L1 monoclonal antibody.
125. The article of manufacture according to embodiment 123 or 124, wherein the formulation comprises:
   (a) 30 mg/mL of the anti-TIGIT monoclonal antibody; and
   (b) 60 mg/mL of the anti-PD-L1 monoclonal antibody.
126. The article of manufacture according to embodiment 123 or 124, wherein the formulation comprises:
   (a) 35 mg/mL of the anti-TIGIT monoclonal antibody; and
   (b) 70 mg/mL of the anti-PD-L1 monoclonal antibody.
127. The article of manufacture according to embodiment 123 or 124, wherein the formulation comprises:
   (a) 40 mg/mL of the anti-TIGIT monoclonal antibody; and
   (b) 80 mg/mL of the anti-PD-L1 monoclonal antibody.
128. The article of manufacture according to embodiment 123 or 124, wherein the formulation comprises:
   (a) 45 mg/mL of the anti-TIGIT monoclonal antibody; and
   (b) 90 mg/mL of the anti-PD-L1 monoclonal antibody.
129. The article of manufacture according to embodiment 123 or 124, wherein the formulation comprises:
   (a) 50 mg/mL of the anti-TIGIT monoclonal antibody; and
   (b) 100 mg/mL of the anti-PD-L1 monoclonal antibody.
130. The article of manufacture according to embodiment 123 or 124, wherein the formulation comprises:
   (a) 55 mg/mL of the anti-TIGIT monoclonal antibody; and
   (b) 110 mg/mL of the anti-PD-L1 monoclonal antibody.
131. An article of manufacture comprising a formulation that comprises 880 mg of an anti-TIGIT monoclonal antibody.
132. The article of manufacture according to embodiment 131, wherein the formulation further comprises 1875 mg or 2000 mg of an anti-PD-L1 monoclonal antibody.
133. The article of manufacture according to embodiment 131, wherein the formulation further comprises 1875 mg of an anti-PD-L1 monoclonal antibody.
134. The article of manufacture according to embodiment 131, wherein the formulation further comprises 2000 mg of an anti-PD-L1 monoclonal antibody.
135. The article of manufacture according to any one of embodiments 131-134, wherein the formulation further comprises an anti-PD-L1 monoclonal antibody.
136. The article of manufacture according to any one of embodiments 131-135, wherein the formulation further comprises hyaluronidase.
137. An article of manufacture comprising a formulation that comprises 880 mg of an anti-TIGIT monoclonal antibody and 1875 mg or 2000 mg of an anti-PD-L1 monoclonal antibody.
138. The article of manufacture according to embodiment 137, wherein the formulation further comprises hyaluronidase.
139. The article of manufacture according to any one of embodiments 118-137, wherein the formulation further comprises hyaluronidase, and wherein the concentration of the hyaluronidase is 500 U/mL to 2600 U/mL.
140. The article of manufacture according to embodiment 139, wherein the concentration of the hyaluronidase is 1400 U/mL to 2600 U/mL.
141. The article of manufacture according to embodiment 139 or 140, wherein the concentration of the hyaluronidase is 2000 U/mL.
142. The article of manufacture according to any one of embodiments 139-141, wherein the hyaluronidase is a recombinant human hyaluronidase.
143. The article of manufacture according to embodiment 142, wherein the recombinant hyaluronidase is a human soluble PH20 hyaluronidase glycoprotein, such as rHuPH20.
144. An article of manufacture comprising a subcutaneous administration device, which contains and delivers to a patient an 880 mg fixed dose of an anti-TIGIT monoclonal antibody.
145. The article of manufacture according to embodiment 144, wherein the subcutaneous administration device further contains and delivers to the patient a 1875 mg or 2000 mg fixed dose of an anti-PD-L1 monoclonal antibody.
146. The article of manufacture according to embodiment 145, wherein the subcutaneous administration device further contains and delivers to the patient a 1875 mg fixed dose of an anti-PD-L1 monoclonal antibody.
147. The article of manufacture according to embodiment 145, wherein the subcutaneous administration device further contains and delivers to the patient a 2000 mg fixed dose of an anti-PD-L1 monoclonal antibody.
148 The article of manufacture according to any one of embodiments 144-147, wherein the subcutaneous administration device further contains and delivers to the patient hyaluronidase.
149. The article of manufacture according to embodiment 148, wherein the hyaluronidase is a human soluble PH20 hyaluronidase glycoprotein, such as rHuPH20.
150. The article of manufacture according to any one of embodiments 144-149, wherein the subcutaneous administration device is selected from the group consisting of a syringe, a syringe pump, an injection device, an infusion pump, an injector pen, a needleless device, an autoinjector, and a subcutaneous patch delivery system.
151. The article of manufacture according to any one of embodiments 144-150, wherein the subcutaneous administration device is a syringe.
152. The article of manufacture according to embodiment 144-150, wherein the subcutaneous administration device is a syringe pump.
153. The article of manufacture according to any one of embodiments 118-152, wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.
154. The article of manufacture according to any one of embodiments 118-153, wherein the heavy chain variable region of the anti-TIGIT monoclonal antibody comprises the amino acid sequence of SEQ ID NO: 7 or SEQ ID NO: 8.
155. The article of manufacture according to any one of embodiments 118-154, wherein the light chain variable region of the anti-TIGIT monoclonal antibody comprises the amino acid sequence of SEQ ID NO: 9.
156. The article of manufacture according to any one of embodiments 118-155, wherein the heavy chain variable region of the anti-TIGIT monoclonal antibody comprises the amino acid sequence of SEQ ID NO: 7 or SEQ ID NO: 8 and the light chain variable region of the anti-TIGIT monoclonal antibody comprises the amino acid sequence of SEQ ID NO: 9.
157. The article of manufacture according to any one of embodiments 118-156, wherein the heavy chain of the anti-TIGIT monoclonal antibody comprises the amino acid sequence of SEQ ID NO: 18 or SEQ ID NO: 24.
158. The article of manufacture according to any one of embodiments 118-156, wherein the heavy chain of the anti-TIGIT monoclonal antibody comprises the amino acid sequence of SEQ ID NO: 19 or SEQ ID NO: 25.
159. The article of manufacture according to any one of embodiments 118-158, wherein the light chain of the anti-TIGIT monoclonal antibody comprises the amino acid sequence of SEQ ID NO: 20.
160. The article of manufacture according to any one of embodiments 118-157 and 159, wherein the heavy chain of the anti-TIGIT monoclonal antibody comprises the amino acid sequence of SEQ ID NO: 18 or SEQ ID NO: 24, and the light chain of the anti-TIGIT monoclonal antibody comprises the amino acid sequence of SEQ ID NO: 20.
161. The article of manufacture according to any one of embodiments 118-156 and 158-159, wherein the heavy chain of the anti-TIGIT monoclonal antibody comprises the amino acid sequence of SEQ ID NO: 19 or SEQ ID NO: 25, and the light chain of the anti-TIGIT monoclonal antibody comprises the amino acid sequence of SEQ ID NO: 20.
162. The article of manufacture according to any one of embodiments 121 and 123-161, wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.
163. The article of manufacture according to any one of embodiments 121 and 123-162, wherein the heavy chain variable region of the anti-PD-L1 monoclonal antibody comprises the amino acid sequence of SEQ ID NO: 16.
164. The article of manufacture according to any one of embodiments 121 and 123-163, wherein the light chain variable region of the anti-PD-L1 monoclonal antibody comprises the amino acid sequence of SEQ ID NO: 17.
165. The article of manufacture according to any one of embodiments 121 and 123-164, wherein the heavy chain variable region of the anti-PD-L1 monoclonal antibody comprises the amino acid sequence of SEQ ID NO: 16, and the light chain variable region of the anti-PD-L1 monoclonal antibody comprises the amino acid sequence of SEQ ID NO: 17.
166. The article of manufacture according to any one of embodiments 121 and 123-165, wherein the heavy chain of the anti-PD-L1 monoclonal antibody comprises the amino acid sequence of SEQ ID NO: 21.
167. The article of manufacture according to any one of embodiments 121 and 123-165, wherein the heavy chain of the anti-PD-L1 monoclonal antibody comprises the amino acid sequence of SEQ ID NO: 22.
168. The article of manufacture according to any one of embodiments 121 and 123-167, wherein the light chain of the anti-PD-L1 monoclonal antibody comprises the amino acid sequence of SEQ ID NO: 23.
169. The article of manufacture according to any one of embodiments 121, 123-166 and 168, wherein the heavy chain of the anti-PD-L1 monoclonal antibody comprises the amino acid sequence of SEQ ID NO: 21, and the light chain of the anti-PD-L1 monoclonal antibody comprises the amino acid sequence of SEQ ID NO: 23.
170. The article of manufacture according to any one of embodiments 121, 123-165 and 167-168, wherein the heavy chain of the anti-PD-L1 monoclonal antibody comprises the amino acid sequence of SEQ ID NO: 22, and the light chain of the anti-PD-L1 monoclonal antibody comprises the amino acid sequence of SEQ ID NO: 23.
171. The article of manufacture according to any one of embodiments 118-170, wherein the anti-TIGIT monoclonal antibody is an IgG antibody.
172. The article of manufacture according to embodiment 171, wherein the anti-TIGIT monoclonal antibody is an IgG1 or an IgG4 antibody.
173. The article of manufacture according to any one of embodiments 118-172, wherein the anti-TIGIT monoclonal antibody is a full-length antibody.
174. The article of manufacture according to any one of embodiments 118-152 and 162-173, wherein the anti-TIGIT monoclonal antibody is a human antibody.
175. The article of manufacture according to any one of embodiments 118-173, wherein the anti-TIGIT monoclonal antibody is a humanized antibody.
176. The article of manufacture according to any one of embodiments 118-175, wherein the anti-TIGIT monoclonal antibody inhibits or blocks the interaction of CD226 with TIGIT.
177. The article of manufacture according to any one of embodiments 118-152 and 162-170, wherein the anti-TIGIT antibody is tiragolumab.
178. The article of manufacture according to any one of embodiments 121 and 123-177, wherein the anti-PD-L1 monoclonal antibody is an IgG antibody.
179. The article of manufacture according to embodiment 178, wherein the anti-PD-L1 monoclonal antibody is an IgG1 or an IgG4 antibody.
180. The article of manufacture according to any one of embodiments 121 and 123-179, wherein the anti-PD-L1 monoclonal antibody is a full-length antibody.
181. The article of manufacture according to any one of embodiments 121, 123-161, and 171-180, wherein the anti-PD-L1 monoclonal antibody is a human antibody.
182. The article of manufacture according to any one of embodiments 121 and 123-180, wherein the anti-PD-L1 monoclonal antibody is a humanized antibody.
183. The article of manufacture according to any one of embodiments 118-182, wherein the article of manufacture is a vial.
184. The article of manufacture according to embodiment 183, wherein the vial is a single dosage vial.
185. The article of manufacture according to embodiment 183 or 184, wherein the vial is stoppered with a chlorobutyl elastomer stopper.
186. The article of manufacture according to any one of embodiments 118-182, wherein the article of manufacture is a pre-filled syringe.
187. The article of manufacture according to any one of embodiments 118-182, wherein the article of manufacture is a syringe pump.
188. The article of manufacture according to any one of embodiments 118-182, wherein the article of manufacture is a subcutaneous administration device.
189. The article of manufacture according to embodiment 188, wherein the subcutaneous administration device is selected from the group consisting of a syringe, a syringe pump, an injection device, an infusion pump, an injector pen, a needleless device, an autoinjector, and a subcutaneous patch delivery system.
190. The article of manufacture according to any one of embodiments 118-189, wherein the article comprises about 3 mL to about 60 mL of the anti-TIGIT monoclonal antibody, and one or more of hyaluronidase, a histidine buffer, sucrose, and polysorbate 20.
191. The article of manufacture according to embodiment 190, wherein the article comprises about 10 mL of the anti-TIGIT monoclonal antibody, and one or more of hyaluronidase, a histidine buffer, sucrose, and polysorbate 20.
192. The article of manufacture according to embodiment 190, wherein the article comprises about 7 mL of the anti-TIGIT monoclonal antibody, and one or more of hyaluronidase, a histidine buffer, sucrose, and polysorbate 20.
193. The article of manufacture according to embodiment 190, wherein the article comprises about 6.5 mL of the anti-TIGIT monoclonal antibody, and one or more of hyaluronidase, a histidine buffer, sucrose, and polysorbate 20.
194. The article of manufacture according to embodiment 190, wherein the article comprises about 21 mL of the anti-TIGIT monoclonal antibody, and one or more of hyaluronidase, a histidine buffer, sucrose, and polysorbate 20.
195. A method of treating cancer in a subject in need thereof comprising administering to the subject a therapeutically effective amount of the formulation comprised in the article of manufacture according to any one of embodiments 118-194.
196. The method according to embodiment 195, wherein the formulation is administered intravenously.
197. The method according to embodiment 195, wherein the formulation is administered subcutaneously.
198. The method according to any one of embodiments 195-197, wherein the formulation is administered every three weeks (Q3W).
199. The method according to any one of embodiments 195-198, wherein the cancer is selected from the group consisting of a lung cancer, a non-small cell lung cancer, a renal cell cancer, a urothelial cancer, a ureter cancer, a urethral cancer, a colorectal cancer, a colon cancer, a rectal cancer, a kidney cancer, a sarcoma, an ovarian cancer, a breast cancer, a cervical cancer, a fallopian tube cancer, an endometrial cancer, a uterine cancer, a pancreatic cancer, a gastric carcinoma, a bladder cancer, an esophageal cancer, a mesothelioma, a melanoma, a head and neck cancer, a thyroid cancer, a sarcoma, a prostate cancer, a penile cancer, a glioblastoma, a thymic carcinoma, an esophageal carcinoma, a nasopharyngeal cancer, a mesothelioma, a liver cancer, a biliary tract cancer, a HPV-positive cancer, a leukemia, a lymphoma, a brain cancer, a neuroendocrine cancer, a myeloma, a mycosis fungoides, a Merkel cell cancer, a hematologic malignancy, a deficient mismatch repair (dMMR) cancer, and a microsatellite instability-high (MSI-H) cancer.
200. The method according to any one of embodiments 195-198, wherein the cancer is selected from the group consisting of a bladder cancer, a muscle-invasive bladder cancer, a urothelial carcinoma, a ureter cancer, a urethral cancer, a ureter urothelial carcinoma, a urethral urothelial carcinoma, a renal cancer, a renal pelvis cancer, a renal cell carcinoma, a clear-cell renal carcinoma, a rectal cancer, a colon cancer, a colorectal cancer, a sarcoma, an osteosarcoma, a leiomyosarcoma, a pleomorphic sarcoma, a myxofibrosarcoma, a liposarcoma, a chondrosarcoma, a lung cancer, a non-small cell lung cancer, a fallopian tube cancer, a peritoneal carcinoma, an esophageal cancer, an esophageal squamous cell carcinoma, a mesothelioma, a pleural mesothelioma, a peritoneal mesothelioma, an ovarian cancer, a cervical cancer, a cervical adenosquamous carcinoma, a breast cancer, a triple-negative breast cancer, a HER2-positive breast cancer, a HER2-negative breast cancer, an estrogen receptor-positive breast cancer, a progesterone receptor-positive breast cancer, a luminal B breast cancer, a lymphoma, a T-cell lymphoma, a B-cell lymphoma, a nasal-type lymphoma, non-Hodgkin's lymphoma, a follicular lymphoma, a penile carcinoma, a prostate cancer, a castration-resistant prostate cancer, an endometrial cancer, a uterine cancer, a myeloma, a multiple myeloma, a head and neck cancer, a prostate cancer, a melanoma, a cutaneous melanoma, a Merkel cell carcinoma, a pancreatic cancer, hepatocellular carcinoma, gastric cancer, a gastroesophageal junction adenocarcinoma, a glioblastoma, a glioblastoma multiforme, a mycosis fungoides, an HPV-positive cancer, a HPV-related cervical carcinoma, a HPV-related anal squamous cell carcinoma, a HPV-related penile squamous cell carcinoma, a HPV-related vulvar squamous cell carcinoma, a vulvar cancer, a vaginal cancer, an anal cancer, an oropharyngeal cancer, an oropharyngeal squamous cell carcinoma, a leukemia, an acute myeloid leukemia, a bone cancer, a solitary bone plasmacytoma, a squamous cell carcinoma, a cutaneous squamous cell carcinoma, a thyroid cancer, a microsatellite stability/proficient mismatch repair (MSS/pMMR) metastatic colorectal cancer, a deficient mismatch repair (dMMR) cancer, a microsatellite instability-high (MSI-H) cancer, a nasal-type extranodal NK/T-Cell lymphoma, a neuroendocrine cancer, a biliary tract cancer, a cholangiocarcinoma, and an intrahepatic cholangiocarcinoma.
201. The method according to any one of embodiments 195-198, wherein the cancer is selected from the group consisting of a Merkel cell carcinoma, a urothelial carcinoma, a renal cell carcinoma, non-small cell lung cancer, a breast cancer, a triple-negative breast cancer, a hepatocellular carcinoma, a melanoma, a Hodgkin's lymphoma, a head and neck cancer, a colorectal cancer, a gastric cancer, a cervical cancer, a primary mediastinal large B-cell lymphoma, a cutaneous squamous-cell carcinoma, a basal cell carcinoma, a bladder cancer, an endometrial cancer, an esophageal cancer, a malignant pleural mesothelioma, a tumor mutational burden (TMB)-high cancer, a deficient mismatch repair (dMMR) cancer, and a microsatellite instability-high (MSI-H) cancer.
202. The method according to any one of embodiments 195-198, wherein the cancer is selected from the group consisting of a lung cancer, a non-small cell lung cancer, a bronchogenic carcinoma, a breast cancer, a triple-negative breast cancer, an estrogen receptor-positive breast cancer, a HER2-positive breast cancer, a lobular metastatic breast cancer, a ductal breast carcinoma, a cervical cancer, a fallopian tube cancer, a fallopian tube serous adenocarcinoma, an ovarian cancer, an ovarian endometrioid tumor, an ovarian serous adenocarcinoma, an ovarian seromucinous carcinoma, a uterine cancer, an endometrial cancer, a skin cancer, a melanoma, a cutaneous melanoma, a Merkel cell carcinoma, a head and neck cancer, squamous cell carcinoma of head and neck, a hematologic malignancy, a leukemia, a myeloid leukemia, an acute myeloid leukemia, a chronic lymphocytic leukemia, a myelomonocytic leukemia, a thyroid cancer, thyroid gland carcinoma, a thymic carcinoma, a neuroendocrine cancer, a pheochromocytoma, a glioma, a glioblastoma multiforme, a paraganglioma, a lymphoma, a B-cell lymphoma, a Hodgkin lymphoma, a B-cell non-Hodgkin lymphoma, a non-Hodgkin's lymphoma, a cutaneous T-cell lymphoma, a diffuse large B-cell lymphoma, a follicular lymphoma, a marginal zone lymphoma, a pancreatic cancer, a pancreatic ductal adenocarcinoma, a rectal cancer, a colon cancer, a colorectal cancer, a urinary tract cancer, a genitourinary cancer, a mesothelioma, a pleural mesothelioma, a peritoneal mesothelioma, a sarcoma, a chondrosarcoma, a clear cell sarcoma, a liposarcoma, a myxoid/round cell liposarcoma, a synovial sarcoma, an alveolar soft part sarcoma, a gliosarcoma, a uterine carcinosarcoma, a kidney cancer, a non-clear cell kidney cancer, a renal cell carcinoma, a bladder cancer, a urothelial carcinoma, a muscle-invasive bladder cancer, a non-muscle invasive bladder cancer, a HER2-positive bladder cancer, a gallbladder carcinoma, a gastric cancer, an esophageal cancer, an esophageal squamous cell carcinoma, a gastrointestinal cancer, a gastroesophageal cancer, a gastroesophageal junction cancer, a HER2-positive gastric cancer, a primary peritoneal cancer, a cutaneous squamous cell carcinoma, a prostate cancer, a prostate adenocarcinoma, a castration-resistant prostate cancer, a urogenital cancer, a ureter urothelial carcinoma, a renal pelvis urothelial carcinoma, a urethral urothelial carcinoma, an appendix carcinoma, a penile cancer, an anal canal cancer, a hepatocellular carcinoma, a hepatobiliary cancer, an unresectable liver and intrahepatic bile duct carcinoma, a biliary tract cancer, a cholangiocarcinoma, an intrahepatic cholangiocarcinoma, an extrahepatic cholangiocarcinoma, an HPV-related cancer, an HPV-related anal squamous cell carcinoma, an HPV-related cervical squamous cell carcinoma, an HPV-related penile squamous cell carcinoma, an HPV-related vulvar squamous cell carcinoma, a nasopharynx carcinoma, a nasopharyngeal carcinoma, a laryngeal squamous cell carcinoma, a hypopharyngeal squamous cell carcinoma, an oral cavity squamous cell carcinoma, and a mycosis fungoides.
203. The method according to any one of embodiments 195-198, wherein the cancer is selected from the group consisting of urothelial carcinoma, non-small cell lung cancer (NSCLC), breast cancer, triple-negative breast cancer, hepatocellular carcinoma and melanoma.
204. The method according to any one of embodiments 195-198, wherein the cancer is selected from the group consisting of a multiple myeloma, a cervical cancer, an esophageal cancer, an esophageal squamous cell carcinoma, a lung cancer, a non-small cell lung cancer, a glioblastoma, an endometrial cancer, an ovarian cancer, a squamous cell cancer, a head and neck cancer.
205. The method according to any one of embodiments 195-198, wherein the cancer is selected from the group consisting of a cervical cancer, a squamous cell carcinoma of head and neck, a head and neck cancer, a non-small cell lung cancer, a non-squamous non-small cell lung cancer, an esophageal squamous cell carcinoma, an esophageal cancer, a breast cancer, a triple-negative breast cancer, a gastric cancer, a gastroesophageal junction adenocarcinoma, a multiple myeloma, a non-Hodgkin lymphoma, a B-cell lymphoma, a liver cancer, a bladder cancer, a urothelial carcinoma, a pancreatic cancer, and a pancreatic adenocarcinoma.
206. The method according to any one of embodiments 195-198, wherein the cancer is a solid tumor.
207. The method according to any one of embodiments 195-198, wherein the cancer is a hematological cancer.

### EXAMPLES

### Example 1: Identifying Stability Risks for Tiragolumab

A stability study was performed to identify stability risks for formulations comprising high concentrations of the anti-TIGIT monoclonal antibody tiragolumab (also known as MTIG7192A). Fourteen different formulations were prepared, and the impact of each component was evaluated with various stress and long-term storage conditions and scored as having a low impact on stability (“−”), an intermediate impact on stability (“0”) or high impact on stability (“+”). See, Table 3, infra (PS20=polysorbate 20; P188=poloxamer 188).

TABLE 3
Stability of tiragolumab formulations

| | | | | | [Histidine | | |
|---|---|---|---|---|---|---|---|
| Formulation | Impact | [Protein] | | [Surfactant] | Acetate] | [Sucrose] | |
| No. | Pattern | (mg/mL) | pH | (% w/v) | (mM) | (mM) | Surfactant |
| 1 | −+++ − − | 18 | 6.0 | 0.05 | 30 | 100 | PS20 |
| 2 | ++++++ | 66 | 6.0 | 0.05 | 30 | 300 | P188 |
| 3 | − + − +++ | 18 | 6.0 | 0.01 | 30 | 300 | P188 |
| 4 | + − − − + − | 66 | 5.0 | 0.01 | 10 | 300 | PS20 |
| 5 | − − + − ++ | 18 | 5.0 | 0.05 | 10 | 300 | P188 |
| 6 | + − − + − + | 66 | 5.0 | 0.01 | 30 | 100 | P188 |
| 7 | ++ − − − + | 66 | 6.0 | 0.01 | 10 | 100 | P188 |
| 8 | 00000 − | 42 | 5.5 | 0.03 | 20 | 200 | PS20 |
| 9 | − + − − + − | 18 | 6.0 | 0.01 | 10 | 300 | PS20 |
| 10 | − − + − − + | 18 | 5.0 | 0.05 | 10 | 100 | P188 |
| 11 | − − − + − − | 18 | 5.0 | 0.01 | 30 | 100 | PS20 |
| 12 | + − +++ − | 66 | 5.0 | 0.05 | 30 | 300 | PS20 |
| 13 | +++ − − − | 66 | 6.0 | 0.05 | 10 | 100 | PS20 |
| 14 | 00000+ | 42 | 5.5 | 0.03 | 20 | 200 | P188 |

Approximately 72.7 mg/mL tiragolumab was buffered and exchanged into water and concentrated to 120 mg/mL using tangential flow filtration. Conditioning buffers were prepared for each individual excipient to spike into the tiragolumab-water solution. The various stock solutions included 1) 200 mM L-histidine; 2) 1800 mM sucrose; 3) 800 mM acetic acid; 4) 1% Polysorbate 20 (PS20); or 5) 1% Poloxamer 188 (P188). The protein-water stock solution and the conditioning buffers were mixed in PETG bottles. The pH, protein concentration, osmolality, and surfactant concentration (SC) of each formulation were confirmed using an evaporative light scattering detector (ELSD) before filling into vials. The material was filtered through a 0.2 μm polyvinylidene fluoride (PVDF) filter and then filled into the following vial configurations according to the required testing and time points as described in Table 4, infra. Minimal testing (“M”) used a 1 mL fill in a 2 cc vial (1 vial). Full testing (“F”) used a 1 mL fill in a 2 cc vial (2 vials) and a 4 mL fill in a 6 cc vial (1 vial). Reserved (“R”) used a 1 mL fill in a 2 cc vial (2 vials). An additional 9-month time point was added as an interim readout using a reserve vial. Full testing was performed, and the assay outputs measured: 1) high molecular weight (HMW) forms, main peaks, low molecular weight (LMW) forms; 2) acidic region; main peak; basic region; 3) sum of LMW forms; main peak; sum of HMW forms; 4) light chain (LC), heavy chain (HC), non-glycosylated heavy chain (NGHC), incompletely reduced species (IRS); 5) pH; 6) polysorbate 20 (PS20) concentration; 7) poloxamer 188 (P188) concentration; 8) number of visible particles; 9) osmolality; 10) color; 11) protein concentration; 12) oxidation hotspots; 13) potency and 14) subvisible particles. Minimal testing was also performed to assess 1) HMW forms, main peaks, LMW forms; 2) Acidic region; main peak; basic region; and 3) Number of visible particles.

TABLE 4
Time points for testing tiragolumab formulations

| | Time point: Days (Months) | | | | | | | | |
|---|---|---|---|---|---|---|---|---|---|
| Temperature | | 7 | 14 | 30 | 91 | 186 | 365 | 730 | Re- |
| (° C.) | 0 | (0.25) | (0.5) | (1) | (3) | (6) | (12) | (24) | serve |
| −20 | F | | | M | M | F | F | F | R |
| 5 | F | | | M | M | F | F | F | R |
| 25 | F | | M | M | F | | | | R |
| 40 | F | M | M | F | | | | | R |

Degradation rates were calculated for each formulation using data from the full study with the following durations: 1) 2 years at −20° C.; 2) 2 years at 5° C.; 3) 3 months at 25° C.; and 4) 1 month at 40° C. A least squares multiple regression was performed using the degradation rates for each assay output observed against the formulation parameters (e.g., pH, protein concentration, surfactant (ST), SC, histidine concentration, and sucrose concentration). A main effects model was constructed and formulation parameters with an estimated p-value less than or equal to 0.05 were considered statistically significant. If multiple parameters were identified as statistically significant, a reduced model was performed to understand the interactions of the formulation parameters. Table 5, infra, demonstrates the significant factors observed in the tiragolumab formulations.

TABLE 5
Statistically significant tiragolumab formulation parameters.

| Temperature | | Attribute | Significant Factor(s) | Two Factor Interactions |
|---|---|---|---|---|
| −20° | C. | HIAC | Sucrose, ST | Two factor interaction observed |
| | | (2 μm particles) | | |
| −20° | C. | HIAC | Sucrose, ST | No statistically significant interaction |
| | | (10 μm particles) | | |
| −20° | C. | IEC | Protein | N/A |
| | | (% acidic region) | concentration | |
| −20° | C. | PS20 | Protein | N/A |
| | | | concentration | |
| −20° | C. | Reduced CE-SDS | pH | N/A |
| | | (% IRS) | | |
| −20° | C. | Reduced CD-SDS | Sucrose | N/A |
| | | (% NGHC) | | |
| 5° | C. | HIAC | ST | N/A |
| | | (5 μm particles) | | |
| 5° | C. | HIAC | ST | N/A |
| | | (10 μm particles) | | |
| 5° | C. | HIAC | pH, protein | No statistically significant interaction |
| | | (25 μm particles) | concentration | |
| 5° | C. | IEC | Histidine | N/A |
| | | (% acidic region) | | |
| 5° | C. | IEC | Histidine | N/A |
| | | (% main peak) | | |
| 5° | C. | NR CE-SDS | pH, protein | No statistically significant interaction |
| | | (% main peak) | concentration | |
| 5° | C. | NR CE-SDS | pH | N/A |
| | | (% sum of LMW | | |
| | | forms) | | |
| 5° | C. | NR CE-SDS | Protein | N/A |
| | | (% sum of HMW | concentration | |
| | | forms) | | |
| 5° | C. | pH | pH | N/A |
| 5° | C. | Protein | SC | N/A |
| | | concentration | | |
| 5° | C. | PS20 | Protein | N/A |
| | | | concentration | |
| 5° | C. | Reduced CD-SDS | pH | N/A |
| | | (% IRS) | | |
| 5° | C. | SEC | Protein | N/A |
| | | (% HMW forms) | concentration | |
| 5° | C. | SEC | pH | N/A |
| | | (% LMW forms) | | |
| 5° | C. | SEC | Protein | N/A |
| | | (% main peak) | concentration | |
| 25° | C. | HIAC | ST | N/A |
| | | (5 μm particles) | | |
| 25° | C. | HIAC | ST | N/A |
| | | (10 μm particles) | | |
| 25° | C. | IEC | pH, protein | No statistically significant interaction |
| | | (% basic region) | concentration, | |
| | | | histidine | |
| 25° | C. | NR CE-SDS | pH | N/A |
| | | (% main peak) | | |
| 25° | C. | NR CE-SDS | pH, histidine | No statistically significant interaction |
| | | (% sum of LMW | | |
| | | forms) | | |
| 25° | C. | NR CE-SDS | pH, protein | Two-factor interaction between protein |
| | | (% sum of HMW | concentration, | concentration and sucrose; three-way |
| | | forms) | sucrose | interaction between sucrose, pH, and |
| | | | | protein concentration; no statistically |
| | | | | significant two-factor interaction between |
| | | | | pH and either protein concentration or |
| | | | | sucrose |
| 25° | C. | pH | pH | N/A |
| 25° | C. | SEC | Protein | No statistically significant interaction |
| | | (% HMW forms) | concentration, | |
| | | | sucrose | |
| 25° | C. | SEC | Protein | No statistically significant interaction |
| | | (% LMW forms) | concentration, | |
| | | | histidine | |
| 25° | C. | SEC | Sucrose | N/A |
| | | (% main peak) | | |
| 40° | C. | HIAC | ST | N/A |
| | | (2 μm particles) | | |
| 40° | C. | IEC | pH | N/A |
| | | (% basic region) | | |
| 40° | C. | IEC | pH, SC, ST | Two-factor interaction between SC and St; |
| | | (% main peak) | | no statistically significant interaction |
| | | | | between pH and either SC or ST |
| 40° | C. | NR CE-SDS | ST | N/A |
| | | (% main peak) | | |
| 40° | C. | NR CE-SDS | pH, ST | No statistically significant interaction |
| | | (% sum of LMW | | |
| | | forms) | | |
| 40° | C. | NR CE-SDS | pH, protein | No statistically significant interaction |
| | | (% sum of HMW | concentration, | |
| | | forms) | SC, ST | |
| 40° | C. | Peptide mapping | ST | N/A |
| | | (% DTLMISR) | | |
| 40° | C. | Peptide mapping | ST | N/A |
| | | (% HEAL) | | |
| 40° | C. | Peptide mapping | SC, sucrose, | Two-factor interaction between SC and ST; |
| | | (% W) | histidine, ST | two-factor interaction between histidine |
| | | | | and ST; no other statistically significant |
| | | | | interactions |
| 40° | C. | Potency | Protein | N/A |
| | | | concentration | |
| 40° | C. | PS20 | SC | N/A |
| 40° | C. | Reduced CE-SDS | pH, ST | Two-factor interaction between pH and ST |
| | | (% IRS) | | |
| 40° | C. | Reduced CE-SDS | pH, histidine | No statistically significant interaction |
| | | (% NGHC) | | |
| 40° | C. | SEC | SC, sucrose, ST | Two-factor interaction between SC and ST; |
| | | (% HMW forms) | | no other statistically significant |
| | | | | interactions |
| 40° | C. | SEC | pH | N/A |
| | | (% LMW forms) | | |
| 40° | C. | SEC | SC, sucrose, | Two-factor interaction between SC and ST; |
| | | (% main peak) | histidine, ST | no other statistically significant |
| | | | | interactions |

CE-SDS = Capillary Electrophoresis-Sodium Dodecyl Sulfate
HIAC = High Accuracy liquid particle counter
HMW = High Molecular Weight
IEC = Ion Exchange Chromatography
IRS = Incompletely Reduced Species
LMW = Low Molecular Weight
N/A = Not Applicable
NGHC = Non-Glycosylated Heavy Chain
NR = Non-Reduced
SC = Surfactant Concentration
SEC = Size-Exclusion Chromatography
ST = Surfactant Type

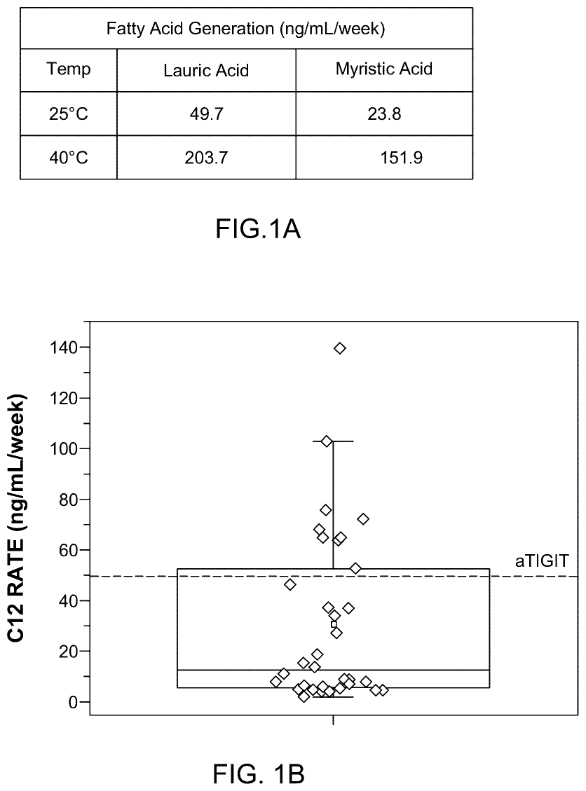

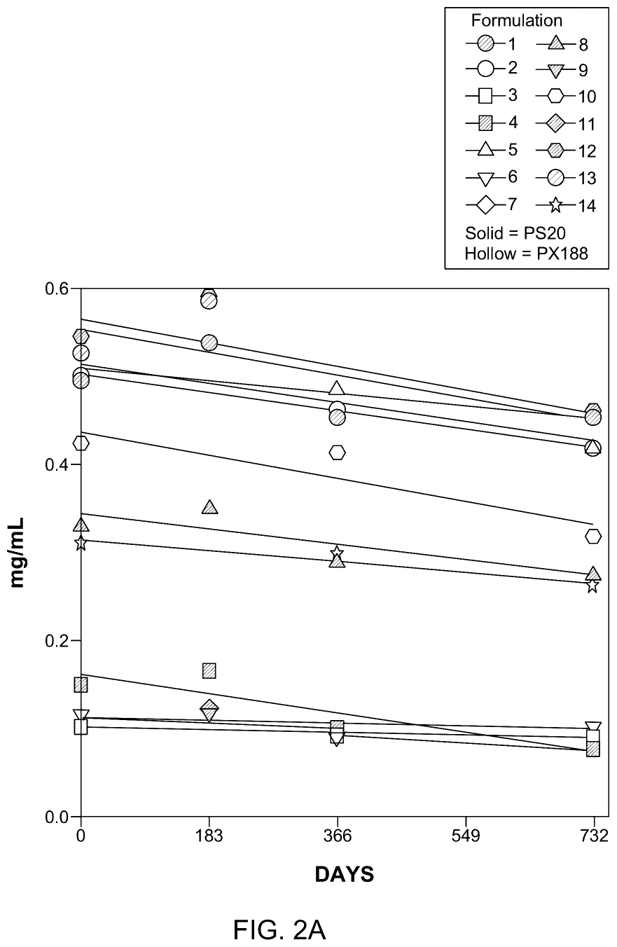

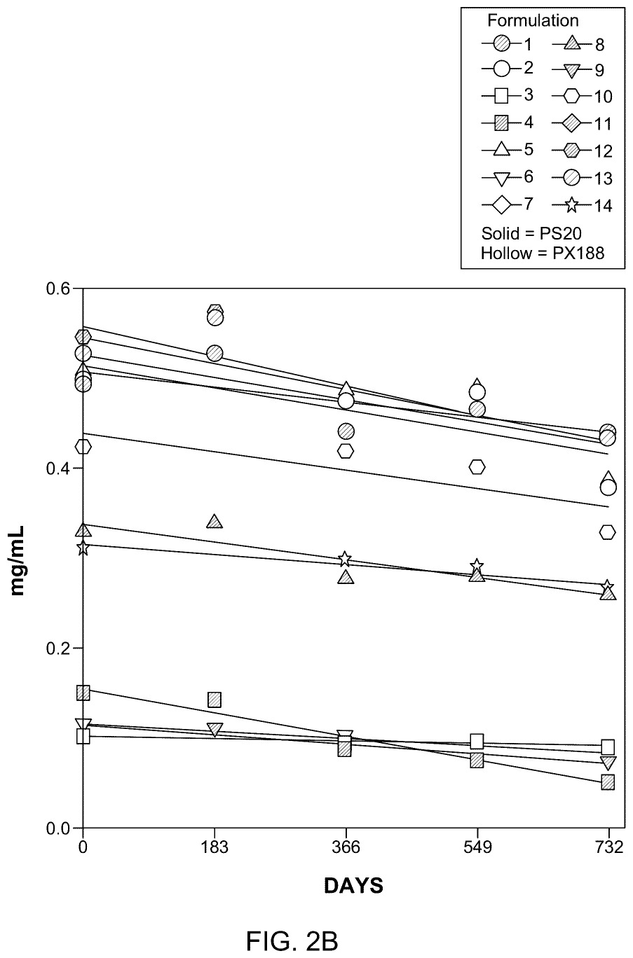

Changes in oxidation of the tiragolumab formulations containing PS20 at 25° C. and 40° C. were observed. Formulations with higher levels of oxidation after 1 month at 40° C. include a greater than 2% change in DLTMISR Fc methionine (“Fc DTLMISR (M_Ox)”) and greater than 1% change in HEAL Fc methionine (“Fc HEAL (M_Ox)”) (Table 6, infra). See, for example, Formulation (Form.) No. 1. Oxidation was exacerbated with high surfactant concentration, high histidine concentration, and low protein concentration. Further, formulations with the highest oxidation correlated with the highest amount of PS20 degradation. PS20 degradation is measurable by fatty acid metabolites using FAMS (FIG. 1). The addition of L-methionine was found to mitigate the risk of PS20-induced oxidation.

TABLE 6
Changes in oxidation compared to T0 after stress for 1 month at 40° C.

| | | | | | | | Increase in % oxidation | | |
|---|---|---|---|---|---|---|---|---|---|
| | | | | | | | after 1 month at 40° C. | | |
| | | | | | | Histidine | Fc | Fc | HC |
| Form. | | Protein | | Surfactant | Sucrose | Acetate | DTLMISR | HEAL | CDR2 |
| No. | pH | (mg/mL) | ST | (% w/v) | (mM) | (mM) | (M_Ox) | (M_Ox) | (W_OX) |
| 1 | 6.0 | 18 | PS20 | 0.05 | 100 | 30 | 46.7 | 18.8 | 1.8 |
| 2 | 6.0 | 66 | P188 | 0.05 | 300 | 30 | 1.2 | 0.4 | 0.2 |
| 3 | 6.0 | 18 | P188 | 0.01 | 300 | 30 | 1.5 | 0.4 | 0.2 |
| 4 | 5.0 | 66 | PS20 | 0.01 | 300 | 10 | 0.8 | 0.4 | 0.2 |
| 5 | 5.0 | 18 | P188 | 0.05 | 300 | 10 | 0.9 | 0.6 | 0.2 |
| 6 | 5.0 | 66 | P188 | 0.01 | 100 | 30 | 1.1 | 0.5 | 0.3 |
| 7 | 6.0 | 66 | P188 | 0.01 | 100 | 10 | 1.0 | 0.5 | 0.3 |
| 8 | 5.5 | 42 | PS20 | 0.03 | 200 | 20 | 13.5 | 4.9 | 1.0 |
| 9 | 6.0 | 18 | PS20 | 0.01 | 300 | 10 | 0.6 | 0.2 | 0.1 |
| 10 | 5.0 | 18 | P188 | 0.05 | 100 | 10 | 0.7 | 0.3 | 0.1 |
| 11 | 5.0 | 18 | PS20 | 0.01 | 100 | 30 | 10.2 | 3.7 | 1.0 |
| 12 | 5.0 | 66 | PS20 | 0.05 | 300 | 30 | 13.6 | 5.2 | 1.2 |
| 13 | 6.0 | 66 | PS20 | 0.05 | 100 | 10 | 11.3 | 3.8 | 0.8 |
| 14 | 5.5 | 42 | P188 | 0.03 | 200 | 20 | 0.9 | 0.3 | 0.2 |

For non-categorical assay outputs, the product of the effect size of the formulation parameter, determined from the multiple regression modeling, was multiplied by the relevant duration for a given storage condition to determine if the formulation parameter was practically meaningful over that duration. A correction factor was also included to adjust for differences between the proposed formulation parameter range (Table 7, infra) and the range studied in the stability study. If the value was greater than the assay variability for a given assay output, then the formulation parameter was deemed practically meaningful (e.g., when compared to other formulations, the formulation parameter at the low or high extreme will cause a difference in stability outside the assay variability after the relevant storage condition/time). The relevant durations used for this calculation were:
1) Drug Substance (DS): 5 years at −20° C., 2 months at 5° C., 1 week at 25° C., and 2 days at 40° C.; and
2) Drug Product (DP): 3 years at 5° C., 1 month at 25° C., and 3 days at 40° C.

TABLE 7
Tested formulation composition ranges for tiragolumab

| Formulation Component | DS | DP |
|---|---|---|
| pH | 5.2-5.8 | 5.2-5.8 |
| Protein Concentration (mg/mL) | 144-176 | 54-66 |
| Histidine Concentration (mM)^{a} | 5-25 | 12-24 |
| Methionine Concentration (mM)^{b} | 5-15 | 5-15 |
| Sucrose Concentration (mM)^{c} | 151-316 | 182-308 |
| PS20 Concentration (mg/mL) | 0.5-0.8 | 0.2-0.6 |

^{a}= Histidine is not measured; 14 mM histidine expected in DS due to Donnan effect; calculated DP range based on dilution buffer and DS acceptable composition ranges
^{b}= Methionine is not measured; calculated ranges based on conditioning and dilution buffers used in process
^{c}= Sucrose range calculated based on osmolality specifications

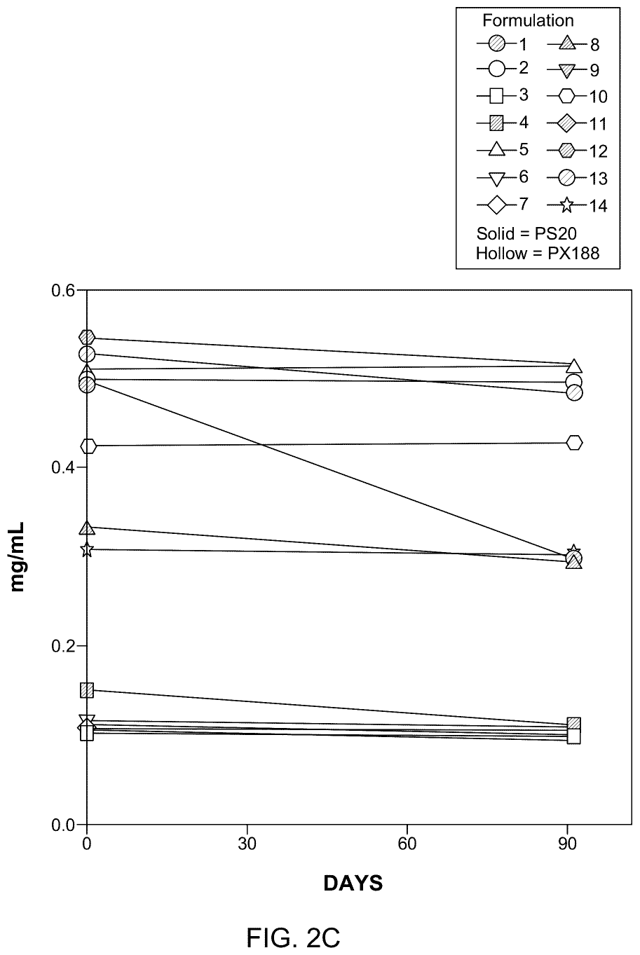

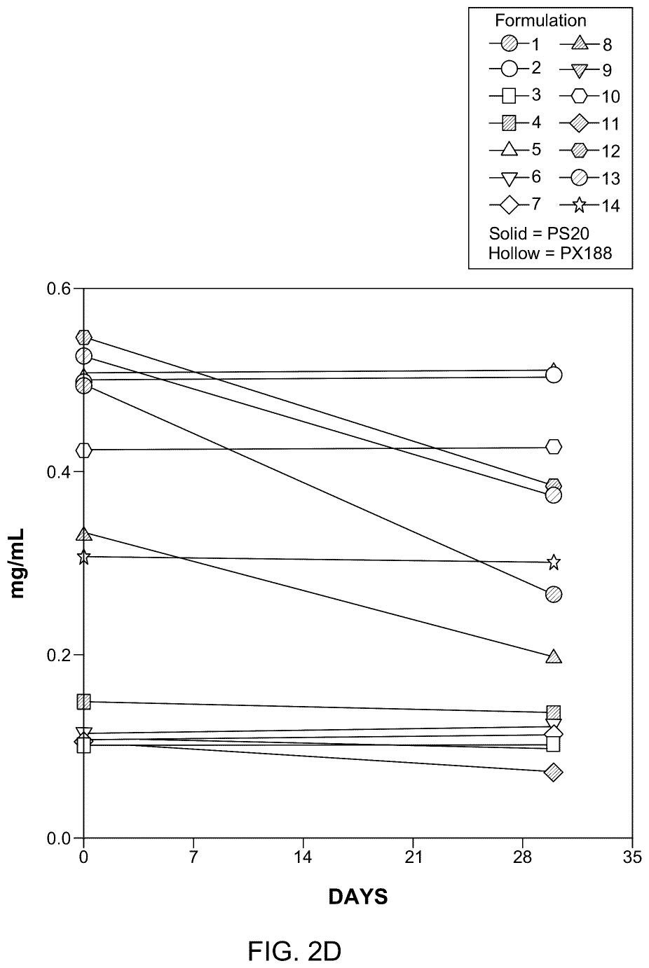

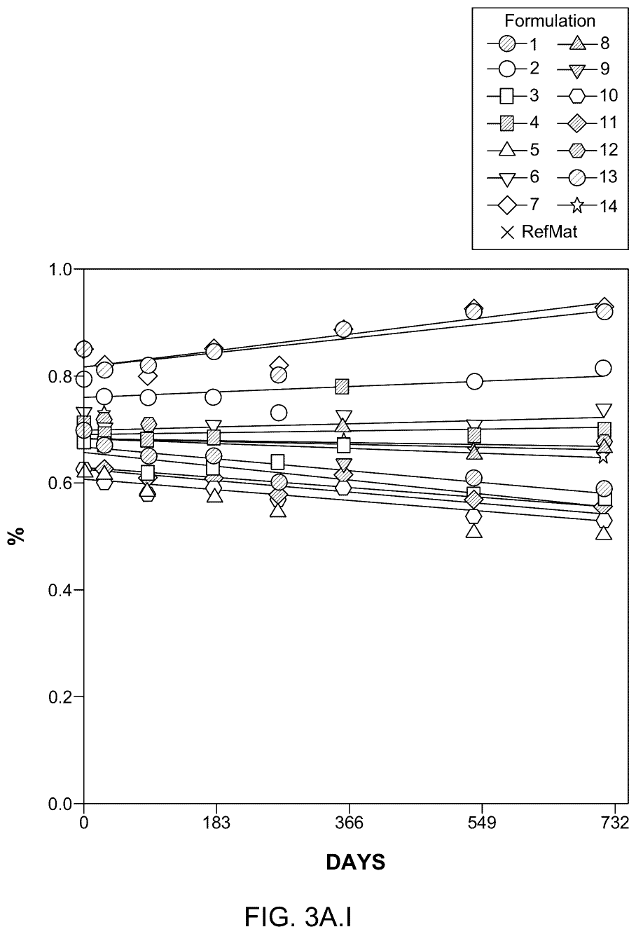

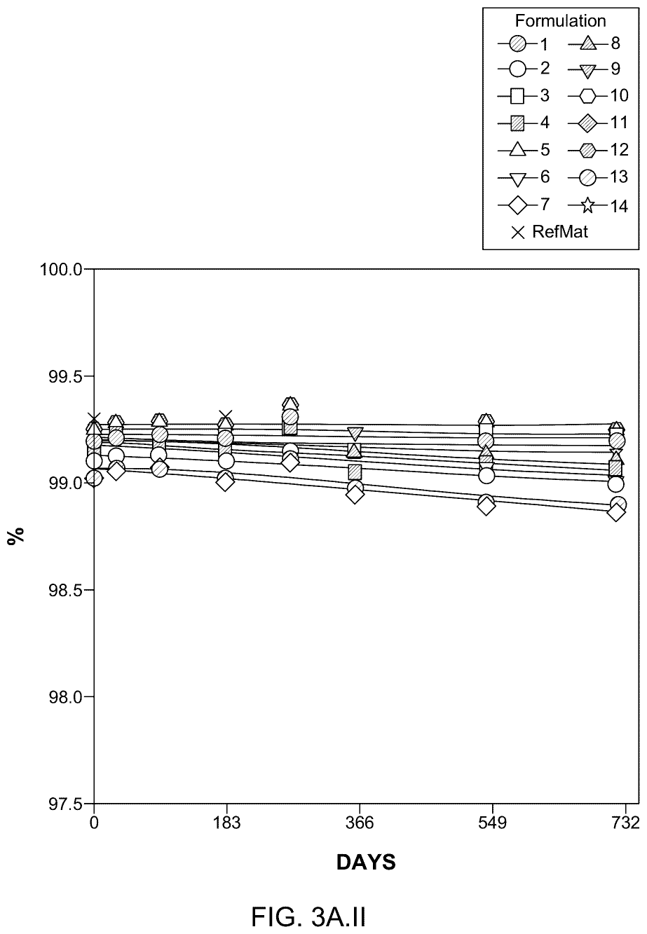

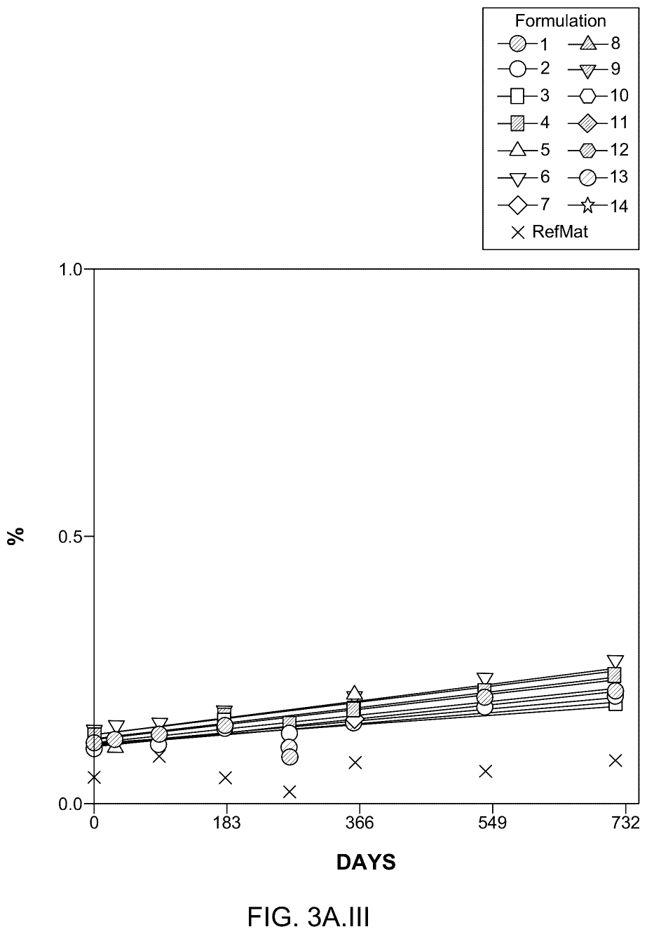

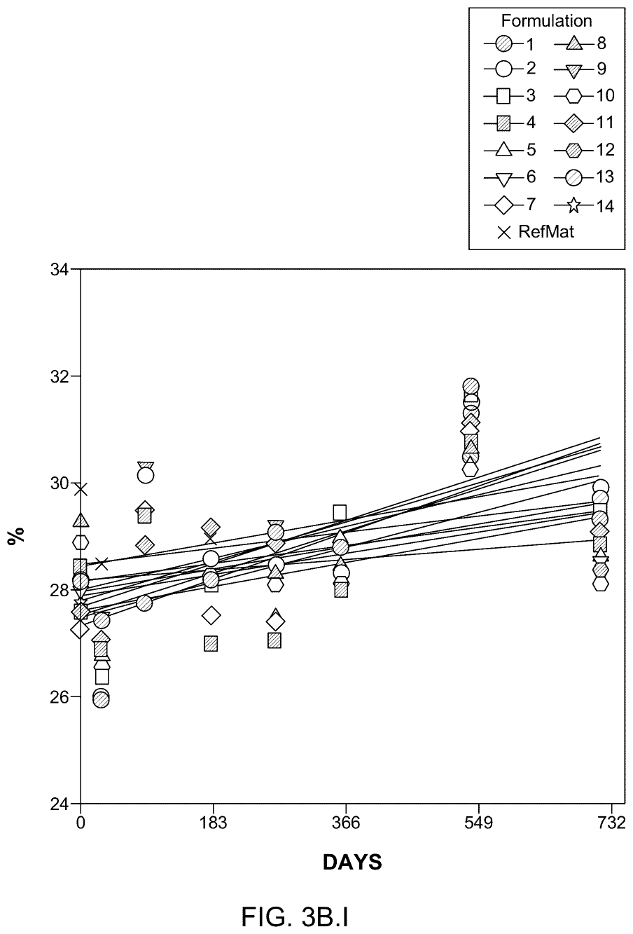

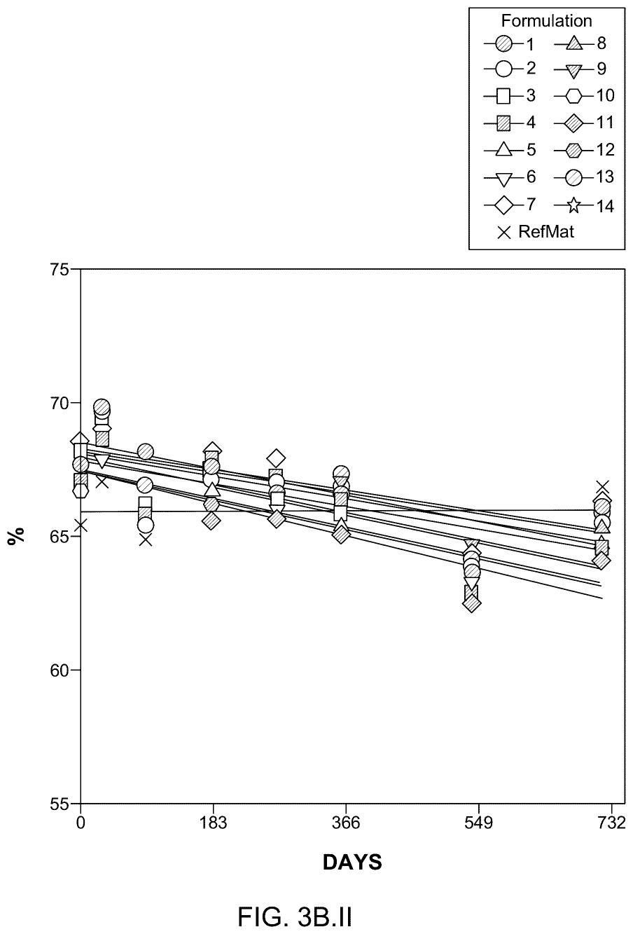

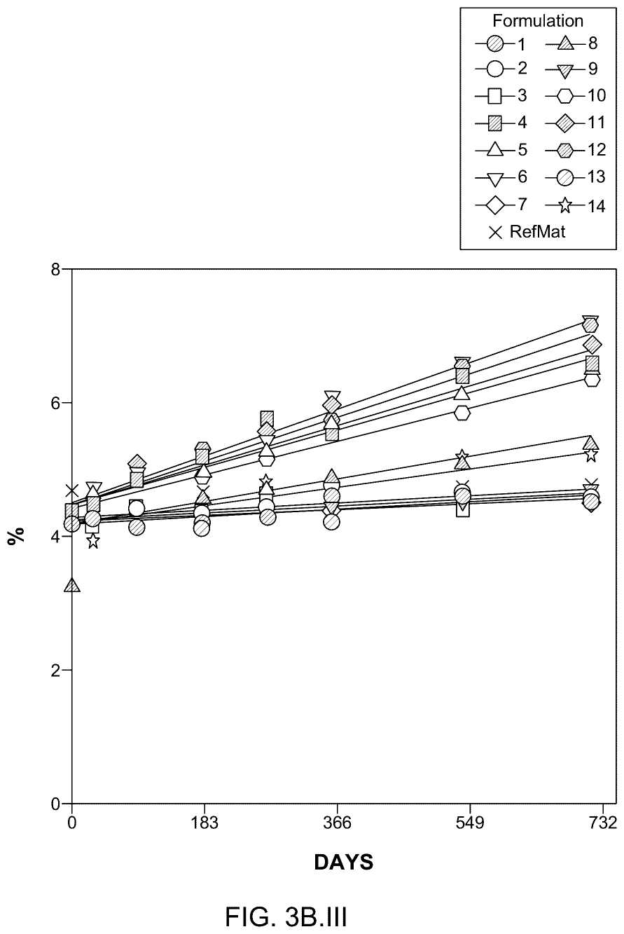

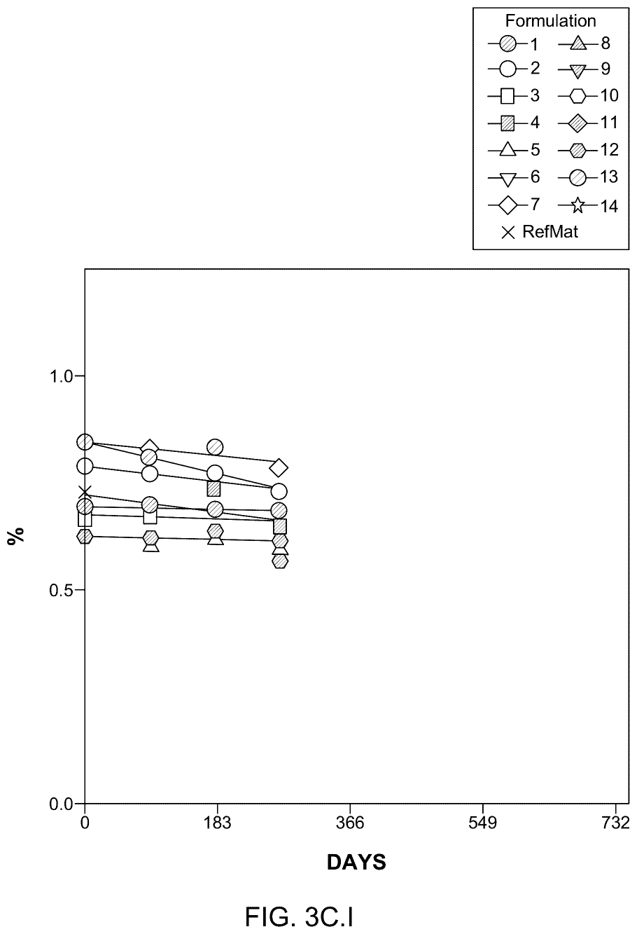

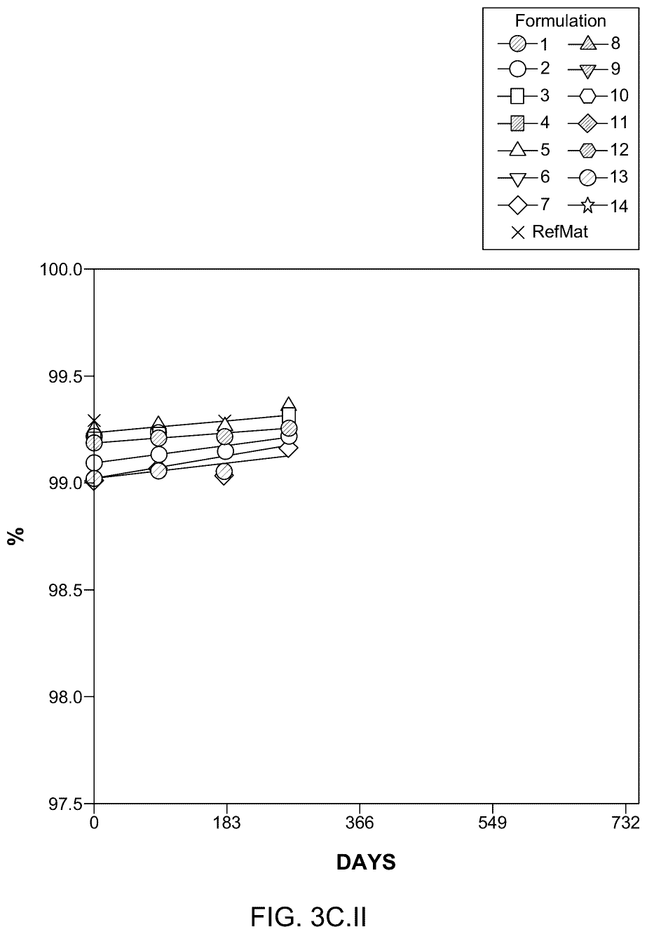

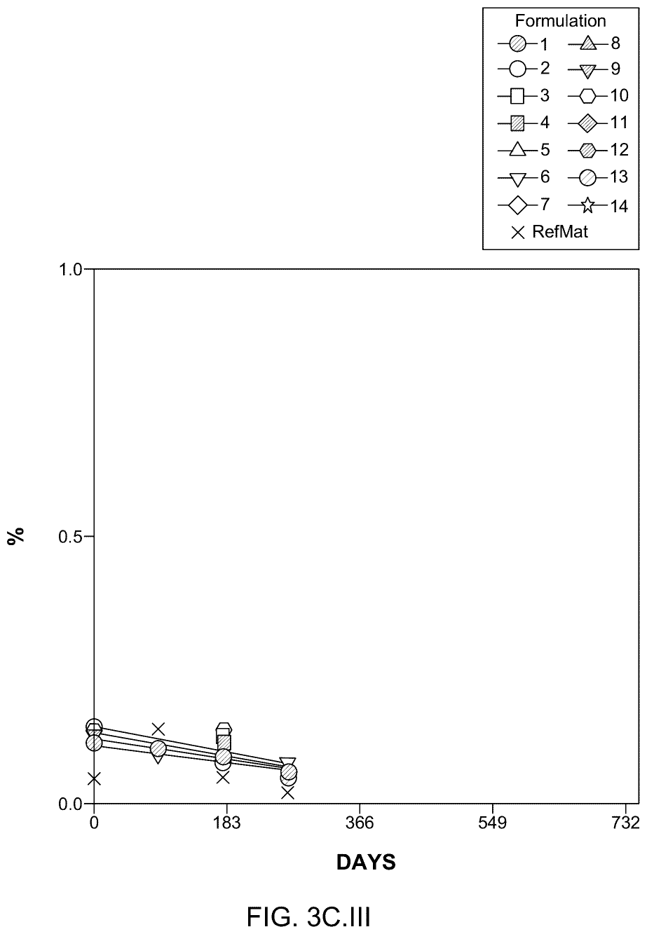

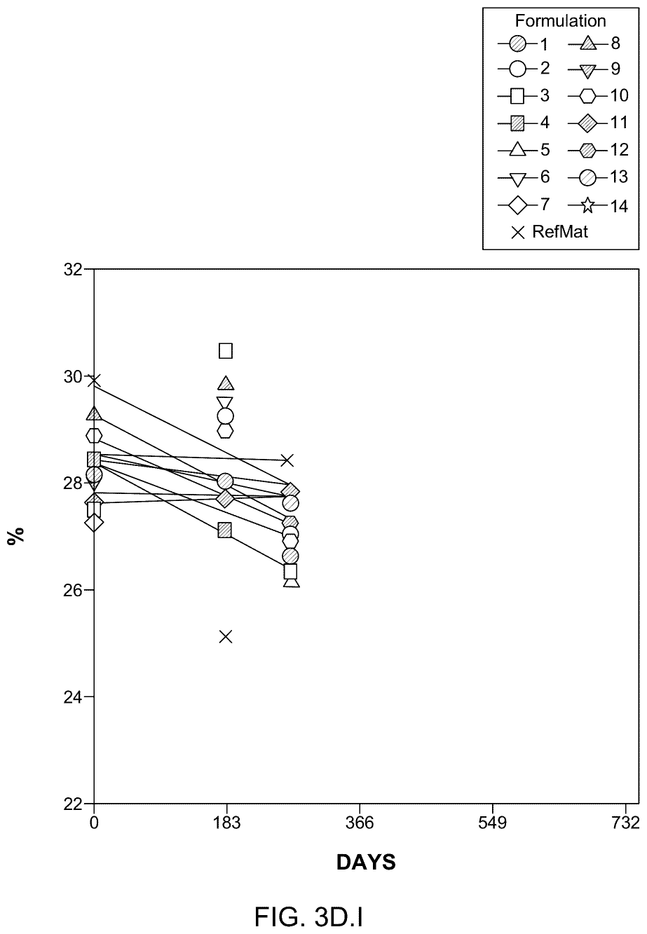

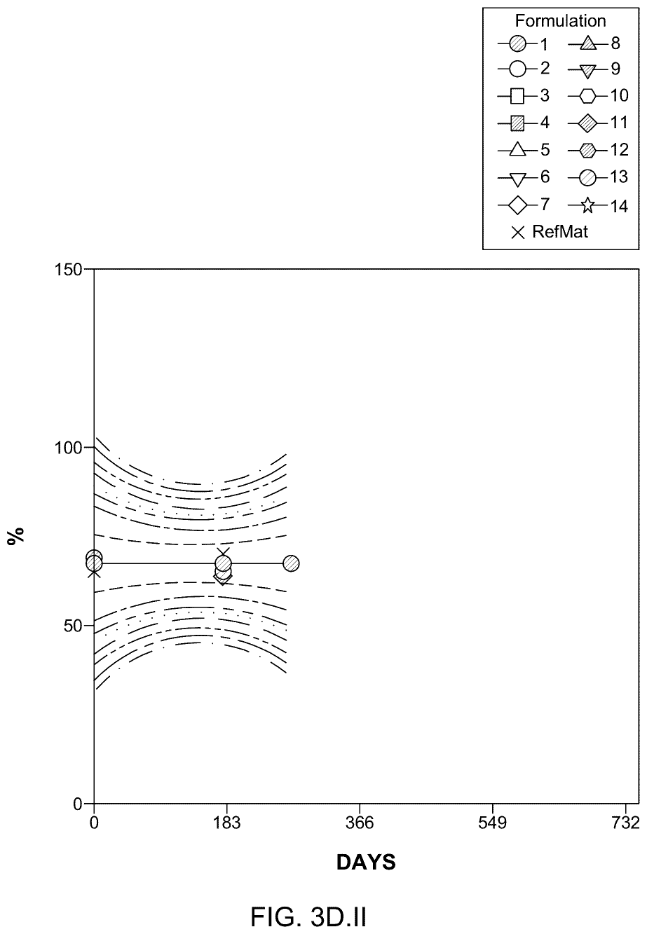

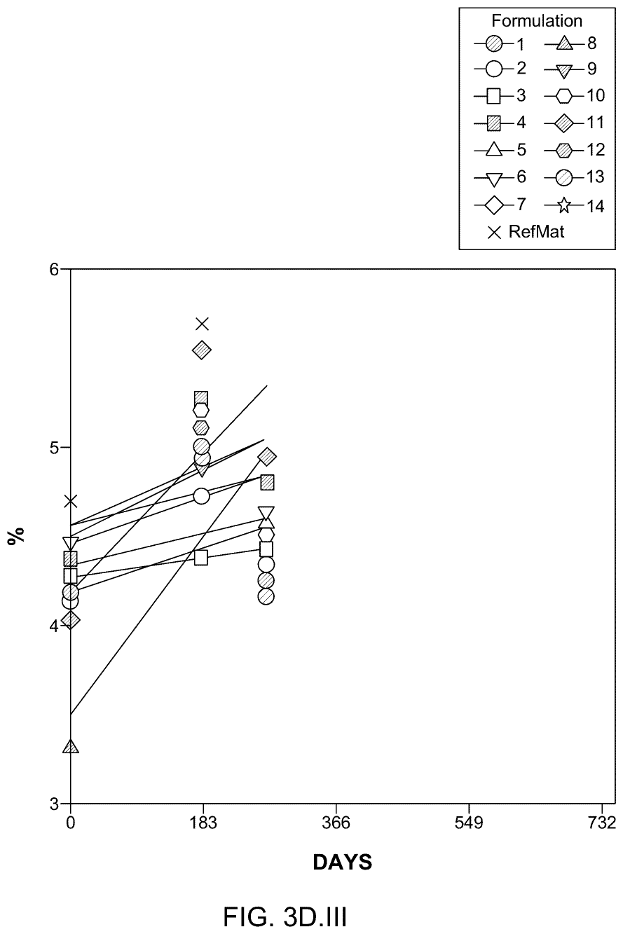

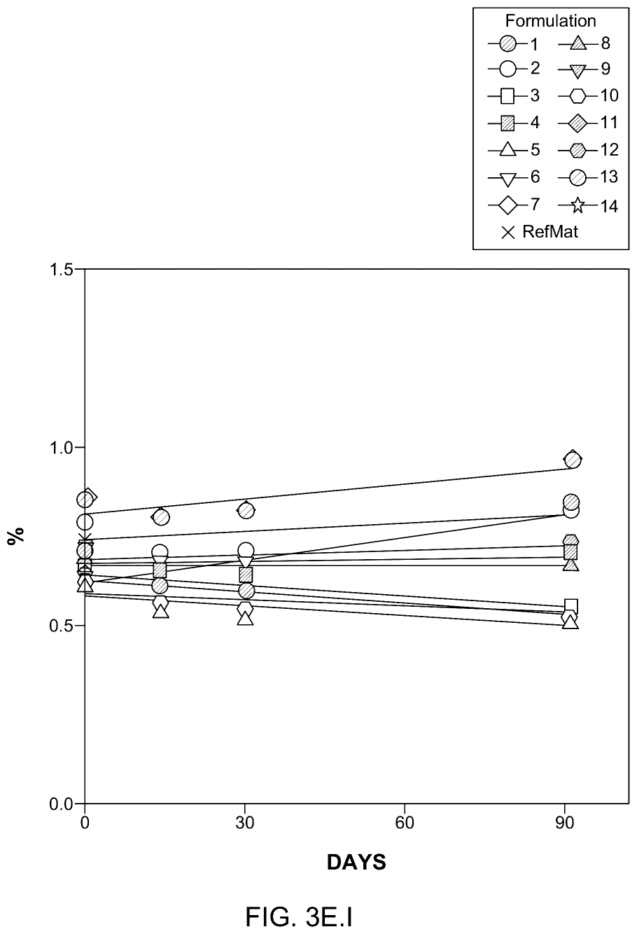

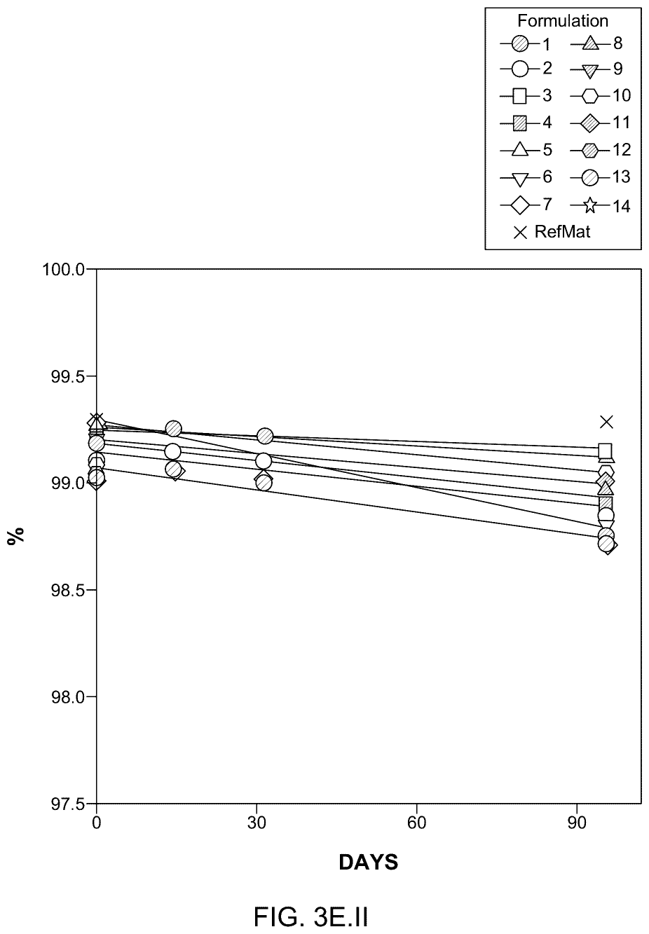

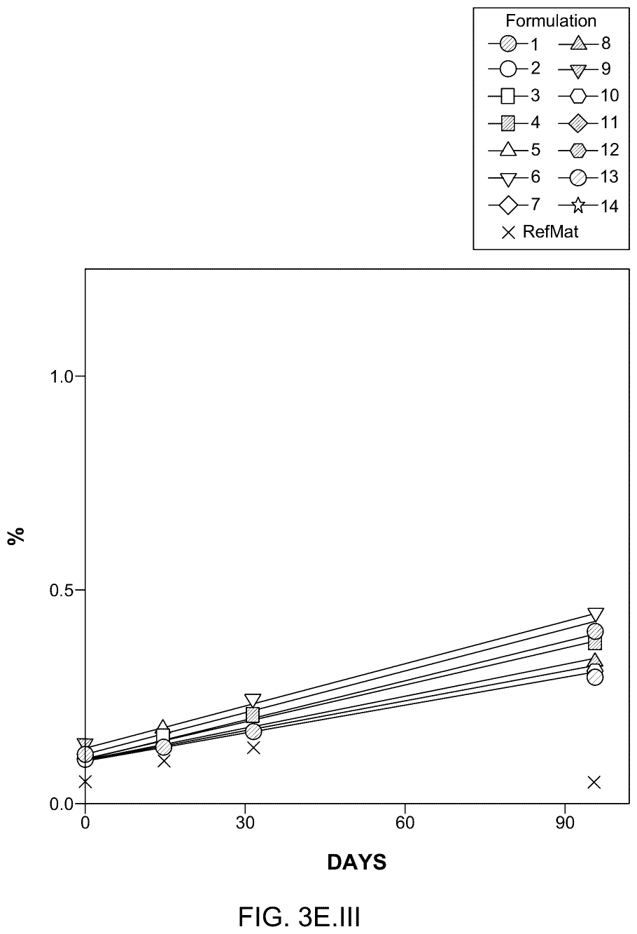

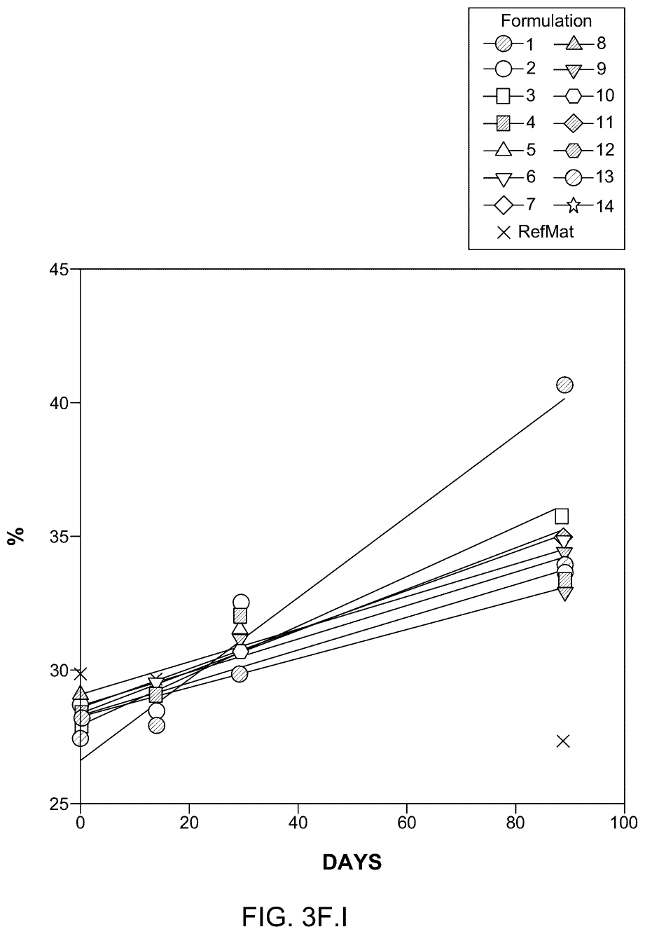

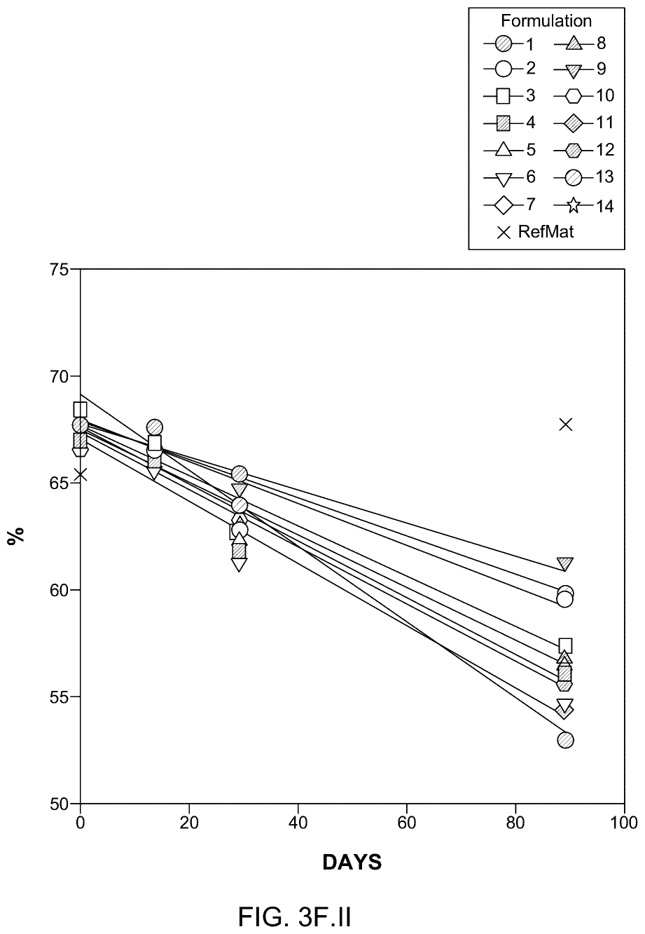

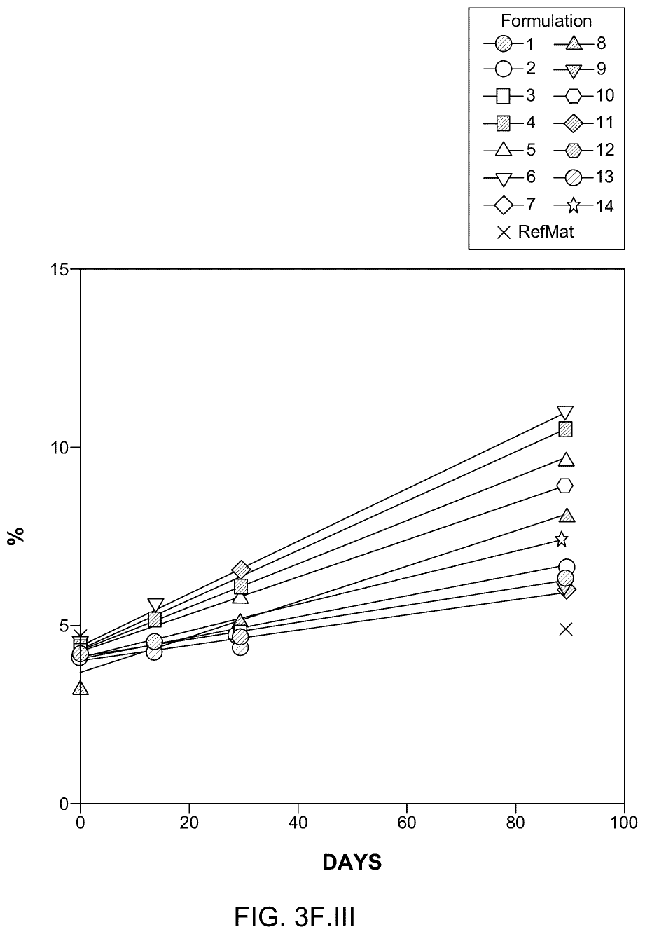

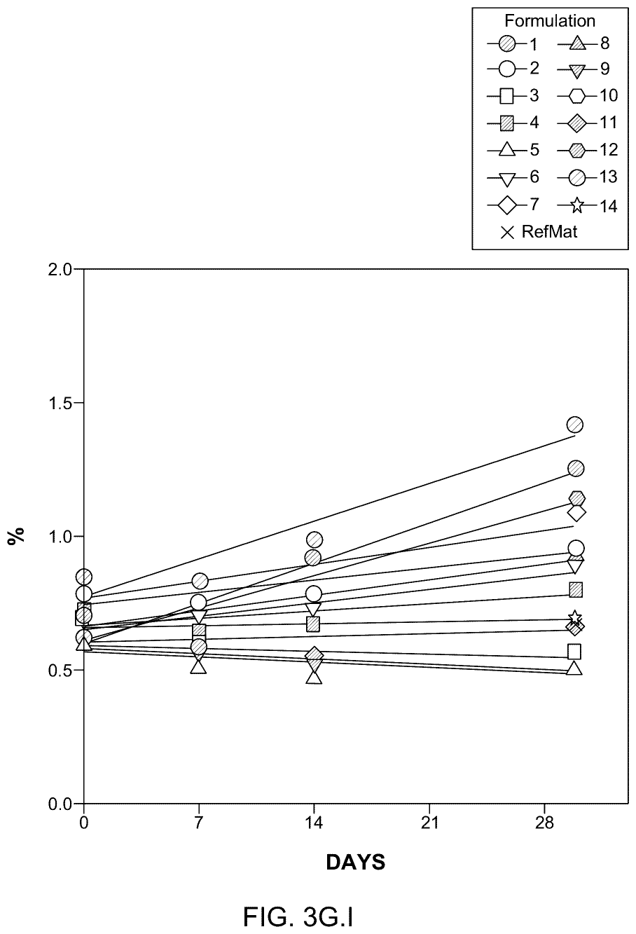

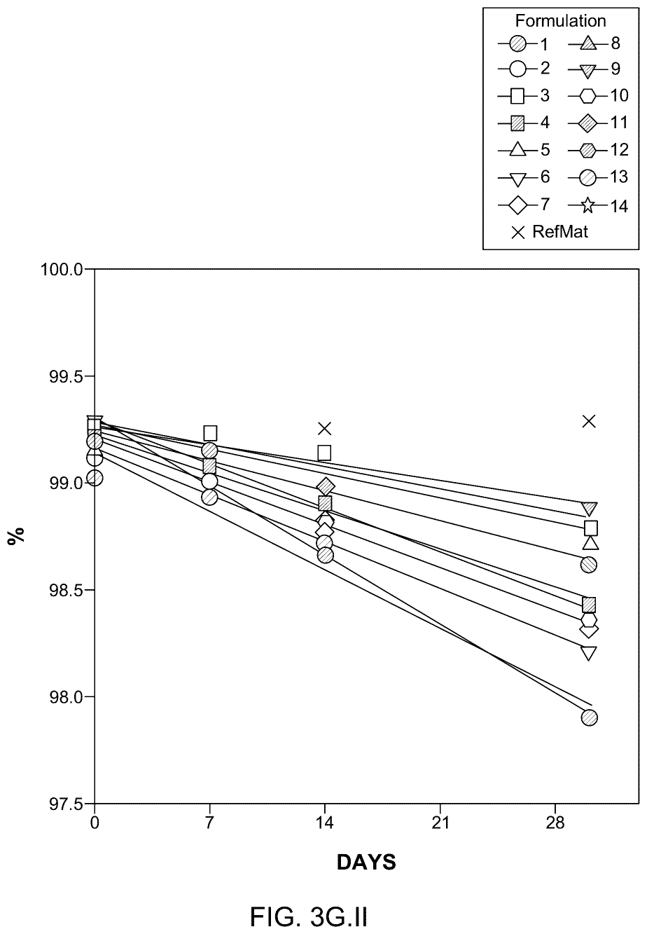

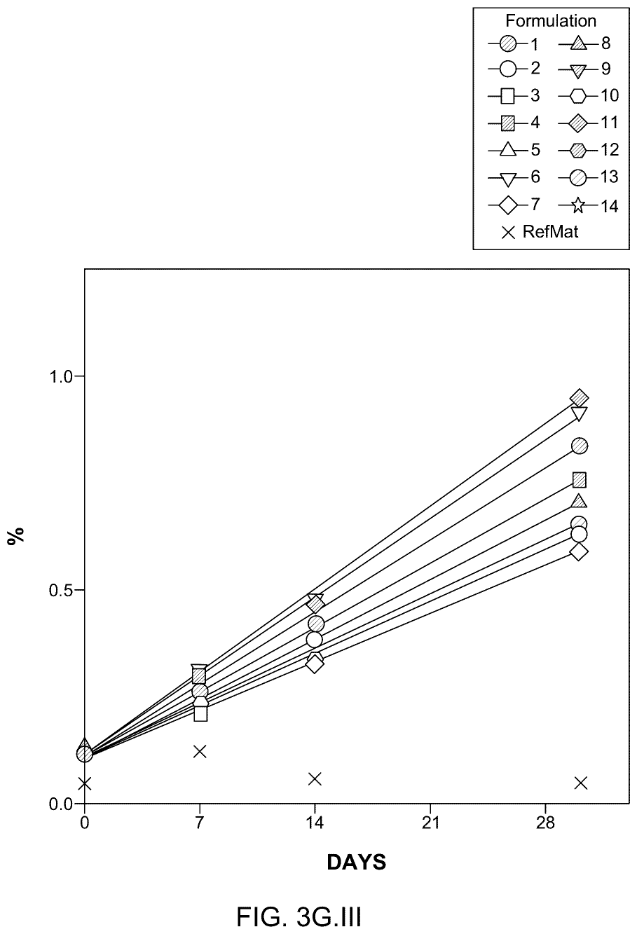

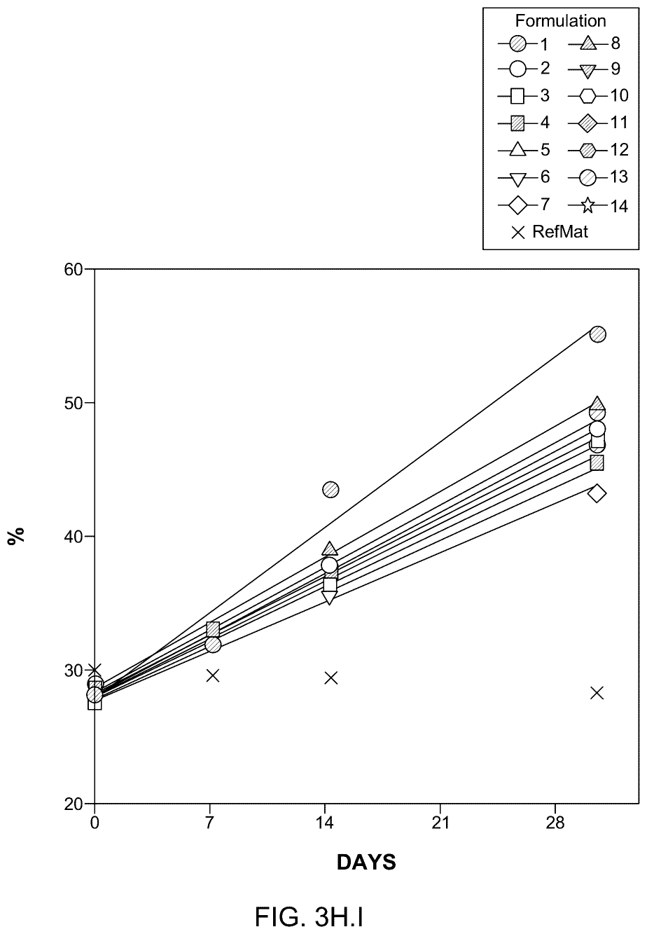

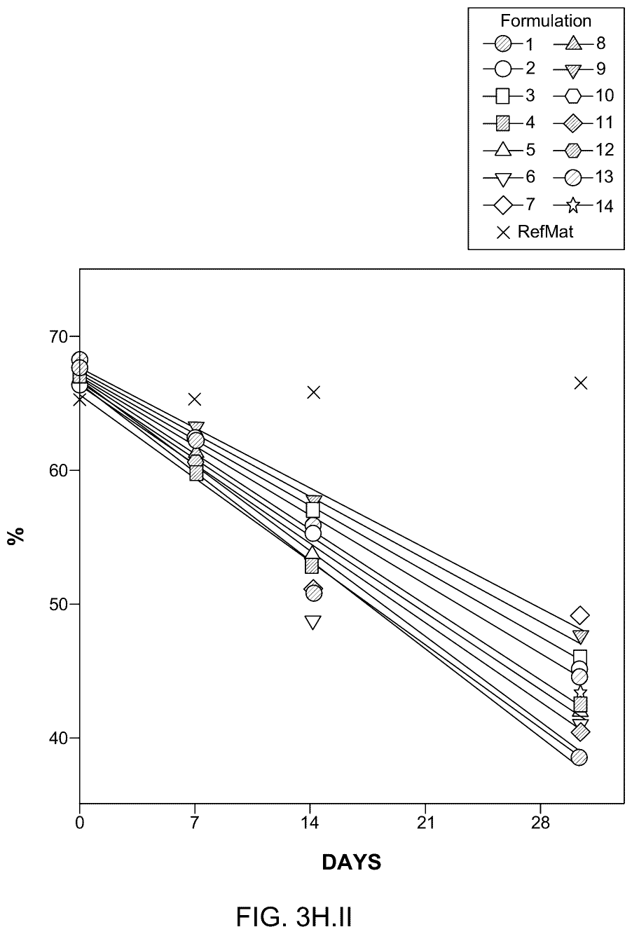

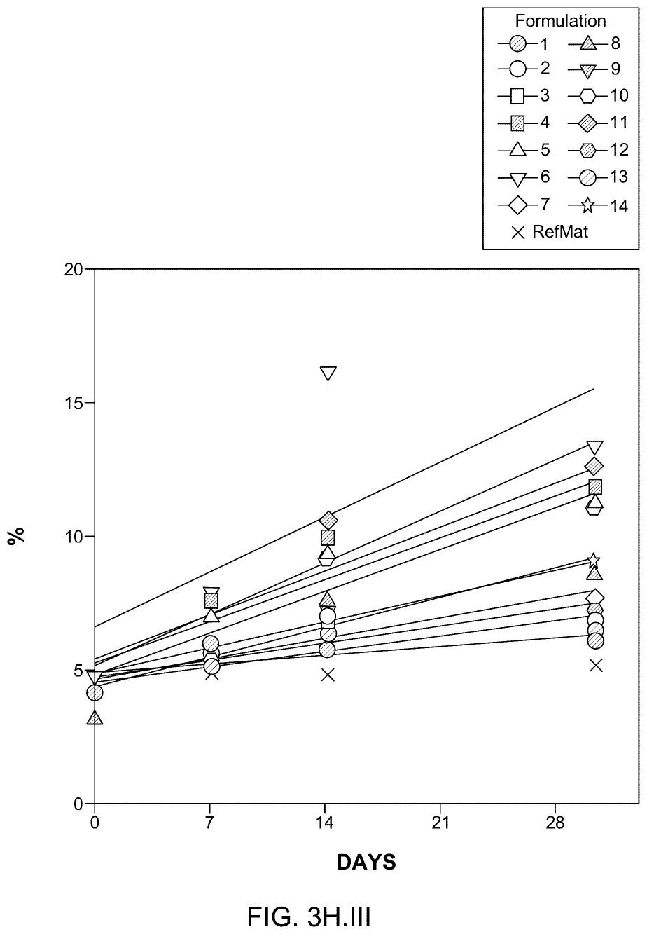

After ELSD was performed, the concentration of tiragolumab was measured over time for each of the 14 formulations to determine the stability of each formulation at the varying temperatures (e.g., −20° C., 5° C., 25° C. and 40° C.) (FIGS. 2A-2D). SEC was performed to test for potential physical degradation of tiragolumab, in which aggregates and low molecular weight species (LMWS) were measured at varying temperatures (e.g., −20° C., 5° C., 25° C. and 40° C.) (FIGS. 3A.I, 3A.II, 3A.III, 3C.I, 3C.II, 3C.III, 3E.I, 3E.II, 3E.III, 3G.I, 3G.II and 3G.III). Imaged capillary isoelectric focusing (ICIEF) was performed to evaluate the formation of charged isomers of tiragolumab when exposed to an acidic or basic condition. The rate of degradation remained similar between formulations at varying temperatures (e.g., −20° C., 5° C., 25° C. and 40° C.) (FIGS. 3B.I, 3B.II, 3B.III, 3D.I, 3D.II, 3D.III, 3F.I, 3F.II, 3F.III, 3H.I, 3H.II, and 3H.III).

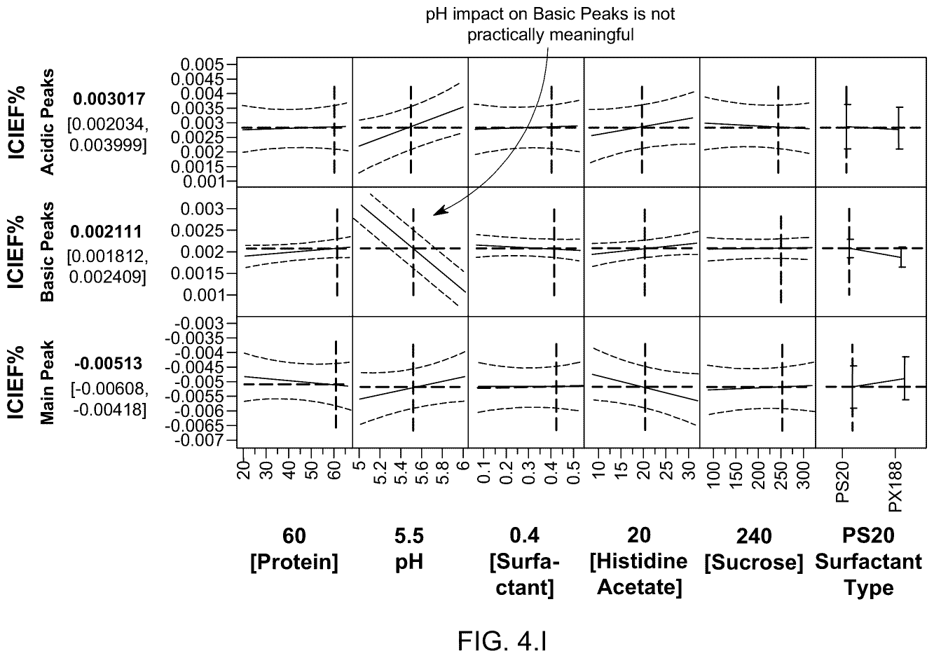

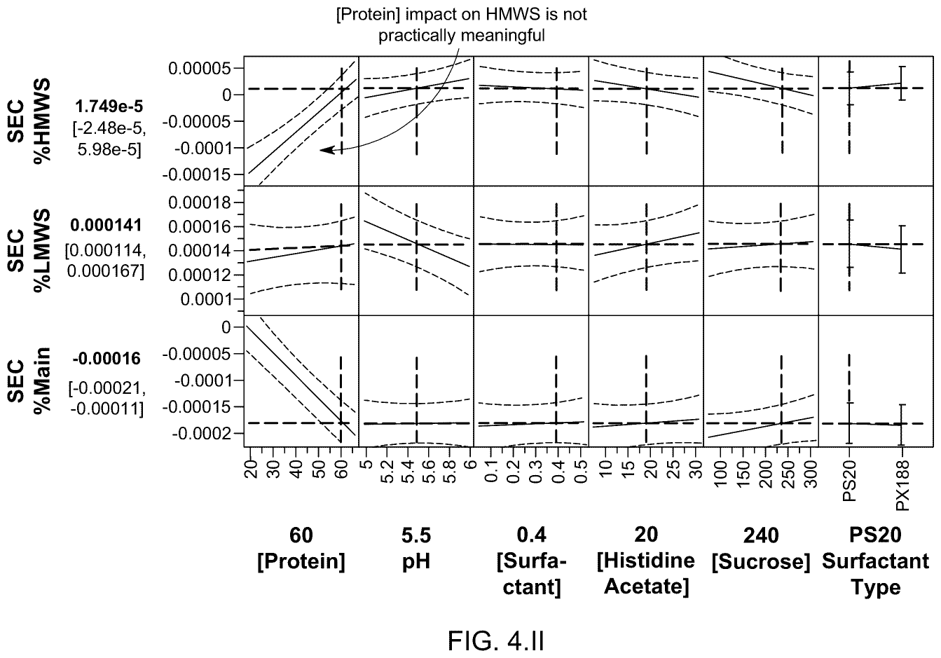

The formulation parameters that caused practically meaningful changes for the assay outputs are given in in Table 8, infra. No formulation parameters were found to cause practically meaningful changes for the DS 2 months at 5° C. (FIGS. 4.I and 4.II) and 1 week at 25° C. conditions. Further, no formulation parameters were found to cause practically meaningful changes for the following attributes: basic region (IED), main peak (IEC), main peak (NR CE-SDS), sum of HMW forms (NR CE-SDS), sum of LMW forms (NR CE-SDS), sum of LC+HC (reduced CE-SDS), IRS (reduced CE-SDS), NGHC (reduced CE-SDS), sum of HMW forms (SEC), main peak (SEC), W59 HC oxidation (peptide mapping), pH, protein concentration, and potency.

TABLE 8
Formulation parameters that caused practically meaningful
changes after the given storage temperatures and times.

| | DS | | DP | | |
|---|---|---|---|---|---|
| | −20° C. | 40° C. | 5° C. | 25° C. | 40° C. |
| Attribute | 5 years | 2 days | 3 years | 1 month | 3 days |
| Acidic region | pH, protein, | | pH, | | |
| (IEC) | SC, ST, | | histidine, | | |
| | sucrose | | ST | | |
| PS20 | Protein, SC | | SC | | |
| Sum of LMW | | | pH | | |
| forms (SEC) | | | | | |
| M261 oxidation | | | | pH, SC, ST, | SC, ST |
| (peptide mapping) | | | | sucrose, | |
| | | | | histidine | |
| M437 oxidation | | | | pH, SC, ST, | |
| (peptide mapping) | | | | sucrose, | |
| | | | | histidine | |
| ≥2 μm particles | pH, SC, ST, | pH, SC, ST, | SC, ST, | pH, SC, | pH, SC, ST, |
| (HIAC) | protein, | protein, | histidine | histidine | sucrose, |
| | sucrose, | sucrose, | | | histidine |
| | histidine | histidine | | | |
| ≥5 μm particles | pH, SC, ST, | pH, SC, ST, | SC, ST, | | pH, SC, ST, |
| (HIAC) | protein, | protein, | histidine, | | protein, |
| | sucrose, | sucrose, | sucrose | | sucrose, |
| | histidine | histidine | | | histidine |
| ≥10 μm particles | SC, sucrose | pH, protein, | ST, sucrose | | pH, SC, ST, |
| (HIAC) | | SC, ST, | | | histidine, |
| | | sucrose, | | | sucrose |
| | | histidine | | | |
| ≥25 μm particles | SC, sucrose | | SC, sucrose | | |
| (HIAC) | | | | | |
| ≥50 μm particles | SC | | | | |
| (HIAC) | | | | | |

Formulation parameters that demonstrated both statistical significance and practically meaningful change after relevant storage times and temperatures were deemed both practically meaningful and statistically significant. Table 9, infra, summarizes the practically meaningful and statistically significant parameters. Surfactant type (ST) and formulation pH were identified as critical formulation parameters. The HIAC (25 μM particles) data at 5° C. did not demonstrate any trends, and therefore, was not further examined.

TABLE 9
Formulation parameters demonstrating both statistical
significance and practically meaningful change

| Temperature/Duration | Attribute | Formulation Parameter(s) |
|---|---|---|
| −20° C. for 5 years | IEC | Protein concentration |
| | (% acidic region) | |
| −20° C. for 5 years | HIAC | Sucrose concentration, ST |
| | (2 μM particles) | |
| −20° C. for 5 years | HIAC | Sucrose concentration, ST |
| | (5 μM particles) | |
| 5° C. for 3 years | IEC | Histidine concentration |
| | (% acidic region) | |
| 5° C. for 3 years | SEC | pH |
| | (% sum of LMW | |
| | forms) | |
| 5° C. for 3 years | HIAC | ST |
| | (5 μM particles) | |
| 5° C. for 3 years | HIAC | ST |
| | (10 μM particles) | |
| 40° C. for 2 days | HIAC | ST |
| | (2 μM particles) | |
| 40° C. for 3 days | Peptide mapping | ST |
| | (% DTLMISR) | |
| 40° C. for 3 days | HIAC | ST |
| | (2 μM particles) | |

The stability study demonstrated that the formulation was robust and stable across the formulation space tested and support a DS and/or DP formulation for tiragolumab of 60 mg/mL in 20 mM histidine acetate, 240 mM sucrose, 10 mM methionine, and 0.04% PS20 at pH 5.5.

### Example 2: Risk Ranking and Filtering Procedure for Tiragolumab Drug Substance (DS) and Drug Product (DP) Formulations

Risk ranking evaluated the effects of various formulation process inputs on product quality and stability of the anti-TIGIT monoclonal antibody tiragolumab (also known as MTIG719A) drug substance (DS) and drug product (DP) formulations. This risk ranking and filtering (RRF) assessment confirmed stability robustness of the DS and DP formulations within the specified formulation composition ranges. Specifically, the risk ranking analysis entails assessing each tiragolumab formulation component (process inputs) for both potential main effects and possible interactions with other formulation components against defined product quality responses such as changes in physical and chemical properties (process outputs).

A first DS formulation of tiragolumab contained 160 mg/mL tiragolumab formulated with 20 mM L-histidine acetate (HisOAc), 240 mM sucrose, 10 mM L-methionine (Met), and 0.6 mg/mL polysorbate 20 (PS20), pH 5.5. A first and second DP formulation of tiragolumab contained 60 mg/mL tiragolumab formulated with 20 mM L-histidine acetate (HisOAc), 240 mM sucrose, 10 mM L-methionine (Met), and 0.4 mg/mL PS20 pH 5.5. A second DS formulation contained 60 mg/mL tiragolumab formulated with 20 mM L-histidine acetate (HisOAc), 240 mM sucrose, 10 mM L-methionine (Met), and 0.4 mg/mL PS20, pH 5.5. The first and second DS formulations were stored in 120 L and 300 L stainless steel tanks. The first DP formulation was stored in a 10 mL (600 mg) nominal fill in a 15-cc vial. The second DP formulation was stored in one of two vial configurations: a 7 mL (420 mg) and a 10 mL (600 mg) nominal fill both in a 15-cc vial.

The formulation composition and corresponding ranges for the various DS and DP formulations are described in Table 7, supra. The formulation composition included each formulation component, also referred to as formulation parameter. The ranges for pH, protein concentration, and PS20 content were determined based on their respective anticipated release specifications. The ranges for buffer (histidine and acetic acid) content, methionine content, and sucrose content were at least as wide as or greater than the expected ranges during routine manufacturing. The buffer content and sucrose content were indirectly measured via the osmolality assay. The pH also helped ensure that correct amounts of histidine and acetic acid were added.

The process outputs evaluated in RRF were assay outputs intended to monitor the potential critical quality attributes (PCQAs) and obligatory critical quality attributes (CQAs). The pCQAs relevant for formulation robustness study are presented in Table 10, infra. Several pCQAs and obligatory CQAs were not included in the risk ranking and filtering assessment for the following reasons:
1) Process related impurities pCQAs, such as host cell proteins, and obligatory CQAs related to adventitious agents, such as bacterial endotoxin, were controlled during processing and not expected to change on stability.
2) Sucrose content is an obligatory CQA that was indirectly measured by osmolality. Sucrose levels were not expected to change upon stability.
3) Methionine content was controlled through a robust manufacturing process. Methionine levels were not expected to change upon stability.
4) Histidine acetate content is an obligatory CQA that was indirectly measured by pH. The histidine acetate acts as a buffer species that controls the pH. Histidine acetate levels were not expected to change upon stability.
5) Structural variants like sequence variants are pCQAs. These variants were controlled during DS production and were not expected to change during DP or processing or in stability testing.
6) Glycosylation is a PCQA that was controlled during the cell culture process and is not expected to change during DS/DP processing or on stability.
7) Extractable volume, sterility, container closure integrity, and primary packaging extractables/leachables are not directly related to the formulation and were assessed in separate studies.

TABLE 10
Tiragolumab pCQAs and assay outputs for formulation robustness study

| pCQA Category | Quality Attribute | Assay Name | Assay Output |
|---|---|---|---|
| Size-related | High Molecular Weight | Multi-Product Size | Sum of HMW Forms |
| variants | Forms (HMWF) | Exclusion Ultra High | by SE-UHPLC |
| | | Performance Liquid | |
| | | Chromatography | |
| | | Method (UHPLC) | |
| | Low Molecular Weight | CE-SDS Using | Sum of LMW Forms |
| | Forms (LMWF) | Fluorogenic FQ Dye | by NR-CE-SDS |
| | | for Process | (% CPA) |
| | | Validation | |
| Charge-related | Glycation in non-CDR | Ion Exchange | Acidic Region by |
| variants | Glycation in CDR | Chromatography of | IE-HPLC (%) |
| | Deamidation in non-CDR | Clinical Products | |
| | Deamidation in CDR | | |
| | Glucuronidation in CDR | | |
| | Glucuronidation in non-CDR | | |
| | N-terminal pyroglutamic acid | | |
| | HC C-terminal lysine | Low pH Peptide Map | HG-K456 C- |
| | | by RP-UHPLC- | Terminal Lysine (%) |
| | | HRMS/MS for | |
| | | Characterization and | |
| | | Multi-Attribute | |
| | | Monitoring | |
| | Aspartic acid | Ion Exchange | Basic Region by IE- |
| | isomerization/succinimide | Chromatography of | HPLC (%) |
| | hotpots in CDR | Clinical Products | |
| | Aspartic acid | | |
| | isomerization/succinimide | | |
| | hotpots in non-CDR | | |
| | C-terminal proline | | |
| | amidation | | |
| | N-terminal leader sequence | | |
| Oxidation- | Oxidation in non-CDR | Low pH Peptide Map | HC-W59 Oxidation |
| related variants | | by RP-UHPLC- | (%) |
| | | HRMS/MS for | HC-W56 Oxidation |
| | | Characterization and | (%) |
| | Oxidation in CDR | Multi-Attribute | HC-M261 Oxidation |
| | | Monitoring | (%) |
| | | | HC-M437 Oxidation |
| | | | (%) |
| Drug | PS20 | Global Method for | PS20 (mg/mL) |
| Substance and | | the Determination of | |
| Drug Product | | Polysorbate | |
| Composition | | Concentration by an | |
| and Strength | | HPLC-ELSD Assay | |
| | Protein Concentration | Protein Concentration | Protein |
| | | by UV (Slope | Concentration |
| | | Spectroscopy) | (mg/mL) |
| | Osmolality | Determination of | Osmolality |
| | | Osmolality by | (mOsm/kg) |
| | | Freezing Point | |
| | | Depression Method | |
| | pH | pH Determination | pH |
| | Appearance | Clarity, Opalescence, | Clarity/Opalescence |
| | (Opalescence, Clarity) | and Coloration of | (≤Ref X) |
| | | Liquids and | |
| | | Appearance of | |
| | | Liquid-Lyophilized | |
| | | Vials | |
| | Appearance (Color) | Visual Color | Color |
| | | Determination for | (≤BX) |
| | | Liquids | |
| Potency | Potency | tiragolumab Potency | Relative Potency (%) |
| | | Assay: Release of | |
| | | Jurkat-TIGIT-Luc T | |
| | | Cell Activation by | |
| | | tiragolumab | |
| Additional | Subvisible particles | Subvisible Particles | Particles/mL |
| Drug Product | | through the Small- | |
| Specific | | Volume Method | |
| Assays | Visible particles | Visible Particle | Number of particles |
| | | Analysis | |
| Purity | Main Peak by SE-UHPLC | Multi-Product Size | Main Peak by SE- |
| | | Exclusion Ultra High | UHPLC (%) |
| | | Performance Liquid | |
| | | Chromatography | |
| | | (UHPLC) Method | |
| | Main Peak by IE-HPLC | Ion Exchange (IE) | Main Peak by IE- |
| | | Chromatography of | HPLC (%) |
| | | Clinical Products | |
| | Main Peak by NR-CE-SDS | CE-SDS Using | Main Peak by NR- |
| | | Fluorogenic FQ Dye | CE-SDS (CPA %) |
| | | for Process | |
| | | Validation | |
| | Sum of LC and HC by R- | CE-SDS Using | Sum of LC and HC |
| | CE-SDS | Fluorogenic FQ Dye | by R-CE-SDS (CPA |
| | | for Process | %) |
| | | Validation | |

The intent of this RRF assessment was to confirm stability robustness of the DS and DP tiragolumab formulations within the specified formulation composition ranges. Process outputs were evaluated with respect to their rate of change on stability. If no change occurred on stability at the target formulation composition, the probability of change occurring within the formulation composition ranges is low. A formulation parameter (also known as process input) was considered to have a practically meaningful effect on a process output if the product of the effect size of the input, as determined from linear regression modeling, multiplied by the relevant duration for a given storage condition, is greater than assay variability (e.g., 3 times assay precision) for a given process output. Additionally, some attributes (e.g., protein concentration, pH and PS20 content) can be both a process input and process output. The relevant durations used for this assessment were based on anticipated shelf-life, ambient processing time, potential temperature excursion times are described for DS as 1) 5 years at recommended storage conditions (−20° C.), 2) 2 months at 5° C. (relevant manufacturing hold time), 3) 1 week at 25° C. (relevant manufacturing hold time and temperature excursion), and 4) 2 days at 40° C. (temperature excursion), and for DP as 1) 3 years at recommended storage conditions (5° C.), 2) 1 month at 25° C. (relevant manufacturing hold time), and 3) 3 days at 40° C. (temperature excursion).

The effect of storage container on DS stability was evaluated in in DS Long-Term Representative and GMP Stability Studies, see Table 11, infra. In stainless steel mini-tanks, the DS formulations were shown to be stable across multiple stability studies on both technical batches and GMP batches (see Table 11, infra), indicating low risk of variability in the primary storage container. Additional studies evaluating the effect of Hastelloy cryovessels were also performed.

TABLE 11
Drug Substance (DS) Long-Term Representative and GMP Stability Studies

| | | First DS | Second DS | Third DS |
|---|---|---|---|---|
| | | Formulation | Formulation | Formulation |
| Formulation | Protein concentration | 50 | 60 | 160 |
| Parameter | (mg/mL) | | | |
| | Nominal histidine acetate | 20 | 20 | 20 |
| | content ^{a} (mM) | | | |
| | Sucrose content (mM) | 240 | 240 | 240 |
| | Methionine content (mM) | 0 | 10 | 10 |
| | PS20 content (mg/mL) | 0.2 | 0.4 | 0.6 |
| | pH | 5.5 | 5.5 | 5.5 |
| Study duration | Long-term (frozen) | 48 months/−20° C. | 60 months/−20° C. | 60 months/−20° C. |
| | Accelerated | 6 months/5° C. | 6 months/5° C. | 6 months/5° C. |
| | Stressed | NA | 3 months/25° C./ | 3 months/25° C./ |
| | | | 65% relative | 65% RH |
| | | | humidity (RH) | |
| | Freeze and thaws | 3 cycles | 3 cycles | 3 cycles |
| Storage container | | Hyclone | 316L | 316L |
| | | Aegis5-14 | stainless steel | stainless steel |
| | | Bag | mini-cans | mini-cans |

^{a} = Nominal histidine content, not accounting for Donnan effect

Various DP formulations were assessed in DP Long-Term Representative and GMP Stability Studies, see Table 12, infra.

TABLE 12
Drug Product (DP) Long-Term Representative and GMP Stability Studies

| | | | Second DP | Second DP |
|---|---|---|---|---|
| | | First DP | Formulation | Formulation |
| | | Formulation | (600 mg/vial) | (420 mg/vial) |
| Formulation | Protein concentration | 20 | 60 | 60 |
| Parameter | (mg/mL) | | | |
| | Nominal histidine acetate | 20 | 20 | 20 |
| | content ^{a} (mM) | | | |
| | Sucrose content (mM) | 240 | 240 | 240 |
| | Methionine content (mM) | 0 | 10 | 10 |
| | PS20 content (mg/mL) | 0.2 | 0.4 | 0.4 |
| | pH | 5.5 | 5.5 | 5.5 |
| Study duration | Long-term (frozen) | 48 months/5° C. | 60 months/5° C. | 60 months/5° C. |
| | Accelerated | 6 months/525° C./ | 6 months/25° C./ | 6 months/25° C./ |
| | | 65% relative | 65% RH | 65% RH |
| | | humidity (RH) | | |
| Storage container | | Glass vial | Glass vial | Glass vial |

^{a} = Nominal histidine content, not accounting for Donnan effect and dilution buffer variability

A predicted change for a given assay output was computed by pooling the available data and linearly extrapolating the averaged degradation at across the relevant durations. An assay output demonstrated practically meaningful change if the extrapolated degradation for a given storage condition exceeded three times the standard deviation of the assay precision. Practically meaningful change was observed in the assay outputs for the DP formulation: 1) After three years at 5° C. for Acidic Region by IE-HPLC and Main Peak by IE-HPLC and 2) After 1 month at 25° C. for Acidic Region by IE-HPLC, Main Peak by IE-HPLC and sum of LMWF by NR-CE-SDS. The practically meaningful factors observed for DS formulations were protein concentration for acidic region formation as measured by IE-HPLC and sucrose content for subvisible particle formation.

### Example 3: Development of High Concentration Tiragolumab Formulations

To improve clinical administration of tiragolumab, high concentration formulations were developed. (Table 13, infra).

TABLE 13
High Concentration Tiragolumab Drug Product Development

| | | High | High |
|---|---|---|---|
| | Original | Concentration | Concentration |
| | Formulation, | Formulation I, | Formulation II, |
| | 400 mg/vial | 600 mg/vial | 600 mg/vial |
| Tiragolumab (mg) | 400.0 | 600.0 | 600.0 |
| L-Histidine (mg) | 62.0 | 31.0 | 31.0 |
| Acetic Acid, | 21.6 | 11.1 | 11.1 |
| Glacial (mg) | | | |
| Sucrose (mg) | 1643 | 410 | 822 |
| L-methionine | — | — | 14.9 |
| Polysorbate 20 | 4.0 (0.02% | 4.0 (0.04% | 4.0 (0.04% |
| (PS20) (mg) | w/v) | w/v) | w/v) |
| Water for | Quantity | QS to 10.0 | QS to 10.0 |
| Injection (mL) | Sufficient | | |
| | (QS) to 20.0 | | |
| [Tiragolumab] | 20 mg/ml | 60 mg/ml | 60 mg/ml |
| (mg/ml) | | | |
| Primary Packaging | 20 mL vials | | 15 mL vials |
| Configuration | | | |

L-Histidine acetate provided buffering capacity at pH 5.5. 20 mM L-histidine acetate was shown to be sufficient to maintain the formulation pH through the manufacturing of the DP as well as during the storage of the DS and DP. The total concentration of the buffering system (L-histidine and acetate) was 38.5 mM. Sucrose functioned as a tonicity agent and a concentration of 240 mM sucrose was able to achieve isotonicity. L-Methionine functioned as a stabilizer, and 10 mM was sufficient to provide stability for the DS and DP. Polysorbate 20 (PS20) functioned as a surfactant to prevent the loss of protein due to surface adsorption and to minimize the potential formation of soluble aggregates and/or insoluable proteinaceous particles. A concentration of 0.04% (w/v) PS20 was shown to be sufficient to protect the tiragolumab DS and DP against stresses that may occur during processing, handling, and storage (e.g., freezing and thawing).

Selection of the high concentration formulation was based on studies varying protein concentration, excipient concentrations, and pH (see Examples 1 and 2, supra). The buffer system of the high concentration formulation was composed of L-histidine and acetic acid and was determined to be suitable based on comparison to the real-time stability data of the lower concentration formulations. Tiragolumab concentration was increased from 20 mg/mL in the low concentration formulation to 60 mg/mL in the high concentration formulation. Aggregation rates were similar for the 20 mg/mL (low conc.) and 60 mg/mL (high conc.) tiragolumab concentrations at 2° C.-8° C. The high conc. formulation remained stable up to two years at a pH between 5.0 and 6.0 and at various excipient concentration ranges, including 100-300 mM sucrose, 10-30 mM L-histidine acetate, 0.01%-0.05% polysorbate 20, and 0 mM or 10 mM L-methionine. Furthermore, 10 mM L-methionine, added as a stabilizer, provided more consistent stability at accelerated temperatures. In addition, PS20 concentration was increased from 0.02% to 0.04% (w/v). Based on the results from these studies, a DS and/or DP liquid formulation comprising 60 mg/mL tiragolumab in 20 mM L-histidine acetate, 240 mM sucrose, 10 mM L-methionine, and 0.04% (w/v) polysorbate 20 at pH 5.5 was selected.

The formulation remained stable at the recommended storage condition of 2° C.-8° C. when protected from light. An in-line filter (0.2 μm) was used for the administration of clinical material, as a measure of precaution. The formulation was monitored for subvisible particles of ≥2 μm and ≥5 μm in size (in addition to subvisible particles of ≥10 μm and ≥25 μm in size, which was a part of the control strategy) by using a light obscuration method through development. These evaluations were conducted as a part of the extended characterization of the tiragolumab DP performed at the time of release and during stability testing.

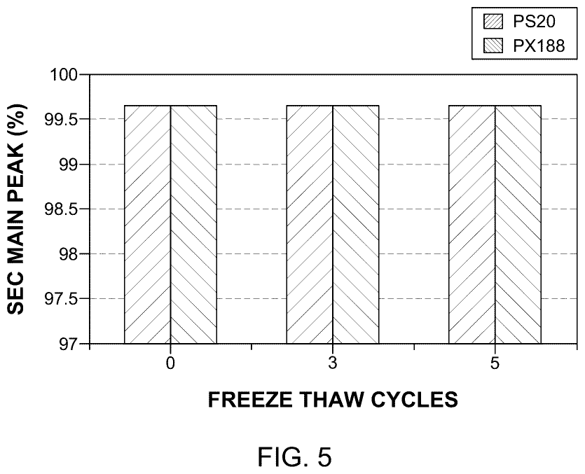

Three freeze/thaw cycles consisting of initial freezing at −40° C., followed by greater than 12 hours of storage at −20° C., and thawing at 5° C. were tested for the tiragolumab DS in 25 mL, 316 L stainless steel mini-tanks. Tiragolumab remained stable up to 7 freeze-thaw cycles at 120 mM sucrose (FIG. 5).

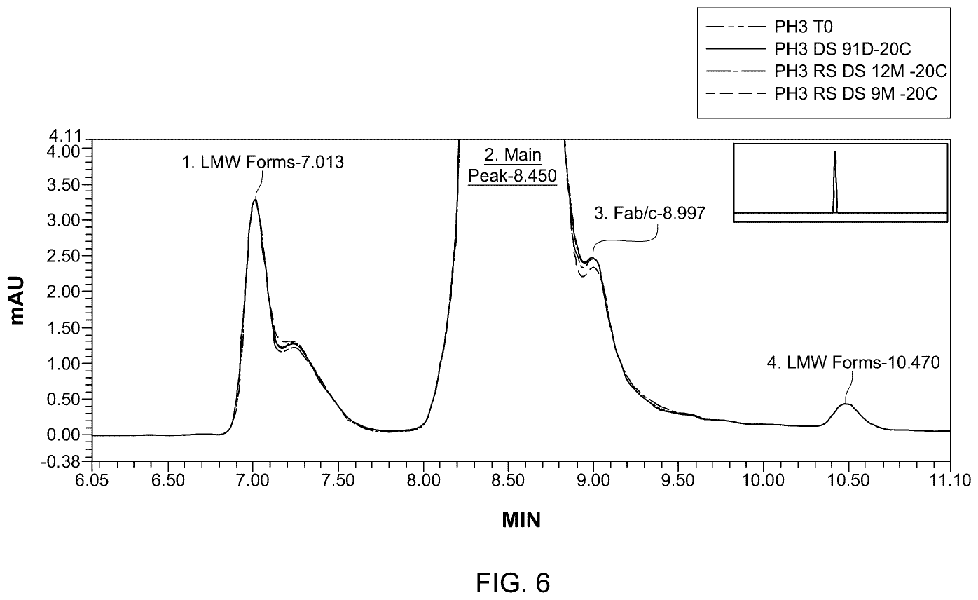

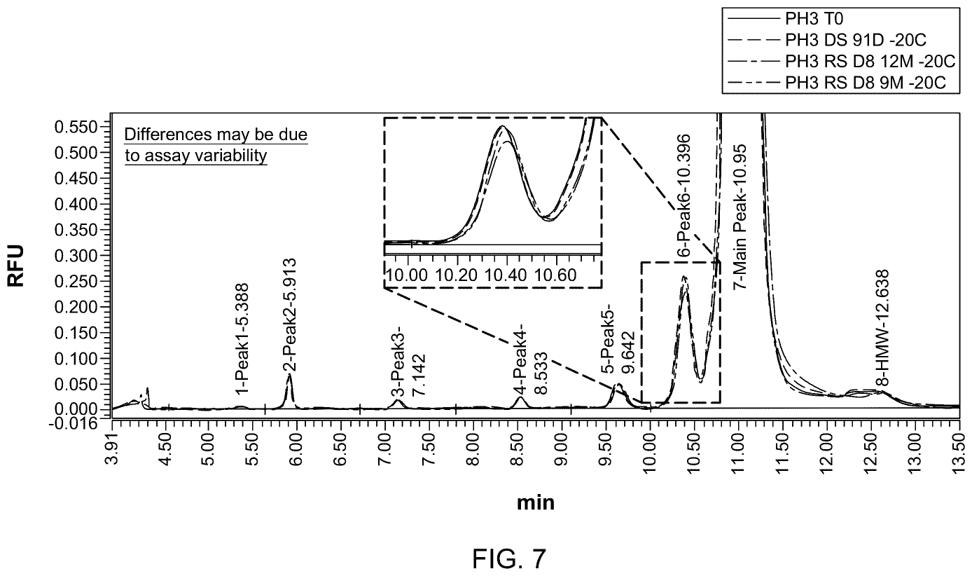

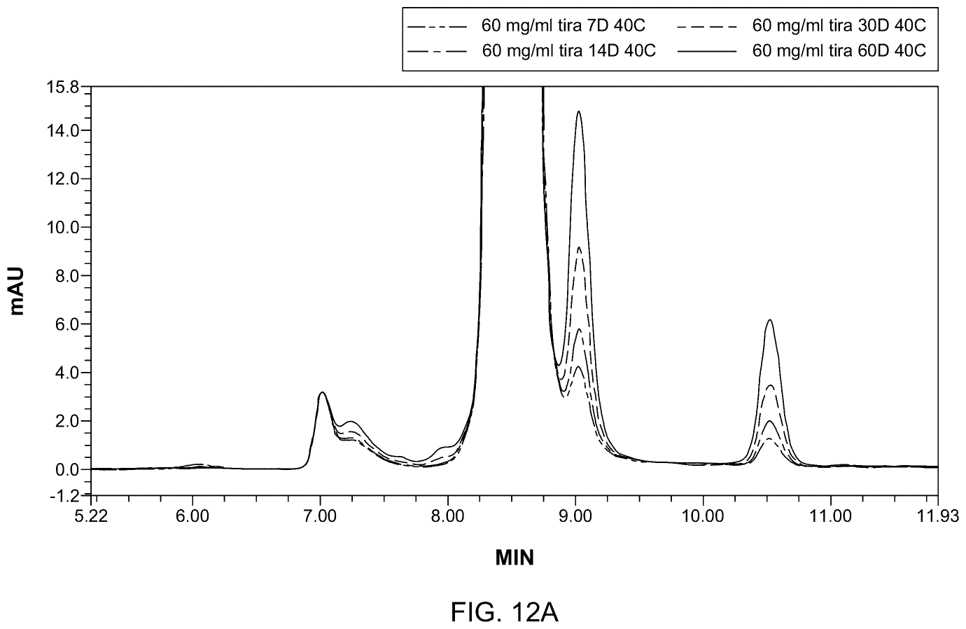

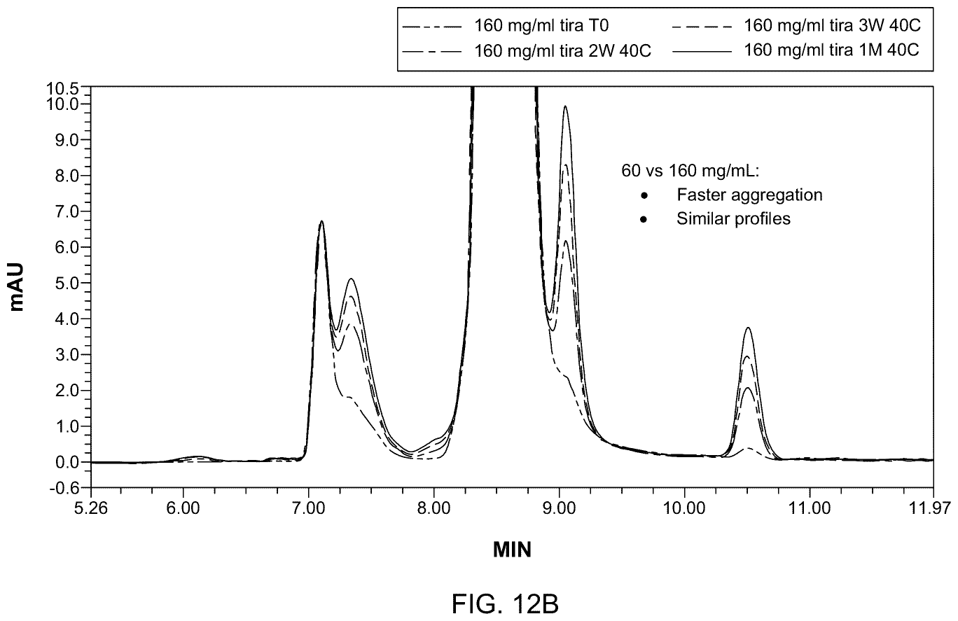

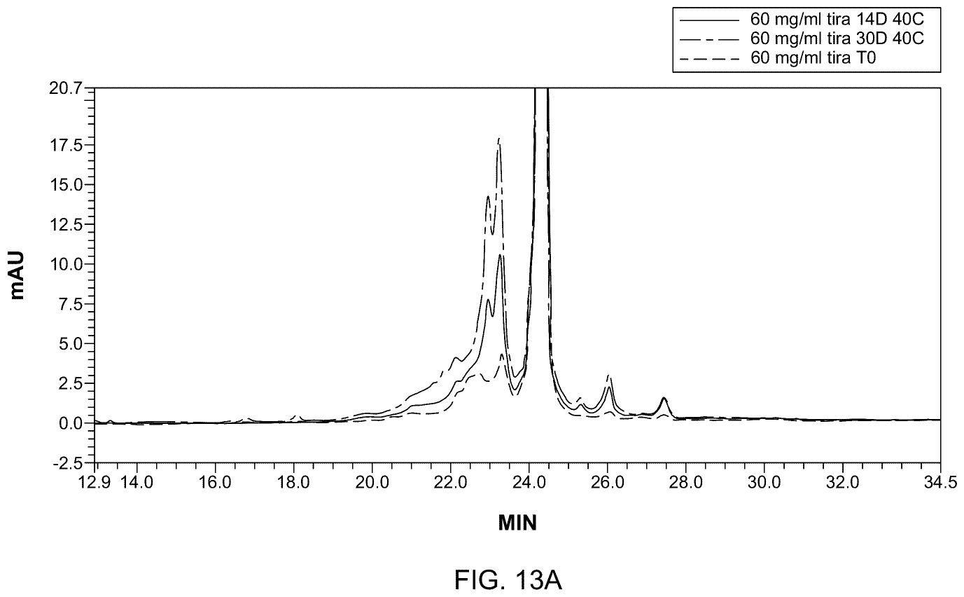

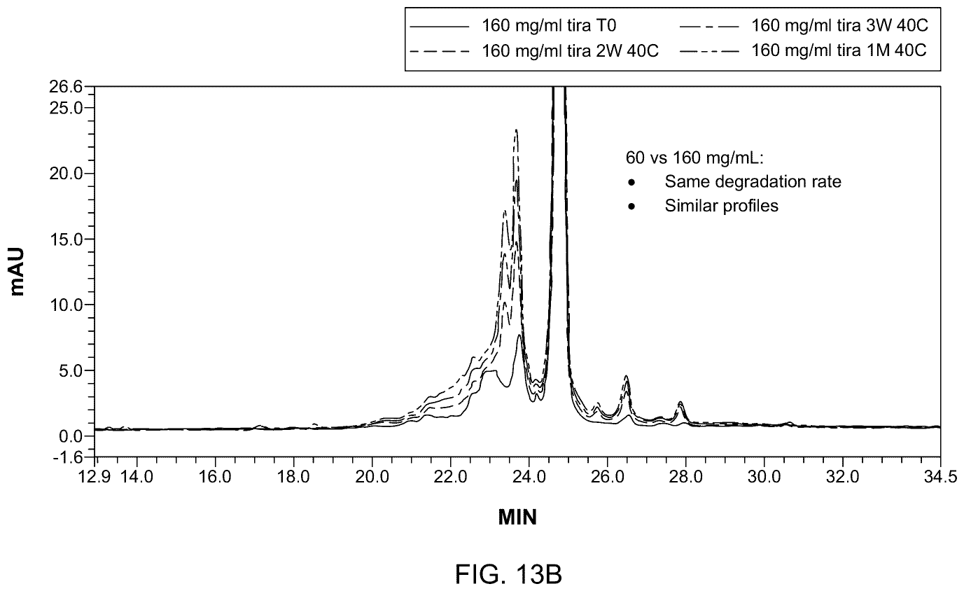

The stability of the high concentration formulation (Formulation II) at −20° C. for one year was evaluated by non-reduced CE-SDS, SEC and CE. The CE-SDS demonstrated that tiragolumab was stably maintained in monomer form over one year of storage at −20° C. See, Table 14, infra. The stability of tiragolumab at −20° C. was also confirmed by SEC (see FIG. 6) and CE (see FIG. 7). The stability of tiragolumab at 40° C. was also confirmed by SEC (see FIG. 12A) and CE (see FIG. 13A).

TABLE 14
Stability of Tiragolumab at −20° C. by Non-Reduced CE-SDS

| Protein Conc. | Time | Sum of LMWF | Main Peak | Sum of HMWF |
|---|---|---|---|---|
| (mg/ml) | (Days) | (%) | (%) | (%) |
| 60 | 0 | 2.1 | 97.4 | 0.5 |
| 60 | 91 | 2.1 | 97.4 | 0.5 |
| 60 | 183 | 2.3 | 97.4 | 0.4 |
| 60 | 274 | 2.3 | 97.4 | 0.6 |
| 60 | 365 | 2.6 | 97.2 | 0.4 |

### Example 4: Stability of High Concentration Tiragolumab Formulations During Clinical Administration

The suitability of tiragolumab DP (60 mg/mL) for administration by IV infusion after dilution in isotonic sodium chloride solution (0.9% NaCl) was tested. Compatibility and stability of the high concentration tiragolumab formulation (Formulation II of Example 3) was evaluated under the following simulated preparation and administration conditions according to:
1. Dilution of the tiragolumab drug product in infusion bags containing 0.9% NaCl in the range of 0.20-12.0 mg/mL (nominal concentration after dilution) to cover a range of doses.
2. Short-term exposure to four types of infusion bags containing 0.9% NaCl; product-contacting surface material of the bags consists of polyvinyl chloride (PVC), polyethylene (PE), polypropylene (PP), and polyolefin (PO) composed of PE and PP.
3. Use of IV infusion lines and infusion aids (product-contacting surfaces of PVC, PE, polybutadiene [PBD], polyurethane [PUR], polycarbonate, polyetherurethane—Vialon, and polytetrafluorethylene, and fluorinated ethylene propylene).
4. Use of 0.2 μm in-line filters (filter membrane of polyethersulfone [PES] and polysulfone [PSU]).

The tiragolumab drug product-contacting materials for each of the four experimental infusion setups are described in Table 15, infra. IV bags were stored for 24 hours at 2° C.-8° C., followed by storage for 24 hours at 30° C., with exposure to diffused light. Samples were tested after simulated administration.

TABLE 15
Product-Contacting Materials for Each Tested Infusion Setup

| | Setup 1 | Setup 2 | Setup 3 | Setup 4 |
|---|---|---|---|---|
| IV Bag | PVC | PO | PE | PP |
| Administration | PVC | PE | PBD | PUR |
| Set | | | | |
| Catheter | PEU-Vialon | PTFE | PUR | FEP |
| Inline Filter | PES | PES | PSU | PES |
| Infusion Aid | PC | PC | PC | PC |

FEP = fluorinated ethylene propylene;
IV = intravenous;
PBD = polybutadiene;
PC = polycarbonate;
PE = polyethylene;
PES = polyethersulfone;
PEU = polyetherurethane;
PO = polyolefin;
PP = polypropylene;
PSU = polysulfone;
PTFE = polytetrafluorethylene (Teflon);
PUR = polyurethane;
PVC = polyvinylchloride

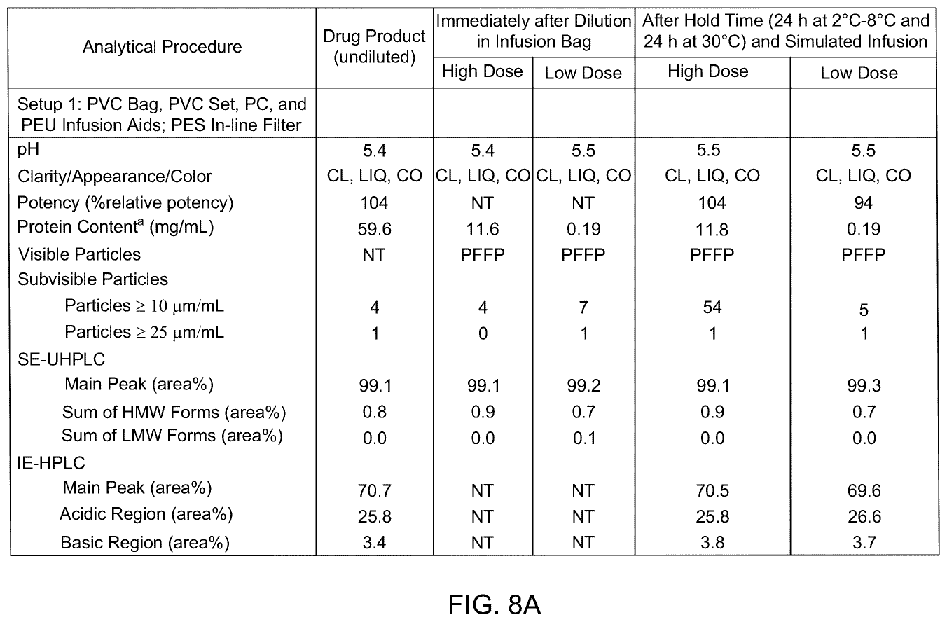

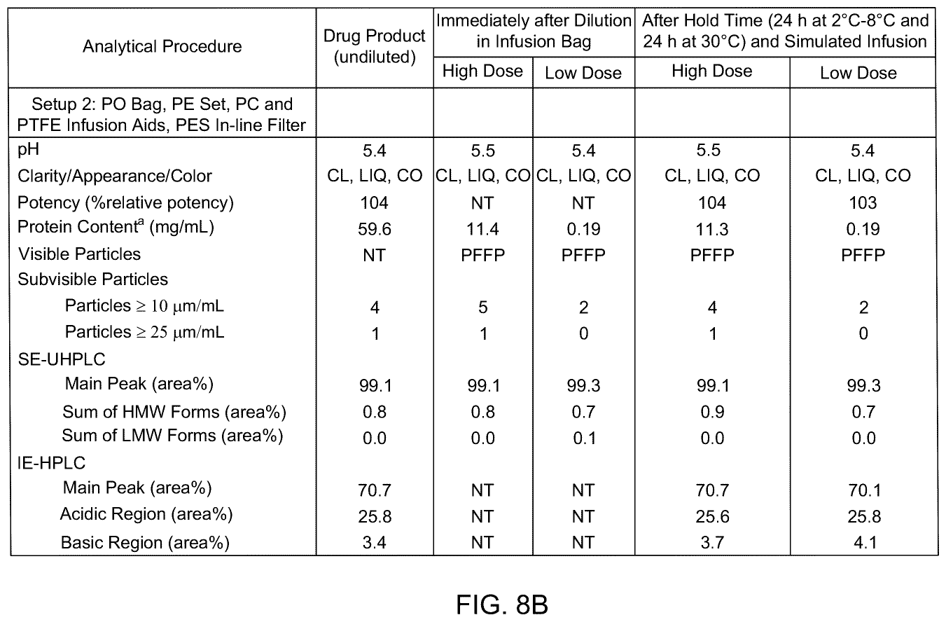

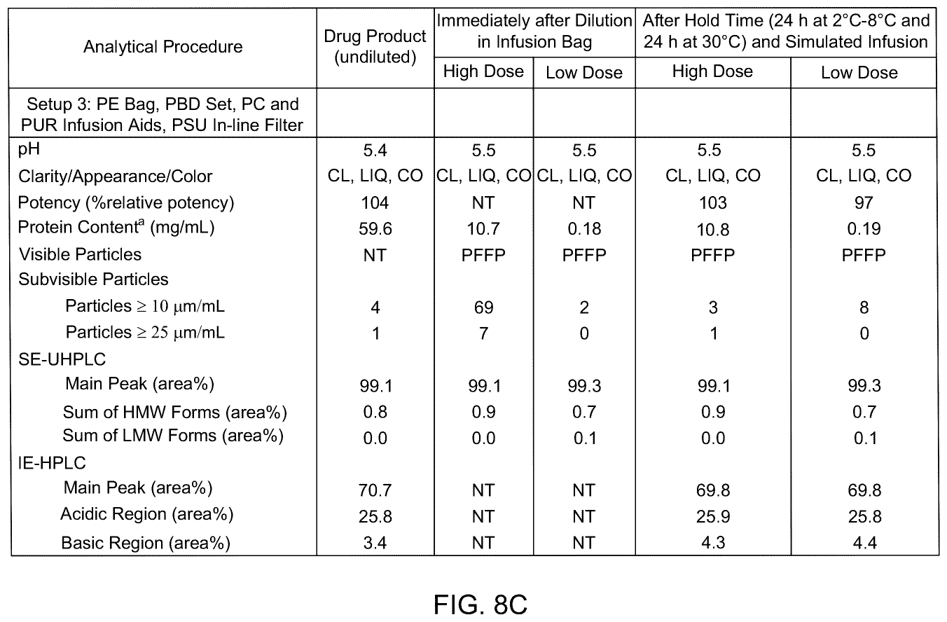

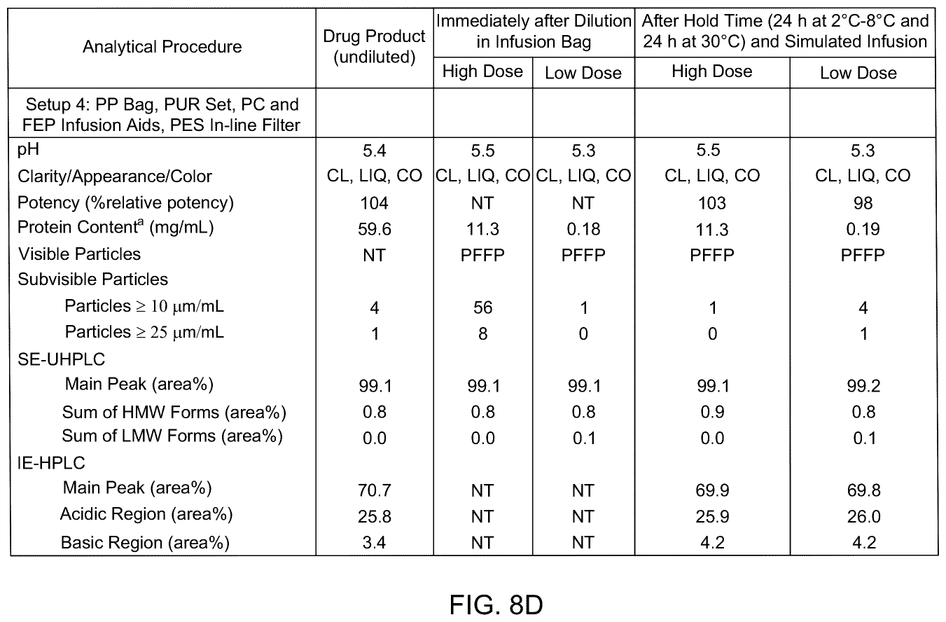

The samples were tested using appropriate stability-indicating analytical methods, including purity by size-exclusion ultra-high-performance liquid chromatography (SE-UHPLC), ion-exchange high-performance liquid chromatography (IE-HPLC), protein content by ultraviolet absorption and SE-UHPLC using a standard curve, visible particles, subvisible particles by light obscuration, color, clarity/opalescence, and pH. The stability results for the tiragolumab DP, neat or diluted into IV bags, followed by cumulative storage and simulated administration are described in FIGS. 8A-8D. Based on the results, the high concentration tiragolumab formulation was compatible with PVC, PE, PP, and PO-PE-PP IV bags after dilution to the concentration range of 0.2-12 mg/mL for up to 24 hours at 5° C., followed by 24 hours at 30° C. with exposure to ambient light, and simulated IV infusion using PVC, PE, PBD, and PUR administration sets equipped with PES and PSU in-line filters. The high concentration tiragolumab formulation, tested in simulated administration studies was physically and chemically stable under the tested conditions.

### Example 5: Stability of High Concentration Tiragolumab Formulations During Clinical Co-Administration with TECENTRIQ®

A suitability of the tiragolumab drug product (60 mg/mL) for co-infusion with the anti-PD-L1 monoclonal antibody atezolizumab (also known as TECENTRIQ®) drug product (60 mg/mL) by IV infusion after dilution in isotonic sodium chloride solution (0.9% NaCl) was evaluated. It is known in the art that the hydrophobicity of an antibody's CDR loops is a key determinant of the propensity of the antibody to aggregate. See, e.g., Perchiacca et al., Annu. Rev. Chem. Biomol. Eng. 3:263-286, 2012. Antibodies that bind to different antigens have different CDR residues and, therefore, different surface chemistries. Due to these different surface chemistries, if two antibodies are mixed for co-administration, differences in their formulations may result in instability (e.g., aggregation) of either or both antibodies. Accordingly, compatibility and stability of tiragolumab and atezolizumab were tested under the following simulated preparation and administration conditions according to:
1. Dilution of the tiragolumab and atezolizumab drug products in infusion bags containing 0.9% NaCl in the ranges of 2-12 mg/mL tiragolumab and of 4-24 mg/mL atezolizumab (nominal concentration after dilution) to cover a range of doses.
2. Short-term exposure to two types of infusion bags containing 0.9% NaCl; product-contacting surface material of the bags consists of polyvinyl chloride (PVC) and polyolefin (PO) composed of polyethylene (PE) and polypropylene (PP).
3. Use of IV infusion lines and infusion aids (product-contacting surfaces of PVC, PE, polybutadiene [PBD], polyurethane [PUR], polycarbonate, polyetherurethane-Vialon, and polytetrafluorethylene).
4. Use of 0.2 μm in-line filters (filter membrane of polyethersulfone [PES] and polysulfone [PSU]).

The tiragolumab high concentration drug product and atezolizumab-contacting materials for each of the three experimental infusion setups are described Table 16, infra. IV bags were stored for 24 hours at 2° C.-8° C., followed by storage for 6 hours at 30° C., with exposure to diffused light. Samples were tested after simulated administration.

TABLE 16
Product-Contacting Materials for Each
Tested Co-Administration Infusion Setup

| | Setup 1 | Setup 2 | Setup 3 |
|---|---|---|---|
| IV Bag | PVC | PO | PO |
| Administration | PVC | PE | PBD |
| Set | | | |
| Catheter | PEU-Vialon | PTFE | PUR |
| Inline Filter | PES | PES | PSU |
| Infusion Aid | PC | PC | PC |

IV = intravenous;
PBD = polybutadiene;
PC = polycarbonate;
PE = polyethylene;
PES = polyethersulfone;
PEU = polyetherurethane;
PO = polyolefin;
PP = polypropylene;
PSU = polysulfone;
PTFE = polytetrafluorethylene (Teflon);
PUR = polyurethane;
PVC = polyvinylchloride

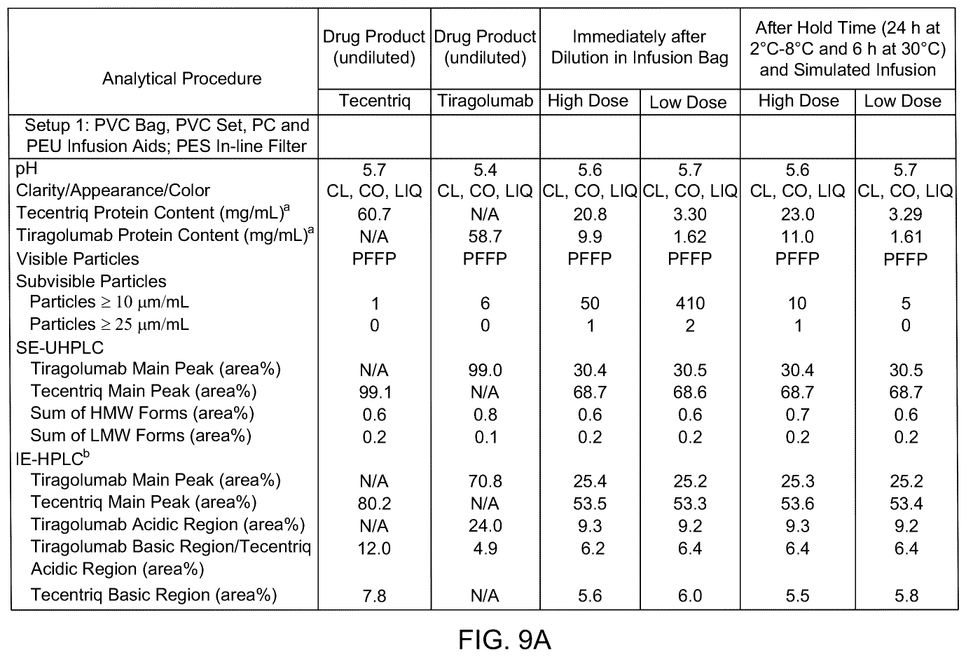

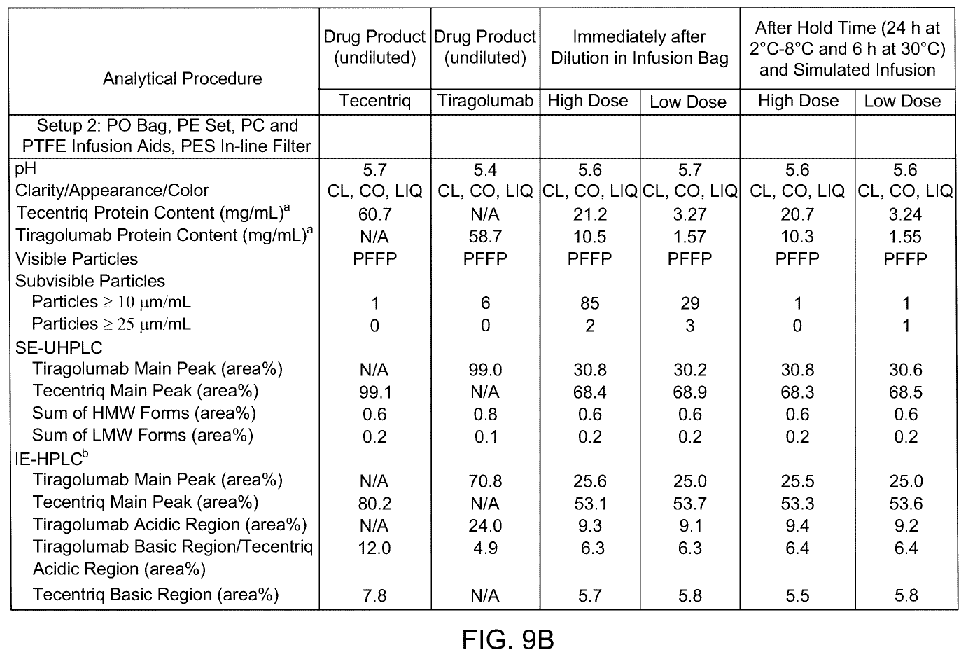

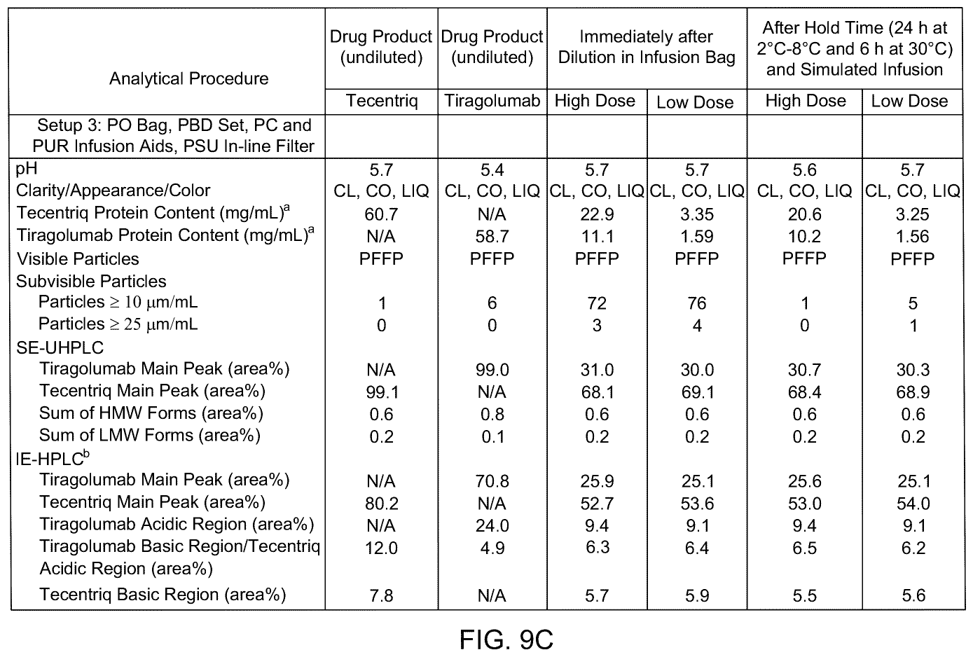

The samples were tested using appropriate stability-indicating analytical methods, including purity by size-exclusion ultra-high-performance liquid chromatography (SE-UHPLC), ion-exchange high-performance liquid chromatography (IE-HPLC), protein content by hydrophilic interaction liquid chromatography, visible particles, subvisible particles by light obscuration, color, appearance, and clarity, and pH. The stability results for the tiragolumab high concentration DP and atezolizumab DP, neat, and the dose solution for co-infusion in the IV bags, followed by cumulative storage and simulated administration, are described in FIGS. 9A-9C. Based on the results, the dose solution for co-infusion of tiragolumab and atezolizumab was compatible with PVC and PO-PE-PP IV bags after dilution to the concentration range of 2 mg/mL-12 mg/mL tiragolumab and 4 mg/mL-24 mg/mL atezolizumab for up to 24 hours at 5° C., followed by 6 hours at 30° C. with exposure to ambient light, and simulated IV infusion using PVC, PE, and PBD administration sets equipped with PES and PSU in-line filters. The tiragolumab high concentration drug product and the atezolizumab drug product tested in simulated co-administration studies were surprisingly physically and chemically stable under the tested conditions.

### Example 6. Very High Concentration Tiragolumab Drug Substance (DS) Formulation

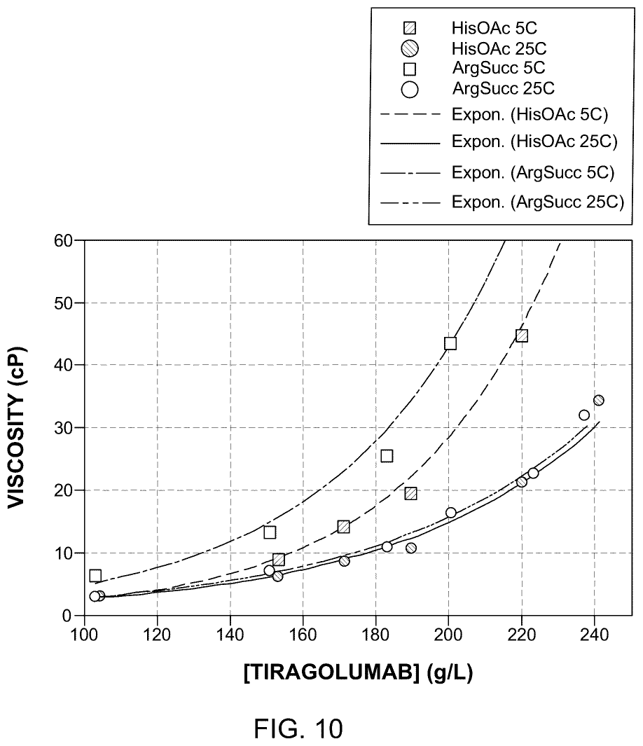

The concentration of tiragolumab (tira) in the drug substance (DS) formulation was increased to a very high concentration (e.g., 160 mg/mL) with a standard ultrafiltration diafiltration (UFDF) process (e.g., no heating of the UFDF skid). Polysorbate 20 (PS20) concentration was also increased to 0.06% to reduce particle risk to prevent enzyme activity loss. The tiragolumab formulation had a lower viscosity at a lower ionic strength, so 20 mM histidine acetate was compatible for the very high concentration of tiragolumab. See, e.g., FIG. 10. The high concentration of tiragolumab was also compatible with 125 mg/mL atezolizumab and 2000 U/mL recombinant human hyaluronidase PH20 enzyme (rHuPH20) in a solution comprising 20 mM HisOAc, 240 mM sucrose, 10 mM methionine and 0.06% PS20 at pH 5.8.

The effect of UFDF on tiragolumab aggregation was evaluated by size exclusion chromatography. Three samples were tested: (1) the loading sample (11 g/L); (2) UFDF Pool-1 (152 g/L); and (3) UFDF Pool-2 (220 g/L). Increasing protein concentration did slightly increase the aggregation rate, but the aggregation rate remained within acceptable parameters. See, Table 17, infra (%=percent relative area; vHMWF=very high molecular weight form).

TABLE 17
Effect of UFDF on Tiragolumab Aggregation by SEC

| | | | | Total | | |
|---|---|---|---|---|---|---|
| | [tira] | vHMWF | HMWF | HMW | Monomer | LMWF |
| Sample | (g/L) | (%) | (%) | (%) | (%) | (%) |
| Load | 10.88 | 0.09 | 0.67 | 0.76 | 99.2 | 0.04 |
| UFDF | 154 | 0.18 | 0.72 | 0.90 | 99.04 | 0.06 |
| Pool-1 | | | | | | |
| UFDF | 220 | 0.27 | 0.73 | 1.00 | 98.92 | 0.07 |
| Pool-2 | | | | | | |

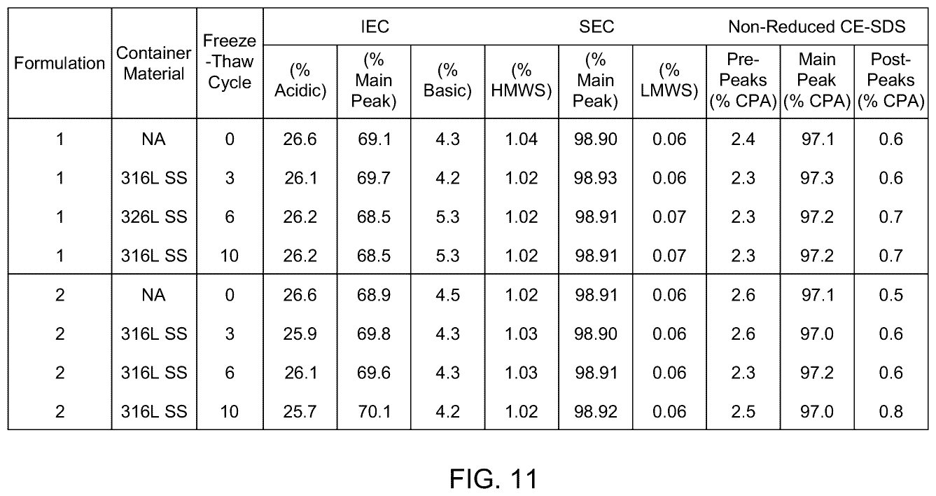

The stability of very high concentration tiragolumab formulations were determined by measuring acid/basic peaks (ion-exchange chromatography), HMWF/LWMF (size-exclusion chromatography), and by non-reduced CD-SDS following 10 freeze-thaw cycles. A proposed formulation (“Formulation 1”) comprising 160 mg/mL tiragolumab in 20 mM HisOAc, 240 mM sucrose, 10 mM methionine, 0.06% PS20 and pH 5.5 was tested. Using the results in Examples 1 and 2, a second formulation (“Formulation 2” comprising 176 mg/mL tiragolumab in 30 mM HisOAc, 180 mM sucrose, 5 mM methionine, 0.08% PS20 and pH 5.5) was prepared by altering each component of the formulation to decrease stability. Freeze-thaw stability of Formulations 1 and 2 were both stable, showing no differences between the two formulations (FIG. 11). These results correlate with long-term stability at −20° C.

The stability of the very high concentration tiragolumab formulation (160 mg/mL tiragolumab in 20 mM HisOAc, 240 mM sucrose, 10 mM methionine, 0.06% PS20 and pH 5.5) was compared to the high concentration tiragolumab formulation of Example 4 (60 mg/mL tiragolumab in 20 mM HisOAc, 240 mM sucrose, 10 mM methionine, 0.04% PS20 and pH 5.5) by size-exclusion chromatography (SEC) and ion-exchange chromatography (IEC). For SEC, the very high concentration formulation demonstrated a similar profile as the high concentration formulation but showed faster aggregation. See, FIGS. 12A and 12B. For IEC, the very high concentration formulation demonstrated a similar profile and the same degradation rate as the high concentration formulation. See, FIGS. 13A and 13B.

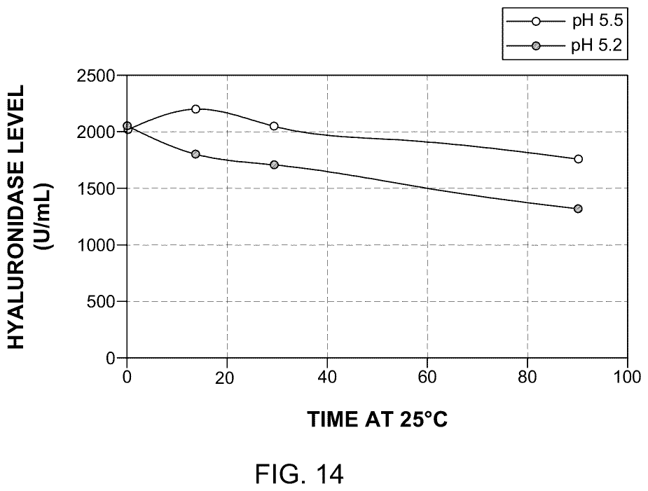

To convert the tiragolumab formulation for intravenous administration to a formulation suitable for subcutaneous administration, hyaluronidase was added to the very high tiragolumab formulation. To determine the effect of pH, hyaluronidase activity was measured over three months of storage at either 5° C. or 25° C. at pH 5.2 or pH 5.5. No change in hyaluronidase activity was observed at either pH following storage at 5° C. Following storage at 25° C., however, a higher rate of decrease in hyaluronidase activity was observed for at the pH 5.2 than at pH 5.5. See, FIG. 14.

The compatibility of very high concentration tiragolumab with hyaluronidase was also determined in an agitation study. Formulations comprising 160 mg/mL tiragolumab, 2000 units/mL hyaluronidase, and varying concentrations of polysorbate 20 were agitated in 6-cc vials (3.0 ml/vial) on an arm shaker at ambient temperature for 24 hours. Aggregation was measured by size-exclusion ultra-high-performance liquid chromatography (SE UPLC), hyaluronidase activity was measured; and polysorbate 20 concentration was measured. See Table 18, infra (PS20=polysorbate 20; CAC=color, appearance and clarity; CL=clear; TU=turbid; SY=slightly yellow; LIQ=liquid; HMWF=high molecular weight forms; LMWF=low molecular weight forms). Tiragolumab was stable under all conditions tested. While agitation disrupted hyaluronidase activity in the control sample, the presence of polysorbate 20 was able to maintain hyaluronidase activity at all concentrations tested and supports the use of polysorbate 20 at a concentration range of 0.4 mg/mL to 0.8 mg/mL (i.e. 0.04% to 0.08%).

TABLE 18
Results of a Tiragolumab (160 mg/mL) + Hyaluronidase Agitation Study

| Added | | | Sum | Main | Sum | Hyaluronidase | Measured |
|---|---|---|---|---|---|---|---|
| PS20 | | | HMWF | Peak | LMWF | Activity | PS20 |
| (mg/mL) | Condition | CAC | (%) | (%) | (%) | (U/mL) | (mg/mL) |
| 0 | Control | CL; SY; LIQ | 0.4 | 99.5 | 0.0 | 1628 | 0.00 |
| 0 | Agitated | TU; SY; LIQ | 0.5 | 99.5 | 0.0 | 2 | 0.00 |
| 0.30 | Control | CL; SY; LIQ | 0.4 | 99.5 | 0.0 | 2371 | 0.32 |
| 0.30 | Agitated | CL; SY; LIQ | 0.4 | 99.5 | 0.0 | 2153 | 0.32 |
| 0.35 | Control | CL; SY; LIQ | 0.4 | 99.5 | 0.0 | 2318 | 0.37 |
| 0.35 | Agitated | CL; SY; LIQ | 0.4 | 99.5 | 0.0 | 2186 | 0.36 |
| 0.4 | Control | CL; SY; LIQ | 0.4 | 99.6 | 0.0 | 2355 | 0.42 |
| 0.4 | Agitated | CL; SY; LIQ | 0.4 | 99.5 | 0.0 | 2330 | 0.41 |
| 0.45 | Control | CL; SY; LIQ | 0.4 | 99.5 | 0.0 | 2352 | 0.47 |
| 0.45 | Agitated | CL; SY; LIQ | 0.4 | 99.5 | 0.0 | 2407 | 0.46 |
| 0.5 | Control | CL; SY; LIQ | 0.4 | 99.5 | 0.0 | 2350 | 0.53 |
| 0.5 | Agitated | CL; SY; LIQ | 0.4 | 99.6 | 0.0 | 2359 | 0.51 |
| 0.6 | Control | CL; SY; LIQ | 0.4 | 99.5 | 0.0 | 2371 | 0.62 |
| 0.6 | Agitated | CL; SY; LIQ | 0.4 | 99.5 | 0.0 | 2484 | 0.60 |

### Example 7. Tiragolumab+Atezolizumab Fixed Dose Combination (FDC) Formulation

Patients receiving combination therapies often have to receive each drug by a separate infusion, which may require long days in or additional visits to the clinic. Due to their different surface chemistries, however, different proteins often have different formulations requirements. See, e.g., Wang, Int. J. Pharmaceutics, 1999, vol. 185:129-188 at 130 (“the structural differences among different proteins are so significant that generalization of universal stabilization strategies has not been successful. . . . Very often, proteins need to be evaluated individually and stabilized on a trial-and-error basis.”). For antibodies, the hydrophobicity of an antibody's CDR loops is a key determinant of the propensity of the antibody to aggregate. See, e.g., Perchiacca et al., Annu. Rev. Chem. Biomol. Eng. 3:263-286, 2012. Antibodies that bind to different antigens have different CDR residues and, therefore, different surface chemistries. Due to these different surface chemistries, it is difficult to co-formulate antibodies that bind different targets because they require different formulation components to maintain stability.

Atezolizumab and tiragolumab are typically administered via separate intravenous infusions in a 2:1 (atezolizumab: tiragolumab) ratio Q3W for the treatment of cancer. To increase patient convenience and, therefore, compliance, fixed-dose combination (FDC) formulations comprising both tiragolumab and atezolizumab, which can be administered in a single infusion, were developed.

Additionally, formulations which permit flexible dosing regimens (e.g. longer than every three weeks) can further increase patient convenience and, therefore, patient compliance. Accordingly, two formulations, a two-week formulation and a three-week formulation, were developed. Such formulations permit any weekly dosing schedule longer than one week. For example, Q4W dosing can be performed using two two-week formulations, Q5W dosing can be performed using one two-week formulation and one three-week formulation, Q6W dosing can be performed using two three-week formulations, and so on.

The two-week formulation comprises 420 mg tiragolumab and 840 mg atezolizumab, and the three-week formulation comprises 600 mg tiragolumab and 1200 mg atezolizumab. The intravenous FDC formulations were also shown to be adaptable for subcutaneous (SC) administration with the addition of hyaluronidase (e.g., rHuPH20).

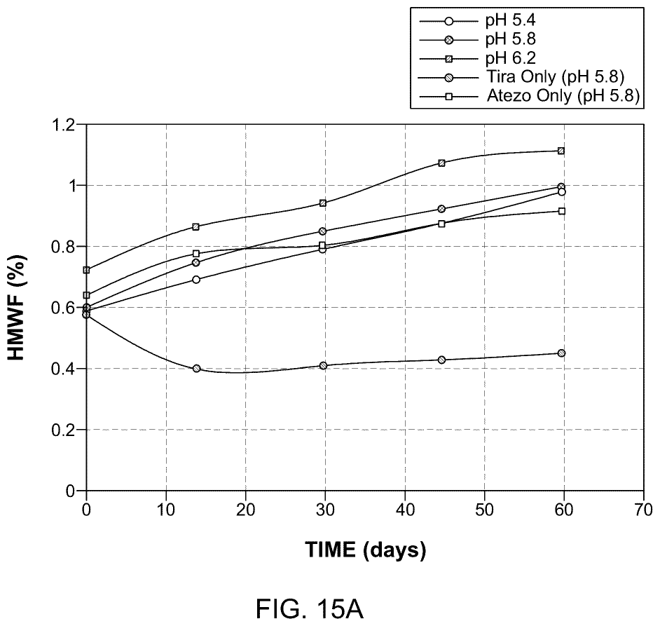

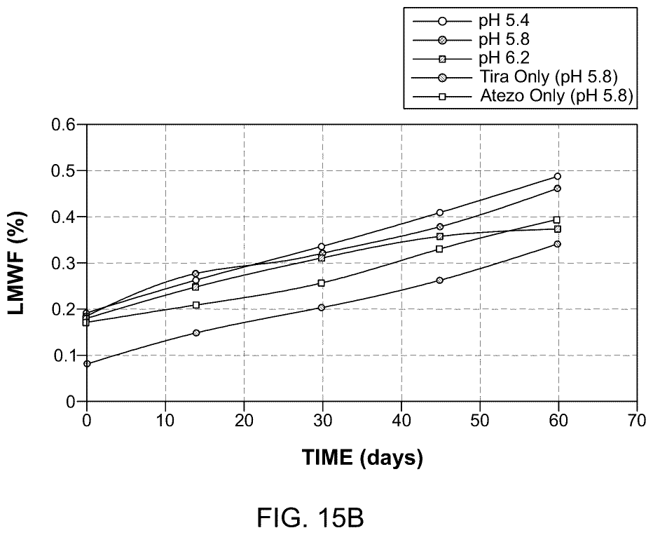

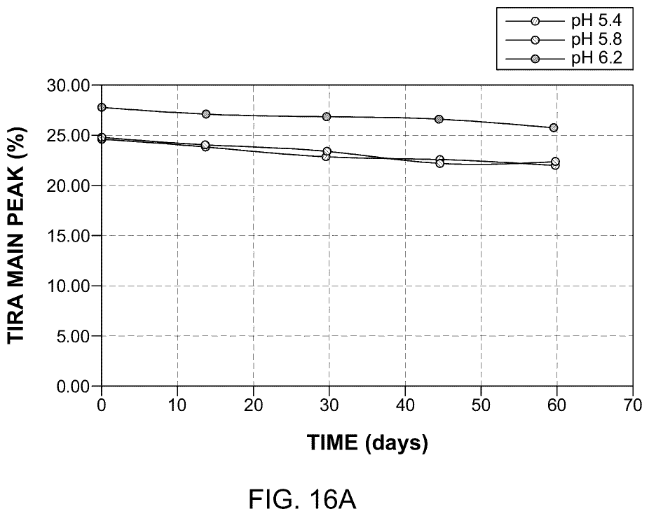

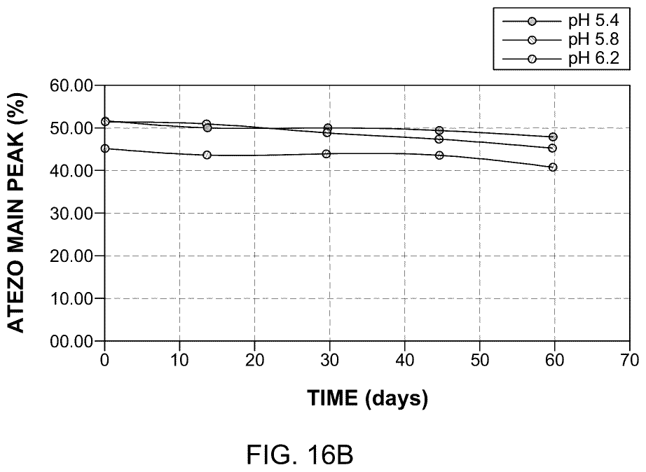

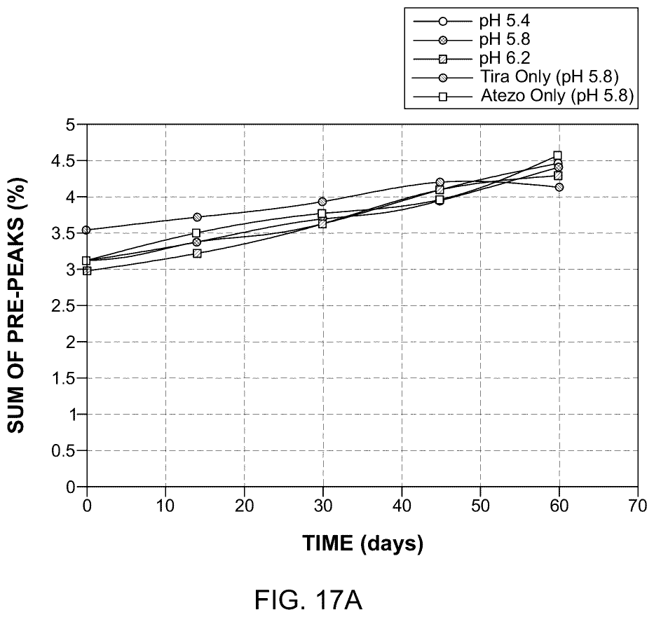

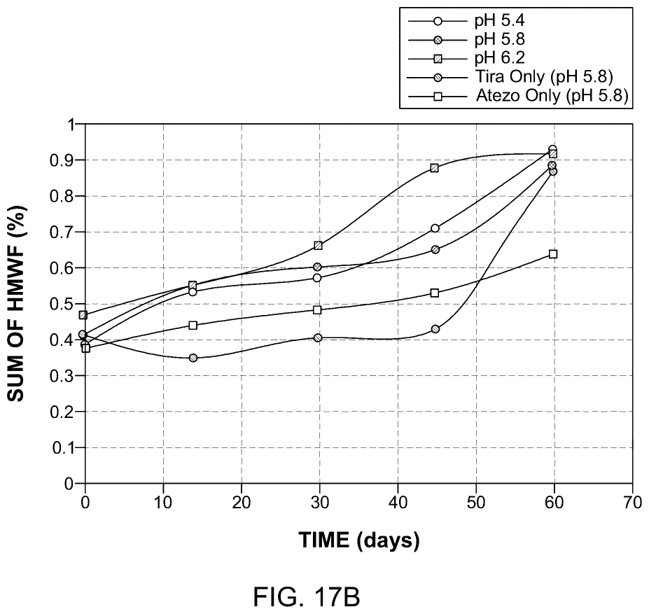

Selection of the excipients in the FDC formulations was based on studies varying protein concentration, excipient concentrations, and pH (see Examples 1 and 2, supra). Each FDC formulation was developed to comprise 80 mg/mL atezolizumab, 40 mg/mL tiragolumab, 20 mM histidine acetate, 240 mM sucrose, 10 mM methionine and 0.06% PS20. As demonstrated supra (see, e.g., Table 8), pH was a parameter that caused practically meaningful changes in tiragolumab stability, with a pH of 5.5 being preferred for tiragolumab. It was known in the art, however, that a pH of 5.5 can increase aggregation of atezolizumab. See, e.g., WO 2021/118930. In separate formulations, atezolizumab DS was manufactured at pH 5.8, while tiragolumab DS was manufactured at pH 5.5, so FDC formulations having a pH of 5.4, 5.8 and 6.2 were assessed to ensure stability of combining atezolizumab and tiragolumab in a single formulation. Size exclusion chromatography (SEC) demonstrated a similar change across all FDC pHs in the high molecular weight form (HMWF; FIG. 15A) and minimal change with increasing pH in the low molecular weight form (LMWF; FIG. 15B) over time. Ion exchange chromatography (IEC) demonstrated a similar change across all formulations tested at pH 5.5, 5.8 and 6.2 for both tiragolumab (FIG. 16A) and atezolizumab (FIG. 16B). CE-SDS demonstrated primarily degradation of LMWFs but minimal differences between the formulations. See, FIGS. 17A (LMWFs) and 17B (HMWFs). Accordingly, the SEC, IEC, and CE-SDS data demonstrated that atezolizumab and tiragolumab can be acceptably co-formulated at a pH of 5.8.

It is known in the art that highly concentrated antibody formulations may demonstrate high viscosities (>20 centipoise (cP)), which may render them unsuitable for SC administration or may cause difficulties in the biomanufacturing process. See, e.g., Tomar D S, et al., MAbs, 2016, vol. 8 (2): 216-228. Accordingly, the viscosities of the FDC formulations at various pHs were evaluated. See, Table 19, infra. The viscosities of the FDC formulations were acceptable across the pH ranges tested.

TABLE 19
Viscosity of FDC Formulations at
Various pH and Temperature Ranges

| | Viscosity at | Viscosity at | Viscosity at |
|---|---|---|---|
| pH | 5° C. (cP) | 19° C. (cP) | 25° C. (cP) |
| 5.4 | 10 | 6.8 | 5.2 |
| 5.8 | 5.8 | 4.4 | 3.2 |
| 6.2 | 4.9 | 3.5 | 2.5 |

The compatibility of the FDC formulations with hyaluronidase was also determined in an agitation study. 2000 units/ml hyaluronidase was added to the FDC formulations along with varying concentrations of polysorbate 20. The samples were agitated in 6-cc vials (3.0 ml/vial) on an arm shaker at ambient temperature for 24 hours. No changes in color, appearance and clarity were observed. Similarly, no changes were observed for particles using HIAC. Aggregation was measured by size-exclusion ultra-high-performance liquid chromatography (SE UPLC), hyaluronidase activity was measured; and polysorbate 20 concentration was measured. See Table 20, infra (PS20=polysorbate 20; HMWF=high molecular weight forms; Tira=tiragolumab; Atezo=atezolizumab; LMWF=low molecular weight forms). The data support the use of polysorbate 20 at a concentration range of 0.4 to 0.8 mg/ml (i.e. 0.04 to 0.08%) for subcutaneous FDC formulations.

TABLE 20
Agitation Screen for FDC Formulations

| | Delta between Agitation and Control | | | |
|---|---|---|---|---|
| PS20 Conc. | HMWF (Δ%) | Tira (Δ %) | Atezo (Δ %) | LMWF (Δ %) |
| 0.0384 | 0.0 | −0.1 | 0.0 | 0.0 |
| 0.045 | 0.0 | −0.1 | 0.1 | 0.0 |
| 0.06 | 0.0 | 0.2 | −0.2 | 0.0 |

The co-formulation of tiragolumab and atezolizumab surprisingly demonstrated a broader pH range than expected. As discussed supra, a pH of 5.5 can increase aggregation of atezolizumab (see, e.g., WO 2021/118930) but when co-formulated with tiragolumab, atezolizumab was unexpectedly stable within the pH range of 5.4 to 6.2.

### Example 8. Combination Therapy, Open-label, Multicenter Dose-Finding Study of Tiragolumab and Atezolizumab

A combination therapy, open-label, and multicenter study to evaluate the safety, pharmacokinetics (PK), and tolerability of subcutaneous administration of tiragolumab in combination with an anti-PD-L1/PD-1 antibody such as atezolizumab will be conducted in subjects with locally advanced or metastatic solid tumors. The study will determine the doses of tiragolumab subcutaneous (SC) that are comparable in exposure with the sequential administration of atezolizumab intravenous (IV) followed by tiragolumab IV on the basis of PK analysis of serum tiragolumab and atezolizumab C_{trough} at Cycle 1 (pre-dose Cycle 2). Tiragolumab SC and atezolizumab SC will be used for all 3 cohorts.

Subjects will be enrolled in one of the 3 cohorts: Cohort 1 will consist of approximately 10-18 evaluable subjects; Cohorts 2 and 3 will consist of approximately 30 evaluable subjects each. The total number will be approximately 70-78 evaluable subjects.

In Cohort 1, Cycle 1: Subjects will receive 880 mg tiragolumab SC co-mixed with 2000 mg atezolizumab SC in the abdomen, at Day 1 of the first cycle (21 days). In Cohort 1, Cycle 2: Subjects will receive 1200 mg atezolizumab IV followed by 600 mg tiragolumab IV every 3 weeks (Q3W) (Day 1 of each cycle) from Cycle 2 onwards until disease progression, loss of clinical benefit, unacceptable toxicity, or withdrawal of consent.

In Cohort 2, Cycle 1 to 3: Subjects will receive tiragolumab SC co-mixed with atezolizumab SC in the thigh Q3W at Day 1 of each cycle for 3 cycles (21 days each) at a dose to be determined following Cohort 1. In Cohort 2, Cycle 4: Subjects will receive 1200 mg atezolizumab IV followed by 600 mg tiragolumab IV Q3W (Day 1 of each cycle) starting from Cycle 4 onwards until disease progression, loss of clinical benefit, unacceptable toxicity, or withdrawal of consent.

In Cohort 3, Cycle 1 to 3: Subjects will receive tiragolumab SC co-mixed with atezolizumab SC in the abdomen Q3W at Day 1 of each cycle for 3 cycles (21 days each) at a dose to be determined following Cohort 1. In Cohort 3, Cycle 4: Subjects will receive 1200 mg atezolizumab IV followed by 600 mg tiragolumab IV Q3W (Day 1 of each cycle) starting from Cycle 4 onwards until disease progression, loss of clinical benefit, unacceptable toxicity, or withdrawal of consent.

The doses for Cohorts 2 and 3 will be selected based on Cohort 1 to determine the predicted exposures are comparable to the exposures tested with IV sequential administration of these pharmaceutical agents or drugs.

Cohorts 2 and 3 may start in parallel or sequentially or enrollment of only one of the cohorts may be conducts if only one may be needed for determination of optimal dose for SC use of the 2 drugs.

Subjects who discontinue study treatment prematurely, defined as discontinuation prior to completion of 2 cycles in Cohort 1, or 4 cycles in Cohorts 2 and 3, for reasons other than toxicity, may be replaced if they are not considered evaluable.

All subjects will be required to report any symptoms and adverse events as soon as possible, particularly during the first 72 hours after the first injection. The first 2 subjects in each cohort (subjects in Cohorts 2 and 3 may be enrolled concurrently) will be observed for 1 week before dosing the next patient. There will be no staggering of patient enrollment after the third subject in each cohort. The cohort review of all safety data begins 7 days after the last patient of the respective cohort receive the second cycle dose.

After initiation of study treatment, all adverse events will be reported until 30 days after the final dose of study treatment or until initiation of a new systemic anti-cancer therapy, whichever occurs first, and serious adverse events will continue to be reported until 90 days after the final dose of study treatment or until initiation of a new systemic anti-cancer therapy, whichever occurs first. In addition, adverse events of special interest will continue to be reported until 90 days after the final dose of study treatment, regardless of initiation of subsequent anti-cancer therapy. After this period, any deaths or serious adverse events that are believed to be related to prior treatment with study pharmaceutical agent(s) or drug(s) should be reported. Adverse events should be followed until the event has resolved to baseline grade or better, the event is assessed as stable, the subject is lost to follow-up or the patient withdraws consent. Every effort should be made to follow all serious adverse events considered to be related to the study treatment or protocol-related procedures until a final outcome can be reported.

Tumor assessments will be performed every 6 weeks (Q6W) (+7 days) for the first 48 weeks and every 9 weeks (Q9W) (+7 days) subsequently until radiographic progressive disease (PD) or loss of clinical benefit. Subjects will be followed for OS until death, lost-to-follow-up, withdrawal from the study, or study termination by the Sponsor, whichever occurs first.

In case of lack of local tolerability in any cohort, another cohort with separate injection sites for atezolizumab SC and tiragolumab SC may be evaluated.

Subjects who do not meet the criteria for participation in this study (i.e., screen failure) may qualify for one re-screening opportunity (for a total of 2 screenings per subject). Subjects are not required to re-sign the consent form if they are re-screened within 28 days after previously signing the consent form.

Additional cohorts to explore additional doses may be added if PK from currently planned cohorts are deemed insufficient for predicting an appropriate Phase III doses for SC administration in combination. In addition, depending on the bioavailability, a loading dose may be needed in Cohorts 2 or 3 to determine overall exposure in Cycle 1 following SC administration is comparable to that following IV administration. The choice of doses will determine that predicted exposures are comparable to the exposures seen with the IV administration of doses previously evaluated for atezolizumab and tiragolumab. In case of tolerability issues, a safety cohort evaluating SC administration of the 2 pharmaceutical agents or drugs (separately on different site or different time schedules) may be evaluated.

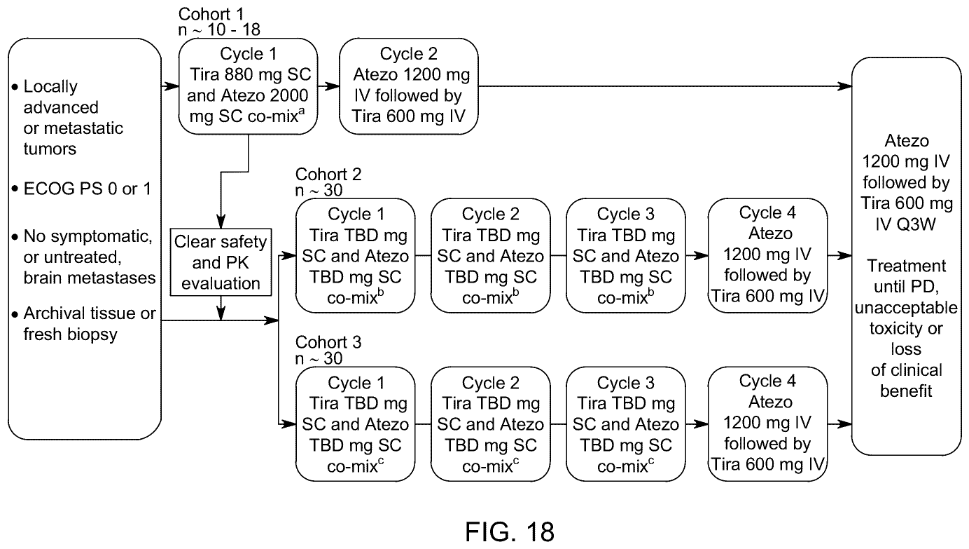

FIG. 18 provides an overview of the study design. During the study, subjects who meet criteria for radiographic disease progression and show evidence of clinical benefit may continue treatment with tiragolumab and atezolizumab, provided that the subject meet all of the following criteria:
1. Evidence of clinical benefit as assessed;
2. Absence of symptoms and signs (including worsening of laboratory values (e.g., new or worsening hypercalcemia)) indicating unequivocal progression of disease;
3. No decline in Eastern Cooperative Oncology Group (ECOG) Performance Status that can be attributed to disease progression;
4. Absence of tumor progression at critical anatomical sites (e.g., leptomeningeal disease) that cannot be managed by protocol-allowed medical interventions; and
5. Patients must provide written informed consent to acknowledge deferring other treatment options in favor of continuing study treatment at the time of initial radiographic progression.

Treatment will be discontinued if clinical deterioration due to disease progression occurs at any time, or if disease progression is confirmed on the follow-up scans performed 4-8 weeks later.

The end of the study is defined as the date when the last subject, last visit, occurs or the date at which the last data point required for statistical analysis (i.e., the last subject in Cohort 2 or 3 depending which cohort has last subject, last visit) or safety follow-up is completed from the last subject, whichever occurs later. The total length of the study, from screening of the first subject to the end of the study, is expected to be approximately 2 years.

### Objective and Endpoints

This study will evaluate the pharmacokinetic (PK), safety, tolerability, and exploratory efficacy of various doses of tiragolumab and atezolizumab administered as a single subcutaneous injection (e.g., a tiragolumab and atezolizumab SC co-mix with rHuPH20) and of the sequential administration of intravenous tiragolumab and atezolizumab in subjects with locally advanced or metastatic solid tumors.

The primary PK objective will be to determine the optimal dose of the tiragolumab and atezolizumab subcutaneous co-mix that provides exposure comparable to tiragolumab and atezolizumab intravenously administered sequentially, as determined by PK analysis on the basis of the endpoint of model-predicted serum tiragolumab and atezolizumab trough concentrations (C_{trough}) at cycle 1 or pre-dose cycle 2. The secondary PK objective is to characterize the PK profile of the tiragolumab and atezolizumab subcutaneous co-mix based on the endpoints of (1) serum tiragolumab concentration at various timepoints during subcutaneous administration and (2) serum atezolizumab concentrations at various timepoints during subcutaneous administration. Other PK parameters, such as area under the concentration curve (AUC) and maximum concentration (Cₘₐₓ) will also be evaluated.

The safety objective will be to evaluate the safety and tolerability of tiragolumab and atezolizumab with the endpoints of incidence and severity of adverse events, in which severity will be determined according to National Cancer Institute Common Terminology Criteria for Adverse Events (NCI CTCAE) v5.0. Severity for cytokine-release syndrome (CRS) will also be determined according to American Society for Transplantation and Cellular Therapy (ASTCT) CRS Consensus Grading scale. The exploratory safety objective will also evaluate the safety and tolerability of subcutaneous co-mix of tiragolumab and atezolizumab based on the endpoint of incidence of injection-site reactions, which will be assessed through the use of the Local Injection-Site Symptom Assessment (LISSA).

The immunogenicity objective will be to evaluate the immune response to tiragolumab, atezolizumab, and recombinant human hyaluronidase (rHuPH20). The endpoints include prevalence of anti-drug antibodies (ADAs) to tiragolumab at baseline and incidence of ADAs to tiragolumab during the study, prevalence of ADAs to atezolizumab at baseline and incidence of ADAs to atezolizumab during the study, and prevalence of ADAs to rHuPH20 at baseline and incidence of ADAs to rHuPH20 during the study. The exploratory immunogenicity objective will also evaluate potential effects of ADAs based on the relationship between tiragolumab ADA status and PK, safety and/or efficacy endpoints, relationship between atezolizumab ADA status and PK, safety, and/or efficacy endpoints, and relationship between rHuPH20 ADA status and PK, safety, and/or efficacy endpoints.

Exploratory efficacy objectives will include evaluating the efficacy response of tiragolumab and atezolizumab based on:
1. Progression-free survival (PFS) defined as the time from study entry to the first occurrence of disease progression or death from any cause according to Response Evaluation Criteria in Solid Tumors version 1.1 (RECIST v1.1);
2. Objective response rate (ORR) defined as the proportion of subjects with a complete response (CR) or partial response (PR) according to RECIST v1.1 and confirmed by repeat assessment ≥4 weeks after initial documentation;
3. Duration of objection response (DOR) defined as the time from the first occurrence of a documented objective response to the time of the first documented disease progression or death from any cause per RECIST v1.1; and
4. Overall survival (OS) defined as the time from study entry to death from any cause.

### Rationale

Atezolizumab SC and tiragolumab SC co-mix will be administered with the aim of selecting a dose that results in serum concentration that are comparable to that with atezolizumab 1200 mg IV Q3W and tiragolumab 600 mg IV Q3W.

An atezolizumab dose of 1875 mg is being investigated in the thigh in Part 2 (Phase III) of Study 1 and is based on the results of Study 1 Part 1 (Phase 1b) in which, a 56% higher dose of atezolizumab SC was needed to achieve AUC in Cycle 1 similar to that of the 1200 mg IV dose. The bioavailability of atezolizumab in abdomen was ˜10% lower than in thigh in the same Phase 1b part of the study. Therefore, in Cohort 1, because the administration is planned to be in the abdomen, a higher dose of 200 mg has been selected.

Because tiragolumab has not been administered using the SC formulation in a clinical trial, for Cohort 1, the dose of 880 mg for the SC formulation has been selected assuming a bioavailability similar to that of atezolizumab. Given that these 2 molecules have not been administered together by SC route before, the bioavailability will be reassessed in Cohort 1, and the dose/regimen for Cohorts 2 and 3 will be based on the PK results from Cohort 1. Doses up to 1200 mg IV tiragolumab and 20 mg/kv IV atezolizumab, are considered well-tolerated when administered individually. Sequential administration of 1200 mg IV atezolizumab and 1200 mg IV tiragolumab has also been evaluated previously.

The dose of tiragolumab (e.g., 880 mg) is based on assumed bioavailability of ˜80% for the SC administration as compared to the 600 mg IV. Because SC absorption causes the drug to appear in circulation more slowly than IV administration, an additional 20 to 30% dose is added to determine the first cycle exposure (AUC) to be comparable to the first cycle exposure with 600 mg IV. If the bioavailability is lower (e.g., as low at 40%) as determined from Cohort 1, a dose of up to 1120 mg may be used for tiragolumab in Cohorts 2 or 3 to determine equivalent exposure. Similarly, for abdominal administration in Cohort 3, if a loading dose is considered as required for Cycle 1 to get the exposures similar to historical data from 600 mg IV in Cohort 1, two doses of tiragolumab will be administered on Cycle 1 Day 1. The doses were selected so that the Cₘₐₓ and AUC will not exceed exposures observed with previously used high doses of tiragolumab in any cycle (e.g., 1200 mg IV Q3W).

The dose of atezolizumab (2000 mg) is based on ˜70% bioavailability observed in a previous study and an additional ˜36% dose to reach exposure (AUC) comparable first cycle following 1200 mg IV dosing. For thigh (Cohort 2), the dose for atezolizumab will be 1875 mg if the bioavailability estimation is confirmed in Cohort 1. For abdominal administration (Cohort 3), if a loading dose is considered as required for Cycle 1 to get exposures similar to historical data from 1200 mg IV in Cycle 1, two doses of 2000 mg will be administered on Cycle 1 Day 1. The doses selected will be such that the Cₘₐₓ and AUC will not exceed exposures seen with previously used higher doses of atezolizumab in any cycle (e.g., 20 mg/kg IV Q3W).

The concentration of rHuPH20 to be used is 2000 U/mL. If a higher maintenance dose of atezolizumab and tiragolumab is needed in Cohorts 2 or 3 to determine adequate exposure in cycle 1 following SC administration, the enzyme levels are expected to be close to the highest dose tested in rHuPH20 due to the higher dose employed using the same formulation. The enzyme concentrations will be measured in Cohorts 2 and 3 on Day 1.

### Analysis

The primary analysis will be performed at the end of Cycle 1 of Cohort 1, 2, and 3 for characterization of pharmacokinetics (PK) following subcutaneous dosing. PK from Cycle 2 to 4 will be used for model-based bioavailability and PK comparison following intravenous and subcutaneous administration.

The study will obtain preliminary PK, safety, pharmacodynamics and activity information in the treated populations. Cohort 1 will enroll approximately 10 to 18 eligible subjects, Cohorts 2 and 3 will enroll approximately 30 subjects.

Baseline characteristics will include, but not be limited to, ECOG Performance Status.

PK analysis will be performed on data from all PK-evaluable subjects as defined as a subject who has received at least one dose of any component of the study treatment and has at least one evaluable post-dose PK sample. Serum samples for PK evaluation will be collected at various timepoints. The primary PK endpoint will be model-predicted serum tiragolumab and atezolizumab C_{trough} at Cycle 1 or pre-dose Cycle 2. The PK analysis population will include subjects with sufficient data that will enable estimation of various parameters (e.g., AUC, tₘₐₓ, Cₘₐₓ, half-life, C_{trough}), with subject groups by treatment.

The safety analysis population will include enrolled subjects who have received at least one dose of any component of the study treatment, with subjects grouped according to treatment received. Pain and injection site symptom evaluation will be performed on subjects who have received at least one dose of any component of the study treatment. All subjects who receive any amount of study treatment will be included in the safety analyses. Safety will be assessed through summaries of adverse events, changes in laboratory test results, changes in vital signs and ECGs, and exposure to any study treatment.

Study treatment exposure (e.g., treatment duration, total dose received, and number of cycles and dose modifications) will be summarized. All verbatim adverse event terms will be mapped to Medical Dictionary for Regulatory Activities thesaurus terms, and adverse event severity will be graded according to scale (e.g., NCI CTCAE v5.0). All adverse events, serious adverse events, adverse events leading to death, adverse events of special interest, and adverse events leading to study treatment discontinuation that occurs on or after the first dose of study treatment (i.e., treatment-emergent adverse events) will be summarized by mapped terms, appropriate thesaurus level, and severity grade. Incidence of injection-site reactions and pain assessments will be performed at various timepoints after completion of each subcutaneous tiragolumab and atezolizumab co-mix injection by Local Injection-Site Symptom Assessment (LISSA). TASQ-SC or TASQ-IV will be used by subjects to assess the intensity of pain and changes from baseline in site pain, redness, and swelling. PPQ will be used to analyze overall preference for an administration method in subjects.

Relevant laboratory, vital sign (e.g., pulse rate, respiratory rate, blood pressure, pulse oximetry, and temperature), and ECG data will be displayed by time, with grades identified where appropriate. Values outside of the normal ranges will be flagged. A shift table of selected laboratory tests will be used to summarize the baseline and maximum post-baseline severity grade. Changes in vital signs and ECGs will be summarized.

The immunogenicity analysis population will include subjects with at least one ADA assessment. Subjects will be grouped according to treatment received or treatment assigned. The main endpoints will be:
1. Prevalence of ADAs to tiragolumab at baseline and incidence of ADAs to tiragolumab during the study;
2. Prevalence of ADAs to atezolizumab at baseline and incidence of ADAs to atezolizumab during the study; and
3. Prevalence of ADAs to rHuPH20 at baseline an incidence of ADAs to rHuPH20 during the study.

The numbers and proportions of ADA-positive subjects and ADA-negative subjects at baseline (baseline prevalence) and after drug administration (treatment emergent ADA incidence) will be summarized by cohort and time. Treatment emergent ADA incidence will be determined when subjects are considered to be ADA positive if they were ADA negative or have missing data at baseline but develop an ADA response following exposure to treatment (treatment-induced ADA response), or if they were ADA positive at baseline and the titer of one or more post-baseline samples is at least 0.60 titer unit greater than the titer of the baseline sample (treatment-enhanced ADA response). Subjects are considered ADA negative if they are ADA negative or have missing data at baseline and all post-baseline samples are negative, or if they are ADA positive at baseline but do not have any post-baseline samples with a titer that is at least 0.60 titer unit greater than the titer of the baseline sample (treatment unaffected).

### Example 9. Combination Therapy, Open-label, Multicenter Fixed-Dose Combination Study of Tiragolumab and Atezolizumab

A combination therapy, single-arm, and open-label study to evaluate the safety, pharmacokinetics (PK), and immunogenicity of an intravenous fixed-dose combination (FDC) administration of the anti-TIGIT monoclonal antibody tiragolumab in combination with the anti-PD-L1 monoclonal antibody atezolizumab will be conducted in subjects with locally advanced, recurrent or metastatic solid tumors. The fixed-dose formulation affords the ability to prepare and administer the combination from one vial and one IV bag, respectively, decreasing the number of infusions and administration time. This will reduce the patient and healthcare resource burden by improving workflow efficiency, reducing treatment and observative time relative to sequential administration, and reducing dosing errors.

Approximately 40-60 subjects with histologically-confirmed PD-L1-selected solid tumors whose disease is locally advanced, recurrent, or metastatic will be enrolled. The subjects enrolled must meet all eligibility criteria for participation and may qualify for one re-screening opportunity. For subjects who are re-screened, all eligibility criteria will be re-evaluated and screening assessment will be repeated as applicable. The enrolled subjects will not have received prior checkpoint inhibitor treatment for their cancer (CPI-Naive) (e.g., anti-PD-L1/PD-1, anti-CTL1-4, and anti-TIGIT), and some subjects (e.g., at least 20 subjects) will be cancer immunotherapy (CIT) naive (CIT-Naive).

During screening, tumor specimens from each potentially eligible subject will be prospectively tested for PD-L1 expression using the VENTANA® PD-L1 (SP263) immunohistochemical (IHC) assay.

Subjects will intravenously receive a fixed-dose combination of 600 mg tiragolumab and 1200 mg atezolizumab on Day 1 of each 21-day cycle (i.e., once every 3 weeks or Q3W). Treatment will continue until radiographic disease progression per investigator-assessed response Evaluation Criteria in Solid Tumors (RECIST) v1.1, there is loss of clinical benefit for subjects continuing treatment after radiographic disease progression, or unacceptable toxicity. Subjects will undergo tumor assessments at screening and during the treatment period.

During the study, serum samples will be collected to monitor tiragolumab and atezolizumab pharmacokinetics and to detect the presence of antibodies to tiragolumab and/or atezolizumab (i.e., anti-drug antibodies or ADAs). Additional tumor tissue and serum samples will be collected for exploratory biomarker assessments.

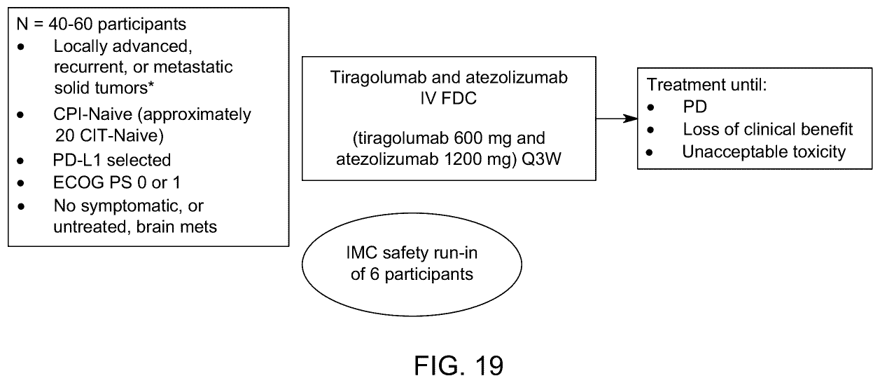

FIG. 19 provides an overview of the study design. During the study, subjects who meet criteria for disease progression per RECIST v1.1 and show evidence of clinical benefit may continue study treatment with a tiragolumab and atezolizumab fixed-dose combination, provided the subject meets the following criteria:
(1) Evidence of clinical benefit;
(2) Absence of symptoms and signs (including worsening of laboratory values (e.g., new or worsening hypercalcemia)) indicating unequivocal progression of disease;
(3) No decline in Eastern Cooperative Oncology Group (ECOG) Performance Status that can be attributed to disease progression;
(4) Absence of tumor progression at critical anatomical sites (e.g., leptomeningeal disease) that cannot be managed by protocol-allowed medical interventions; and
(5) Subjects must provide written consent to acknowledge deferring other treatment options in favor of continuing study treatment at the time of initial radiographic progression per RECIST v.1.1.

### Objectives and Endpoints

Objectives and corresponding endpoints are described in Table 21 infra.

TABLE 21
Objectives and Endpoints

| Primary Objective | Corresponding Endpoint |
|---|---|
| 1. To evaluate the safety and tolerability of | 1. Incidence and severity of adverse events, |
| tiragolumab and atezolizumab IV FDC | with severity determined according to the NCI |
| | CTCAE v5.0 (up to approximately 24 months). |
| | a. The severity of CRS will also be |
| | determine according to the ASTCT CRS |
| | Consensus Grading Scale |
| Secondary Objectives | Corresponding Endpoints |
| 1. To characterize the pharmacokinetics of | 1. Serum concentrations of tiragolumab and |
| tiragolumab and atezolizumab following | atezolizumab at specified timepoints for the |
| administration of IV FDC | following parameters: |
| | a. Area under the concentration-time curve |
| | at Cycle 1 (Days 1, 2, 8 and 15) |
| | b. Cₘₐₓ at Cycle 1 (Days 1, 2, 8 and 15) |
| | c. Additional PK parameters such as Cₘᵢₙ at |
| | Cycle 1 and CL will be reported as |
| | appropriate |
| 2. To evaluate the immune response to | 2. Prevalence of ADAs to tiragolumab at |
| tiragolumab and atezolizumab following IV | baseline and the incidence of treatment |
| FDC administration by measuring anti- | emergent ADAs to tiragolumab during the |
| tiragolumab and anti-atezolizumab antibodies | study (Day 1 of Cycles 1-4, 8, 12, 16 and |
| | treatment discontinuation visit (up to |
| | approximately 24 months)). |
| | 3. Prevalence of ADAs to atezolizumab at |
| | baseline and the incidence of treatment |
| | emergent ADAs to atezolizumab during the |
| | study. (Day 1 of Cycles 1-4, 8, 12, 16 and |
| | treatment discontinuation visit (up to |
| | approximately 24 months)). |
| Exploratory Objectives | Corresponding Endpoints |
| 1. To evaluate the efficacy response of | 1. PFS, defined as the proportion of |
| tiragolumab and atezolizumab IV FDC | participants who do not experience disease |
| | progression or die from any cause according to |
| | RECIST v1.1 |
| | 2. ORR, defined as the proportion of |
| | participants with a CR or a PR on two |
| | consecutive occasions ≥4 weeks apart |
| | according to RECIST v1.1 |
| | 3. DOR, defined as the time from the first |
| | occurrence of a documented objective |
| | response to disease progression or death from |
| | any cause, whichever occurs first, according to |
| | RECIST v1.1 |
| | 4. OS after enrollment, defined as the time |
| | from enrollment to death from any cause |
| 2. To characterize exploratory PK parameters | 5. Serum concentrations of tiragolumab and |
| of tiragolumab and atezolizumab following | atezolizumab at various timepoints for the |
| administration of IV FDC | following parameters: |
| | a. Cₘₐₓ and Cₘᵢₙ at Cycles 2, 3, 4, 8, 12 and 16 |
| | b. Vₛₛ |
| | c. Half-life |
| | d. Accumulation ratio |
| | e. Time to reach steady state |
| 3. The exploratory immunogenicity objective | 6. Evaluate efficacy, safety, or PK endpoints |
| for this study is to evaluate potential effects of | by tiragolumab ADA status as appropriate |
| ADAs in tiragolumab and atezolizumab IV FDC | 7. Evaluate efficacy, safety, or PK endpoints |
| | by atezolizumab ADA status as appropriate |

ADA = anti-drug antibody;
ASTCT = American Society for Transplantation and Cellular Therapy;
CL = clearance;
Cₘₐₓ = maximum serum concentration;
Cₘᵢₙ = minimum serum concentration;
CR = complete response;
CRS = cytokine release syndrome;
DOR = duration of response;
FDC = fixed dose combination;
NCI CTCAE = National Cancer Institute Common Terminology Criteria for Adverse Events;
ORR = objective response rate;
OS = overall survival;
PD = pharmacodynamic;
PFS = progression-free survival;
PK = pharmacokinetic;
PR = partial response;
RECIST = Response Evaluation Criteria in Solid Tumors;
TIGIT = T-cell Immunoreceptor with immunoglobulin and immunoreceptor tyrosine-based inhibition motif domains;
Vₛₛ = volume of distribution at steady state

### Rationale

The FDC of tiragolumab and atezolizumab will be administered with the aim of assessing safety, pharmacokinetics, and immunogenicity in subjects with histologically-confirmed PD-L1-selected solid tumors whose disease is locally advanced, recurrent, or metastatic. The subjects may have one of the following indications: esophageal adenocarcinoma (EAC), esophageal squamous cell carcinoma (ESCC), gastroesophageal junction cancer (GEJ), hepatocellular carcinoma (HCC), melanoma, non-small cell lung cancer (NSCLC), renal cell cancer (RCC), squamous cell carcinoma of the head and neck (SCCHN), and urothelial bladder cancer (UBC).

The IV FDC formulation affords the ability to prepare and administer both drugs from one vial and one IV bag, respectively, decreasing the number of infusions and administration time. This formulation can reduce the patient healthcare resource burden by: (1) improving workflow efficiency, (2) reducing treatment and observation time relative to sequential administration, and (3) reducing dosing errors arising from different doses and/or volumes of tiragolumab and atezolizumab to be administered from individual vials.

The VENTANA® PD-L1 (SP263) IHC assay is intended for the qualitative IHC assessment of the PD-L1 protein and it is used to identify subjects who are PD-L1-positive based on pre-specified cutoffs for the indications mentioned in Table 22.

TABLE 22
VENTANA ® PD-L1 (SP263) IHC Assay Cutoffs by Solid Tumor

| Indication | Cutoff |
|---|---|
| ESCC | 10% TAP score |
| GEJ, EAC | 1% TAP score |
| HCC | 1% TAP score |
| Melanoma | 1% TC |
| NSCLC | 1% TC |
| RCC | 1% TC |
| SCCHN | 1% TC |
| UBC | 25% TC or 25% IC |

EAC = esophageal adenocarcinoma;
ESCC = esophageal squamous cell carcinoma;
GEJ = gastroesophageal junction cancer;
IC = immune cell;
IHC = immunohistochemical;
NSCLC = non-small cell lung cancer;
RCC = renal cell cancer;
SCCHN = squamous cell carcinoma of the head and neck;
TAP = tumor area positivity;
TC = tumor cell;
UBC = urothelial bladder cancer

### Study Assessments and Procedures

Efficacy assessments to be performed in this study include tumor evaluations (e.g., radiographic assessments) and response evaluations. Subjects will undergo tumor assessment at screening, every 6 weeks (+7 days) for the first 48 weeks following treatment initiation (Day 1 of Cycle 1), and every 9 weeks (+7 days) thereafter, regardless of dose delays. Subjects will continue to undergo tumor assessments until radiographic disease progression per RECIST v1.1 or loss of clinical benefit, withdrawal of consent, death, or study termination occurs. All measurable and/or evaluable lesions will be assessed and documented at screening. Screening assessments will include CT scans with contrast of the chest, abdomen, and pelvis. If a CT scan with contrast is contraindicated (e.g., a subject with contrast allergy or impaired renal clearance), a non-contrast CT scan of the chest will be performed, and magnetic resonance imaging (MRI) scans of the abdomen and pelvis will be performed. All known or suspected sites of disease will be assessed at screening. All participants will undergo an MRI or CT scan (with contrast) of the brain at screening to evaluate central nervous system (CNS) metastasis. An objective response will be determined according to RECIST v1.1 and assessment will be performed by the same subject to determine internal consistency. Exploratory endpoints (e.g., objective response rate (ORR), duration of response (DOR), progression-free survival (PFS), and overall survival (OS)) will be evaluated.

Safety assessments that will be performed in this study include physical examinations, measurement of vital signs (e.g., tympanic temperature, pulse rate, respiratory rate, and blood pressure), performance status, electrocardiograms (ECGs), clinical safety laboratory tests, pregnancy testing and auto-antibody testing.

Performance status will be measured using the ECOG Performance Status Scale.

Single 12-lead ECGs will be obtained using an ECG machine that calculates the heart rate and measures PR interval and QRS interval. ECGs will be obtained prior to other procedures scheduled at the same time (e.g., vital sign measurements and blood draws) and will not be obtained within 3 hours after any meal. Circumstances that may induce changes in heart rate, including environmental distractions, will be avoided during the pre-ECG resting period and during ECG recording.

Clinical safety laboratory tests that will be performed include:
1. Hematology: white blood cell (WBC) count, red blood cell (RBC) count, hemoglobin, hematocrit, platelet count, and differential count (neutrophils, eosinophils, basophils, monocytes, and lymphocytes);
2. Chemistry panel (serum or plasma): bicarbonate or total carbon dioxide, sodium, potassium, magnesium, chloride, glucose, BUN or urea, creatinine, total protein, albumin, phosphate, calcium, total bilirubin, ALP, ALT, AST, creatinine phosphokinase, and lactate dehydrogenase;
3. Coagulation: international normalized ratio (INR) and activated partial thromboplastin clotting time (aPTT);
4. Thyroid function testing: TSH, free T3 (or total T3 for sites where free T3 is not performed), and free T4;
5. Serum ferritin and C-reactive protein;
6. HIV serology;
7. Hepatitis B virus (HBV) serology: Hepatitis B surface antigen (HBsAg), hepatitis B surface antibody (HBsAb), and total hepatitis B core antibody (HBcAb) for all subjects; HBV DNA for subjects with negative HBsAg test and a positive total HBcAb test;
8. Hepatitis C virus (HCV) serology: HCV antibody for all subjects; HCV RNA for subjects with a positive HCV antibody test;
9. Epstein-Barr virus (EBV) serology;
10. Pregnancy test: all female subjects of childbearing potential will have a serum pregnancy test at screening. Urine pregnancy tests will be performed at subsequence visits; and
11. Urinalysis, including dipstick (pH, specific gravity, glucose, protein, ketones, and blood).

Auto-antibody testing of serum samples will include anti-nuclear antibody, anti-double-stranded DNA, anti-neutrophil cytoplasmic antibodies, and thyroid peroxidase antibody testing. If inflammatory arthritis develops in a subject, cyclic citrullinated peptide and rheumatoid factor antibody titers will also be evaluated.

All adverse events will be reported from the start of treatment until 30 days after the final dose of study treatment at various timepoints, or until initiation of new systemic anti-cancer therapy, whichever occurs first. All serious adverse events will be collected and followed until 90 days after the final dose of study treatment or until initiation of new systemic anti-cancer therapy, whichever occurs first. All adverse events or serious adverse events will be followed until the event has resolved to baseline grade or better, the event is assessed as stable, the subject is lost to follow-up, or the subject withdraws consent. Adverse event severity is graded on a scale of 1-5, in which Grades 4 and 5 will be reported as serious adverse events. Grade 1 severity includes mild; asymptomatic or mild symptoms; clinical or diagnostic observations only; or intervention not indicated. Grade 2 severity includes moderate; minimal, local or non-invasive intervention indicates; or limiting age-appropriate instrumental activities of daily life (e.g., preparing meals, shopping for groceries or clothes, using the telephone, and managing money). Grade 3 severity includes severe or medically significant, but not immediately life-threatening (if the event is a “significant medical event,” it is reported as a serious adverse event); hospitalization or prolongation of hospitalization indicated; disabling; or limiting self-care activities (e.g., bathing, dressing and undressing, feeding oneself, using the toilet, and taking medications) of daily living. Grade 4 severity includes life-threatening consequences or urgent intervention indicated. Grade 5 severity includes death related to an adverse event.

Pharmacokinetic (PK) analysis to evaluate tiragolumab and atezolizumab in serum samples will be performed by measuring serum concentrations, clearance (CL), volume of distribution at steady state (Vₛₛ), and area under the curve (AUC).

Immunogenicity will be assessed by measurement of anti-drug antibodies (ADAs) to tiragolumab and atezolizumab in serum samples collected from subjects. Samples will be screened for ADAs, and the titer will be reported for confirmed positive samples.

### Example 10. An Open-label, Multicenter Dose-Finding Study of Tiragolumab and Atezolizumab

A three single-arm, open-label, multicenter dose-finding study to determine the doses of subcutaneous (SC) administration of the anti-TIGIT monoclonal antibody tiragolumab in combination with subcutaneous administration of the anti-PD-L1 monoclonal antibody atezolizumab in comparison to sequential intravenous (IV) administration of atezolizumab followed by tiragolumab was initiated and is ongoing conducted in subjects with locally advanced or metastatic solid tumors on the basis of pharmacokinetic (PK) analysis of serum tiragolumab and atezolizumab (i.e., C_{trough} at Cycle 1 (pre-dose Cycle 2)).

The study consists of three cohorts, as follows:
Cohort 1: Cycle 1: Subjects received either (1) 880 mg tiragolumab SC co-mixed with 2000 mg atezolizumab SC in the abdomen, or (2) 880 mg tiragolumab SC co-mixed with 1875 mg atezolizumab SC in the thigh, at Day 1 of the first cycle (21 days). Cycle 2 onwards: Subjects will receive 1200 mg atezolizumab IV followed by 600 mg tiragolumab IV every 3 weeks (Q3W) (Day 1 of each cycle) from Cycle 2 onwards until disease progression, loss of clinical benefit, unacceptable toxicity, or withdrawal of consent.
Cohort 2: Cycle 1 to 3: Subjects will receive tiragolumab SC co-mixed with atezolizumab SC in the thigh Q3W at Day 1 of each cycle for 3 cycles (21 days each) at a dose to be determined following Cohort 1. Cycle 4 onwards: Subjects will receive 1200 mg atezolizumab IV followed by 600 mg tiragolumab IV Q3W (Day 1 of each cycle) starting from Cycle 4 onwards until disease progression, loss of clinical benefit, unacceptable toxicity, or withdrawal of consent.
Cohort 3: Cycle 1 to 3: Subjects will receive tiragolumab SC co-mixed with atezolizumab SC in the abdomen Q3W at Day 1 of each cycle for 3 cycles (21 days each) at a dose to be determined following Cohort 1. Cycle 4 onwards: Subjects will receive 1200 mg atezolizumab IV followed by 600 mg tiragolumab IV Q3W (Day 1 of each cycle) starting from Cycle 4 onwards until disease progression, loss of clinical benefit, unacceptable toxicity, or withdrawal of consent.

The doses for Cohorts 2 and 3 will be selected based on Cohort 1, which is designed to determine whether the predicted exposures are comparable to the exposures tested with IV sequential administration of these drugs.

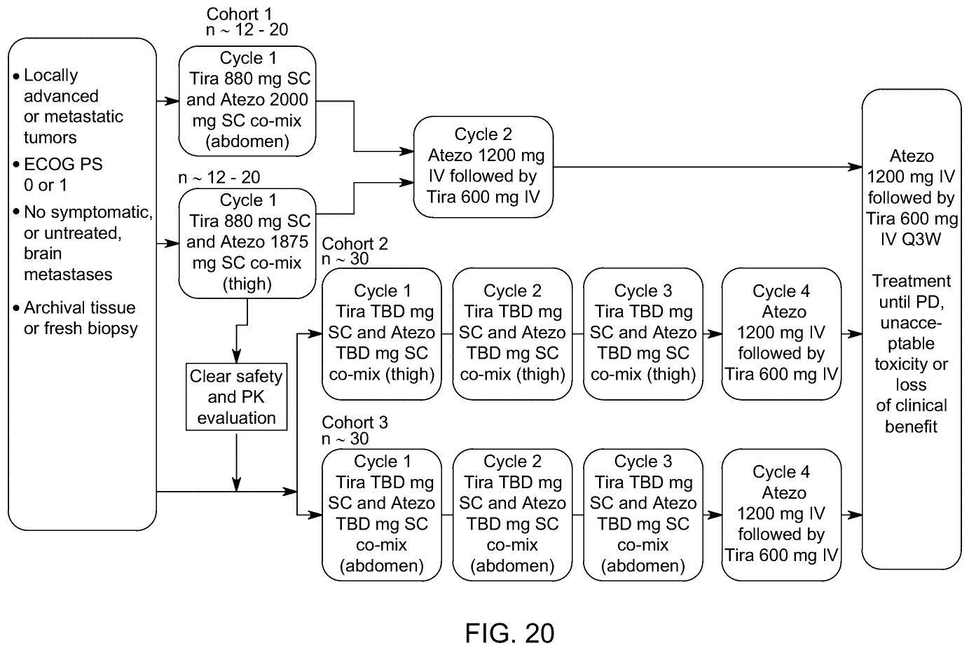

Tumor assessments were performed every 6 weeks (Q6W) (+7 days) for the first 48 weeks and every 9 weeks (Q9W) (+7 days) subsequently until radiographic progressive disease (PD) or loss of clinical benefit. Subjects continue to be followed for overall survival (OS) until death, lost-to-follow-up, withdrawal from the study, or study termination, whichever occurs first. FIG. 20 provides an overview of the study design.

### Objectives and Endpoints

This study was designed to evaluate the pharmacokinetics (PK), safety, tolerability, and exploratory efficacy of various doses of tiragolumab and atezolizumab administered as a single SC injection (i.e., a tiragolumab and atezolizumab SC co-mix with rHuPH20) and of the sequential administration of tiragolumab and atezolizumab IV in subjects with locally advanced or metastatic solid tumors. Objectives and corresponding endpoints are described in Table 23 infra.

TABLE 23
Objectives and Endpoints

| Primary PK Objective | Corresponding Endpoint |
|---|---|
| 1. To determine the optimal dose of the | 1. Model-predicted serum tiragolumab and |
| tiragolumab and atezolizumab SC co-mix that | atezolizumab trough concentrations (Ctrough) at |
| provides exposure comparable to tiragolumab | Cycle 1 (i.e., pre-dose Cycle 2) |
| and atezolizumab IV administered sequentially | |
| Secondary PK Objective | Corresponding Endpoints |
| 1. To characterize the PK profile of the | 1. Serum tiragolumab concentrations at |
| tiragolumab and atezolizumab SC co-mix | specified timepoints during SC administration |
| | 2. Serum atezolizumab concentrations at |
| | specified timepoints during SC administration |
| Safety Objective | Corresponding Endpoint |
| 1. To evaluate the safety and tolerability of | 1. Incidence and severity of adverse events, |
| tiragolumab and atezolizumab | with severity determined according to National |
| | Cancer Institute Common Terminology |
| | Criteria for Adverse Events (NCI CTCAE) v5.0. |
| | a. Severity for cytokine-release syndrome |
| | (CRS) will also be determined according |
| | to the American Society for |
| | Transplantation and Cellular Therapy |
| | (ASTCT) CRS Consensus Grading scale. |
| Exploratory Safety Objective | Corresponding Endpoint |
| 1. To evaluate the safety and tolerability of the | 1. Incidence of injection-site reactions, as |
| SC co-mix of tiragolumab and atezolizumab | assessed through the use of the Local |
| | Injection-Site Symptom Assessment (LISSA) |
| Immunogenicity Objective | Corresponding Endpoint |
| 1. To evaluate the immune response to | 1. Prevalence of anti-drug antibodies (ADAs) |
| tiragolumab, atezolizumab, and rHuPH20 | to tiragolumab at baseline and incidence of |
| | ADAs to tiragolumab during the study |
| | 2. Prevalence of ADAs to atezolizumab at |
| | baseline and incidence of ADAs to |
| | atezolizumab during the study |
| | 3. Prevalence of ADAs to rHuPH20 at baseline |
| | and incidence of ADAs to rHuPH20 during the |
| | study |
| Exploratory Immunogenicity Objective | Corresponding Endpoints |
| 1. To evaluate potential effects of ADAs on | 1. Relationship between tiragolumab ADA |
| the basis of the following endpoints: | status and PK, safety, and/or efficacy |
| | endpoints |
| | 2. Relationship between atezolizumab ADA |
| | status and PK, safety, and/or efficacy |
| | endpoints |
| | 3. Relationship between rHuPH20 ADA |
| | status and PK, safety, and/or efficacy |
| | endpoints |
| Exploratory Patient-reported Outcome (PRO) Objective | Corresponding Endpoints |
| 1. To evaluate patient preference for either SC | 1. Proportion of patients indicating an overall |
| or IV route of administration and patient- | preference, as assessed through the use of the |
| reported safety and tolerability on the basis of | Patient Preference Questionnaire (PPQ) for |
| the following endpoints: | either SC or IV route of administration for |
| | tiragolumab at various doses in combination |
| | with atezolizumab |
| | 2. Change from baseline in patient-reported |
| | injection-site pain, swelling, and redness as |
| | assessed by the Therapy Administration |
| | Satisfaction Questionnaire-Subcutaneous |
| | (TASQ-SC) and TASQ-Intravenous (TASQ-IV) |
| Exploratory Efficacy Objectives | Corresponding Endpoints |
| 1. To evaluate the efficacy response of | 1. PFS, defined as the time from study entry |
| tiragolumab and atezolizumab on the basis of: | to the first occurrence of disease progression |
| | or death from any cause (whichever occurs |
| | first), as determined by the investigator |
| | according to Response Evaluation Criteria in |
| | Solid Tumors, Version 1.1 (RECIST v1.1) |
| | 2. ORR, defined as the proportion of patients |
| | with a CR or PR, as determined by the |
| | investigator according to RECIST v1.1 and |
| | confirmed by repeat assessment ≥4 weeks |
| | after initial documentation |
| | 3. Duration of objective response (DOR), |
| | defined as the time from the first occurrence |
| | of a documented objective response to the |
| | time of the first documented disease |
| | progression or death from any cause, |
| | whichever occurs first, per RECIST v1.1 as |
| | determined by the investigator |
| 2. To evaluate OS on the basis of: | 4. OS, defined as the time from study entry to |
| | death from any cause |

ADA = anti-drug antibody;
ASTCT = American Society for Transplantation and Cellular Therapy;
CR = complete response;
CRS = cytokine-release syndrome;
C_{trough} = trough concentration;
DOR = duration of response;
IV = intravenous;
LISSA = Local Injection-Site Symptom Assessment;
NCI CTCAE = National Cancer Institute Common Terminology Criteria for Adverse Events;
OS = overall survival;
PFS = progression-free survival;
PK = pharmacokinetic;
PPQ = Patient Preference Questionnaire;
PR = partial response;
rHuPH20 = recombinant human hyaluronidase;
SC = subcutaneous;
TASQ = Therapy Administration Satisfaction Questionnaire

Subjects underwent tumor assessments at baseline and Q6W (±7 days) for 48 weeks following Day 1 of Cycle 1. After the completion of the Week 48 tumor assessment, tumor assessments were performed every 9 weeks (±7 days), until radiographic disease progression per RECIST v1.1, withdrawal of consent, death, or study termination, whichever occurs first. Subjects who are treated beyond disease progression per RECIST v1.1 will undergo tumor assessments Q6W (±2 weeks) after initial documentation of progression, or more frequently if clinically indicated, regardless of time in study, until treatment is discontinued. Scans may be performed at any time if progressive disease or loss of clinical benefit is suspected.

Response was assessed on the imaging modalities described above (e.g., CT scan and MRI), with use of RECIST v1.1. The assessment of overall tumor response at all timepoints will be based on RECIST v1.1. Results were reviewed before dosing at the next cycle. Study treatment with tiragolumab and atezolizumab will be continued as long as subjects are experiencing clinical benefit in the absence of unacceptable toxicity or symptomatic deterioration attributed to disease progression after an integrated assessment of radiographic data, biopsy results, and clinical status. Subjects who meet criteria for disease progression per RECIST v1.1 will be permitted to continue study treatment if they meet all of the criteria and provide written consent.

An objective response will be assessed according to RECIST v1.1 and determined by repeat assessment at ≥4 weeks after initial documentation of CR and PR.

Laboratory Tests that Will be Performed Include:
1. Serology: HIV serology, HBV serology (e.g., HBsAg, HBsAb, and total HBcAb); HBV DNA for subjects with negative HBsAg and HBsAb tests and a positive total HBcAb; HCV serology (e.g., HCV antibody and (if HCV antibody test is positive) HCV RNA); EBV serology (e.g., EBV viral capsid antigen (VCA) IgM; EBV VCA IgG or EBV nuclear antigen (EBNA) IgG; and EBV PCR);
2. Hematology: white blood cell (WBC) count with differential (neutrophils, eosinophils, basophils, monocytes, lymphocytes), red blood cell (RBC) count, hemoglobin, hematocrit, platelet count, and differential count;
3. Chemistry panel (serum or plasma): bicarbonate or total carbon dioxide (if considered the standard of care for the region), sodium, magnesium, potassium, calcium, chloride, glucose, blood urea nitrogen (BUN) or urea, creatinine, total protein, albumin, phosphate, total bilirubin, ALP, ALT, AST, and lactate dehydrogenase (LDH);
4. Urinalysis, including dipstick (pH, specific gravity, glucose, protein, ketones, blood;
5. Pregnancy test: All women of childbearing potential will have a serum pregnancy test during screening 14 days prior to the initiation of study drug. During the study, urine pregnancy tests will be performed within 96 hours of dosing on Day 1 of every cycle, and after study treatment is discontinued;
6. Coagulation (INR and aPTT) or prothrombin time (PTT); and
7. Thyroid-stimulating hormone (TSH), free T3 (or total T3 at sites where free T3 is not performed), and free T4.

Safety assessments performed in this study included monitoring and recording adverse events (e.g., serious adverse events and adverse events of special interest), performing safety laboratory assessments, measuring vital signs, and conducting other tests that are deemed critical to the safety evaluation of the study.

1. A liquid pharmaceutical formulation comprising:
(a) 18 mg/mL to 176 mg/mL of an anti-TIGIT monoclonal antibody;
(b) 54 mg/mL to 137.5 mg/mL of an anti-PD-L1 monoclonal antibody;
(c) 5 mM to 30 mM of a histidine buffer;
(d) 120 mM to 320 mM of sucrose; and
(e) 0.02% (w/v) to 0.08% (w/v) polysorbate 20,
wherein the liquid pharmaceutical formulation is characterized by a pH of about 5.2-6.1;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

2.-3. (canceled)

4. A liquid pharmaceutical formulation comprising:
(a) 18 mg/mL to 75 mg/mL of an anti-TIGIT monoclonal antibody;
(b) 54 mg/mL to 137.5 mg/mL of an anti-PD-L1 monoclonal antibody;
(c) 12 mM to 28 mM of a histidine buffer;
(d) 100 mM to 300 mM of sucrose; and
(e) 0.02% (w/v) to 0.08% (w/v) polysorbate 20,
wherein the liquid pharmaceutical formulation is characterized by a pH of about 5.4-6.2;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

5.-7. (canceled)

8. A liquid pharmaceutical formulation comprising:
(a) 18 mg/mL to 176 mg/mL of an anti-TIGIT monoclonal antibody;
(b) 500 U/mL to 2600 U/mL of a hyaluronidase;
(c) 5 mM to 30 mM of a histidine buffer;
(d) 180 mM to 320 mM of sucrose; and
(e) 0.03% (w/v) to 0.08% (w/v) polysorbate 20,
wherein the liquid pharmaceutical formulation is characterized by a pH of about 5.2-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

9.-10. (canceled)

11. A liquid pharmaceutical formulation comprising:
(a) 30 mg/mL to 60 mg/mL of an anti-TIGIT monoclonal antibody;
(b) 60 mg/mL to 120 mg/mL of an anti-PD-L1 monoclonal antibody;
(c) 500 U/mL to 2600 U/mL of a hyaluronidase;
(d) 5 mM to 30 mM of a histidine buffer;
(e) 180 mM to 320 mM of sucrose; and
(f) 0.03% (w/v) to 0.08% (w/v) polysorbate 20,
wherein the liquid pharmaceutical formulation is characterized by a pH of about 5.2-6.1;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6; and
wherein the anti-PD-L1 monoclonal antibody comprises a heavy chain variable region comprising a HVR-H1 comprising the amino acid sequence of SEQ ID NO: 10; a HVR-H2 comprising the amino acid sequence of SEQ ID NO: 11; and a HVR-H3 comprising the amino acid sequence of SEQ ID NO: 12; and a light chain variable region comprising a HVR-L1 comprising the amino acid sequence of SEQ ID NO: 13; a HVR-L2 comprising the amino acid sequence of SEQ ID NO: 14; and a HVR-L3 comprising the amino acid sequence of SEQ ID NO: 15.

12.-18. (canceled)

19. A liquid pharmaceutical formulation comprising:
(a) 18 mg/mL to 176 mg/mL of an anti-TIGIT monoclonal antibody;
(b) 5 mM to 30 mM of histidine acetate;
(c) 100 mM to 320 mM of sucrose; and
(d) 0.01% (w/v) to 0.08% (w/v) polysorbate 20,
wherein the liquid pharmaceutical formulation is characterized by a pH of about 5.0-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

20.-21. (canceled)

22. A liquid pharmaceutical formulation comprising:
(a) 18 mg/mL to 75 mg/mL of an anti-TIGIT monoclonal antibody;
(b) 5 mM to 30 mM of histidine acetate;
(c) 100 mM to 320 mM of sucrose; and
(d) 0.01% (w/v) to 0.08% (w/v) polysorbate 20,
wherein the liquid pharmaceutical formulation is characterized by a pH of about 5.0-6.0;
wherein the anti-TIGIT monoclonal antibody comprises a heavy chain variable region comprising a HVR-H1 comprising the amino acid sequence of SEQ ID NO: 1; a HVR-H2 comprising the amino acid sequence of SEQ ID NO: 2; and a HVR-H3 comprising the amino acid sequence of SEQ ID NO: 3; and a light chain variable region comprising a HVR-L1 comprising the amino acid sequence of SEQ ID NO: 4; a HVR-L2 comprising the amino acid sequence of SEQ ID NO: 5; and a HVR-L3 comprising the amino acid sequence of SEQ ID NO: 6.

23.-62. (canceled)

63. An article of manufacture comprising the liquid pharmaceutical formulation according to any one of claims 1, 4, 8, 11, 19, and 22.

64.-72. (canceled)

73. A method of treating cancer in a subject in need thereof comprising administering to the subject a therapeutically effective amount of the liquid pharmaceutical formulation according to any one of claims 1, 4, 8, 11, 19, and 22.

74.-87. (canceled)

88. A method of treating cancer in a subject in need thereof comprising administering to the subject a therapeutically effective amount of the liquid pharmaceutical formulation according to any one of claims 8, 11, 19, and 22 and administering to the subject a therapeutically effective amount of an anti-PD-1 monoclonal antibody, or an anti-PD-L1 monoclonal antibody.

89.-117. (canceled)

118. An article of manufacture comprising a formulation that comprises 18 mg/mL to 176 mg/mL of an anti-TIGIT monoclonal antibody.

119.-122. (canceled)

123. An article of manufacture comprising a formulation that comprises:
(a) 30 mg/mL to 60 mg/mL of an anti-TIGIT monoclonal antibody; and
(b) 60 mg/mL to 120 mg/mL of an anti-PD-L1 monoclonal antibody.

124.-130. (canceled)

131. An article of manufacture comprising a formulation that comprises 880 mg of an anti-TIGIT monoclonal antibody.

132.-136. (canceled)

137. An article of manufacture comprising a formulation that comprises 880 mg of an anti-TIGIT monoclonal antibody and 1875 mg or 2000 mg of an anti-PD-L1 monoclonal antibody.

138.-143. (canceled)

144. An article of manufacture comprising a subcutaneous administration device, which contains and delivers to a patient an 880 mg fixed dose of an anti-TIGIT monoclonal antibody.

145.-194. (canceled)

195. A method of treating cancer in a subject in need thereof comprising administering to the subject a therapeutically effective amount of the formulation comprised in the article of manufacture according to any one of claims 118, 123, 131, 137, and 144.

196.-207. (canceled)


SEQUENCE LISTING (25 sequences)

SEQ ID NO: 1 - protein, 7 aa, synthetic construct
SNSAAWN

SEQ ID NO: 2 - protein, 18 aa, synthetic construct
KTYYRFKWYSDYAVSVKG

SEQ ID NO: 3 - protein, 14 aa, synthetic construct
ESTTYDLLAGPFDY

SEQ ID NO: 4 - protein, 17 aa, synthetic construct
KSSQTVLYSSNNKKYLA

SEQ ID NO: 5 - protein, 7 aa, synthetic construct
WASTRES

SEQ ID NO: 6 - protein, 9 aa, synthetic construct
QQYYSTPFT

SEQ ID NO: 7 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 8 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 9 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 10 - protein, 10 aa, synthetic construct
GFTFSDSWIH

SEQ ID NO: 11 - protein, 18 aa, synthetic construct
AWISPYGGSTYYADSVKG

SEQ ID NO: 12 - protein, 9 aa, synthetic construct
RHWPGGFDY

SEQ ID NO: 13 - protein, 11 aa, synthetic construct
RASQDVSTAVA

SEQ ID NO: 14 - protein, 7 aa, synthetic construct
SASFLYS

SEQ ID NO: 15 - protein, 9 aa, synthetic construct
QQYLYHPAT

SEQ ID NO: 16 - protein, 118 aa, synthetic construct [sequence omitted]

SEQ ID NO: 17 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 18 - protein, 455 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 455 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 456 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK